(19) United States
(12) Patent Application Publication
(10) Pub. No.: US 2006/0167057 A1
(43) Pub. Date: Jul. 27, 2006

(54) Compounds for the treatment of CNS and amyloid associated diseases

(76) Inventors: Xianqi Kong, Dollard-des-Ormeaux (CA); Xinfu Wu, Laval (CA); Isabelle Valade, Laval (CA); Francine Gervais, Ile Bizard (CA)

Correspondence Address: LAHIVE & COCKFIELD, 28 STATE STREET, BOSTON, MA, 02109, US

(21) Appl. No.: 11/281,950

(22) Filed: Nov. 16, 2005

Related U.S. Application Data
(60) Provisional application No. 60/628,631, filed on Nov. 16, 2004.

(51) Int. Cl. A61K 31/4439; C07D 417/02; C07D 403/02

(52) U.S. Cl. 514/338; 514/341; 546/270.1; 546/269.1; 546/273.4; 546/272.7; 546/271.4

(57) ABSTRACT

Methods, compounds, pharmaceutical compositions and kits are described for treating or preventing CNS and amyloid associated disease. Also described are methods, compounds, pharmaceutical compositions and kits for detecting, diagnosing, monitoring and treating or preventing CNS and amyloid associated disease.

### RELATED APPLICATIONS

This application is related and claims priority to U.S. Provisional Application Ser. No. 60/628,631, filed Nov. 16, 2004.

### BACKGROUND

Amyloidosis refers to a pathological condition characterized by the presence of amyloid fibrils. Amyloid is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common X-ray diffraction and infrared spectra.

Amyloid associated diseases can either be restricted to one organ or spread to several organs. The first instance is referred to as “localized amyloidosis” while the second is referred to as “systemic amyloidosis.”

Some amyloid diseases can be idiopathic, but most of these diseases appear as a complication of a previously existing disorder. For example, primary amyloidosis (AL amyloid) can appear without any other pathology or can follow plasma cell dyscrasia or multiple myeloma.

Secondary amyloidosis is usually seen associated with chronic infection (such as tuberculosis) or chronic inflammation (such as rheumatoid arthritis). A familial form of secondary amyloidosis is also seen in other types of familial amyloidosis, e.g., Familial Mediterranean Fever (FMF). This familial type of amyloidosis is genetically inherited and is found in specific population groups. In both primary and secondary amyloidosis, deposits are found in several organs and are thus considered systemic amyloid diseases.

“Localized amyloidoses” are those that tend to involve a single organ system. Different amyloids are also characterized by the type of protein present in the deposit. For example, neurodegenerative diseases such as scrapie, bovine spongiform encephalitis, Creutzfeldt-Jakob disease, and the like are characterized by the appearance and accumulation of a protease-resistant form of a prion protein (referred to as AScr or PrP-27) in the central nervous system. Similarly, Alzheimer's disease, another neurodegenerative disorder, is characterized by neuritic plaques and neurofibrillary tangles. In this case, the amyloid plaques found in the parenchyma and the blood vessel are formed by the deposition of fibrillar Aβ amyloid protein. Other diseases such as adult-onset diabetes (type II diabetes) are characterized by the localized accumulation of amyloid fibrils in the pancreas.

Once these amyloids have formed, there is no known, widely accepted therapy or treatment which significantly dissolves amyloid deposits in situ, prevents further amyloid deposition or prevents the initiation of amyloid deposition.

Each amyloidogenic protein has the ability to undergo a conformational change and to organize into β-sheets and form insoluble fibrils which may be deposited extracellularly or intracellularly. Each amyloidogenic protein, although different in amino acid sequence, has the same property of forming fibrils and binding to other elements such as proteoglycan, amyloid P and complement component. Moreover, each amyloidogenic protein has amino acid sequences which, although different, show similarities such as regions with the ability to bind to the glycosaminoglycan (GAG) portion of proteoglycan (referred to as the GAG binding site) as well as other regions which promote β-sheet formation. Proteoglycans are macromolecules of various sizes and structures that are distributed almost everywhere in the body. They can be found in the intracellular compartment, on the surface of cells, and as part of the extracellular matrix. The basic structure of all proteoglycans is comprised of a core protein and at least one, but frequently more, polysaccharide chains (GAGs) attached to the core protein. Many different GAGs have been discovered including chondroitin sulfate, dermatan sulfate, keratan sulfate, heparin, and hyaluronan.

In specific cases, amyloid fibrils, once deposited, can become toxic to the surrounding cells. For example, the Aβ fibrils organized as senile plaques have been shown to be associated with dead neuronal cells, dystrophic neurites, astrocytosis, and microgliosis in patients with Alzheimer's disease. When tested in vitro, oligomeric (soluble) as well as fibrillar Aβ peptide was shown to be capable of triggering an activation process of microglia (brain macrophages), which would explain the presence of microgliosis and brain inflammation found in the brain of patients with Alzheimer's disease. Both oligomeric and fibrillar Aβ peptide can also induce neuronal cell death in vitro. See, e.g., M P Lambert, et al., Proc. Natl. Acad. Sci. USA 95, 6448-53 (1998).

In another type of amyloidosis seen in patients with type II diabetes, the amyloidogenic protein IAPP, when organized in oligomeric forms or in fibrils, has been shown to induce β-islet cell toxicity in vitro. Hence, appearance of IAPP fibrils in the pancreas of type II diabetic patients contributes to the loss of the β islet cells (Langerhans) and organ dysfunction which can lead to insulinemia.

Another type of amyloidosis is related to β₂ microglobulin and is found in long-term hemodialysis patients. Patients undergoing long term hemodialysis will develop β₂-microglobulin fibrils in the carpal tunnel and in the collagen rich tissues in several joints. This causes severe pains, joint stiffness and swelling.

Amyloidosis is also characteristic of Alzheimer's disease. Alzheimer's disease is a devastating disease of the brain that results in progressive memory loss leading to dementia, physical disability, and death over a relatively long period of time. With the aging populations in developed countries, the number of Alzheimer's patients is reaching epidemic proportions.

People suffering from Alzheimer's disease develop a progressive dementia in adulthood, accompanied by three main structural changes in the brain: diffuse loss of neurons in multiple parts of the brain; accumulation of intracellular protein deposits termed neurofibrillary tangles; and accumulation of extracellular protein deposits termed amyloid or senile plaques, surrounded by misshapen nerve terminals (dystrophic neurites) and activated microglia (microgliosis and astrocytosis). A main constituent of these amyloid plaques is the amyloid-β peptide (Aβ), a 39-43 amino-acid protein that is produced through cleavage of the β-amyloid precursor protein (APP). Extensive research has been conducted on the relevance of Aβ deposits in Alzheimer's disease, see, e.g., Selkoe, Trends in Cell Biology 8, 447-453 (1998). Aβ naturally arises from the metabolic processing of the amyloid precursor protein (“APP”) in the endoplasmic reticulum (“ER”), the Golgi apparatus, or the endosomal-lysosomal pathway, and most is normally secreted as a 40 (“Aβ1 -40”) or 42 (“Aβ1-42”) amino acid peptide (Selkoe, Annu. Rev. Cell Biol. 10, 373-403 (1994)). A role for Aβ as a primary cause for Alzheimer's disease is supported by the presence of extracellular Aβ deposits in senile plaques of Alzheimer's disease, the increased production of Aβ in cells harboring mutant Alzheimer's disease associated genes, e.g., amyloid precursor protein, presenilin I and presenilin II; and the toxicity of extracellular soluble (e.g., oligomeric) or fibrillar Aβ to cells in culture. See, e.g., Gervais, Eur. Biopharm. Review, 40-42 (Autumn 2001); May, DDT 6, 459-62 (2001). Although symptomatic treatments exist for Alzheimer's disease, this disease cannot be prevented or cured at this time.

Alzheimer's disease is characterized by diffuse and neuritic plaques, cerebral angiopathy, and neurofibrillary tangles. Plaque and blood vessel amyloid is believed to be formed by the deposition of insoluble Aβ amyloid protein, which may be described as diffuse or fibrillary. Both soluble oligomeric Aβ and fibrillar Aβ are also believed to be neurotoxic and inflammatory.

Another type of amyloidosis is cerebral amyloid angiopathy (CAA). CAA is the specific deposition of amyloid-P fibrils in the walls of leptomingeal and cortical arteries, arterioles and veins. It is commonly associated with Alzheimer's disease, Down's syndrome and normal aging, as well as with a variety of familial conditions related to stroke or dementia (see Frangione et al., Amyloid: J. Protein Folding Disord. 8, Suppl. 1, 36-42 (2001)).

Presently available therapies for treatment of 0-amyloid diseases are almost entirely symptomatic, providing only temporary or partial clinical benefit. Although some pharmaceutical agents have been described that offer partial symptomatic relief, no comprehensive pharmacological therapy is currently available for the prevention or treatment of, for example, Alzheimer's disease.

Central nervous system (CNS) diseases or disorders are a type of neurological disorder. CNS diseases can be drug induced; can be attributed to genetic predisposition, infection or trauma; or can be of unknown etiology. CNS diseases may include neuropsychiatric disorders, neurological diseases and mental illnesses; and include neurodegenerative diseases, behavioral disorders, cognitive disorders and cognitive affective disorders. There are several CNS diseases whose clinical manifestations have been attributed to CNS dysfunction (i.e., disorders resulting from inappropriate levels of neurotransmitter release, inappropriate properties of neurotransmitter receptors, and/or inappropriate interaction between neurotransmitters and neurotransmitter receptors). Several CNS diseases can be attributed to a cholinergic deficiency, a dopaminergic deficiency, an adrenergic deficiency and/or a serotonergic deficiency. CNS diseases may or may not be associated with or due to amyloid deposition.

### SUMMARY OF THE INVENTION

A continuing problem in the treatment of both CNS diseases and some amyloid associated diseases is the delivery of the therapeutic agent into the brain. It is an object of the present invention to provide compounds and compositions for the treatment of CNS diseases and amyloid associated diseases which facilitate passage through the blood brain barrier. As such, there are two general methods for crossing the blood brain barrier. The first, passive diffusion, requires a highly lipophilic structure to cross the barrier. The second uses an active transporter to facilitate transportation across the BBB. The present invention attempts to engage the active transporters in the BBB by incorporating both a BBB transport vector, such as a large neutral amino acid, and a therapeutic agent useful for the treatment of amyloidosis into a single compound.

Accordingly, in one embodiment, the present invention is directed to compounds of Formula I:
A-Y-Q

wherein:

Q is a BBB transport vector;

Y is a direct bond or a linker group;

A is hydrogen, alkyl, alkyloxy, alkenyl, alkenyloxy, alkynyl, alkynyloxy, carbocyclic, heterocyclic, bicyclic, aryl, heteroaryl, fused-ring aryl or heteroaryl, aryloxy, arylalkyl, arylalkyloxy, alkylcarbonyl, arylcarbonyl, alkoxycarbonyl, thiazolyl, triazolyl, imidazolyl, benzothiazolyl, benzoimidazolyl, 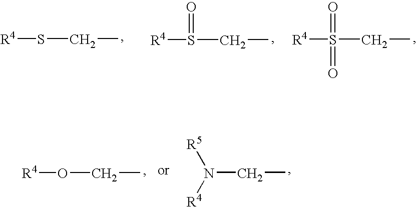
each of which may be optionally substituted; and

R⁴ and R⁵ together with the nitrogen form a 5 or 6 membered heterocyclic ring, or are each independently selected from the group consisting of hydrogen, alkyl, alkyloxy, alkenyl, alkenyloxy, alkynyl, alkynyloxy, cycloalkyl, aryl, aryloxy, arylalkyl, arylalkyloxy, alkylcarbonyl, arylcarbonyl, alkoxycarbonyl, thiazolyl, triazolyl, imidazolyl, benzothiazolyl, and benzoimidazolyl, each of which may be optionally substituted;

or a pharmaceutically acceptable salt, ester or prodrug thereof.

In some embodiments, Q is a 5 or 6 membered aromatic or heteroaromatic moiety, which may be further substituted. In other embodiments, Q is an amino acid moiety or analog thereof. Q may be a basic amino acid moiety or analog thereof, e.g., arginine, lysine, ornithine, and/or analogs thereof. Q may also be an acidic amino acid moiety or analog thereof, e.g., aspartic acid, glutamic acid, and/or analogs thereof. Furthermore, Q may be a small neutral amino acid moiety or analog thereof, e.g. glycine, alanine, serine, cysteine, and/or analogs thereof. Q may also be a large neutral amino acid moiety or analog thereof, e.g., phenylalanine, tryptophan, leucine, methionine, isoleucine, tyrosine, histidine, valine, threonine, proline, asparagine, glutamine, and/or analogs thereof. In other embodiments, the linker group is a disulfide bond, an ether linkage, a thioether linkage, an alkylene or alkenylene linkage, an amino or a hydrozino linkage, an ester-based linkage, a thioester linkage, an amide bond, an acid-labile linkage, or a Schiff base linkage.

In another embodiment, the present invention is directed to compounds of Formula II: 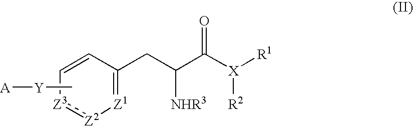
wherein:

X is oxygen, nitrogen, or sulfur;

Y is a direct bond or a linker group;

Z¹, Z², Z³ are each independently C, CH, CH₂, P, N, NH, S, or absent;

R¹ and R² are independently absent, hydrogen, alkyl, cycloalkyl, alkenyl, alkylnyl, aryl, arylalkyl, or acyl, each of which may be optionally substituted;

R³ is selected from the group consisting of hydrogen, alkyl, aryl, amido, arylamido, alkylcarbonyl, arylcarbonyl, arylaminocarbonyl, alkoxycarbonyl, alkanesulfonyl, arenesulfonyl, cycloalkanesulfonyl, and heteroarenesulfonyl, each of which may be optionally substituted;

A is hydrogen, alkyl, alkyloxy, alkenyl, alkenyloxy, alkynyl, alkynyloxy, carbocyclic, heterocyclic, bicyclic, aryl, heteroaryl, fused-ring aryl or heteroaryl, aryloxy, arylalkyl, arylalkyloxy, alkylcarbonyl, arylcarbonyl, alkoxycarbonyl, thiazolyl, triazolyl, imidazolyl, benzothiazolyl, benzoimidazolyl, 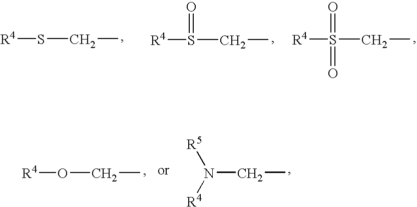
each of which may be optionally substituted; and

R⁴ and R⁵ together with the nitrogen form a 5 or 6 membered heterocyclic ring, or are each independently hydrogen, alkyl, alkyloxy, alkenyl, alkenyloxy, alkynyl, alkynyloxy, cycloalkyl, aryl, aryloxy, arylalkyl, arylalkyloxy, alkylcarbonyl, arylcarbonyl, alkoxycarbonyl, thiazolyl, triazolyl, imidazolyl, benzothiazolyl, or benzoimidazolyl, each of which may be optionally substituted;

or a pharmaceutically acceptable salt, ester or prodrug thereof.

In one embodiment, X is oxygen or nitrogen. In another embodiment, Y is a direct bond. In yet another embodiment, Z¹, Z² and Z³ are N, C or CH. In still another embodiment, R¹ and R² are independently absent or hydrogen. In another embodiment, R³ is hydrogen, arylamido, arylaminocarbonyl or arenesulfonyl, each of which may be optionally substituted. In yet another embodiment, A is one of the following groups:, R⁴—S—CH₂—, 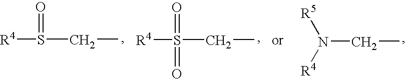
each of which may be optionally substituted.

In still another embodiment, R₄ and R₅ are each independently cycloalkyl, aryl, aryloxy, arylalkyl, arylalkyloxy, alkylcarbonyl, arylcarbonyl, alkoxycarbonyl, thiazolyl, triazolyl, imidazolyl, benzothiazolyl, or benzoimidazolyl, each of which may be optionally substituted. In some embodiments, R⁴ and R⁵ are each independently pyridine, pyrimidine, pyrimidinone, tetrahydropyridine, piperidine, piperazine, imidazole, benzoimidazole, oxazole, oxadiazole, benzooxazole, triazole, thiazole, benzothiazole, tetrazole, thiadiazole, pyrazolopyrimidine, isoquinoline, or tetrahydroisoquinoline, each of which may be optionally substituted. In another embodiment, R⁴ and R⁵ together with the nitrogen form a 6 membered ring optionally interrupted with one or more additional heteroatoms. In some embodiments the resultant 6 membered ring is a non-fused ring. In other embodiments, the linker group is a disulfide bond, an ether linkage, a thioether linkage, an alkylene or alkenylene linkage, an amino or a hydrozino linkage, an ester-based linkage, a thioester linkage, an amide bond, an acid-labile linkage, or a Schiff base linkage. In some embodiments, the compounds of the present invention are the compounds shown in Tables 2 or 3 or both.

In one embodiment, the compounds disclosed herein are used to treat CNS diseases or amyloid associated diseases. Exemplary diseases that may be treated with the compounds of the instant invention include, but are not limited to Alzheimer's disease, cerebral amyloid angiopathy, inclusion body myositis, macular degeneration, MCI, Down's syndrome, seizure, neuropathic pain, Abercrombie's degeneration, Acquired epileptiform aphasia, Landau-Kleffner Syndrome, Acute Disseminated Encephalomyelitis, Adrenoleukodystrophy, Leukodystrophy, Agnosia, Alexander Disease, Alpers' Disease, Progressive Sclerosing Poliodystrophy, Alternating Hemiplegia, Amyotrophic Lateral Sclerosis, Lou Gehrig's disease, Angelman Syndrome, Ataxia Telangiectasia, Ataxias and Cerebellar/Spinocerebellar Degeneration, Attention Deficit Disorder, Binswanger's Disease, subcortical dementia, Canavan Disease, Cerebral Hypoxia, Cerebro-Oculo-Facio-Skeletal Syndrome, Pena Shokeir II Syndrome, Charcot-Marie-Tooth, Chronic Inflammatory Demyelinating Polyneuropathy (CIDP), Corticobasal Degeneration, Creutzfeldt-Jakob Disease, Degenerative knee arthritis, Diabetic neuropathy, Early Infantile Epileptic Encephalopathy, Ohtahara Syndrome, Epilepsy, Friedreich's Ataxia, Guillain-Barre Syndrome (GBS), Acute Idiopathic Polyneuritis, Hallervorden-Spatz Disease, Neurodegeneration with Brain Iron Accumulation, Huntington's Disease, Krabbe Disease, Kugelberg-Welander Disease, Spinal Muscular Atrophy (SMA), SMA type I, SMA type II, SMA type III, Kennedy syndrome, progressive spinobulbar muscular atrophy, Congenital SMA with arthrogryposis, Adult SMA, Leigh's Disease, Lennox-Gastaut Syndrome, Machado-Joseph Disease, spinocerebellar ataxia type 3, Monomelic Amyotrophy, Multiple Sclerosis, Neuroacanthocytosis, Niemann-Pick disease, Olivopontocerebellar Atrophy, Paraneoplastic Syndromes, Neurologic paraneoplastic syndromes, Lambert-Eaton myasthenic syndrome, stiff-person syndrome, encephalomyelitis, myasthenia gravis, cerebellar degeneration, limbic and/or brainstem encephalitis, neuromyotonia, opsoclonus and sensory neuropathy, Parkinson's Disease, Pelizaeus-Merzbacher Disease, Pick's Disease, Primary Lateral Sclerosis, Progressive Locomotor Ataxia, Syphilitic Spinal Sclerosis, Tabes Dorsalis, Progressive Supranuclear Palsy, Rasmussen's Encephalitis, Rett Syndrome, Tourette's Syndrome, Usher syndrome, West syndrome, Infantile Spasms, Wilson Disease, and hepatolenticular degeneration.

In one embodiment, the compounds disclosed herein prevent or inhibit amyloid protein assembly into insoluble fibrils which, in vivo, are deposited in various organs, or they favor clearance of pre-formed deposits or slow deposition in patients already having deposits. In another embodiment, the compound may also prevent the amyloid protein, in its soluble, oligomeric form or in its fibrillar form, from binding or adhering to a cell surface and causing cell damage or toxicity. In yet another embodiment, the compound may block amyloid-induced cellular toxicity or macrophage activation. In another embodiment, the compound may block amyloid-induced neurotoxicity or microglial activation. In another embodiment, the compound protects cells from amyloid induced cytotoxicity of B-islet cells. In another embodiment, the compound may enhance clearance from a specific organ, e.g., the brain or it decreases concentration of the amyloid protein in such a way that amyloid fibril formation is prevented in the targeted organ.

The compounds of the invention may be administered therapeutically or prophylactically to treat diseases associated with amyloid fibril formation, aggregation or deposition. The compounds of the invention may act to ameliorate the course of an amyloid related disease using any of the following mechanisms (this list is meant to be illustrative and not limiting): slowing the rate of amyloid fibril formation or deposition; lessening the degree of amyloid deposition; inhibiting, reducing, or preventing amyloid fibril formation; inhibiting neurodegeneration or cellular toxicity induced by amyloid; inhibiting amyloid induced inflammation; enhancing the clearance of amyloid; or favoring the degradation of amyloid protein prior to its organization in fibrils. The compounds of the instant invention may also act to ameliorate the course of a CNS disease, including but not limited to reducing the intensity of a seizure, preventing a seizure or reducing neuropathic pain.

The compounds of the invention may be administered therapeutically or prophylactically to treat diseases associated with amyloid-, fibril formation, aggregation or deposition. The compounds of the invention may act to ameliorate the course of an amyloid-β related disease using any of the following mechanisms (this list is meant to be illustrative and not limiting): slowing the rate of amyloid-β fibril formation or deposition; lessening the degree of amyloid-β deposition; inhibiting, reducing, or preventing amyloid-β fibril formation; inhibiting neurodegeneration or cellular toxicity induced by amyloid-β; inhibiting amyloid-β induced inflammation; enhancing the clearance of amyloid-β from the brain; or favoring the degradation of amyloid-β protein prior to its organization in fibrils.

Therapeutic compounds of the invention may be effective in controlling amyloid-β deposition either following their entry into the brain (following penetration of the blood brain barrier) or from the periphery. When acting from the periphery, a compound may alter the equilibrium of Aβ between the brain and the plasma so as to favor the exit of Aβ from the brain. It may also increase the catabolism of neuronal Aβ and change the rate of exit from the brain. An increase in the exit of Aβ from the brain would result in a decrease in Aβ brain and cerebral spinal fluid (CSF) concentration and therefore favor a decrease in Aβ deposition. Alternatively, compounds that penetrate the brain could control deposition by acting directly on brain Aβ e.g., by maintaining it in a non-fibrillar form, favoring its clearance from the brain, or by slowing down APP processing. These compounds could also prevent Aβ in the brain from interacting with the cell surface and therefore prevent neurotoxicity, neurodegeneration or inflammation. They may also decrease Aβ production by activated microglia. The compounds may also increase degradation by macrophages or neuronal cells.

Similarly, therapeutic compounds of the invention may be effective in treating a CNS disease or an amyloid related disease either following their entry into the brain (following penetration of the blood brain barrier) or from the periphery. Preferably, the therapeutic compounds of the invention facilitate transport across the BBB and may generally be more effective following entry into the brain.

In one embodiment, the method is used to treat Alzheimer's disease (e.g., sporadic, familial, or early AD). The method can also be used prophylactically or therapeutically to treat other clinical occurrences of amyloid-β deposition, such as in Down's syndrome individuals and in patients with cerebral amyloid angiopathy (“CAA”) or hereditary cerebral hemorrhage.

In another embodiment, the method is used to treat mild cognitive impairment. Mild Cognitive Impairment (“MCI”) is a condition characterized by a state of mild but measurable impairment in thinking skills, which is not necessarily associated with the presence of dementia. MCI frequently, but not necessarily, precedes Alzheimer's disease.

Additionally, abnormal accumulation of APP and of amyloid-β protein in muscle fibers has been implicated in the pathology of sporadic inclusion body myositis (IBM) (Askanas, et al., Proc. Natl. Acad. Sci. USA 93, 1314-1319 (1996); Askanas, et al., Current Opinion in Rheumatology 7, 486-496 (1995)). Accordingly, the compounds of the invention can be used prophylactically or therapeutically in the treatment of disorders in which amyloid-beta protein is abnormally deposited at non-neurological locations, such as treatment of IBM by delivery of the compounds to muscle fibers.

Additionally, it has been shown that Aβ is associated with abnormal extracellular deposits, known as drusen, that accumulate along the basal surface of the retinal pigmented epithelium in individuals with age-related macular degeneration (AMD). AMD is a cause of irreversible vision loss in older individuals. It is believed that Aβ deposition could be an important component of the local inflammatory events that contribute to atrophy of the retinal pigmented epithelium, drusen biogenesis, and the pathogenesis of AMD (Johnson, et al., Proc. Natl. Acad. Sci. USA 99(18), 11830-5 (2002)).

The present invention therefore relates to the use of compounds of Formula I, Formula II, or compounds otherwise described herein in the prevention or treatment of CNS diseases or amyloid-related diseases, including, inter alia, Alzheimer's disease, cerebral amyloid angiopathy, mild cognitive impairment, inclusion body myositis, Down's syndrome, macular degeneration, as well as other types of amyloidosis like IAPP-related amyloidosis (e.g., diabetes), primary (AL) amyloidosis, secondary (AA) amyloidosis and β₂ microglobulin-related (dialysis-related) amyloidosis; seizure, neuropathic pain, Abercrombie's degeneration, Acquired epileptiform aphasia, Landau-Kleffner Syndrome, Acute Disseminated Encephalomyelitis, Adrenoleukodystrophy, Leukodystrophy, Agnosia, Alexander Disease, Alpers' Disease, Progressive Sclerosing Poliodystrophy, Alternating Hemiplegia, Amyotrophic Lateral Sclerosis, Lou Gehrig's disease, Angelman Syndrome, Ataxia Telangiectasia, Ataxias and Cerebellar/Spinocerebellar Degeneration, Attention Deficit Disorder, Binswanger's Disease, subcortical dementia, Canavan Disease, Cerebral Hypoxia, Cerebro-Oculo-Facio-Skeletal Syndrome, Pena Shokeir II Syndrome, Charcot-Marie-Tooth, Chronic Inflammatory Demyelinating Polyneuropathy (CIDP), Corticobasal Degeneration, Creutzfeldt-Jakob Disease, Degenerative knee arthritis, Diabetic neuropathy, Early Infantile Epileptic Encephalopathy, Ohtahara Syndrome, Epilepsy, Friedreich's Ataxia, Guillain-Barre Syndrome (GBS), Acute Idiopathic Polyneuritis, Hallervorden-Spatz Disease, Neurodegeneration with Brain Iron Accumulation, Huntington's Disease, Krabbe Disease, Kugelberg-Welander Disease, Spinal Muscular Atrophy (SMA), SMA type I, SMA type II, SMA type III, Kennedy syndrome, progressive spinobulbar muscular atrophy, Congenital SMA with arthrogryposis, Adult SMA, Leigh's Disease, Lennox-Gastaut Syndrome, Machado-Joseph Disease, spinocerebellar ataxia type 3, Monomelic Amyotrophy, Multiple Sclerosis, Neuroacanthocytosis, Niemann-Pick disease, Olivopontocerebellar Atrophy, Paraneoplastic Syndromes, Neurologic paraneoplastic syndromes, Lambert-Eaton myasthenic syndrome, stiff-person syndrome, encephalomyelitis, myasthenia gravis, cerebellar degeneration, limbic and/or brainstem encephalitis, neuromyotonia, opsoclonus and sensory neuropathy, Parkinson's Disease, Pelizaeus-Merzbacher Disease, Pick's Disease, Primary Lateral Sclerosis, Progressive Locomotor Ataxia, Syphilitic Spinal Sclerosis, Tabes Dorsalis, Progressive Supranuclear Palsy, Rasmussen's Encephalitis, Rett Syndrome, Tourette's Syndrome, Usher syndrome, West syndrome, Infantile Spasms, Wilson Disease, and hepatolenticular degeneration.

In Type II diabetes related amyloidosis (IAPP), the amyloidogenic protein IAPP induces β-islet cell toxicity when organized in oligomeric forms or in fibrils. Hence, appearance of IAPP fibrils in the pancreas of type II diabetic patients contributes to the loss of the β islet cells (Langerhans) and organ dysfunction which leads to insulinemia.

Primary amyloidosis (AL amyloid) is usually found associated with plasma cell dyscrasia and multiple myeloma. It can also be found as an idiopathic disease.

Secondary (AA) amyloidosis is usually seen associated with chronic infection (such as tuberculosis) or chronic inflammation (such as rheumatoid arthritis). A familial form of secondary amyloidosis is also seen in Familial Mediterranean Fever (FMF).

β₂ microglobulin-related (dialysis-related) amyloidosis is found in long-term hemodialysis patients. Patients undergoing long term hemodialysis will develop β₂-microglobulin fibrils in the carpal tunnel and in the collagen rich tissues in several joints. This causes severe pains, joint stiffness and swelling. These deposits are due to the inability to maintain low levels of β₂M in plasma of dialyzed patients. Increased plasma concentrations of β₂M protein will induce structural changes and may lead to the deposition of modified β₂M as insoluble fibrils in the joints.

### DETAILED DESCRIPTION OF THE INVENTION

The present invention relates to the use of compounds of Formula I, Formula II, or compounds otherwise described herein in the treatment of central nervous system (CNS) diseases and/or amyloid associated diseases. For convenience, some definitions of terms referred to herein are set forth below.

Central Nervous System Diseases

As used herein, the terms “central nervous system disease” and “CNS disease” refer to neurological and/or psychiatric changes in the CNS, e.g., brain and spinal cord, which manifest in a variety of symptoms. Examples of CNS disease states include, but are not limited to: migraine headache; cerebrovascular deficiency; psychoses including paranoia, schizophrenia, attention deficiency, and autism; obsessive/compulsive disorders including anorexia and bulimia; convulsive disorders including epilepsy and withdrawal from addictive substances; cognitive diseases including Parkinson's disease and dementia; and anxiety/depression disorders such as anticipatory anxiety (e.g., prior to surgery, dental work and the like), depression, mania, seasonal affective disorder (SAD); and convulsions and anxiety caused by withdrawal from addictive substances such as opiates, benzodiazepines, nicotine, alcohol, cocaine, and other substances of abuse. Further non-limiting examples of CNS diseases include, but are not limited to Abercrombie's degeneration, Acquired epileptiform aphasia (Landau-Kleffner Syndrome), Acute Disseminated Encephalomyelitis, Adrenoleukodystrophy, Agnosia, Alexander Disease, Alpers' Disease, Alternating Hemiplegia, Amyotrophic Lateral Sclerosis, Angelman Syndrome, Ataxia Telangiectasia, Ataxias and Cerebellar/Spinocerebellar Degeneration, Attention Deficit Disorder, Binswanger's Disease, Canavan Disease, Cerebral Hypoxia, Cerebro-Oculo-Facio-Skeletal Syndrome, Charcot-Marie-Tooth, Chronic Inflammatory Demyelinating Polyneuropathy (CIDP), Corticobasal Degeneration, Creutzfeldt-Jakob disease, Degenerative knee arthritis, Diabetic neuropathy, Early Infantile Epileptic Encephalopathy (Ohtahara Syndrome), Epilepsy, Friedreich's Ataxia, Guillain-Barre Syndrome (GBS), Hallervorden-Spatz Disease, Huntington's Disease, Krabbe Disease, Kugelberg-Welander Disease (Spinal Muscular Atrophy), Leigh's Disease, Lennox-Gastaut Syndrome, Machado-Joseph Disease, Macular degeneration, Monomelic Amyotrophy, Multiple Sclerosis, Neuroacanthocytosis, Niemann-Pick disease, Olivopontocerebellar Atrophy, Paraneoplastic Syndromes, Parkinson's Disease, Pelizaeus-Merzbacher Disease, Pick's Disease, Primary Lateral Sclerosis, Progressive Locomotor Ataxia (Syphilitic Spinal Sclerosis, Tabes Dorsalis), Progressive Supranuclear Palsy, Rasmussen's Encephalitis, Rett Syndrome, Tourette's Syndrome, and Usher syndrome, West syndrome (Infantile Spasms), and Wilson Disease. General characteristics of such diseases are known in the art. The skilled artisan would be able to identify further CNS diseases known in the art without undue experimentation.

Amyloid Associated Diseases

Below are nonlimiting examples of amyloid associated diseases. Some, but not all, amyloid associated diseases are also CNS diseases. Similarly, some, but not all, CNS diseases are amyloid associated diseases. These lists of diseases are not meant to be mutually exclusive or all-encompassing.

AA (Reactive) Amyloidosis

Generally, AA amyloidosis is a manifestation of a number of diseases that provoke a sustained acute phase response. Such diseases include chronic inflammatory disorders, chronic local or systemic microbial infections, and malignant neoplasms. The most common form of reactive or secondary (AA) amyloidosis is seen as the result of long-standing inflammatory conditions. For example, patients with Rheumatoid Arthritis or Familial Mediterranean Fever (which is a genetic disease) can develop AA amyloidosis. The terms “AA amyloidosis” and “secondary (AA) amyloidosis” are used interchangeably.

AA fibrils are generally composed of 8,000 Dalton fragments (AA peptide or protein) formed by proteolytic cleavage of serum amyloid A protein (ApoSAA), a circulating apolipoprotein which is mainly synthesized in hepatocytes in response to such cytokines as IL-1, IL-6 and TNF. Once secreted, ApoSAA is complexed with HDL. Deposition of AA fibrils can be widespread in the body, with a preference for parenchymal organs. The kidneys are usually a deposition site, and the liver and the spleen may also be affected. Deposition is also seen in the heart, gastrointestinal tract, and the skin.

Underlying diseases which can lead to the development of AA amyloidosis include, but are not limited to inflammatory diseases, such as rheumatoid arthritis, juvenile chronic arthritis, ankylosing spondylitis, psoriasis, psoriatic arthropathy, Reiter's syndrome, Adult Still's disease, Behcet's syndrome, and Crohn's disease. AA deposits are also produced as a result of chronic microbial infections, such as leprosy, tuberculosis, bronchiectasis, decubitus ulcers, chronic pyelonephritis, osteomyelitis, and Whipple's disease. Certain malignant neoplasms can also result in AA fibril amyloid deposits. These include such conditions as Hodgkin's lymphoma, renal carcinoma, carcinomas of gut, lung and urogenital tract, basal cell carcinoma, and hairy cell leukemia. Other underlying conditions that may be associated with AA amyloidosis are Castleman's disease and Schnitzler's syndrome.

AL Amyloidoses (Primary Amyloidosis)

AL amyloid deposition is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia. AL amyloidosis is also described in detail in Current Drug Targets, 2004, 5 159-171.

Fibrils of AL amyloid deposits are composed of monoclonal immunoglobulin light chains or fragments thereof. More specifically, the fragments are derived from the N-terminal region of the light chain (kappa or lambda) and contain all or part of the variable (V_{L}) domain thereof. Deposits generally occur in the mesenchymal tissues, causing peripheral and autonomic neuropathy, carpal tunnel syndrome, macroglossia, restrictive cardiomyopathy, arthropathy of large joints, immune dyscrasias, myelomas, as well as occult dyscrasias. However, it should be noted that almost any tissue, particularly visceral organs such as the kidney, liver, spleen and heart, may be involved.

Hereditary Systemic Amyloidoses

There are many forms of hereditary systemic amyloidoses. Although they are relatively rare conditions, adult onset of symptoms and their inheritance patterns (usually autosomal dominant) lead to persistence of such disorders in the general population. Generally, the syndromes are attributable to point mutations in the precursor protein leading to production of variant amyloidogenic peptides or proteins. For example, point mutations in ATTR protein from Transthyretin and fragments, N-terminal fragment of Apolipoprotein A1 (apoAI), AapoAII from Apolipoprotein AII, Lysozyme (Alys), Fibrogen alpha chain fragment, Gelsolin fragment (Agel), Cystatin C fragment (ACys), β-amyloid protein (AP) derived from Amyloid Precursor Protein (APP), Prion Protein (PrP, APrP^{SC}) derived from Prp precursor protein (51-91 insert), AA derived from Serum amyloid A protein (ApoSAA), AH amyloid protein, derived from immunoglobulin heavy chain (gamma I), ACal amyloid protein from (pro)calcitonin, AANF amyloid protein from atrial natriuretic factor, Apro from Prolactin, or Abri/ADan from ABri peptide can lead to clinical syndromes which include, but are not limited to, familial amyloid polyneuropathy (FAP), cardiac involvement predominant without neuropathy, senile systemic amyloidosis, Tenosynovium, non-neuropathic Ostertag-type amyloidosis, familial amyloidosis, cranial neuropathy with lattice corneal dystrophy, hereditary cerebral hemorrhage (CAA)—Icelandic type, familial Alzheimer's Disease, Alzheimer's disease, Down's syndrome, hereditary cerebral hemorrhage with amyloidosis—Dutch type, familial Dementia, familial Creutzfeldt-Jakob disease; Gerstmann-Straussler-Scheinker syndrome, hereditary spongiform encephalopathies, prion diseases, familial Mediterranean fever with predominant renal involvement, Muckle-Well's syndrome, nephropathy, deafness, urticaria, limb pain, cardiomyopathy with persistent atrial standstill, cutaneous deposits (bullous, papular, pustulodermal), myeloma associated amyloidosis, medullary carcinomas of the thyroid, isolated atrial amyloid, prolactinomas, and/or British and Danish familial Dementia. General characteristics of such diseases are known in the art. These point mutations and clinical syndromes are exemplary and are not intended to limit the scope of the invention. For example, more than 40 separate point mutations in the transthyretin gene have been described, all of which give rise to clinically similar forms of familial amyloid polyneuropathy.

In general, any hereditary amyloid disorder can also occur sporadically, and both hereditary and sporadic forms of a disease present with the same characteristics with regard to amyloid. For example, the most prevalent form of secondary AA amyloidosis occurs sporadically, e.g. as a result of ongoing inflammation, and is not associated with Familial Mediterranean Fever. Thus general discussion relating to hereditary amyloid disorders below can also be applied to sporadic amyloidoses.

Transthyretin (TTR) is a 14 kiloDalton protein that is also sometimes referred to as prealbumin. It is produced by the liver and choroid plexus, and it functions in transporting thyroid hormones and vitamin A. At least 50 variant forms of the protein, each characterized by a single amino acid change, are responsible for various forms of familial amyloid polyneuropathy. For example, substitution of proline for leucine at position 55 results in a particularly progressive form of neuropathy; substitution of methionine for leucine at position 111 resulted in a severe cardiopathy in Danish patients.

Amyloid deposits isolated from heart tissue of patients with systemic amyloidosis have revealed that the deposits are composed of a heterogeneous mixture of TTR and fragments thereof, collectively referred to as ATTR, the full length sequences of which have been characterized. ATTR fibril components can be extracted from such plaques and their structure and sequence determined according to the methods known in the art (e.g., Gustavsson, A., et al., Laboratory Invest. 73: 703-708, 1995; Kametani, F., et al., Biochem. Biophys. Res. Commun. 125: 622-628, 1984; Pras, M., et al., PNAS 80: 539-42, 1983).

Persons having point mutations in the molecule apolipoprotein Al (e.g., Gly→Arg26; Trp→Arg50; Leu→Arg60) exhibit a form of amyloidosis (“Östertag type”) characterized by deposits of the protein apolipoprotein AI or fragments thereof (AApoAI). These patients have low levels of high density lipoprotein (HDL) and present with a peripheral neuropathy or renal failure.

A mutation in the alpha chain of the enzyme lysozyme (e.g., Ile→Thr56 or Asp→His57) is the basis of another form of Östertag-type non-neuropathic hereditary amyloid reported in English families. Here, fibrils of the mutant lysozyme protein (Alys) are deposited, and patients generally exhibit impaired renal function. This protein, unlike most of the fibril-forming proteins described herein, is usually present in whole (unfragmented) form (Benson, M. D., et al. CIBA Fdn. Symp. 199: 104-131, 1996).

Immunoglobulin light chains tend to form aggregates in various morphologies, including fibrillar (e.g., AL amyloidosis and AH amyloidosis), granular (e.g., light chain deposition disease (LCDD), heavy chain deposition disease (HCDD), and light-heavy chain deposition disease (LHCDD)), crystalline (e.g., Acquired Farconi's Syndome), and microtubular (e.g., Cryoglobulinemia). AL and AH amyloidosis is indicated by the formation of insoluble fibrils of immunoglobulin light chains and heavy chain, respectively, and/or their fragments. In AL fibrils, lambda (λ) chains such as λ VI chains (λ6 chains), are found in greater concentrations than kappa (κ) chains. λIII chains are also slightly elevated. Merlini et al., CLIN CHEM LAB MED 39(11):1065-75 (2001). Heavy chain amyloidosis (AH) is generally characterized by aggregates of gamma chain amyloid proteins of the IgG1 subclass. Eulitz et al., PROC NATL ACAD SCI USA 87:6542-46 (1990).

Comparison of amyloidogenic to non-amyloidogenic light chains has revealed that the former can include replacements or substitutions that appear to destabilize the folding of the protein and promote aggregation. AL and LCDD have been distinguished from other amyloid diseases due to their relatively small population monoclonal light chains, which are manufactured by neoplastic expansion of an antibody-producing B cell. AL aggregates typically are well-ordered fibrils of lambda chains. LCDD aggregates are relatively amorphous aggregations of both kappa and lambda chains, with a majority being kappa, in some cases κIV. Bellotti et al., JOURNAL OF STRUCTURAL BIOLOGY 13:280-89 (2000). Comparison of amyloidogenic and non-amyloidogenic heavy chains in patients having AH amyloidosis has revealed missing and/or altered components. Eulitz et al., PROC NATL ACAD SCI USA 87:6542-46 (1990) (pathogenic heavy chain characterized by significantly lower molecular mass than non-amyloidogenic heavy chains); and Solomon et al. AM J HEMAT 45(2) 171-6 (1994) (amyloidogenic heavy chain characterized as consisting solely of the VH-D portion of the non-amyloidogenic heavy chain).

Accordingly, potential methods of detecting and monitoring treatment of subjects having or at risk of having AL, LCDD, AH, and the like, include but are not limited to immunoassaying plasma or urine for the presence or depressed deposition of amyloidogenic light or heavy chains, e.g., amyloid λ, amyloid κ, amyloid κIV, amyloid γ, or amyloid γ1.

Brain Amyloidosis

The most frequent type of amyloid in the brain is composed primarily of Aβ peptide fibrils, resulting in dementia associated with sporadic (non-hereditary) Alzheimer's disease. In fact, the incidence of sporadic Alzheimer's disease greatly exceeds forms shown to be hereditary. Nevertheless, fibril peptides forming plaques are very similar in both types. Brain amyloidosis includes those diseases, conditions, pathologies, and other abnormalities of the structure or function of the brain, including components thereof, in which the causative agent is amyloid. The area of the brain affected in an amyloid associated disease may be the stroma including the vasculature or the parenchyma including functional or anatomical regions, or neurons themselves. A subject need not have received a definitive diagnosis of a specifically recognized amyloid associated disease. The term “amyloid related disease” includes brain amyloidosis.

Amyloid-β peptide (“AP”) is a 39-43 amino acid peptide derived by proteolysis from a large protein known as Beta Amyloid Precursor Protein (“βAPP”). Mutations in βAPP result in familial forms of Alzheimer's disease, Down's syndrome, cerebral amyloid angiopathy, and senile dementia, characterized by cerebral deposition of plaques composed of Aβ fibrils and other components, which are described in further detail below. Known mutations in APP associated with Alzheimer's disease occur proximate to the cleavage sites of β or γ-secretase, or within Aβ. For example, position 717 is proximate to the site of gamma-secretase cleavage of APP in its processing to Aβ, and positions 670/671 are proximate to the site of β-secretase cleavage. Mutations at any of these residues may result in Alzheimer's disease, presumably by causing an increase in the amount of the 42/43 amino acid form of Aβ generated from APP. The familial form of Alzheimer's disease represents only 10% of the subject population. Most occurrences of Alzheimer's disease are sporadic cases where APP and Aβ do not possess any mutation. The structure and sequence of Aβ peptides of various lengths are well known in the art. Such peptides can be made according to methods known in the art, or extracted from the brain according to known methods (e.g., Glenner and Wong, Biochem. Biophys. Res. Comm. 129, 885-90 (1984); Glenner and Wong, Biochem. Biophys. Res. Comm. 122, 1131-35 (1984)). In addition, various forms of the peptides are commercially available. APP is expressed and constitutively catabolized in most cells. The dominant catabolic pathway appears to be cleavage of APP within the Aβ sequence by an enzyme provisionally termed α-secretase, leading to release of a soluble ectodomain fragment known as APPsα. This cleavage precludes the formation of Aβ peptide. In contrast to this non-amyloidogenic pathway, APP can also be cleaved by enzymes known as β- and γ-secretase at the N- and C-termini of the Aβ, respectively, followed by release of Aβ into the extracellular space. To date, BACE has been identified as β-secretase (Vasser, et al., Science 286:735-741, 1999) and presenilins have been implicated in γ-secretase activity (De Strooper, et al., Nature 391, 387-90 (1998)). The 39-43 amino acid Aβ peptide is produced by sequential proteolytic cleavage of the amyloid precursor protein (APP) by the β and γ secretases enzyme. Although Aβ40 is the predominant form produced, 5-7% of total Aβ exists as Aβ42 (Cappai et al., Int. J. Biochem. Cell Biol. 31. 885-89 (1999)).

The length of the Aβ peptide appears to dramatically alter its biochemical/biophysical properties. Specifically, the additional two amino acids at the C-terminus of Aβ42 are very hydrophobic, presumably increasing the propensity of Aβ42 to aggregate. For example, Jarrett, et al. demonstrated that Aβ42 aggregates very rapidly in vitro compared to Aβ40, suggesting that the longer forms of Aβ may be the important pathological proteins that are involved in the initial seeding of the neuritic plaques in Alzheimer's disease (Jarrett, et al., Biochemistry 32, 4693-97 (1993); Jarrett, et al., Ann. N. Y. Acad Sci. 695, 144-48 (1993)). This hypothesis has been further substantiated by the recent analysis of the contributions of specific forms of Aβ in cases of genetic familial forms of Alzheimer's disease (“FAD”). For example, the “London” mutant form of APP (APPV7171) linked to FAD selectively increases the production of Aβ 42/43 forms versus Aβ 40 (Suzuki, et al., Science 264, 1336-40 (1994)) while the “Swedish” mutant form of APP (APPK670N/M671 L) increases levels of both Aβ40 and Aβ42/43 (Citron, et al., Nature 360, 672-674 (1992); Cai, et al., Science 259, 514-16, (1993)). Also, it has been observed that FAD-linked mutations in the Presenilin-1 (“PS1”) or Presenilin-2 (“PS2”) genes will lead to a selective increase in Aβ42/43 production but not Aβ40 (Borchelt, et al., Neuron 17, 1005-13 (1996)). This finding was corroborated in transgenic mouse models expressing PS mutants that demonstrate a selective increase in brain Aβ42 (Borchelt, op cit.; Duff, et al., Neurodegeneration 5(4), 293-98 (1996)). Thus the leading hypothesis regarding the etiology of Alzheimer's disease is that an increase in Aβ42 brain concentration due to an increased production and release of Aβ42 or a decrease in clearance (degradation or brain clearance) is a causative event in the disease pathology.

Multiple mutation sites in either Aβ or the APP gene have been identified and are clinically associated with either dementia or cerebral hemorrhage. Exemplary CAA disorders include, but are not limited to, hereditary cerebral hemorrhage with amyloidosis of Icelandic type (HCHWA-I); the Dutch variant of HCHWA (HCHWA-D; a mutation in Aβ); the Flemish mutation of Aβ; the Arctic mutation of Aβ; the Italian mutation of Aβ; the Iowa mutation of Aβ; familial British dementia; and familial Danish dementia. CAA may also be sporadic.

As used herein, the terms “β amyloid,” “amyloid-β,” and the like refer to amyloid β proteins or peptides, amyloid β precursor proteins or peptides, intermediates, and modifications and fragments thereof, unless otherwise specifically indicated. In particular, “Aβ” refers to any peptide produced by proteolytic processing of the APP gene product, especially peptides which are associated with amyloid pathologies, including Aβ1-39, Aβ1-40, Aβ1-41, Aβ1-42, and Aβ1-43. For convenience of nomenclature, “Aβ1-42” may be referred to herein as “Aβ(1-42)” or simply as “Aβ42” or “Aβ₄₂” (and likewise for any other amyloid peptides discussed herein). As used herein, the terms “β amyloid,” “amyloid-β,” and “Aβ” are synonymous.

Unless otherwise specified, the term “amyloid” refers to amyloidogenic proteins, peptides, or fragments thereof which can be soluble (e.g., monomeric or oligomeric) or insoluble (e.g., having fibrillary structure or in amyloid plaque). See, e.g., M P Lambert, et al., Proc. Nat'l Acad. Sci. USA 95, 6448-53 (1998). “Amyloidosis” or “amyloid disease” or “amyloid associated disease” refers to a pathological condition characterized by the presence of amyloid fibers. “Amyloid” is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common X-ray diffraction and infrared spectra.

Gelsolin is a calcium binding protein that binds to fragments and actin filaments. Mutations at position 187 (e.g., Asp→Asn; Asp→Tyr) of the protein result in a form of hereditary systemic amyloidosis, usually found in patients from Finland, as well as persons of Dutch or Japanese origin. In afflicted individuals, fibrils formed from gelsolin fragments (Agel), usually consist of amino acids 173-243 (68 kDa carboxyterminal fragment) and are deposited in blood vessels and basement membranes, resulting in corneal dystrophy and cranial neuropathy which progresses to peripheral neuropathy, dystrophic skin changes and deposition in other organs. (Kangas, H., et al. Human Mol. Genet. 5(9): 1237-1243, 1996).

Other mutated proteins, such as mutant alpha chain of fibrinogen (AfibA) and mutant cystatin C (Acys) also form fibrils and produce characteristic hereditary disorders. AfibA fibrils form deposits characteristic of a nonneuropathic hereditary amyloid with renal disease; Acys deposits are characteristic of a hereditary cerebral amyloid angiopathy reported in Iceland (Isselbacher, Harrison's Principles of Internal Medicine, McGraw-Hill, San Francisco, 1995; Benson, et al.). In at least some cases, patients with cerebral amyloid angiopathy (CAA) have been shown to have amyloid fibrils containing a non-mutant form of cystatin C in conjunction with amyloid beta protein (Nagai, A., et al. Molec. Chem. Neuropathol. 33: 63-78, 1998).

Cerebral Amyloidosis

Local deposition of amyloid is common in the brain, particularly in elderly individuals. The most frequent type of amyloid in the brain is composed primarily of Aβ peptide fibrils, resulting in dementia or sporadic (non-hereditary) Alzheimer's disease. The most common occurrences of cerebral amyloidosis are sporadic and not familial. For example, the incidence of sporadic Alzheimer's disease and sporadic CAA greatly exceeds the incidence of familial AD and CAA. Moreover, sporadic and familial forms of the disease cannot be distinguished from each other (they differ only in the presence or absence of an inherited genetic mutation); for example, the clinical symptoms and the amyloid plaques formed in both sporadic and familial AD are very similar, if not identical.

Cerebral amyloid angiopathy (CAA) refers to the specific deposition of amyloid fibrils in the walls of leptomingeal and cortical arteries, arterioles and veins. It is commonly associated with Alzheimer's disease, Down's syndrome and normal aging, as well as with a variety of familial conditions related to stroke or dementia (see Frangione et al., Amyloid: J. Protein Folding Disord. 8, Suppl. 1, 36-42 (2001)). CAA can occur sporadically or be hereditary.

Senile Systemic Amyloidosis

Amyloid deposition, either systemic or focal, increases with age. For example, fibrils of wild type transthyretin (TTR) are commonly found in the heart tissue of elderly individuals. These may be asymptomatic, clinically silent, or may result in heart failure. Asymptomatic fibrillar focal deposits may also occur in the brain (Aβ), corpora amylacea of the prostate (β₂ microglobulin), joints and seminal vesicles.

Dialysis-Related Amyloidosis (DRA)

Plaques composed of β₂ microglobulin (β₂M) fibrils commonly develop in patients receiving long term hemodialysis or peritoneal dialysis. β₂ microglobulin is a 11.8 kiloDalton polypeptide and is the light chain of Class I MHC antigens, which are present on all nucleated cells. Under normal circumstances, β₂M is usually distributed in the extracellular space unless there is an impaired renal function, in which case β₂M is transported into tissues where it polymerizes to form amyloid fibrils. Failure of clearance such as in the case of impaired renal function, leads to deposition in the carpal tunnel and other sites (primarily in collagen-rich tissues of the joints). Unlike other fibril proteins, β₂M molecules are not produced by cleavage of a longer precursor protein and are generally present in unfragmented form in the fibrils. (Benson, supra). Retention and accumulation of this amyloid precursor has been shown to be the main pathogenic process underlying DRA. DRA is characterized by peripheral joint osteoarthropathy (e.g., joint stiffness, pain, swelling, etc.). Isoforms of β₂M, glycated β₂M, or polymers of β₂M in tissue are the most amyloidogenic form (as opposed to native β₂M). Unlike other types of amyloidosis, β₂M is confined largely to osteoarticular sites. Visceral depositions are rare. Occasionally, these deposits may involve blood vessels and other important anatomic sites.

Despite improved dialysis methods for removal of β₂M, the majority of patients have plasmatic β₂M concentrations that remain dramatically higher than normal. These elevated β₂M concentrations generally lead to Diabetes-Related Amyloidosis (DRA) and cormorbidities that contribute to mortality.

Islet Amyloid Polypeptide and Diabetes

Islet hyalinosis (amyloid deposition) was first described over a century ago as the presence of fibrous protein aggregates in the pancreas of patients with severe hyperglycemia (Opie, E L., J Exp. Med 5: 397-428, 1901). Today, islet amyloid, composed predominantly of islet amyloid polypeptide (IAPP), or amylin, is a characteristic histopathological marker in over 90% of all cases of Type II diabetes (also known as Non-Insulin Dependent Diabetes, or NIDDM). These fibrillar accumulations result from the aggregation of the islet amyloid polypeptide (IAPP) or amylin, which is a 37 amino acid peptide, derived from a larger precursor peptide, called pro-IAPP.

IAPP is co-secreted with insulin in response to β-cell secretagogues. This pathological feature is not associated with insulin-dependent (Type I) diabetes and is a unifying characteristic for the heterogeneous clinical phenotypes diagnosed as NIDDM (Type II diabetes).

Longitudinal studies in cats and immunocytochemical investigations in monkeys have shown that a progressive increase in islet amyloid is associated with a dramatic decrease in the population of insulin-secreting β-cells and increased severity of the disease. More recently, transgenic studies have strengthened the relationship between IAPP plaque formation and β-cell apoptosis and dysfunction, indicating that amyloid deposition is a principal factor in increasing severity of Type II diabetes.

IAPP has also been shown to induce β-islet cell toxicity in vitro, indicating that appearance of IAPP fibrils in the pancreas of Type II or Type I diabetic patients (post-islet transplantation) could contribute to the loss of the β-cell islets (Langerhans) and organ dysfunction. In patients with Type II diabetes, the accumulation of pancreatic IAPP leads to formation of oligomeric IAPP, leading to a buildup of IAPP-amyloid as insoluble fibrous deposits which eventually destroy the insulin-producing β cells of the islet, resulting in β cell depletion and failure (Westermark, P., Grimelius, L., Acta Path. Microbiol. Scand, sect. A. 81: 291-300, 1973; de Koning, E J P., et al., Diabetologia 36: 378-384, 1993; and Lorenzo, A., et al., Nature 368: 756-760, 1994). Accumulation of IAPP as fibrous deposits can also have an impact on the ratio of pro-IAPP to IAPP normally found in plasma by increasing this ratio due to the trapping of IAPP in deposits. Reduction of β cell mass can be manifested by hyperglycemia and insulinemia. This β-cell mass loss can lead to a need for insulin therapy.

Diseases caused by the death or malfunctioning of a particular type or types of cells can be treated by transplanting into the patient healthy cells of the relevant type of cell. This approach has been used for Type I diabetes patients. Often pancreatic islet cells from a donor are cultured in vitro prior to transplantation, to allow them to recover after the isolation procedure or to reduce their immunogenicity. However, in many instances islet cell transplantation is unsuccessful, due to death of the transplanted cells. One reason for this poor success rate is IAPP, which organizes into toxic oligomers. Toxic effects may result from intracellular and extracellular accumulation of fibril oligomers. The IAPP oligomers can form fibrils and become toxic to the cells in vitro. In addition, IAPP fibrils are likely to continue to grow after the cells are transplanted and cause death or dysfunction of the cells. This may occur even when the cells are from a healthy donor and the patient receiving the transplant does not have a disease that is characterized by the presence of fibrils. For example, compounds of the present invention may also be used in preparing tissues or cells for transplantation according to the methods described in International Patent Application (PCT) number WO 01/003680.

The compounds of the invention may also stabilize the ratio of the concentrations of Pro-IAPP/IAPP, pro-Insulin/Insulin and C-peptide levels. In addition, as biological markers of efficacy, the results of the different tests, such as the arginine-insulin secretion test, the glucose tolerance test, insulin tolerance and sensitivity tests, could all be used as markers of reduced β-cell mass and/or accumulation of amyloid deposits. Such class of drugs could be used together with other drugs targeting insulin resistance, hepatic glucose production, and insulin secretion. Such compounds might prevent insulin therapy by preserving β-cell function and be applicable to preserving islet transplants.

Hormone-Derived Amyloidoses

Endocrine organs may harbor amyloid deposits, particularly in aged individuals. Hormone-secreting tumors may also contain hormone-derived amyloid plaques, the fibrils of which are made up of polypeptide hormones such as calcitonin (medullary carcinoma of the thyroid), and atrial natriuretic peptide (isolated atrial amyloidosis). Sequences and structures of these proteins are well known in the art.

Miscellaneous Amyloidoses

There are a variety of other forms of amyloid disease that are normally manifest as localized deposits of amyloid. In general, these diseases are probably the result of the localized production or lack of catabolism of specific fibril precursors or a predisposition of a particular tissue (such as the joint) for fibril deposition. Examples of such idiopathic deposition include nodular AL amyloid, cutaneous amyloid, endocrine amyloid, and tumor-related amyloid. Other amyloid related diseases include those described above, such as familial amyloid polyneuropathy (FAP), senile systemic amyloidosis, Tenosynovium, familial amyloidosis, Ostertag-type, non-neuropathic amyloidosis, cranial neuropathy, hereditary cerebral hemorrhage, familial dementia, chronic dialysis, familial Creutzfeldt-Jakob disease; Gerstmann-Straussler-Scheinker syndrome, hereditary spongiform encephalopathies, prion diseases, familial Mediterranean fever, Muckle-Well's syndrome, nephropathy, deafness, urticaria, limb pain, cardiomyopathy, cutaneous deposits, multiple myeloma, benign monoclonal gammopathy, maccoglobulinaemia, myeloma associated amyloidosis, medullary carcinomas of the thyroid, isolated atrial amyloid, and diabetes.

The compounds of the invention may be administered therapeutically or prophylactically to treat diseases associated with amyloid fibril formation, aggregation or deposition, regardless of the clinical setting. The compounds of the invention may act to ameliorate the course of an amyloid related disease using any of the following mechanisms, such as, for example but not limited to: slowing the rate of amyloid fibril formation or deposition; lessening the degree of amyloid deposition; inhibiting, reducing, or preventing amyloid fibril formation; inhibiting amyloid induced inflammation; enhancing the clearance of amyloid from, for example, the brain; or protecting cells from amyloid induced (oligomers or fibrillar) toxicity.

In an embodiment, the compounds of the invention may be administered therapeutically or prophylactically to treat diseases associated with amyloid-P fibril formation, aggregation or deposition. The compounds of the invention may act to ameliorate the course of an amyloid-β related disease using any of the following mechanisms (this list is meant to be illustrative and not limiting): slowing the rate of amyloid-P fibril formation or deposition; lessening the degree of amyloid-β deposition; inhibiting, reducing, or preventing amyloid-β fibril formation; inhibiting neurodegeneration or cellular toxicity induced by amyloid-β; inhibiting amyloid-β induced inflammation; enhancing the clearance of amyloid-β from the brain; or favoring greater catabolism of Aβ.

Compounds of the invention may be effective in controlling amyloid-β deposition either following their entry into the brain (following penetration of the blood brain barrier) or from the periphery. When acting from the periphery, a compound may alter the equilibrium of Aβ between the brain and the plasma so as to favor the exit of Aβ from the brain. An increase in the exit of Aβ from the brain would result in a decrease in Aβ brain concentration and therefore favor a decrease in Aβ deposition. In addition, compounds that penetrate the brain may control deposition by acting directly on brain Aβ, e.g., by maintaining it in a non-fibrillar form or favoring its clearance from the brain. The compounds may slow down APP processing; may increase degradation of Aβ fibrils by macrophages or by neuronal cells; or may decrease Aβ production by activated microglia. These compounds could also prevent Aβ in the brain from interacting with the cell surface and therefore prevent neurotoxicity, neurodegeneration, or inflammation.

In one embodiment, the method is used to treat Alzheimer's disease (e.g., sporadic or familial AD). The method can also be used prophylactically or therapeutically to treat other clinical occurrences of amyloid-β deposition, such as in Down's syndrome individuals and in patients with cerebral amyloid angiopathy (“CAA”), hereditary cerebral hemorrhage, or early Alzheimer's disease.

In another embodiment, the method is used to treat mild cognitive impairment. Mild Cognitive Impairment (“MCI”) is a condition characterized by a state of mild but measurable impairment in thinking skills, which is not necessarily associated with the presence of dementia. MCI frequently, but not necessarily, precedes Alzheimer's disease.

Additionally, abnormal accumulation of APP and of amyloid-β protein in muscle fibers has been implicated in the pathology of sporadic inclusion body myositis (IBM) (Askanas, V., et al. (1996) Proc. Natl. Acad Sci. USA 93: 1314-1319; Askanas, V. et al. (1995) Current Opinion in Rheumatology 7: 486-496). Accordingly, the compounds of the invention can be used prophylactically or therapeutically in the treatment of disorders in which amyloid-beta protein is abnormally deposited at non-neurological locations, such as treatment of IBM by delivery of the compounds to muscle fibers.

Additionally, it has been shown that Aβ is associated with abnormal extracellular deposits, known as drusen, that accumulate along the basal surface of the retinal pigmented epithelium in individuals with age-related macular degeneration (ARMD). ARMD is a cause of irreversible vision loss in older individuals. It is believed that Aβ deposition could be an important component of the local inflammatory events that contribute to atrophy of the retinal pigmented epithelium, drusen biogenesis, and the pathogenesis of ARMD (Johnson, et al., Proc. Natl. Acad. Sci. USA 99(18), 11830-5 (2002)). Therefore, the invention also relates to the treatment or prevention of age-related macular degeneration.

In another embodiment, the invention also relates to a method of treating or preventing an amyloid associated disease in a subject (preferably a human) comprising administering to the subject a therapeutic amount of a compound according to the following Formulae or otherwise described herein, such that amyloid fibril formation or deposition, neurodegeneration, or cellular toxicity is reduced or inhibited. In another embodiment, the invention relates to a method of treating or preventing an amyloid associated disease in a subject (preferably a human) comprising administering to the subject a therapeutic amount of a compound according to the following Formulae or otherwise described herein, such that cognitive function is improved or stabilized or further deterioration in cognitive function is prevented, slowed, or stopped in patients with brain amyloidosis, e.g., Alzheimer's disease, Down's syndrome or cerebral amyloid angiopathy. These compounds can also improve quality of daily living in these subjects.

The therapeutic compounds of the invention may treat amyloidosis related to type II diabetes by, for example, stabilizing glycemia, preventing or reducing the loss of β cell mass, reducing or preventing hyperglycemia due to loss of β cell mass, and modulating (e.g, increasing or stabilizing) insulin production. The compounds of the invention may also stabilize the ratio of the concentrations of pro-IAPP/IAPP.

The therapeutic compounds of the invention may treat AA (secondary) amyloidosis and/or AL (primary) amyloidosis, by stabilizing renal function, decreasing proteinuria, increasing creatinine clearance (e.g., by at least 50% or greater or by at least 100% or greater), by leading to remission of chronic diarrhea or weight gain (e.g., 10% or greater), or by reducing serum creatinine. Visceral amyloid content as determined, e.g., by SAP scintigraphy may also be reduced.

Neuroprotection

The Aβ peptide has been shown by several groups to be highly toxic to neurons. Amyloid plaques are directly associated with reactive gliosis, dystrophic neurites and apoptotic cells, suggesting that plaques induce neurodegenerative changes. Neurotoxicity may eventually disrupt or even kill neurons. In vitro, Aβ has been shown to induce apoptosis in many different neuronal cell types, such as rat PC-12 cells, primary rat hippocampal and cortical cultures, and the predifferentiated human neurotype SH-SY5Y cell line (Dickson D W (2004) J Clin Invest 114:23-7; Canu et al. (2003) Cerebellum 2:270-278; Li et al. (1996) Brain Research 738:196-204). Numerous reports have shown that Aβ fibrils can induce neurodegeneration, and it has been shown that neuronal cells exposed in vitro to Aβ can become apoptotic (Morgan et al. (2004) Prog. Neurobiol. 74:323-349; Stefani et al. (2003) J. Mol. Med. 81:678-99; La Ferla et al. (1997) J. Clin. Invest. 100(2):310-320). In Alzheimer's disease, a progressive neuronal cell loss accompanies the deposition of Aβ amyloid fibrils in senile plaques.

In yet another aspect, the invention pertains to a method for inhibiting Aβ-induced neuronal cell death by administering an effective amount of a compound of the present invention.

Another aspect of the invention pertains to a method for providing neuroprotection to a subject having an Aβ-amyloid related disease, e.g. Alzheimer's disease, that includes administering an effective amount of a compound of the present invention to the subject, such that neuroprotection is provided.

In another aspect, methods for inhibiting Aβ-induced neuronal cell death are provided that include administration of an effective amount of a compound of the present invention to a subject such that neuronal cell death is inhibited.

In another aspect, methods for treating a disease state characterized by Aβ-induced neuronal cell death in a subject are provided, e.g., by administering an effective amount of a compound of the present invention. Non-limiting examples of such disease states include Alzheimer's disease and Aβ-amyloid related diseases.

The term “neuroprotection” includes protection of neuronal cells of a subject from Aβ-induced cell death, e.g., cell death induced directly or indirectly by an Aβ peptide. Aβ-induced cell death may result in initiation of processes such as, for example: the destabilization of the cytoskeleton; DNA fragmentation; the activation of hydrolytic enzymes, such as phospholipase A2; activation of caspases, calcium-activated proteases and/or calcium-activated endonucleases; inflammation mediated by macrophages; calcium influx into a cell; membrane potential changes in a cell; the disruption of cell junctions leading to decreased or absent cell-cell communication; and the activation of expression of genes involved in cell death, e.g., immediate-early genes.

Tau Assembly or Aggregation

In yet another aspect, the compounds and methods of the invention are administered to a subject to inhibit, prevent or reduce tau assembly or aggregation. Without wishing to be bound by any particular theory, it is believed that Aβ accumulation triggers a cascade which includes tau hyperphosphorylation leading to neurofibrillary tangle formation, and ultimately cell death. Oddo et al., Neuron 43(2):321-332,327 (2004); Hardy and Selko Science 297:353-356 (2002). Compounds effective at reducing, inhibiting or preventing Aβ aggregation may also be effective at reducing, inhibiting or preventing tau aggregation. Accordingly, not only can the compounds and the methods of the present invention be employed to treat amyloid related disorders (e.g., Aβ-related disorders such as Alzheimer's Disease, adult-onset diabetes, and age-related macular degeneration), but additionally or alternatively to treat tauopathies (e.g., Progressive Supernuclear Palsy (PSP), Corticobasal Degeneration (CBD), and frontotemporal dementia with Parkinsonism (FTDP) in a subject.

Accordingly, in one embodiment the invention provides a method of treating or preventing a tauopathy in a subject comprising administering a therapeutic amount of a compound of the invention such that the tauopathy is treated or prevented.

The compounds of the invention may be administered therapeutically or prophylactically to treat diseases associated with tau formation, aggregation or deposition. The compounds may act to inhibit, prevent and/or reverse tau aggregation by one or more of the following mechanisms: slowing the rate or preventing Aβ fibril formation or deposition; lessening the degree of Aβ deposition; inhibiting, reducing or preventing amyloid-β fibril formation; inhibiting neurodegeneration or cellular toxicity induced by amyloid-β or tau aggregates; inhibiting inflammation related to the presence of Aβ or tau; enhancing clearance of Aβ or tau from the brain or other organs; favoring the degradation of amyloid-O protein prior to its organization into fibrils; slowing the rate of tau formation or aggregation; inhibiting or reversing tau aggregation; inhibiting neuronal cell death; inhibiting, reducing or preventing neurofibrillary tangle, neuritic plaque, neuritic thread, globose tangle, or Pick Body formation; and inhibiting, reducing or preventing the formation or presence of thorny astrocytes, tufted astrocytes, astrocytic plaques, coiled bodies, glial threads, and microglia.

“Tau” or “tau protein” refers to the tau protein which is associated with the stabilization of microtubules in nerve cells and a component of a broad range of tau aggregates, e.g., neurofibrillary tangles. The term, unless otherwise indicated herein, refers to tau in all of its isoforms with or without modifications, including phosphorylation, truncation and conformation.

“Tauopathy” refers to tau-related disorders, e.g., tau-related neurodegenerative disorders, e.g., Alzheimer's Disease, Progressive Supranuclear Palsy (PSP), Corticobasal Degeneration (CBD), Pick's Disease, Frontotemporal dementia and Parkinsonism associated with chromosome 17 (FTDP-17).

“Tau aggregates” or “tau aggregation” refers to tau aggregates or aggregation associated with a broad range of disorders, primarily neurodegenerative disorders. Tau aggregates exist in many forms that include, but are not limited to, neurofibrillary tangles (pyramidal cells, or the extracellular remnants of such cells after degradation of the neuron, that include helical and straight filament pairs of aggregated tau), neuritic plaques (dystrophic neurites that contain a core of amyloid surrounded at least in part by tau aggregates typically in the straight filament form), neuritic threads (related to dystrophic neurites, but not organized in a plaque), globose tangles (accumulations of tau in neuronal cytoplasm associated with Progressive Supernuclear Palsy), and Pick Bodies (disordered tau fibrils associated with Pick's Disease that generally include tau protein as a major component and typically are found in neurons). Tau aggregates also include aggregates within cells, including: thorny astocytes (generally characterized by tau aggregates in and around the perinuclear cytoplasm found in subjects with PSP), tufted astrocytes (generally characterized by tau aggregates through grey matters cells and associated with PSP and AD), astrocytic plaques (plaques found in grey matter and associated with CBD), coiled bodies (generally characterized by comma-shaped or coiled structures that include tau filaments wrapped around the nucleus of an oligodendrocyte and found in subjects with FTDP-17, PSP and CBD), glial threads (generally characterized by tau inclusions in the myelin sheath of oligodendocytes found in subjects having PSP), and tau aggregates associated with microglia.

“Inhibition” of tau aggregation includes preventing or stopping of tau formation, clearance of tau, inhibiting or slowing down of tau deposition in a subject with tauopathy, and reducing or reversing neurofibrillary tangles or tau deposits in a subject. Inhibition of tau aggregation is determined relative to an untreated subject, or relative to the treated subject prior to treatment, or, e.g., determined by clinically measurable improvement, e.g., or in the case of a subject with brain amyloidosis, e.g., an Alzheimer's or cerebral amyloid angiopathy subject, stabilization of cognitive function or prevention of a further decrease in cognitive function (i.e., preventing, slowing, or stopping disease progression), or improvement of parameters such as the concentration of Aβ or tau in the CSF.

Blood-Brain Barrier

Regardless of the particular mechanism by which the compound exerts its biological effects, the compound prevents or treats amyloid associated diseases, such as for example Alzheimer's disease, CAA, MCI, diabetes related amyloidosis, AL amyloidosis, Down's syndrome, or β₂M amyloidosis. The compound may reverse or favor deposition of amyloid or the compound may favor plaque clearance or slow deposition. For example, the compound may decrease the amyloid concentration in the brain of a subject versus an untreated subject. The compound may penetrate into the brain by crossing the blood-brain barrier (“BBB”) to exert its biological effect. The compound may maintain soluble amyloid in a non-fibrillar form, or alternatively, the compound may increase the rate of clearance of soluble amyloid from the brain of a subject versus an untreated subject. The compound may also increase the rate of degradation of Aβ in the brain prior to organization into fibrils. A compound may also act in the periphery, causing a change in the equilibrium of the amyloid protein concentration in the two compartments (i.e., systemic vs. central), in which case a compound may not be required to penetrate the brain to decrease the concentration of Aβ in the brain (a “sink” effect).

Agents of the invention that exert their physiological effect in vivo in the brain may be more useful if they gain access to target cells in the brain. Non-limiting examples of brain cells are neurons, glial cells (astrocytes, oligodendrocytes, microglia), cerebrovascular cells (muscle cells, endothelial cells), and cells that comprise the meninges. The blood brain barrier (“BBB”) typically restricts access to brain cells by acting as a physical and functional blockade that separates the brain parenchyma from the systemic circulation (see, e.g., Pardridge, et al., J. Neurovirol. 5(6), 556-69 (1999); Rubin, et al., Rev. Neurosci. 22, 11-28 (1999)). Circulating molecules are generally able to gain access to brain cells via one of two processes: lipid-mediated transport through the BBB by free diffusion, or active (or catalyzed) transport.

The agents of the invention may be formulated to improve distribution in vivo, for example as powdered or liquid tablet or solution for oral administration or as a nasal spray, nose drops, a gel or ointment, through a tube or catheter, by syringe, by packtail, by pledget, or by submucosal infusion. Generally the blood-brain barrier (BBB) excludes many highly hydrophilic agents. To ensure that the more hydrophilic therapeutic agents of the invention cross the BBB, they may be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties” or “targeting groups” or “transporting vectors”), thus providing targeted drug delivery (see, e.g., V. V. Ranade J. Clin. Pharmacol. 29, 685 (1989)). Likewise, the agents may be linked to targeting groups that facilitate penetration of the blood brain barrier. In one embodiment, the method of the present invention employs a naturally occurring polyamine linked to an agent that is a small molecule and is useful for inhibiting e.g., Aβ deposition.

To facilitate transport of agents of the invention across the BBB, they may be coupled to a BBB transport vector (for review of BBB transport vectors and mechanisms, see Bickel, et al., Adv. Drug Delivery Reviews 46, 247-79 (2001)). Exemplary transport vectors include cationized albumin or the OX26 monoclonal antibody to the transferrin receptor; these proteins undergo absorptive-mediated and receptor-mediated transcytosis through the BBB, respectively. Natural cell metabolites that may be used as targeting groups include, inter alia, putrescine, spermidine, spermine, or DHA. Other exemplary targeting moieties include folate or biotin (see, e.g., U.S. Pat. No. 5,416,016); mannosides (Umezawa, et al., Biochem. Biophys. Res. Commun. 153, 1038 (1988)); antibodies (P. G. Bloeman, et al., FEBS Lett. 357, 140 (1995); M. Owais, et al., Antimicrob. Agents Chemother. 39, 180 (1995)); surfactant protein A receptor (Briscoe, et al., Am. J Physiol. 1233, 134 (1995)); gp120 (Schreier, et al., J. Biol. Chem. 269, 9090 (1994)); see also, K. Keinanen and M. L. Laukkanen, FEBS Lett. 346, 123 (1994); J. J. Killion and I. J. Fidler, Immunomethods 4, 273 (1994).

Examples of other BBB transport vectors that target receptor-mediated transport systems into the brain include factors such as insulin, insulin-like growth factors (“IGF-I,” and “IGF-II”), angiotensin II, atrial and brain natriuretic peptide (“ANP,” and “BNP”), interleukin I (“IL-1”) and transferrin. Monoclonal antibodies to the receptors that bind these factors may also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized immunoglobulins. Small basic oligopeptides such as the dynorphin analogue E-2078 and the ACTH analogue ebiratide may also cross the brain via absorptive-mediated transcytosis and are potential transport vectors.

Other BBB transport vectors target systems for transporting nutrients into the brain. Examples of such BBB transport vectors include hexose moieties, e.g., glucose and monocarboxylic acids, e.g., lactic acid and neutral amino acids, e.g., phenylalanine, and amines, e.g., choline and basic amino acids, e.g., arginine, nucleosides, e.g., adenosine and purine bases, e.g., adenine, and thyroid hormone, e.g., triiodothyridine. Antibodies to the extracellular domain of nutrient transporters may also be used as transport vectors. Other possible vectors include angiotensin II and ANP, which may be involved in regulating BBB permeability.

In some cases, the bond linking the therapeutic agent to the transport vector may be cleaved following transport into the brain in order to liberate the biologically active agent. Exemplary linkers or “linker groups” include disulfide bonds, an ether linkage, a thioether linkage, an alkylene or alkenylene linkage, an amino or a hydrozino linkage, ester-based linkages, thioether linkages, amide bonds, acid-labile linkages, and Schiff base linkages. Avidin/biotin linkers, in which avidin is covalently coupled to the BBB drug transport vector, may also be used. Avidin itself may be a drug transport vector.

Transcytosis, including receptor-mediated transport of compositions across the blood brain barrier, may also be suitable for the agents of the invention. Transferrin receptor-mediated delivery is disclosed in U.S. Pat. Nos. 5,672,683; 5,383,988; 5,527,527; 5,977,307; and 6,015,555. Transferrin-mediated transport is also known. P. M. Friden, et al., Pharmacol. Exp. Ther. 278, 1491-98 (1996); H. J. Lee, J. Pharmacol. Exp. Ther. 292, 1048-52 (2000). EGF receptor-mediated delivery is disclosed in Y. Deguchi, et al., Bioconjug. Chem. 10, 32-37 (1999), and transcytosis is described in A. Cerletti, et al., J. Drug Target. 8, 435-46 (2000). Insulin fragments have also been used as carriers for delivery across the blood brain barrier. M. Fukuta, et al., Pharm. Res. 11. 1681-88 (1994). Delivery of agents via a conjugate of neutral avidin and cationized human albumin has also been described. Y. S. Kang, et al., Pharm. Res. 1, 1257-64 (1994).

Nitric oxide is a vasodilator of the peripheral vasculature in normal tissue of the body. Increasing generation of nitric oxide by nitric oxide synthase causes vasodilation without loss of blood pressure. The blood-pressure-independent increase in blood flow through brain tissue increases cerebral bioavailability of blood-born compositions. This increase in nitric oxide may be stimulated by administering L-arginine. As nitric oxide is increased, -cerebral blood flow is consequently increased, and drugs in the blood stream are carried along with the increased flow into brain tissue. Therefore, L-arginine may be used in the pharmaceutical compositions of the invention to enhance delivery of agents to brain tissue before, after, or while introducing a pharmaceutical composition into the blood stream of the subject substantially contemporaneously with a blood flow enhancing amount of L-arginine, as described in WO 00/56328.

Other modifications in order to enhance penetration of the agents of the invention across the blood brain barrier may be accomplished using methods and derivatives known in the art.

BBB Amino Acid Transport Systems

One of the primary interfaces between the central nervous system and the peripheral circulation is the blood brain barrier (BBB). The BBB is composed of a monolayer of brain capillary endothelial cells that are fused together by tight junctions. The endothelial cells of the BBB contain membrane transport systems, such as the amino acid transport sytems, involved in the influx/efflux of compounds. Nine such amino acid transport systems have been identified which are present in the endothelium of the blood brain barrier. These systems include System y⁺, which transports amino acids with positively charged side chains and their analogs (i.e., basic amino acids and their analogs, e.g., arginine, lysine, and ornithine), System L1, which transports neutral amino acids and their analogs (e.g., phenylalanine, leucine, glycine, alanine, serine, cysteine, tryptophan, methionine, isoleucine, tyrosine, histidine, valine, threonine, proline, asparagine, and glutamine), and System X⁻, which transports amino acids with negatively charged amino acids and their analogs (i.e., acidic amino acids and their analogs, e.g., glutamic acid and aspartic acid). Blood brain barrier transport vectors, e.g., amino acids, need not function in the confines of the presently described systems. The skilled artisan would understand that the specific transporter system which carries the transport vector may be useful in designing the compounds of the invention, but does not limit the scope of the invention.

Large neutral amino acids (LNAAs) such as phenylalanine reach the brain by means of the transporters found in both membranes of endothelial cells. For LNAAs, net uptake through the BBB is determined by their ratio in plasma and their different affinity to the stereospecific L-type AA carrier system. System L mediates high affinity sodium-independent uptake of amino acids with large neutral side chains. System L at the BBB shares many characteristics with the L system transporter in other tissues, thus it has been proposed that the BBB system represents a different isoform, designated L1.

In one aspect, the present invention is directed to a bifunctional compound which includes a BBB transport vector and a moiety for the treatment of a CNS disease or an amyloid associated disease, or a pharmacologically acceptable salt thereof. In some embodiments, the BBB transport vector is an amino acid or an amino acid analog.

In some embodiments, the BBB transport vector is a basic amino acid or a basic amino acid analog, for example, arginine, lysine, ornithine, and/or analogs thereof. In other embodiments, the BBB transport vector is an acidic amino acid or an acidic acid analog, for example, aspartic acid, glutamic acid, and/or analogs thereof. In yet other embodiments, the BBB transport vector is a small neutral amino acid or a small neutral amino acid analog, for example, glycine, alanine, serine, cysteine, and/or analogs thereof. In still other embodiments, the BBB transport vector is a large neutral amino acid or a large neutral amino acid analog. Exemplary large neutral amino acids include phenylalanine, tryptophan, leucine, methionine, isoleucine, tyrosine, histidine, valine, threonine, proline, asparagine, glutamine, and/or analogs thereof.

In one embodiment, the amino acid or amino acid analog is substituted with the moiety for the treatment of a CNS disease or an amyloid associated disease at the nitrogen. In some embodiments, where the amino acid includes an aromatic side chain, the amino acid or amino acid analog is substituted on the aromatic side chain. In another embodiment, the amino acid or amino acid analog is substituted both at the nitrogen and on the aromatic side chain. In still further embodiments, the amino acid or amino acid analog is substituted at the oxygen.

In some embodiments, the substitution comprises a direct covalent bond to the amino acid or amino acid analog. In other embodiments, the substitution comprises a linker group, which connects the moiety for the treatment of a CNS disease or an amyloid associated disease to the amino acid or amino acid analog. In some embodiments, the linker groups is a disulfide bond, an ether linkage, a thioether linkage, an alkylene or alkenylene linkage, an amino or a hydrozino linkage, an ester-based linkage, a thioester linkage, an amide bond, an acid-labile linkage, or a Schiff base linkage.

Compounds of the Invention

The present invention relates, at least in part, to the use of certain chemical compounds (and pharmaceutical formulations thereof) in the prevention or treatment of CNS diseases and/or amyloid associated diseases, including, inter alia, Alzheimer's disease, cerebral amyloid angiopathy, inclusion body myositis, Down's syndrome, diabetes related amyloidosis, hemodialysis-related amyloidosis (β₂M), primary amyloidosis (e.g., λ or κ chain-related), familial amyloid polyneuropathy (FAP), senile systemic amyloidosis, familial amyloidosis, Ostertag-type non-neuropathic amyloidosis, cranial neuropathy, hereditary cerebral hemorrhage, familial dementia, chronic dialysis, familial Creutzfeldt-Jakob disease, Gerstmann-Straussler-Scheinker syndrome, hereditary spongiform encephalopathies, prion diseases, familial Mediterranean fever, Muckle-Well's syndrome, nephropathy, deafness, urticaria, limb pain, cardiomyopathy, cutaneous deposits, multiple myeloma, benign monoclonal gammopathy, maccoglobulinaemia, myeloma associated amyloidosis, medullary carcinomas of the thyroid, and isolated atrial amyloid, seizure, neuropathic pain, Abercrombie s degeneration, Acquired epileptiform aphasia, Landau-Kleffner Syndrome, Acute Disseminated Encephalomyelitis, Adrenoleukodystrophy, Leukodystrophy, Agnosia, Alexander Disease, Alpers Disease, Progressive Sclerosing Poliodystrophy, Alternating Hemiplegia, Amyotrophic Lateral Sclerosis, Lou Gehrig's disease, Angelman Syndrome, Ataxia Telangiectasia, Ataxias and Cerebellar/Spinocerebellar Degeneration, Attention Deficit Disorder, Binswanger's Disease, subcortical dementia, Canavan Disease, Cerebral Hypoxia, Cerebro-Oculo-Facio-Skeletal Syndrome, Pena Shokeir II Syndrome, Charcot-Marie-Tooth, Chronic Inflammatory Demyelinating Polyneuropathy (CIDP), Corticobasal Degeneration, Degenerative knee arthritis, Diabetic neuropathy, Early Infantile Epileptic Encephalopathy, Ohtahara Syndrome, Epilepsy, Friedreich's Ataxia, Guillain-Barre Syndrome (GBS), Acute Idiopathic Polyneuritis, Hallervorden-Spatz Disease, Neurodegeneration with Brain Iron Accumulation, Huntington s Disease, Krabbe Disease, Kugelberg-Welander Disease, Spinal Muscular Atrophy (SMA), SMA type I, SMA type II, SMA type III, Kennedy syndrome, progressive spinobulbar muscular atrophy, Congenital SMA with arthrogryposis, Adult SMA, Leigh's Disease, Lennox-Gastaut Syndrome, Machado-Joseph Disease, spinocerebellar ataxia type 3, Monomelic Amyotrophy, Multiple Sclerosis, Neuroacanthocytosis, Niemann-Pick disease, Olivopontocerebellar Atrophy, Paraneoplastic Syndromes, Neurologic paraneoplastic syndromes, Lambert-Eaton myasthenic syndrome, stiff-person syndrome, encephalomyelitis, myasthenia gravis, cerebellar degeneration, limbic and/or brainstem encephalitis, neuromyotonia, opsoclonus and sensory neuropathy, Parkinson s Disease, Pelizaeus-Merzbacher Disease, Pick's Disease, Primary Lateral Sclerosis, Progressive Locomotor Ataxia, Syphilitic Spinal Sclerosis, Tabes Dorsalis, Progressive Supranuclear Palsy, Rasmussen's Encephalitis, Rett Syndrome, Tourette's Syndrome, Usher syndrome, West syndrome, Infantile Spasms, Wilson Disease, and/or hepatolenticular degeneration.

The chemical structures herein are drawn according to the conventional standards known in the art. Thus, where an atom, such as a carbon atom, as drawn appears to have an unsatisfied valency, then that valency is assumed to be satisfied by a hydrogen atom even though that hydrogen atom is not necessarily explicitly drawn. The structures of some of the compounds of this invention include stereogenic carbon atoms. It is to be understood that isomers arising from such asymmetry (e.g., all enantiomers and diastereomers) are included within the scope of this invention unless indicated otherwise. That is, unless otherwise stipulated, any chiral carbon center may be of either (R)- or (S)-stereochemistry. Such isomers can be obtained in substantially pure form by classical separation techniques and by stereochemically-controlled synthesis. Furthermore, alkenes can include either the E- or Z-geometry, where appropriate. In addition, the compounds of the present invention may exist in unsolvated as well as solvated forms with acceptable solvents such as water, THF, ethanol, and the like. In general, the solvated forms are considered equivalent to the unsolvated forms for the purposes of the present invention.

A “small molecule” refers to a compound that is not itself the product of gene transcription or translation (e.g., protein, RNA, or DNA) and preferably has a low molecular weight, e.g., less than about 2500 amu.

The terms “moiety” and “group,” as used herein, are used interchangeably to mean, in their broadest sense, a portion of a compound, such as a substituent in an organic compound or a radical of a molecule that is attached to another moiety. As a nonlimiting example, an amino acid moiety may be any natural or synthetic amino acid as defined herein, which is covalently bonded, e.g., through the nitrogen, to another organic moiety. Examples of moieties are known to those skilled in the art and are intended to be included within the meaning of the term so long as they fall within the scope of the compounds defined herein.

As used herein, the term “compound” is intended to mean a substance made up of molecules that further consist of atoms. A compound may be any natural or non-natural material, for example, peptide or polypeptide sequences, organic or inorganic molecules or compositions, nucleic acid molecules, carbohydrates, lipids or combinations thereof. A compound generally refers to a chemical entity, whether in the solid, liquid or gaseous phase, and whether in a crude mixture or purified and isolated. Compounds encompass the chemical compound itself as well as, where applicable: amorphous and crystalline forms of the compound, including polymorphic forms, said forms in mixture or in isolation; free acid and free base forms of the compound; isomers of the compound, including geometric isomers, optical isomers, and tautomeric isomers, said optical isomers to include enantiomers and diastereomers, chiral isomers and non-chiral isomers, said optical isomers to include isolated optical isomers or mixtures of optical isomers including racemic and non-racemic mixtures; said geometric isomers to include transoid and cisoid forms, where an isomer may be in isolated form or in admixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, including acid addition salts and base addition salts, including organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different.

As used herein, “alkyl” groups include saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (isopropyl, tert-butyl, sec-butyl, isobutyl, etc.), and alkyl-substituted alkyl groups (e.g., alkyl-substituted cycloalkyl groups and cycloalkyl-substituted alkyl groups). The term “aliphatic group” includes organic moieties characterized by straight or branched-chains, typically having between 1 and 22 carbon atoms. In complex structures, the chains may be branched, bridged, or cross-linked. Aliphatic groups include alkyl groups, alkenyl groups, and alkynyl groups.

In certain embodiments, a straight-chain or branched-chain alkyl group may have 30 or fewer carbon atoms in its backbone, e.g., C₁-C₃₀ for straight-chain or C₃-C₃₀ for branched-chain. In certain embodiments, a straight-chain or branched-chain alkyl group may have 20 or fewer carbon atoms in its backbone, e.g., C₁-C₂₀ for straight-chain or C₃-C₂₀ for branched-chain, and more preferably 18 or fewer. Likewise, preferred cycloalkyl groups have from 4-10 carbon atoms in their ring structure, and more preferably have 4-7 carbon atoms in the ring structure. The term “lower alkyl” refers to alkyl groups having from 1 to 6 carbons in the chain, and to cycloalkyl groups having from 3 to 6 carbons in the ring structure.

Unless the number of carbons is otherwise specified, “lower” as in “lower aliphatic,” “lower alkyl,” “lower alkenyl,” etc. as used herein means that the moiety has at least one and less than about 8 carbon atoms. In certain embodiments, a straight-chain or branched-chain lower alkyl group has 6 or fewer carbon atoms in its backbone (e.g., C₁-C₆ for straight-chain, C₃-C₆ for branched-chain), and more preferably 4 or fewer. Likewise, preferred cycloalkyl groups have from 3-8 carbon atoms in their ring structure, and more preferably have 5 or 6 carbons in the ring structure. The term “C₁-C₆” as in “C₁-C₆ alkyl” means alkyl groups containing 1 to 6 carbon atoms.

Moreover, unless otherwise specified the term alkyl includes both “unsubstituted alkyls” and “substituted alkyls,” the latter of which refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulflbydryl, alkylthio, arylthio, thiocarboxylate, sulfates, alkylsulfinyl, sulfonato, sulfamoyl, sulfonamido, nitro, trifluoromethyl, cyano, azido, heterocyclic, alkylaryl, or aromatic (including heteroaromatic) groups.

An “arylalkyl” group is an alkyl group substituted with an aryl group (e.g., phenylmethyl (i.e., benzyl)). An “alkylaryl” moiety is an aryl group substituted with an alkyl group (e.g.,p-methylphenyl (i.e.,p-tolyl)). The term “n-alkyl” means a straight-chain (i.e., unbranched) unsubstituted alkyl group. An “alkylene” group is a divalent analog of the corresponding alkyl group. The terms “alkenyl” and “alkynyl” refer to unsaturated aliphatic groups analogous to alkyls, but which contain at least one double or triple carbon-carbon bond respectively. Suitable alkenyl and alkynyl groups include groups having 2 to about 12 carbon atoms, preferably from 2 to about 6 carbon atoms.

The term “aromatic group” or “aryl group” includes unsaturated and aromatic cyclic hydrocarbons as well as unsaturated and aromatic heterocycles containing one or more rings. Aryl groups may also be fused or bridged with alicyclic or heterocyclic rings that are not aromatic so as to form a polycycle (e.g., tetralin). An “arylene” group is a divalent analog of an aryl group. Aryl groups can also be fused or bridged with alicyclic or heterocyclic rings which are not aromatic so as to form a polycycle (e.g., tetralin).

The term “heterocyclic group” includes closed ring structures analogous to carbocyclic groups in which one or more of the carbon atoms in the ring is an element other than carbon, for example, nitrogen, sulfur, or oxygen. Heterocyclic groups may be saturated or unsaturated. Additionally, heterocyclic groups (such as pyrrolyl, pyridyl, isoquinolyl, quinolyl, purinyl, and furyl) may have aromatic character, in which case they may be referred to as “heteroaryl” or “heteroaromatic” groups.

Unless otherwise stipulated, aryl and heterocyclic (including heteroaryl) groups may also be substituted at one or more constituent atoms. Examples of heteroaromatic and heteroalicyclic groups may have 1 to 3 separate or fused rings with 3 to about 8 members per ring and one or more N, O, or S heteroatoms. In general, the term “heteroatom” includes atoms of any element other than carbon or hydrogen, preferred examples of which include nitrogen, oxygen, sulfur, and phosphorus. Heterocyclic groups may be saturated or unsaturated or aromatic.

Examples of heterocycles include, but are not limited to, acridinyl; azocinyl; benzimidazolyl; benzofuranyl; berizothiofuranyl; benzothiophenyl; benzoxazolyl; benzthiazolyl; benztriazolyl; benztetrazolyl; benzisoxazolyl; benzisothiazolyl; benzimidazolinyl; carbazolyl; 4aH-carbazolyl; carbolinyl; chromanyl; chromenyl; cinnolinyl; decahydroquinolinyl; 2H,6H-1,5,2-dithiazinyl; dihydrofuro[2,3-b]tetrahydrofuran; furanyl; furazanyl; imidazolidinyl; imidazolinyl; imidazolyl; 1H-indazolyl; indolenyl; indolinyl; indolizinyl; indolyl; 3H-indolyl; isobenzofuranyl; isochromanyl; isoindazolyl; isoindolinyl; isoindolyl; isoquinolinyl; isothiazolyl; isoxazolyl; methylenedioxyphenyl; morpholinyl; naphthyridinyl; octahydroisoquinolinyl; oxadiazolyl; 1,2,3-oxadiazolyl; 1,2,4-oxadiazolyl; 1,2,5-oxadiazolyl; 1,3,4-oxadiazolyl; oxazolidinyl; oxazolyl; oxazolidinyl; pyrimidinyl; phenanthridinyl; phenanthrolinyl; phenazinyl; phenothiazinyl; phenoxathiinyl; phenoxazinyl; phthalazinyl; piperazinyl; piperidinyl; piperidonyl; 4-piperidonyl; piperonyl; pteridinyl; purinyl; pyranyl; pyrazinyl; pyrazolidinyl; pyrazolinyl; pyrazolyl; pyridazinyl; pyridooxazole; pyridoimidazole; pyridothiazole; pyridinyl; pyridyl; pyrimidinyl; pyrrolidinyl; pyrrolinyl; 2H-pyrrolyl; pyrrolyl; quinazolinyl; quinolinyl; 4H-quinolizinyl; quinoxalinyl; quinuclidinyl; tetrahydrofuranyl; tetrahydroisoquinolinyl; tetrahydroquinolinyl; tetrazolyl; 6H-1,2,5-thiadiazinyl; 1,2,3-thiadiazolyl; 1,2,4-thiadiazolyl; 1,2,5-thiadiazolyl; 1,3,4-thiadiazolyl; thianthrenyl; thiazolyl; thienyl; thienothiazolyl; thienooxazolyl; thienoimidazolyl; thiophenyl; triazinyl; 1,2,3-triazolyl; 1,2,4-triazolyl; 1,2,5-triazolyl; 1,3,4-triazolyl; and xanthenyl. Preferred heterocycles include, but are not limited to, pyridinyl; furanyl; thienyl; pyrrolyl; pyrazolyl; pyrrolidinyl; imidazolyl; indolyl; benzimidazolyl; 1H-indazolyl; oxazolidinyl; benzotriazolyl; benzisoxazolyl; oxindolyl; benzoxazolinyl; and isatinoyl groups. Also included are fused ring and spiro compounds containing, for example, the above heterocycles.

A common hydrocarbon aryl group is a phenyl group having one ring. Two-ring hydrocarbon aryl groups include naphthyl, indenyl, benzocyclooctenyl, benzocycloheptenyl, pentalenyl, and azulenyl groups, as well as the partially hydrogenated analogs thereof such as indanyl and tetrahydronaphthyl. Exemplary three-ring hydrocarbon aryl groups include acephthylenyl, fluorenyl, phenalenyl, phenanthrenyl, and anthracenyl groups.

Aryl groups also include heteromonocyclic aryl groups, i.e., single-ring heteroaryl groups, such as thienyl, furyl, pyranyl, pyrrolyl, imidazolyl, pyrazolyl, pyridinyl, pyrazinyl, pyrimidinyl, and pyridazinyl groups; and oxidized analogs thereof such as pyridonyl, oxazolonyl, pyrazolonyl, isoxazolonyl, and thiazolonyl groups. The corresponding hydrogenated (i.e., non-aromatic) heteromonocylic groups include pyrrolidinyl, pyrrolinyl, imidazolidinyl, imidazolinyl, pyrazolidinyl, pyrazolinyl, piperidyl and piperidino, piperazinyl, and morpholino and morpholinyl groups.

Aryl groups also include fused two-ring heteroaryls such as indolyl, isoindolyl, indolizinyl, indazolyl, quinolinyl, isoquinolinyl, phthalazinyl, quinoxalinyl, quinazolinyl, cinnolinyl, chromenyl, isochromenyl, benzothienyl, benzimidazolyl, benzothiazolyl, purinyl, quinolizinyl, isoquinolonyl, quinolonyl, naphthyridinyl, and pteridinyl groups, as well as the partially hydrogenated analogs such as chromanyl, isochromanyl, indolinyl, isoindolinyl, and tetrahydroindolyl groups. Aryl groups also include fused three-ring groups such as phenoxathiinyl, carbazolyl, phenanthridinyl, acridinyl, perimidinyl, phenanthrolinyl, phenazinyl, phenothiazinyl, phenoxazinyl, and dibenzofuranyl groups.

Some typical aryl groups include substituted or unsubstituted 5- and 6-membered single-ring groups. In another aspect, each Ar group may be selected from the group consisting of substituted or unsubstituted phenyl, pyrrolyl, furyl, thienyl, thiazolyl, isothiaozolyl, imidazolyl, triazolyl, tetrazolyl, pyrazolyl, oxazolyl, isooxazolyl, pyridinyl, pyrazinyl, pyridazinyl, and pyrimidinyl groups. Further examples include substituted or unsubstituted phenyl, 1-naphthyl, 2-naphthyl, biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl groups.

The term “amine” or “amino,” as used herein, refers to an unsubstituted or substituted moiety of the formula —NR^{a}R^{b}, in which R^{a} and R^{b} are each independently hydrogen, alkyl, aryl, or heterocyclyl, or R^{a} and R^{b}, taken together with the nitrogen atom to which they are attached, form a cyclic moiety having from 3 to 8 atoms in the ring. Thus, the term amino includes cyclic amino moieties such as piperidinyl or pyrrolidinyl groups, unless otherwise stated. Thus, the term “alkylamino” as used herein means an alkyl group having an amino group attached thereto. Suitable alkylamino groups include groups having 1 to about 12 carbon atoms, preferably from 1 to about 6 carbon atoms. The term amino includes compounds or moieties in which a nitrogen atom is covalently bonded to at least one carbon or heteroatom. The term “dialkylamino” includes groups wherein the nitrogen atom is bound to at least two alkyl groups. The term “arylamino” and “diarylamino” include groups wherein the nitrogen is bound to at least one or two aryl groups, respectively. The term “alkylarylamino” refers to an amino group which is bound to at least one alkyl group and at least one aryl group. The term “alkaminoalkyl” refers to an alkyl, alkenyl, or alkynyl group substituted with an alkylamino group. The term “amide” or “aminocarbonyl” includes compounds or moieties which contain a nitrogen atom which is bound to the carbon of a carbonyl or a thiocarbonyl group.

The term “alkylthio” refers to an alkyl group, having a sulfhydryl group attached thereto. Suitable alkylthio groups include groups having 1 to about 12 carbon atoms, preferably from 1 to about 6 carbon atoms.

The term “alkylcarboxyl” as used herein means an alkyl group having a carboxyl group attached thereto.

The term “alkoxy” as used herein means an alkyl group having an oxygen atom attached thereto. Representative alkoxy groups include groups having 1 to about 12 carbon atoms, preferably 1 to about 6 carbon atoms, e.g., methoxy, ethoxy, propoxy, tert-butoxy and the like. Examples of alkoxy groups include methoxy, ethoxy, isopropyloxy, propoxy, butoxy, and pentoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc., as well as perhalogenated alkyloxy groups.

The term “acylamino” includes moieties wherein an amino moiety is bonded to an acyl group. For example, the acylamino group includes alkylcarbonylamino, arylcarbonylamino, carbamoyl and ureido groups.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone.

The term “carbonyl” or “carboxy” includes compounds and moieties which contain a carbon connected with a double bond to an oxygen atom. Examples of moieties which contain a carbonyl include aldehydes, ketones, carboxylic acids, amides, esters, anhydrides, etc.

The term “ether” or “ethereal” includes compounds or moieties which contain an oxygen bonded to two carbon atoms. For example, an ether or ethereal group includes “alkoxyalkyl” which refers to an alkyl, alkenyl, or alkynyl group substituted with an alkoxy group.

A “sulfonate” group is a —SO₃H or —SO₃⁻X⁺ group bonded to a carbon atom, where X⁺ is a cationic counter ion group. Similarly, a “sulfonic acid” compound has a —SO₃H or —SO₃⁻X⁺ group bonded to a carbon atom, where X+ is a cationic group. A “sulfate” as used herein is a —OSO₃H or —OSO₃⁻X⁺ group bonded to a carbon atom, and a “sulfuric acid” compound has a —SO₃H or —OSO₃⁻X⁺ group bonded to a carbon atom, where X⁺ is a cationic group. According to the invention, a suitable cationic group may be a hydrogen atom. In certain cases, the cationic group may actually be another group on the therapeutic compound that is positively charged at physiological pH, for example an amino group.

A “counter ion” is required to maintain electroneutrality. Examples of anionic counter ions include halide, triflate, sulfate, nitrate, hydroxide, carbonate, bicarbonate, acetate, phosphate, oxalate, cyanide, alkylcarboxylate, N-hydroxysuccinimide, N-hydroxybenzotriazole, alkoxide, thioalkoxide, alkane sulfonyloxy, halogenated alkane sulfonyloxy, arylsulfonyloxy, bisulfate, oxalate, valerate, oleate, palmitate, stearate, laurate, borate, benzoate, lactate, citrate, maleate, fumarate, succinate, tartrate, naphthylate mesylate, glucoheptonate, or lactobionate. Compounds containing a cationic group covalently bonded to an anionic group may be referred to as an “internal salt.”

The term “nitro” means —NO₂; the term “halogen” or “halogeno” or “halo” designates —F, —Cl, —Br or —I; the term “thiol,” “thio,” or “mercapto” means SH; and the term “hydroxyl” or “hydroxy” means —OH.

The term “acyl” refers to a carbonyl group that is attached through its carbon atom to a hydrogen (i.e., a formyl), an aliphatic group (e.g., acetyl), an aromatic group (e.g., benzoyl), and the like. That is, acyl refers to a group desived from a carboxylic acid (RC(O)OH) with the following general formula: R—C(O)—, wherein R is a alkyl or aryl as defined herein. When R is an alkyl group, the “acyl” is equivalent to “alkylcarbonyl”; when R is an aryl group, the “acyl” is equivalent to “arylcarbonyl”. The term “substituted acyl” includes acyl groups where one or more of the hydrogen atoms on one or more carbon atoms are replaced by, for example, an alkyl group, alkynyl group, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used in the description and drawings herein, an optional single/double bond is represented by a solid line together with a dashed line, and refers to a covalent linkage between two carbon atoms which can be either a single bond or a double bond. For example, the structure: 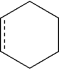
can represent either cyclohexane or cyclohexene.

Unless otherwise specified, the chemical moieties of the compounds of the invention, including those groups discussed above, may be “substituted or unsubstituted.” In some embodiments, the term “substituted” means that the moiety has substituents placed on the moiety other than hydrogen (i.e., in most cases, replacing a hydrogen), which allow the molecule to perform its intended function. Examples of substituents include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g, phenyloxyalkyl), arylacetamidoyl, alkylaryl, heteroaralkyl, alkylcarbonyl and arylcarbonyl or other such acyl group, heteroarylcarbonyl, and heteroaryl groups, as well as (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), and (CR′R″)₀₋₃OR′ groups, wherein R′ and R″ are each independently hydrogen, a C₁-C₅ alkyl, C₂-C₅ alkenyl, C₂-C₅ alkynyl, or aryl group; or the side chain of any naturally occurring amino acid.

In another embodiment, a substituent may be selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g, benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₁₀NR′R″ (e.g., —NH₂), (CR′R″)₀₋₁₀CN (e.g., —CN), NO₂, halogen (e.g., F, Cl, Br, or I), (CR′R″)₀₋₁₀C(halogen)₃ (e.g., —CF₃), (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′″, (CR′R″)₀₋₁₀CHO, (CR′R″)₀₋₁₀O(CR′R″)₀₋₁₀H, (CR′R″)₀₋₁₀S(O)₀₋₃R′ (e.g., —SO₃H), (CR′R″)₀₋₁₀O(CR′R″)₀₋₁₀H (e.g., —CH₂OCH₃ and —OCH₃), (CR′R″)₀₋₁₀S(CR′R″)₀₋₃H (e.g., —SH and —SCH₃), (CR′R″)₀₋₁₀OH (e.g., —OH), (CR′R″)₀₋₁₀COR′, (CR′R″)₀₋₁₀(substituted or unsubstituted phenyl), (CR′R″)₀₋₁₀O(C₃-C₈ cycloalkyl), (CR′R″)₀₋₁₀CO₂R′ (e.g., —CO₂H), or (CR′R″)₀₋₁₀OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more.

In some embodiments, a “substituent” may be selected from the group consisting of, for example, halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl (including heteroaryl) groups.

It will also be inderstood that the term “analog” refers to a chemical compound that is structurally related to the parent compound and retains at least a measurable amount of its activity. That is, an analog may be a compound or composition that varies from an original or primary compound or composition by the presence of one or more chemical additions, deletions, substituents, or substitutions as described above, which are not present in the structure of the primary compound or composition. An analog as used herein may generally have at least 10%, 20%, 30%, 40%, or 50% of the activity of the primary compound or composition, and preferably more, up to and exceeding 100% of the activity of the primary compound or composition. An analog may have physical or functional characteristics that differ from those of the primary compound or composition, for example, different or enhanced solubility, membrane permeability, or biological half-life, while retaining anti-viral or anti-tumor activity. The term “analog” also refers to a different enantiomeric form of a given compound, such as the dextrorotatory or levorotatory form of a molecule or a compound made using one or more enantiomeric forms of a given constituent. An analog may have, for example, a modification in one or more of the rings, and/or one or more of its substitutes, alone or in combination. Analogs include double-bond isomers, reduction products, side-chain modifications and stereoisomers of any of the preceding molecules. The term analog refers to any substance which has substantially similar compositional and/or functional characteristics, preferably both substantially similar compositional and functional characteristics, as does the substance for which it is an analog. Analogs may be naturally occurring or synthetically produced. Additionally the term analog may include compounds where one or more atoms have been substituted with a different, preferably isoelectronic, atom.

The term “amino acid” refers to any compound containing both an amino group and a carboxylic acid group. Although the amino group most commonly occurs at the position adjacent to the carboxy function, the amino group may be positioned at any location within the molecule. The amino acid may also contain additional functional groups, such as amino, thio, carboxyl, carboxamide, imidazole, etc. An amino acid may be synthetic or naturally occurring, and may be used in either its racemic or optically active (D-, or L-) forms, including various ratios of stereoisomers.

In one embodiment, the present invention is directed to compounds of Formula I:
A-Y-Q

wherein:

Q is a BBB transport vector;

Y is a direct bond or a linker group;

A is hydrogen, alkyl, alkyloxy, alkenyl, alkenyloxy, alkynyl, alkynyloxy, carbocyclic, heterocyclic, bicyclic, aryl, heteroaryl, fused-ring aryl or heteroaryl, aryloxy, arylalkyl, arylalkyloxy, alkylcarbonyl, arylcarbonyl, alkoxycarbonyl, thiazolyl, triazolyl, imidazolyl, benzothiazolyl, benzoimidazolyl, 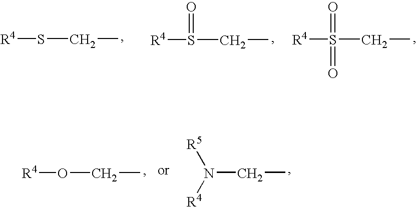
each of which may be optionally substituted; and

R⁴ and R⁵ together with the nitrogen form a 5 or 6 membered heterocyclic ring, or are each independently selected from the group consisting of hydrogen, alkyl, alkyloxy, alkenyl, alkenyloxy, alkynyl, alkynyloxy, cycloalkyl, aryl, aryloxy, arylalkyl, arylalkyloxy, alkylcarbonyl, arylcarbonyl, alkoxycarbonyl, thiazolyl, triazolyl, imidazolyl, benzothiazolyl, and benzoimidazolyl, each of which may be optionally substituted;

or a pharmaceutically acceptable salt, ester or prodrug thereof.

In some embodiments, Q is a 5 or 6 membered aromatic or heteroaromatic moiety, which may be further substituted. In other embodiments, Q is an amino acid moiety or analog thereof. Q may be a basic amino acid moiety or analog thereof, e.g., arginine, lysine, ornithine, and/or analogs thereof. Q may also be an acidic amino acid moiety or analog thereof, e.g., aspartic acid, glutamic acid, and/or analogs thereof. Furthermore, Q may be a small neutral amino acid moiety or analog thereof, e.g., glycine, alanine, serine, cysteine, and/or analogs thereof. Q may also be a large neutral amino acid moiety or analog thereof, e.g., phenylalanine, tryptophan, leucine, methionine, isoleucine, tyrosine, histidine, valine, threonine, proline, asparagine, glutamine, and/or analogs thereof. In other embodiments, the linker group is a disulfide bond, an ether linkage, a thioether linkage, an alkylene or alkenylene linkage, an amino or a hydrozino linkage, an ester-based linkage, a thioester linkage, an amide bond, an acid-labile linkage, or a Schiff base linkage.

In another embodiment, the present invention is directed to compounds of Formula II:

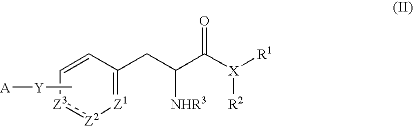

wherein:

X is oxygen, nitrogen, or sulfur;

Y is a direct bond or a linker group;

Z¹, Z², Z³ are each independently C, CH, CH₂, P, N, NH, S, or absent;

R¹ and R² are independently absent, hydrogen, alkyl, cycloalkyl, alkenyl, alkylnyl, aryl, arylalkyl, or acyl, each of which may be optionally substituted;

R³ is selected from the group consisting of hydrogen, alkyl, aryl, amido, arylamido, alkylcarbonyl, arylcarbonyl, arylaminocarbonyl, alkoxycarbonyl, alkanesulfonyl, arenesulfonyl, cycloalkanesulfonyl, and heteroarenesulfonyl, each of which may be optionally substituted;

A is hydrogen, alkyl, alkyloxy, alkenyl, alkenyloxy, alkynyl, alkynyloxy, carbocyclic, heterocyclic, bicyclic, aryl, heteroaryl, fused-ring aryl or heteroaryl, aryloxy, arylalkyl, arylalkyloxy, alkylcarbonyl, arylcarbonyl, alkoxycarbonyl, thiazolyl, triazolyl, imidazolyl, benzothiazolyl, benzoimidazolyl, 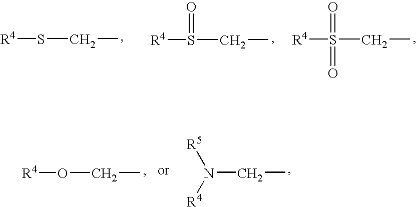
each of which may be optionally substituted; and

R⁴ and R⁵ together with the nitrogen form a 5 or 6 membered heterocyclic ring, or are each independently hydrogen, alkyl, alkyloxy, alkenyl, alkenyloxy, alkynyl, alkynyloxy, cycloalkyl, aryl, aryloxy, arylalkyl, arylalkyloxy, alkylcarbonyl, arylcarbonyl, alkoxycarbonyl, thiazolyl, triazolyl, imidazolyl, benzothiazolyl, or benzoimidazolyl, each of which may be optionally substituted;

or a pharmaceutically acceptable salt, ester or prodrug thereof.

In one embodiment, X is oxygen or nitrogen. In another embodiment, Y is a direct bond. In yet another embodiment, Z¹, Z² and Z³ are N, C or CH. In still another embodiment, R¹ and R² are independently absent or hydrogen. In another embodiment, R³ is hydrogen, arylamido, arylaminocarbonyl or arenesulfonyl, each of which may be optionally substituted. In yet another embodiment, A is one of the following groups: R⁴—S—CH₂, 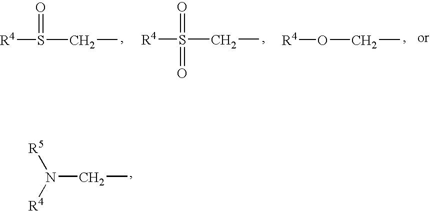
each of which may be optionally substituted.

In still another embodiment, R⁴ and R⁵ are each independently cycloalkyl, aryl, aryloxy, arylalkyl, arylalkyloxy, alkylcarbonyl, arylcarbonyl, alkoxycarbonyl, thiazolyl, triazolyl, imidazolyl, benzothiazolyl, or benzoimidazolyl, each of which may be optionally substituted. In some embodiments, R⁴ and R⁵ are each independently pyridine, pyrimidine, pyrimidinone, tetrahydropyridine, piperidine, piperazine, imidazole, benzoimidazole, oxazole, oxadiazole, benzooxazole, triazole, thiazole, benzothiazole, tetrazole, thiadiazole, pyrazolopyrimidine, isoquinoline, or tetrahydroisoquinoline, each of which may be optionally substituted. In another embodiment, R⁴ and R⁵ together with the nitrogen form a 6 membered ring optionally interrupted with one or more additional heteroatoms. In some embodiments the resultant 6 membered ring is non-fused ring. In other embodiments, the linker group is a disulfide bond, an ether linkage, a thioether linkage, an alkylene or alkenylene linkage, an amino or a hydrozino linkage, an ester-based linkage, a thioester linkage, an amide bond, an acid-labile linkage, or a Schiff base linkage.

In a further embodiment, the compound is at least one compound selected from the compounds of Table 1 and pharmaceutically acceptable salts, esters, and prodrugs thereof.

TABLE 1
Exemplary Compounds of the present invention.

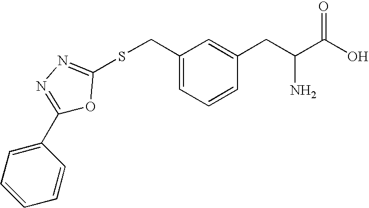

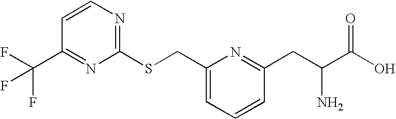

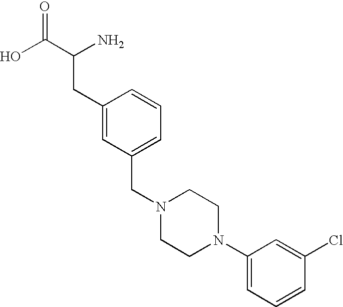

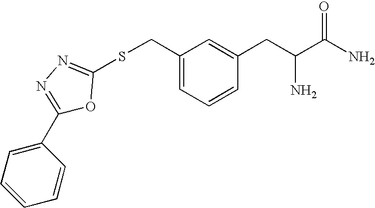

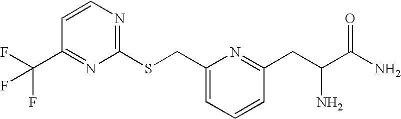

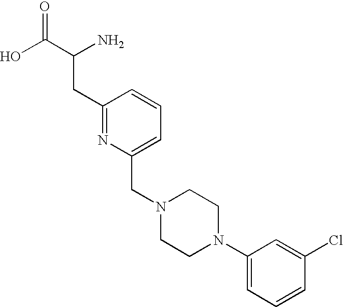

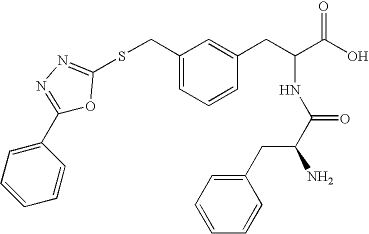

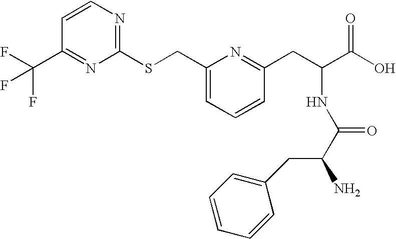

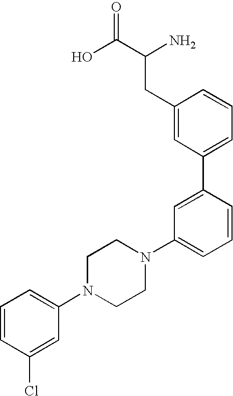

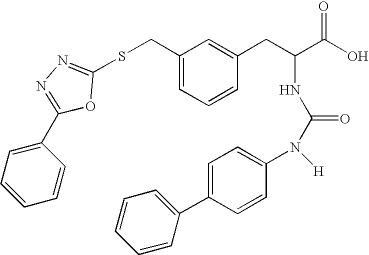

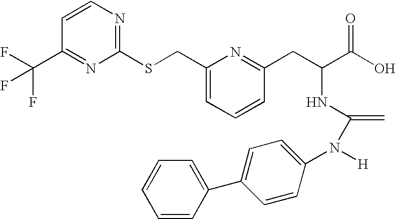

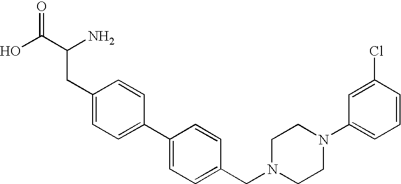

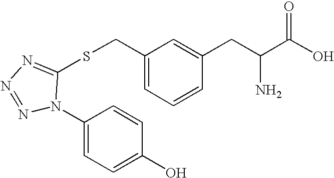

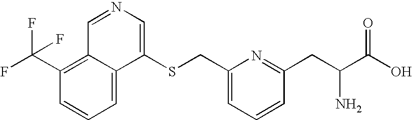

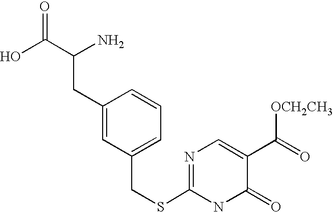

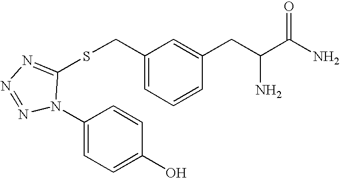

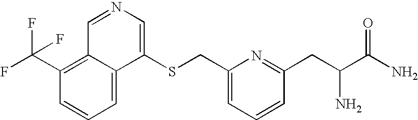

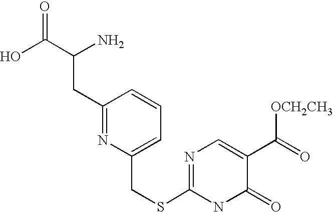

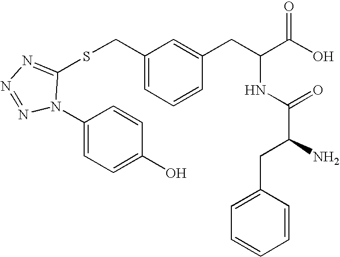

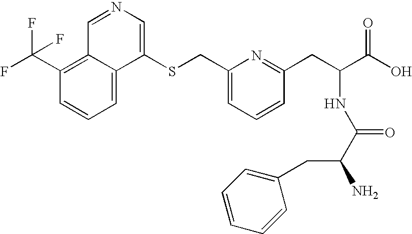

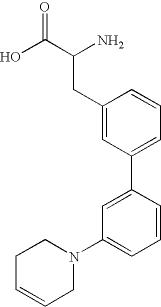

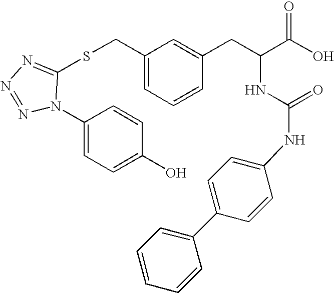

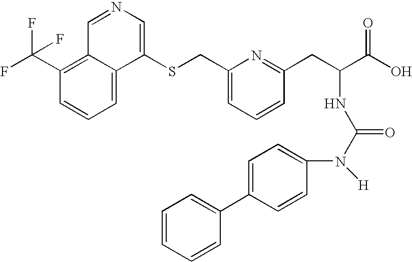

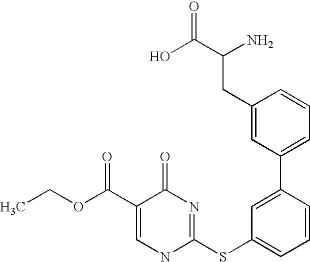

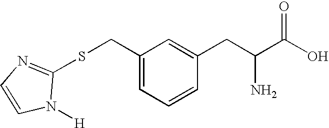

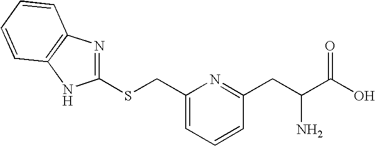

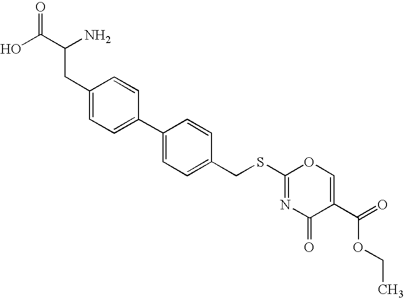

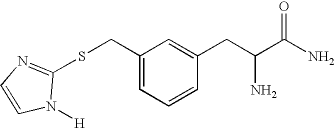

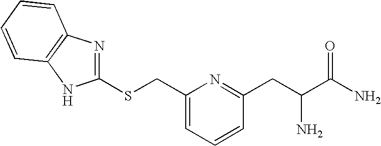

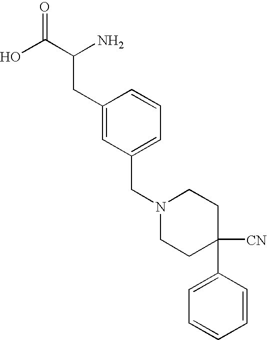

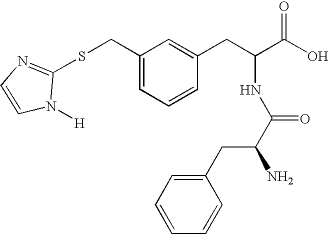

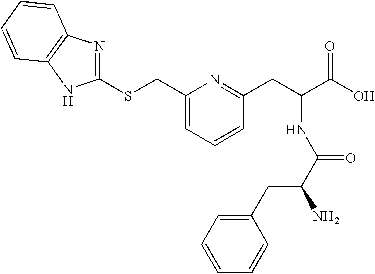

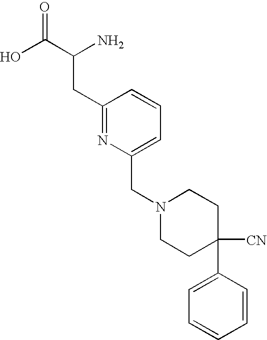

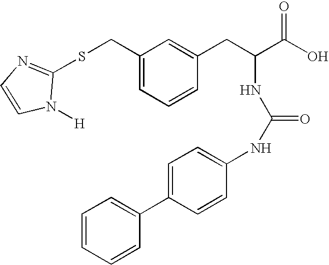

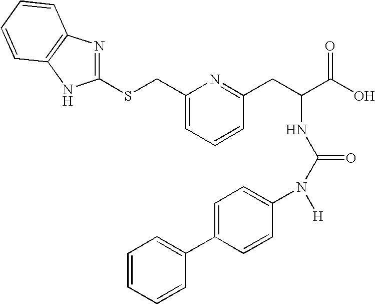

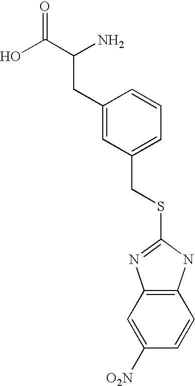

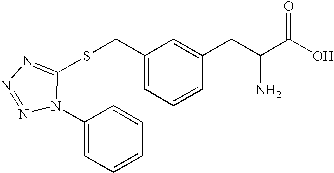

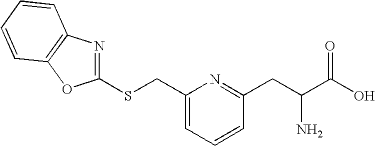

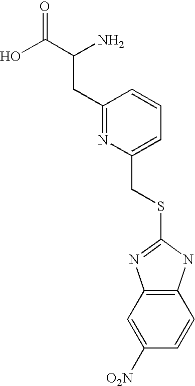

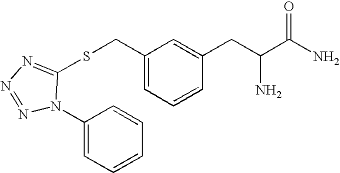

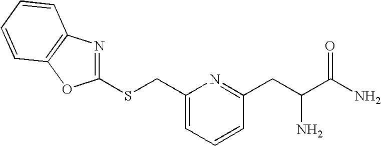

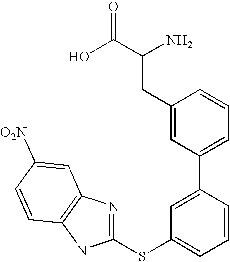

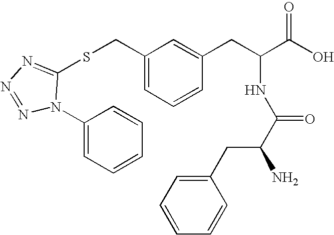

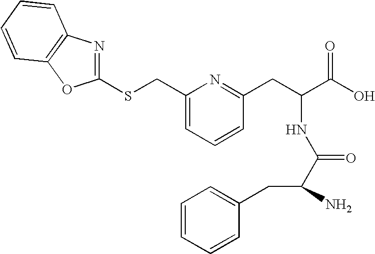

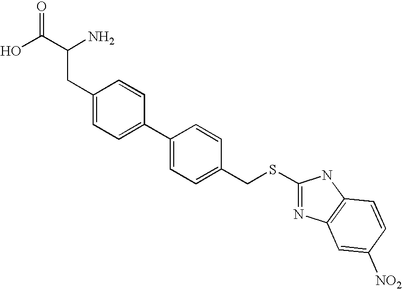

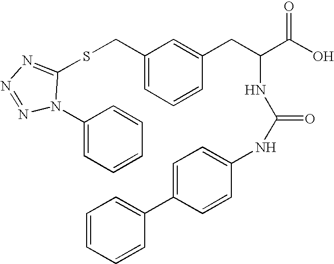

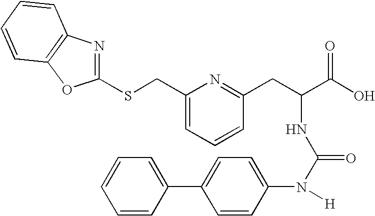

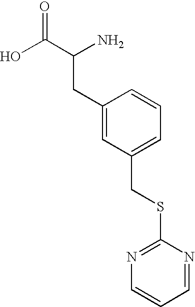

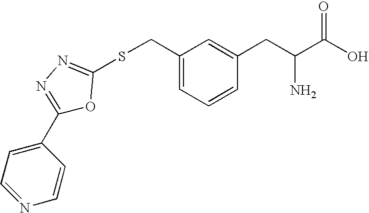

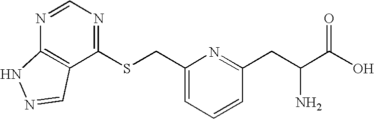

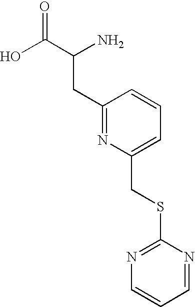

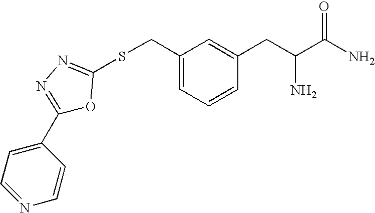

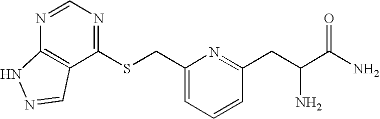

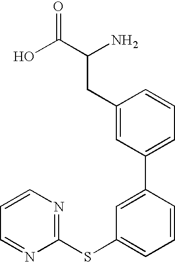

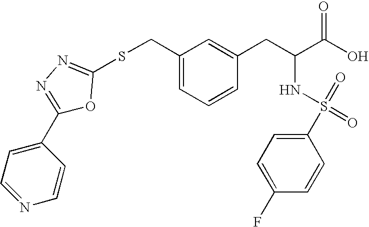

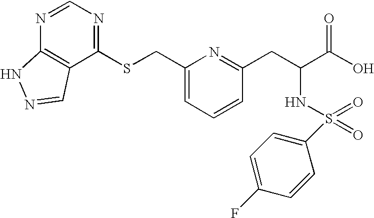

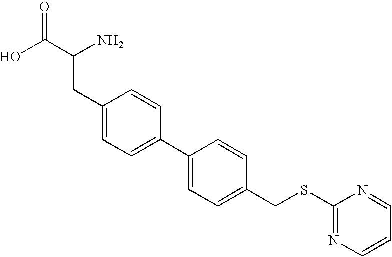

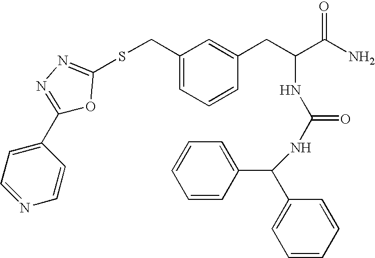

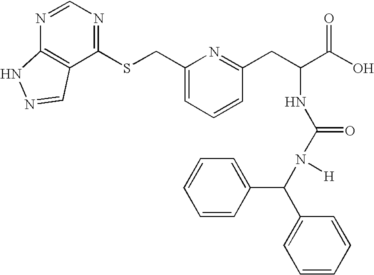

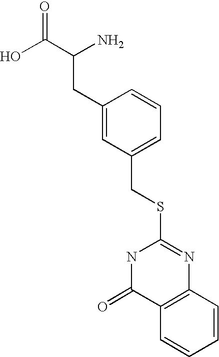

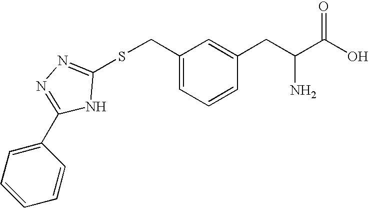

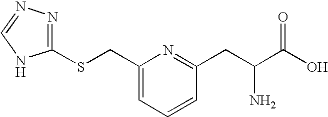

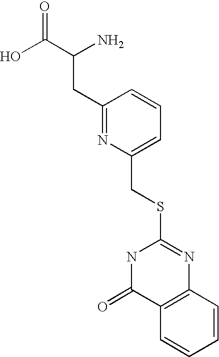

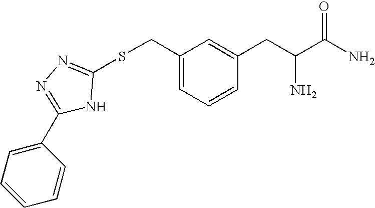

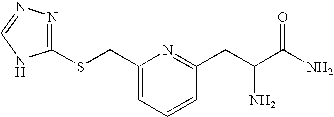

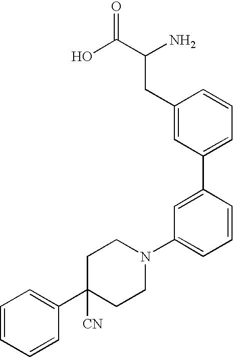

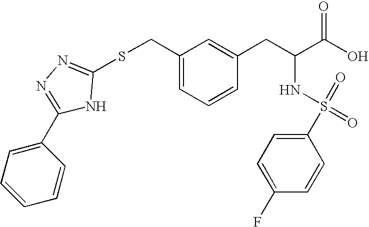

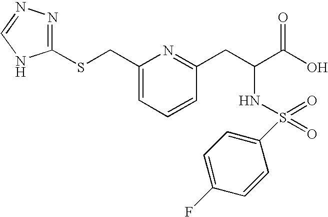

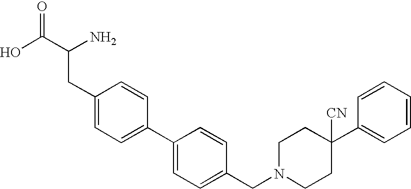

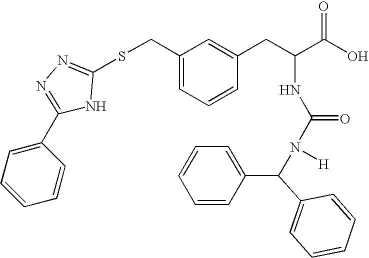

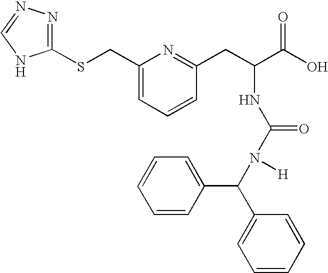

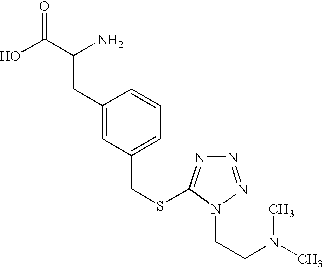

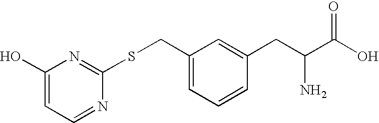

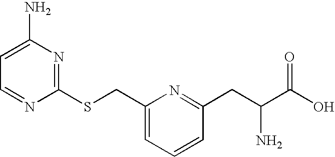

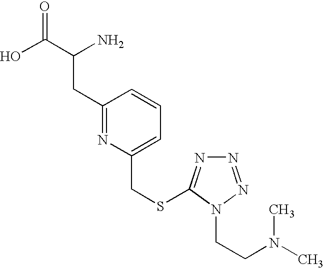

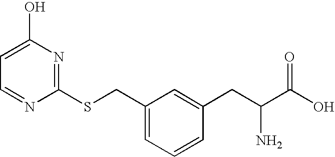

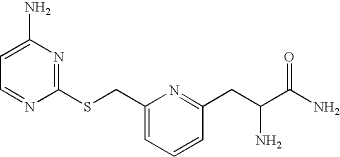

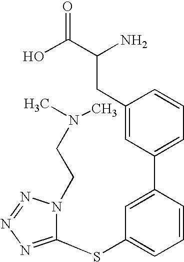

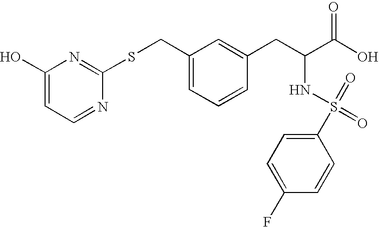

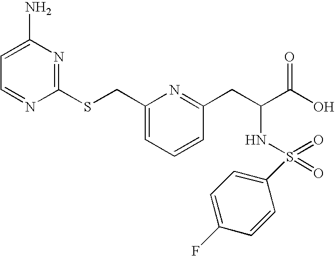

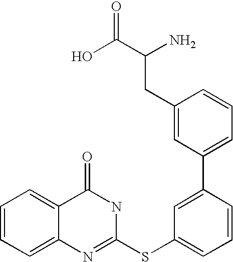

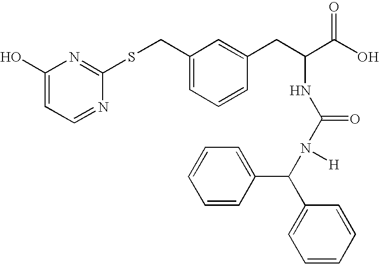

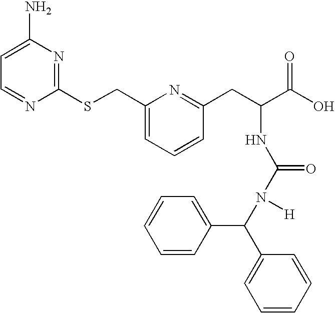

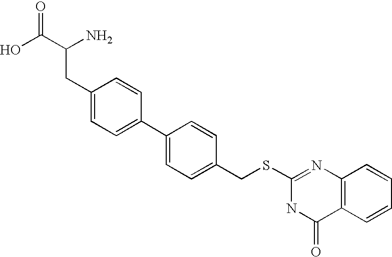

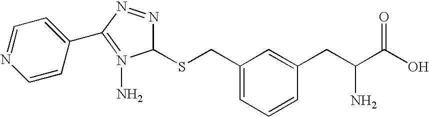

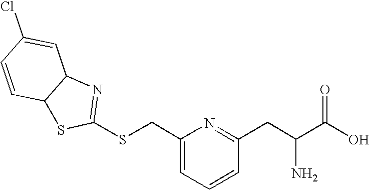

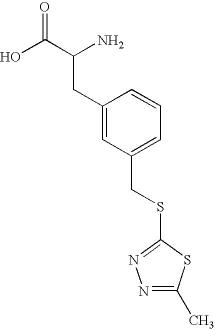

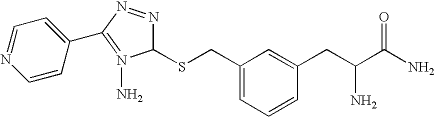

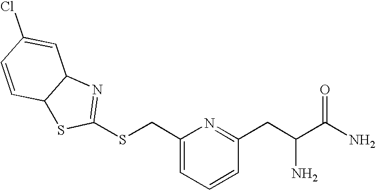

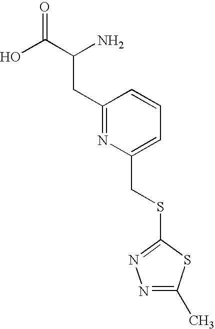

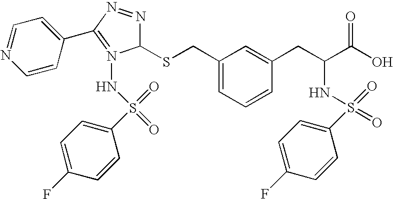

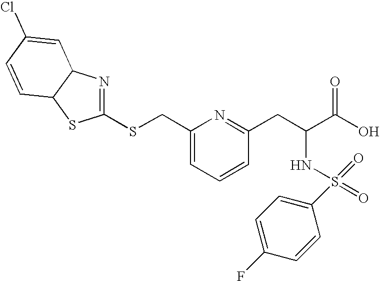

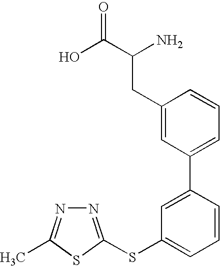

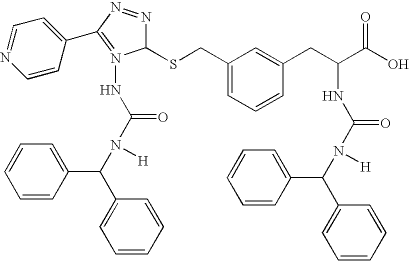

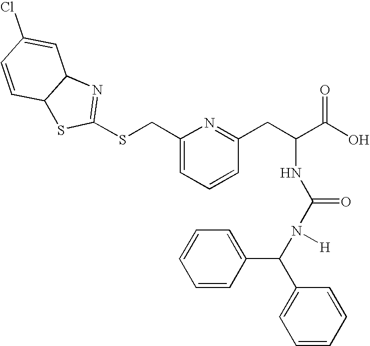

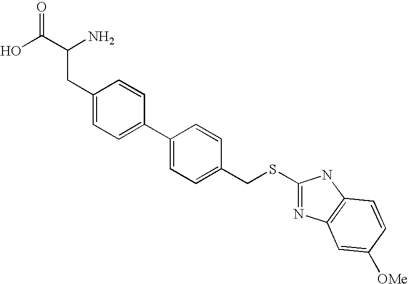

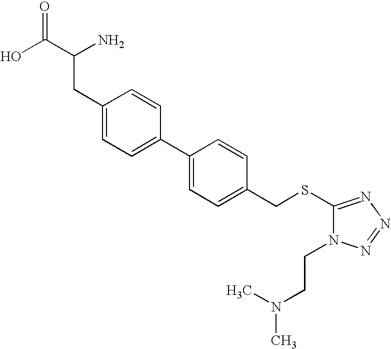

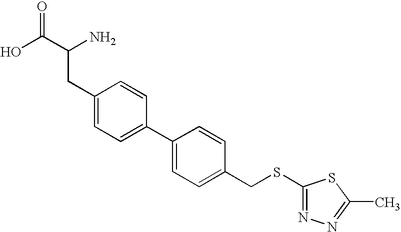

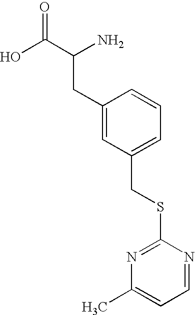

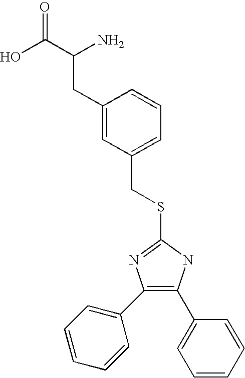

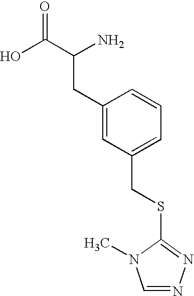

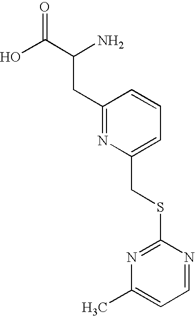

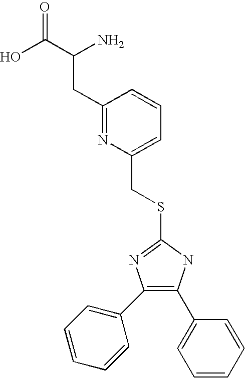

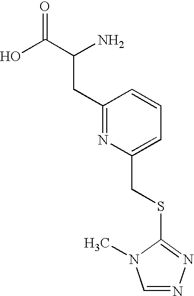

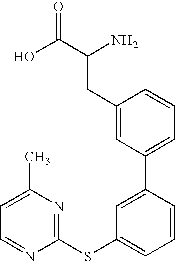

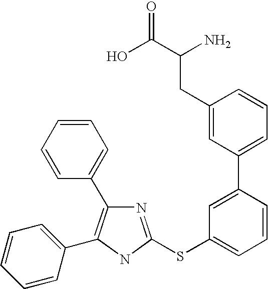

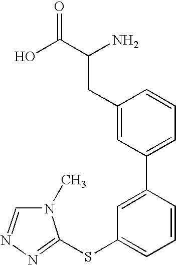

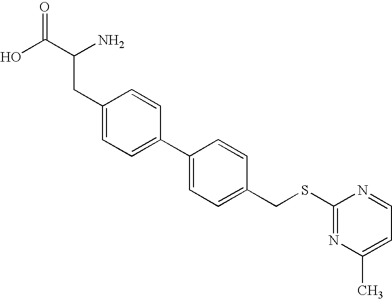

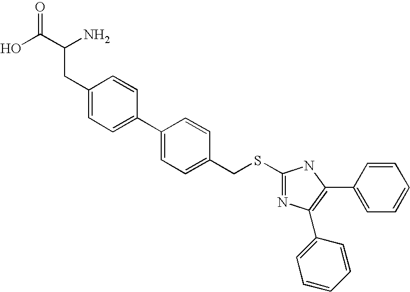

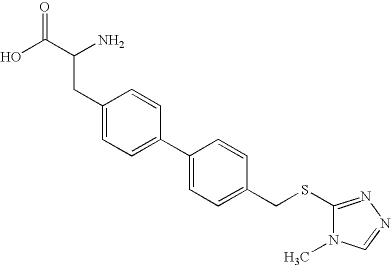

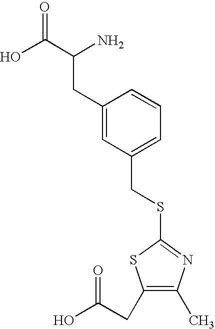

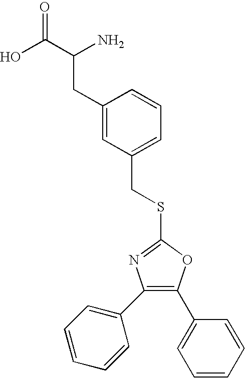

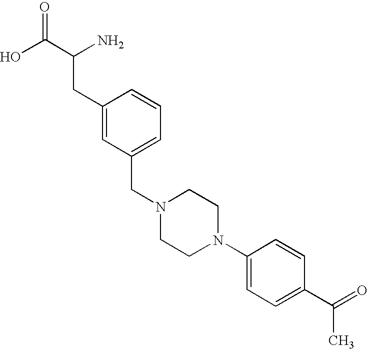

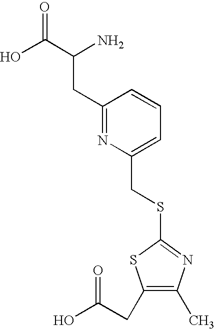

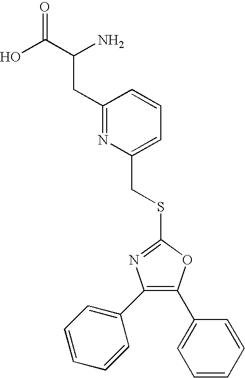

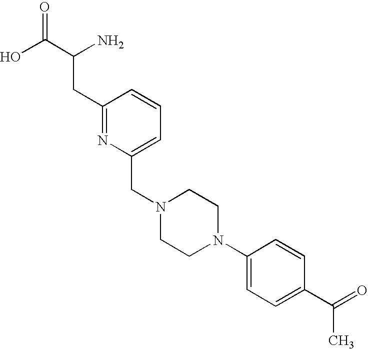

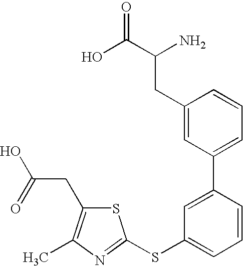

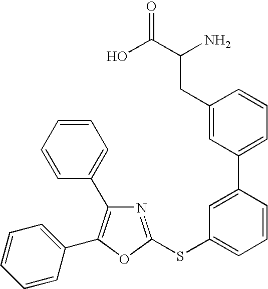

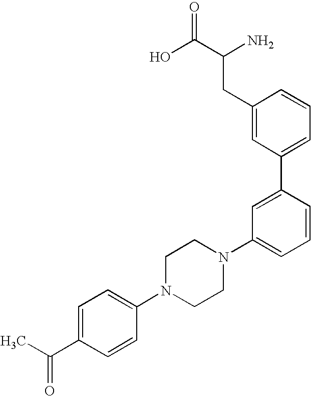

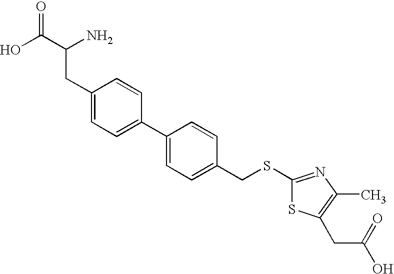

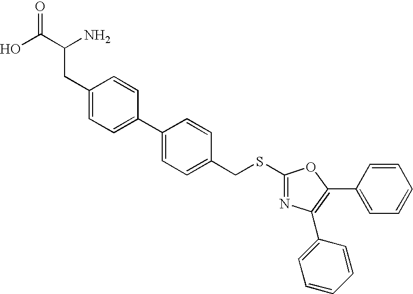

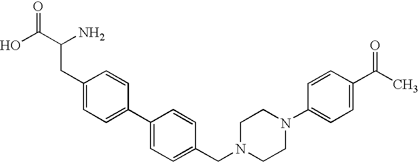

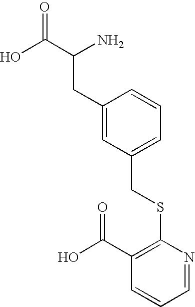

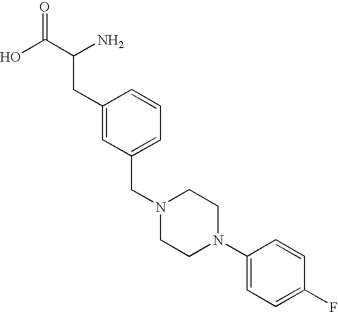

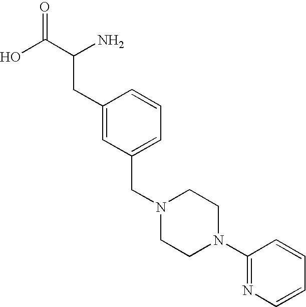

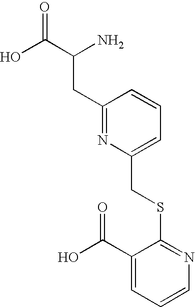

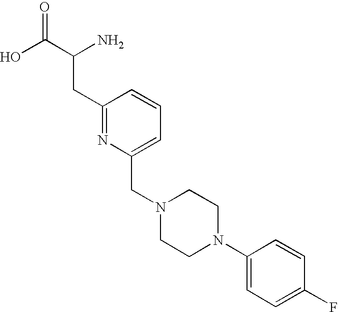

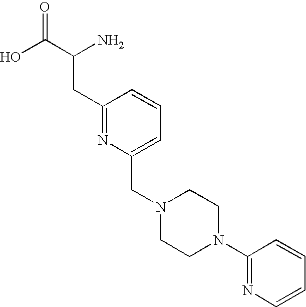

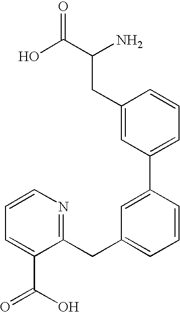

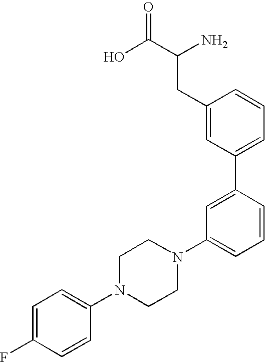

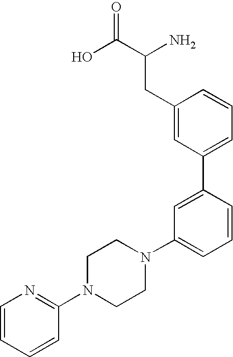

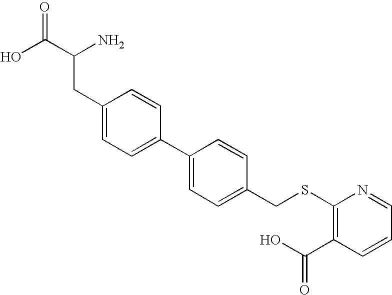

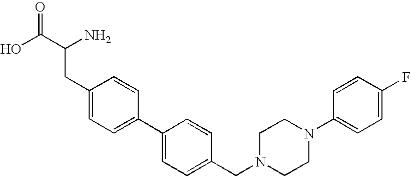

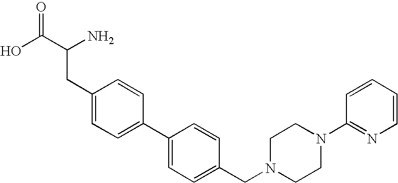

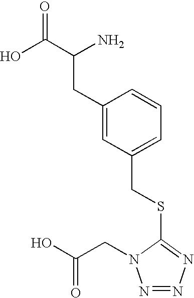

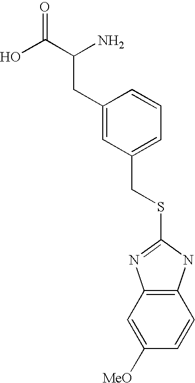

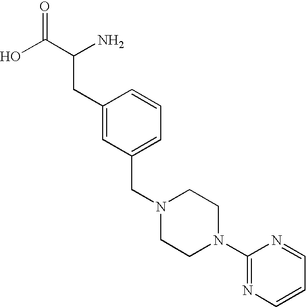

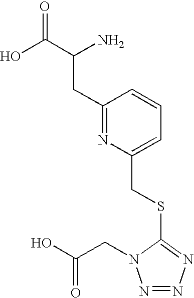

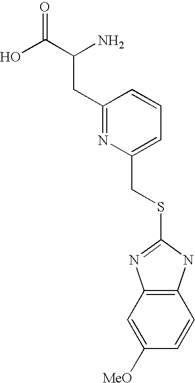

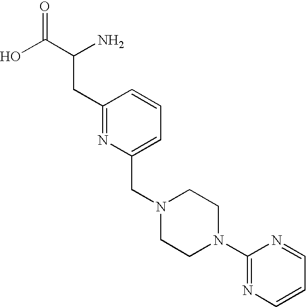

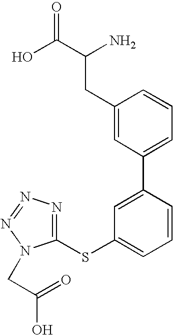

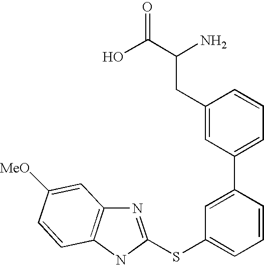

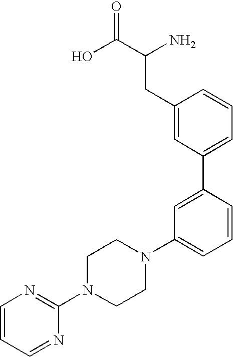

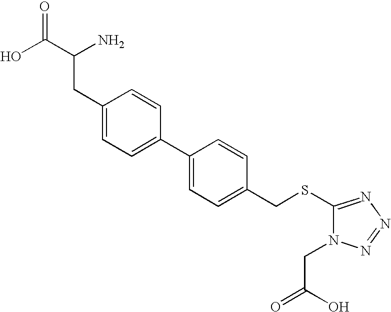

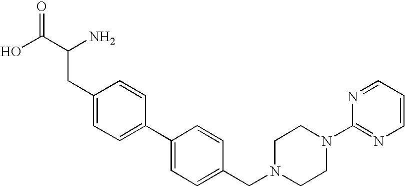

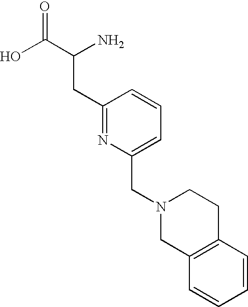

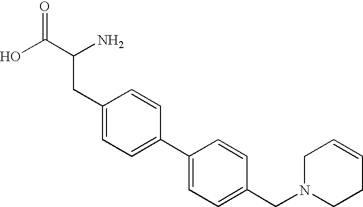

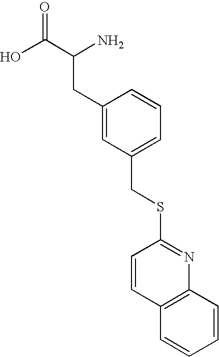

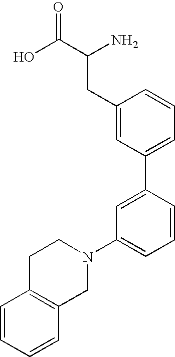

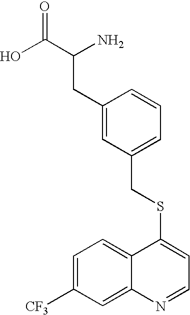

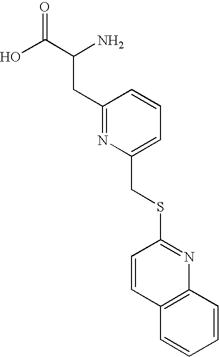

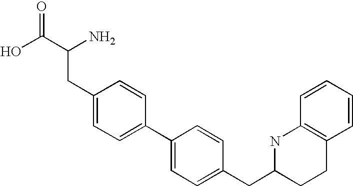

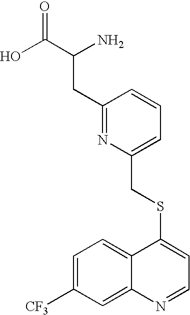

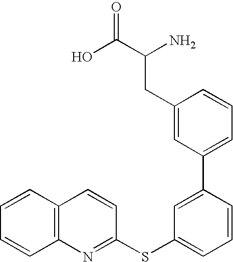

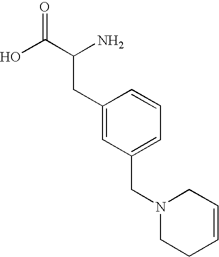

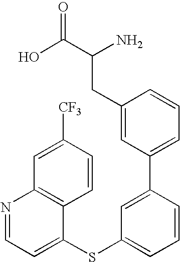

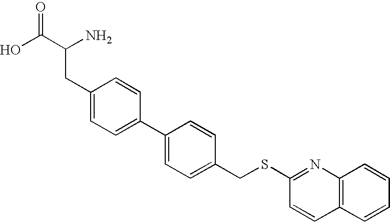

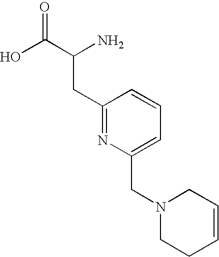

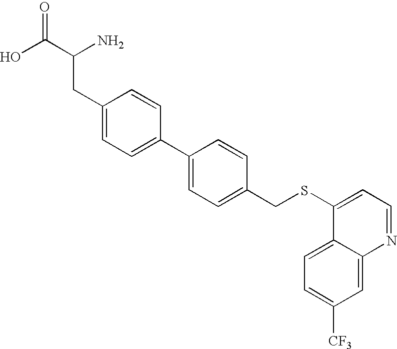

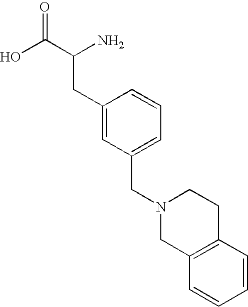

In yet another embodiment, the compound is at least one compound selected from the compounds of Table 2 and pharmaceutically acceptable salts, esters, and prodrugs thereof.

TABLE 2
Exemplary Compounds of the present invention.

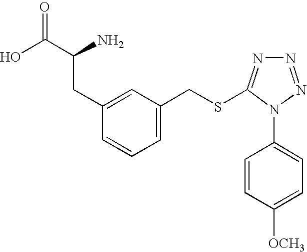

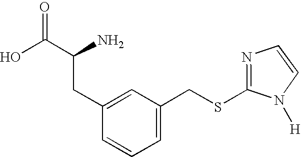

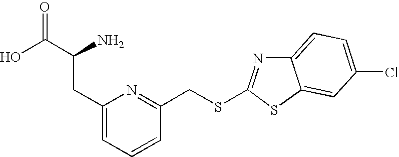

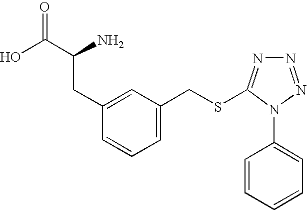

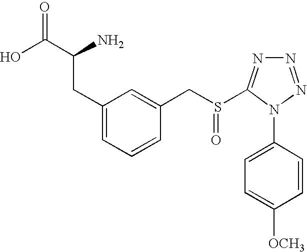

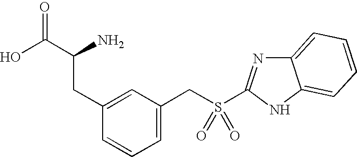

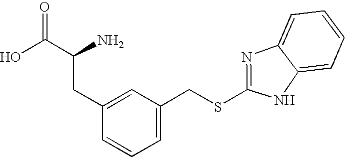

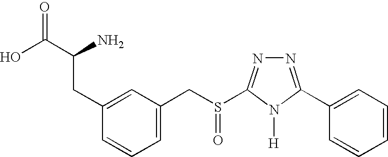

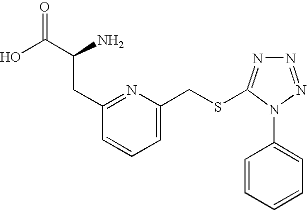

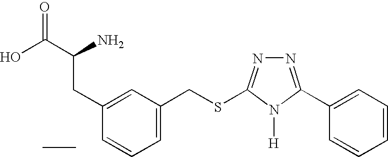

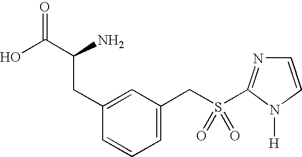

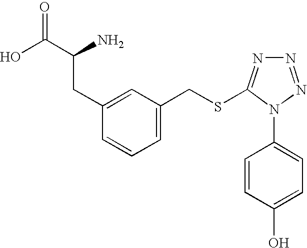

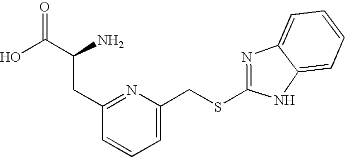

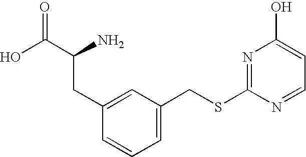

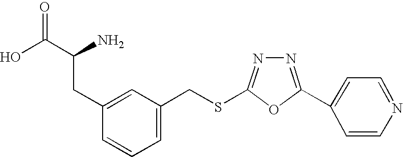

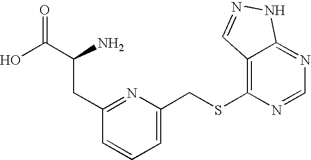

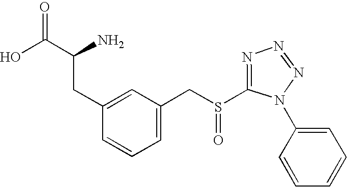

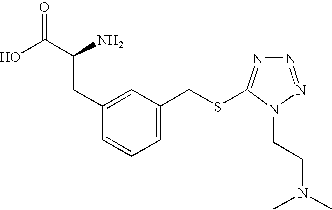

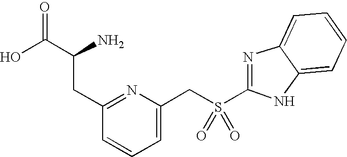

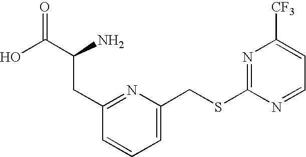

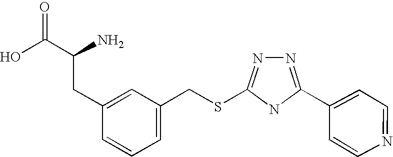

In some embodiments the compounds of the present invention are not the compounds of Table 3 and pharmaceutically acceptable salts thereof.

TABLE 3
Exemplary Compounds.

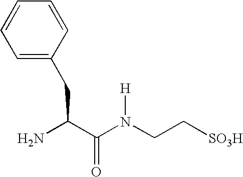

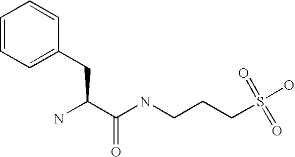

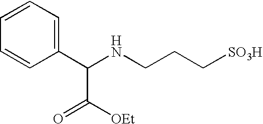

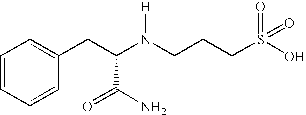

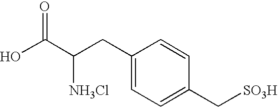

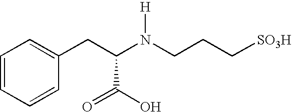

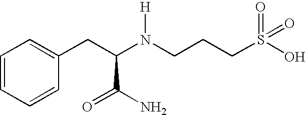

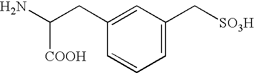

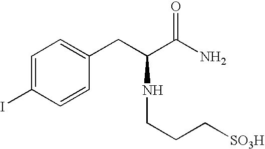

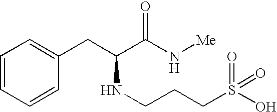

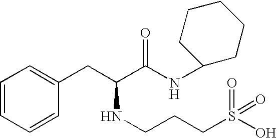

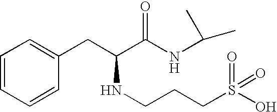

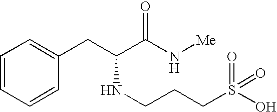

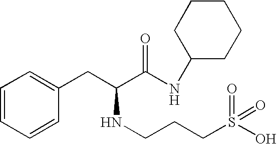

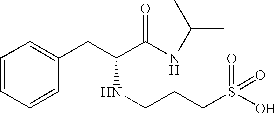

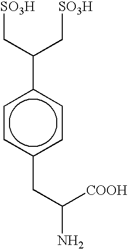

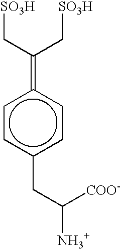

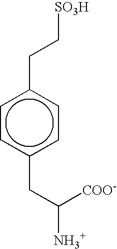

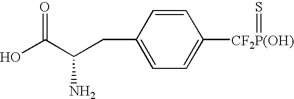

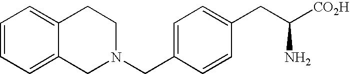

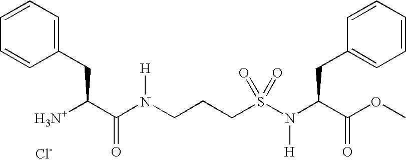

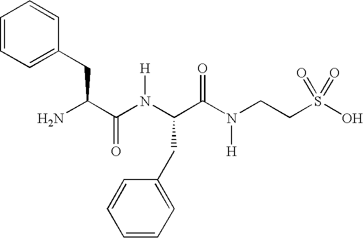

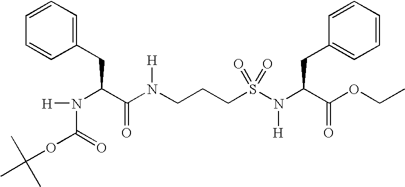

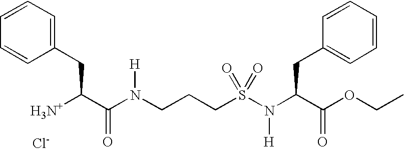

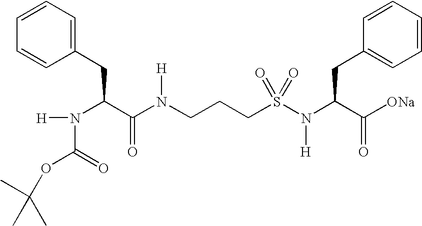

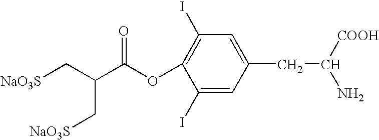

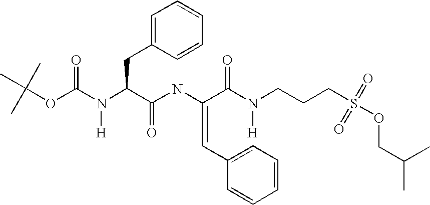

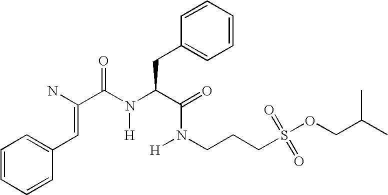

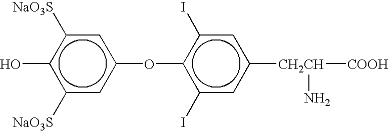

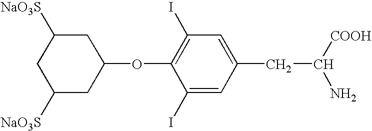

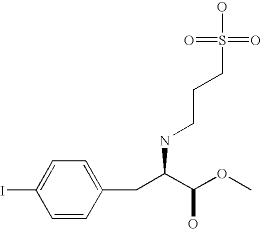

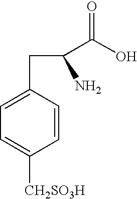

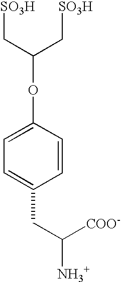

In some embodiments the compounds of the present invention include the compounds of Table 3 and pharmaceutically acceptable salts thereof.

It should be understood that the use of any of the compounds described herein is within the scope of the present invention and is intended to be encompassed by the present invention.

Libraries

In another aspect, the invention provides libraries of compounds of Formula I and/or Formula II, and methods of preparing such libraries. The synthesis of combinatorial libraries is well known in the art and has been reviewed (see, e.g., E. M. Gordon et al., J. Med Chem. 37:1385-1401 (1994)). Thus, the subject invention contemplates methods for synthesis of combinatorial libraries of compounds of Formula I and/or Formula II.

In some embodiments, libraries of compounds of the invention contain at least 30 compounds, at least 100 compounds, or at least 500 compounds. In some embodiments, the libraries of compounds of the invention contain fewer than 10⁹ compounds, fewer than 10⁸ compounds, or fewer than 10⁷ compounds.

A library of compounds may be substantially pure, i.e., substantially free of compounds other than the intended products, e.g., members of the library. In some embodiments, the purity of a library produced according to the methods of the invention is at least about 50%, at least about 70%, at least about 90%, or at least about 95%.

The libraries of compounds of the invention can be prepared according to the methods of the invention. In general, at least one starting material used for synthesis of the libraries of the invention is provided as a variegated population. The term “variegated population”, as used herein, refers to a population including at least two different chemical entities, e.g., of different chemical structure. For example, a “variegated population” of compounds of Formula II would comprise at least two different compounds of Formula II. Use of a variegated population of linkers to immobilize compounds to the solid support can produce a variety of compounds upon cleavage of the linkers.

Libraries of the invention are useful, e.g., for drug discovery. For example, a library of the invention can be screened (e.g., according to the methods described herein) to determine whether the library includes compounds having a pre-selected activity (e.g., useful for treating CNS diseases or amyloid associated diseases).

Isolation of Rat Primary Cerebrovascular Endothelial Cells

Of concern in the development of drugs targeting the central nervous system (CNS) is their ability to penetrate into the brain. The present invention provides an in vitro assay to predict the likelihood of a given drug to cross the blood-brain barrier (BBB) via specific carrier-mediated transport systems. Isolation and culture of primary rat brain endothelial cells (RBEC) have previously been reported as laborious procedures. High variations in yield and quality of cells are all factors that have blocked their use in the development of a medium throughput screening assay for testing compounds. In certain aspects, the present invention is directed to a reproducible method for isolating and culturing enriched RBEC from microcapillaries for their use, e.g., in screening compounds for their ability to bind to the large neutral amino acid carrier (L1-system carrier). Compared to previously described protocols, the present methods have several advantages. After only 5 days of culture, endothelial cells can be characterized and used immediately for screening. The present method provides high yields, e.g., the RBEC from 36 brains provides enough cells to screen simultaneously 7 compounds per plate in 2 to 3 96-well plates. During this short term culture, primary RBEC retain their morphology as well as their endothelial characteristics such as the expression of the von Willebrand factor (Factor VIII-related antigen), the specific lectin binding, and the uptake of acetylated low density lipoprotein (Ac-LDL). Innovative characteristics of this new isolation procedure are 1) the optimization of a two-stage enzymatic digestion to produce partially digested microcapillaries mostly depleted of non-endothelial cells, 2) the improved selective growth of RBEC by the short initial adherence period, and 3) the lack of cloning procedure resulting from these previous steps.

Accordingly, in one aspect, the present invention is directed to a method for isolating Rat Primary Cerebrovascular Endothelial Cells. In some embodiments, the method includes one or more of the following steps: removing cortices from rats; digesting the cortices; isolating the microcapillaries; digesting the microcapillaries; isolating the microcapillaries again; and incubating the microcapillaries until the endothelial cells establish themselves. In one embodiment, the method produces enriched brain endothelial cell cultures. In other embodiments, the present method for isolating and culturing enriched primary endothelial cell retains the characteristics of the RBEC and the functionality of their endogenous transporters such as the L1-system carrier.

In yet another aspect, the rat primary cerebrovascular endothelial cells isolated as described by the methods herein are used in an assay to test compounds of the present invention. For example, they may be used to determine the indirect ability of specific compounds to cross the BBB using active transporter systems such as the L1-system. In some embodiments, the RBEC cultures retaining their endothelial transporter system functionality are used in a rapid, reliable, and reproducible competitive binding assay to screen drugs. In some embodiments, this competitive binding assay can be employed to identify compounds that bind to the L1-system carrier and provide parameters to select CNS drug candidates designed to penetrate the brain using a specific active transporter.

Subjects and Patient Populations

The term “subject” includes living organisms in which amyloidosis can occur, or which are susceptible to amyloid diseases, e.g., Alzheimer's disease, Down's syndrome, CAA, dialysis-related (β₂M) amyloidosis, secondary (AA) amyloidosis, primary (AL) amyloidosis, hereditary amyloidosis, diabetes, etc. Examples of subjects include humans, chickens, ducks, peking ducks, geese, monkeys, deer, cows, rabbits, sheep, goats, dogs, cats, mice, rats, and transgenic species thereof. Administration of the compositions of the present invention to a subject to be treated can be carried out using known procedures, at dosages and for periods of time effective to modulate amyloid aggregation or amyloid-induced toxicity in the subject as further described herein. An effective amount of the therapeutic compound necessary to achieve a therapeutic effect may vary according to factors such as the amount of amyloid already deposited at the clinical site in the subject, the age, sex, and weight of the subject, and the ability of the therapeutic compound to modulate amyloid aggregation in the subject. Dosage regimens can be adjusted to provide the optimum therapeutic response. For example, several divided doses may be administered daily or the dose may be proportionally reduced as indicated by the exigencies of the therapeutic situation.

In certain embodiments of the invention, the subject is in need of treatment by the methods of the invention, and is selected for treatment based on this need. A subject in need of treatment is art-recognized, and includes subjects that have been identified as having a disease or disorder related to amyloid-deposition or amyloidosis, has a symptom of such a disease or disorder, or is at risk of such a disease or disorder, and would be expected, based on diagnosis, e.g., medical diagnosis, to benefit from treatment (e.g., curing, healing, preventing, alleviating, relieving, altering, remedying, ameliorating, improving, or affecting the disease or disorder, the symptom of the disease or disorder, or the risk of the disease or disorder).

In an exemplary aspect of the invention, the subject is a human. For example, the subject may be a human over 30 years old, human over 40 years old, a human over 50 years old, a human over 60 years old, a human over 70 years old, a human over 80 years old, a human over 85 years old, a human over 90 years old, or a human over 95 years old. The subject may be a female human, including a postmenopausal female human, who may be on hormone (estrogen) replacement therapy. The subject may also be a male human. In another embodiment, the subject is under 40 years old.

A subject may be a human at risk for Alzheimer's disease, e.g., being over the age of 40 or having a predisposition for Alzheimer's disease. Alzheimer's disease predisposing factors identified or proposed in the scientific literature include, among others, a genotype predisposing a subject to Alzheimer's disease; environmental factors predisposing a subject to Alzheimer's disease; past history of infection by viral and bacterial agents predisposing a subject to Alzheimer's disease; and vascular factors predisposing a subject to Alzheimer's disease. A subject may also have one or more risk factors for cardiovascular disease (e.g., atherosclerosis of the coronary arteries, angina pectoris, and myocardial infarction) or cerebrovascular disease (e.g., atherosclerosis of the intracranial or extracranial arteries, stroke, syncope, and transient ischemic attacks), such as hypercholesterolemia, hypertension, diabetes, cigarette smoking, familial or previous history of coronary artery disease, cerebrovascular disease, and cardiovascular disease. Hypercholesterolemia typically is defined as a serum total cholesterol concentration of greater than about 5.2 mmol/L (about 200 mg/dL).

Several genotypes are believed to predispose a subject to Alzheimer's disease. These include the genotypes such as presenilin-1, presenilin-2, and amyloid precursor protein (APP) missense mutations associated with familial Alzheimer's disease, and a-2-macroglobulin and LRP-1 genotypes, which are thought to increase the risk of acquiring sporadic (late-onset) Alzheimer's disease. E. van Uden, et al., J. Neurosci. 22(21), 9298-304 (2002); J. J. Goto, et al., J. Mol. Neurosci. 19(1-2), 37-41 (2002). Another genetic risk factor for the development of Alzheimer's disease are variants of ApoE, the gene that encodes apolipoprotein E (particularly the apoE4 genotype), a constituent of the low-density lipoprotein particle. W J Strittmatter, et al., Annu. Rev. Neurosci. 19, 53-77 (1996). The molecular mechanisms by which the various ApoE alleles alter the likelihood of developing Alzheimer's disease are unknown, however the role of ApoE in cholesterol metabolism is consistent with the growing body of evidence linking cholesterol metabolism to Alzheimer's disease. For example, chronic use of cholesterol-lowering drugs such as statins has recently been associated with a lower incidence of Alzheimer's disease, and cholesterol-lowering drugs have been shown to reduce pathology in APP transgenic mice. These and other studies suggest that cholesterol may affect APP processing. ApoE4 has been suggested to alter Aβ trafficking (in and out of the brain), and favor retention of Aβ in the brain. ApoE4 has also been suggested to favor APP processing toward Aβ formation. Environmental factors have been proposed as predisposing a subject to Alzheimer's disease, including exposure to aluminum, although the epidemiological evidence is ambiguous. In addition, prior infection by certain viral or bacterial agents may predispose a subject to Alzheimer's disease, including the herpes simplex virus and chlamydia pneumoniae. Finally, other predisposing factors for Alzheimer's disease can include risk factors for cardiovascular or cerebrovascular disease, including cigarette smoking, hypertension and diabetes. “At risk for Alzheimer's disease” also encompasses any other predisposing factors not listed above or as yet identified and includes an increased risk for Alzheimer's disease caused by head injury, medications, diet, or lifestyle.

The methods of the present invention can be used for one or more of the following: to prevent Alzheimer's disease, to treat Alzheimer's disease, or ameliorate symptoms of Alzheimer's disease, or to regulate production of or levels of amyloid β (Aβ) peptides. In an embodiment, the human carries one or more mutations in the genes that encode β-amyloid precursor protein, presenilin-1 or presenilin-2. In another embodiment, the human carries the Apolipoprotein ε4 gene. In another embodiment, the human has a family history of Alzheimer's Disease or a dementia illness. In another embodiment, the human has trisomy 21 (Down's Syndrome). In another embodiment, the subject has a normal or low serum total blood cholesterol level. In another embodiment, the serum total blood cholesterol level is less than about 200 mg/dL, or less than about 180, and it can range from about 150 to about 200 mg/dL. In another embodiment, the total LDL cholesterol level is less than about 100 mg/dL, or less than about 90 mg/dL and can range from about 30 to about 100 mg/dL. Methods of measuring serum total blood cholesterol and total LDL cholesterol are well known to those skilled in the art and for example include those disclosed in WO 99/38498 at p. 11, incorporated by reference herein. Methods of determining levels of other sterols in serum are disclosed in H. Gylling, et al., “Serum Sterols During Stanol Ester Feeding in a Mildly Hypercholesterolemic Population”, J. Lipid Res. 40: 593-600 (1999).

In another embodiment, the subject has an elevated serum total blood cholesterol level. In another embodiment, the serum total cholesterol level is at least about 200 mg/dL, or at least about 220 mg/dL and can range from about 200 to about 1000 mg/dL. In another embodiment, the subject has an elevated total LDL cholesterol level. In another embodiment, the total LDL cholesterol level is greater than about 100 mg/dL, or even greater than about 110 mg/dL and can range from about 100 to about 1000 mg/dL.

In another embodiment, the human is at least about 40 years of age. In another embodiment, the human is at least about 60 years of age. In another embodiment, the human is at least about 70 years of age. In another embodiment, the human is at least about 80 years of age. In another embodiment, the human is at least about 85 years of age. In one embodiment, the human is between about 60 and about 100 years of age.

In still a further embodiment, the subject is shown to be at risk by a diagnostic brain imaging technique, for example, one that measures brain activity, plaque deposition, or brain atrophy.

In still a further embodiment, the subject is shown to be at risk by a cognitive test such as Clinical Dementia Rating (“CDR”), Alzheimer's Disease Assessment Scale-Cognition (“ADAS-Cog”), or Mini-Mental State Examination (“MMSE”). The subject may exhibit a below average score on a cognitive test, as compared to a historical control of similar age and educational background. The subject may also exhibit a reduction in score as compared to previous scores of the subject on the same or similar cognition tests.

In determining the CDR, a subject is typically assessed and rated in each of six cognitive and behavioural categories: memory, orientation, judgement and problem solving, community affairs, home and hobbies, and personal care. The assessment may include historical information provided by the subject, or preferably, a corroborator who knows the subject well. The subject is assessed and rated in each of these areas and the overall rating, (0, 0.5, 1.0, 2.0 or 3.0) determined. A rating of 0 is considered normal. A rating of 1.0 is considered to correspond to mild dementia. A subject with a CDR of 0.5 is characterized by mild consistent forgetfulness, partial recollection of events and “benign” forgetfulness. In one embodiment the subject is assessed with a rating on the CDR of above 0, of above about 0.5, of above about 1.0, of above about 1.5, of above about 2.0, of above about 2.5, or at about 3.0.

Another test is the Mini-Mental State Examination (MMSE), as described by Folstein “Mini-mental state. A practical method for grading the cognitive state of patients for the clinician.” J. Psychiatr. Res. 12:189-198, 1975. The MMSE evaluates the presence of global intellectual deterioration. See also Folstein “Differential diagnosis of dementia. The clinical process.” Psychiatr Clin North Am. 20:45-57, 1997. The MMSE is a means to evaluate the onset of dementia and the presence of global intellectual deterioration, as seen in Alzheimer's disease and multi-infart dementia. The MMSE is scored from 1 to 30. The MMSE does not evaluate basic cognitive potential, as, for example, the so-called IQ test. Instead, it tests intellectual skills. A person of “normal” intellectual capabilities will score a “30” on the MMSE objective test (however, a person with a MMSE score of 30 could also score well below “normal” on an IQ test). See, e.g., Kaufer, J. Neuropsychiatry Clin. Neurosci. 10:55-63, 1998; Becke, Alzheimer Dis Assoc Disord. 12:54-57, 1998; Ellis, Arch. Neurol. 55:360-365, 1998; Magni, Int. Psychogeriatr. 8:127-134, 1996; Monsch, ActaNeurol. Scand. 92:145-150, 1995. In one embodiment, the subject scores below 30 at least once on the MMSE. In another embodiment, the subject scores below about 28, below about 26, below about 24, below about 22, below about 20, below about 18, below about 16, below about 14, below about 12, below about 10, below about 8, below about 6, below about 4, below about 2, or below about 1.

Another means to evaluate cognition, particularly Alzheimer's disease, is the Alzheimer's Disease Assessment Scale (ADAS-Cog), or a variation termed the Standardized Alzheimer's Disease Assessment Scale (SADAS). It is commonly used as an efficacy measure in clinical drug trials of Alzheimer's disease and related disorders characterized by cognitive decline. SADAS and ADAS-Cog were not designed to diagnose Alzheimer's disease; they are useful in characterizing symptoms of dementia and are a relatively sensitive indicator of dementia progression. (See, e.g., Doraiswamy, Neurology 48:1511-1517, 1997; and Standish, J. Am. Geriatr. Soc. 44:712-716, 1996.) Annual deterioration in untreated Alzheimer's disease patients is approximately 8 points per year (See, eg., Raskind, M Prim. Care Companion J Clin Psychiatry August 2000; 2(4):134-138).

The ADAS-cog is designed to measure, with the use of questionnaires, the progression and the severity of cognitive decline as seen in AD on a 70-point scale. The ADAS-cog scale quantifies the number of wrong answers. Consequently, a high score on the scale indicates a more severe case of cognitive decline. In one embodiment, a subject exhibits a score of greater than 0, greater than about 5, greater than about 10, greater than about 15, greater than about 20, greater than about 25, greater than about 30, greater than about 35, greater than about 40, greater than about 45, greater than about 50, greater than about 55, greater than about 60, greater than about 65, greater than about 68, or about 70.

In another embodiment, the subject exhibits no symptoms of Alzheimer's Disease. In another embodiment, the subject is a human who is at least 40 years of age and exhibits no symptoms of Alzheimer's Disease. In another embodiment, the subject is a human who is at least 40 years of age and exhibits one or more symptoms of Alzheimer's Disease.

In another embodiment, the subject has Mild Cognitive Impairment. In a further embodiment, the subject has a CDR rating of about 0.5. In another embodiment, the subject has early Alzheimer's disease. In another embodiment, the subject has cerebral amyloid angiopathy.

By using the methods of the present invention, the levels of amyloid β peptides in a subject's plasma or cerebrospinal fluid (CSF) can be reduced from levels prior to treatment from about 10 to about 100 percent, or even about 50 to about 100 percent.

In an alternative embodiment, the subject can have an elevated level of amyloid Aβ₄₀ and Aβ₄₂ peptide in the blood and CSF prior to treatment, according to the present methods, of greater than about 10 pg/mL, or greater than about 20 pg/mL, or greater than about 35 pg/mL, or even greater than about 40 pg/mL. In another embodiment, the elevated level of amyloid Aβ₄₂ peptide can range from about 30 pg/mL to about 200 pg/mL, or even to about 500 pg/mL. One skilled in the art would understand that as Alzheimer's disease progresses, the measurable levels of amyloid 0 peptide in the CSF may decrease from elevated levels present before onset of the disease. This effect is attributed to increased deposition, i.e., trapping of Aβ peptide in the brain instead of normal clearance from the brain into the CSF.

In an alternative embodiment, the subject can have an elevated level of amyloid Aβ₄₀ peptide in the blood and CSF prior to treatment, according to the present methods, of greater than about 5 pg Aβ₄₂/mL or greater than about 50 pg Aβ₄₀/mL, or greater than about 400 pg/mL. In another embodiment, the elevated level of amyloid Aβ₄₀ peptide can range from about 200 pg/mL to about 800 pg/mL, to even about 1000 pg/mL.

In another embodiment, the subject can have an elevated level of amyloid Aβ₄₂ peptide in the CSF prior to treatment, according to the present methods, of greater than about 5 pg/mL, or greater than about 10 pg/mL, or greater than about 200 pg/mL, or greater than about 500 pg/mL. In another embodiment, the level of amyloid β peptide can range from about 10 pg/mL to about 1,000 pg/mL, or even about 100 pg/mL to about 1,000 pg/mL.

In another embodiment, the subject can have an elevated level of amyloid Aβ₄₀ peptide in the CSF prior to treatment according to the present methods of greater than about 10 pg/mL, or greater than about 50 pg/mL, or even greater than about 100 pg/mL. In another embodiment, the level of amyloid β peptide can range from about 10 pg/mL to about 1,000 pg/mL.

The amount of amyloid P peptide in the brain, CSF, blood, or plasma of a subject can be evaluated by enzyme-linked immunosorbent assay (“ELISA”) or quantitative immunoblotting test methods or by quantitative SELDI-TOF which are well known to those skilled in the art, such as is disclosed by Zhang, et al., J. Biol. Chem. 274, 8966-72 (1999) and Zhang, et al., Biochemistry 40, 5049-55 (2001). See also, A. K. Vehmas, et al., DNA Cell Biol. 20(11), 713-21 (2001), P. Lewczuk, et al., Rapid Commun. Mass Spectrom. 17(12), 1291-96 (2003); B. M. Austen, et al., J. Peptide Sci. 6, 459-69 (2000); and H. Davies, et al., BioTechniques 27, 1258-62 (1999). These tests are performed on samples of the brain or blood which have been prepared in a manner well known to one skilled in the art. Another example of a useful method for measuring levels of amyloid P peptides is by Europium immunoassay (EIA). See, e.g., WO 99/38498 at p. 11.

The methods of the invention may be applied as a therapy for a subject having Alzheimer's disease or a dementia, or the methods of the invention may be applied as a prophylaxis against Alzheimer's disease or dementia for subject with such a predisposition, as in a subject, e.g., with a genomic mutation in the APP gene, the ApoE gene, or a presenilin gene. The subject may have (or may be predisposed to developing or may be suspected of having) vascular dementia, or senile dementia, Mild Cognitive Impairment, or early Alzheimer's disease. In addition to Alzheimer's disease, the subject may have another amyloid associated disease such as cerebral amyloid angiopathy, or the subject may have amyloid deposits, especially amyloid-β amyloid deposits in the brain.

Treatment of Central Nervous System Disorders and/or Amyloid Associated Diseases

The present invention pertains to methods of using the compounds and pharmaceutical compositions thereof in the treatment and prevention of central nervous system disorders and/or amyloid associated diseases. The pharmaceutical compositions of the invention may be administered therapeutically or prophylactically to treat diseases associated with amyloid (e.g., AL amyloid protein (λ or κ-chain related, e.g., amyloid λ, amyloid κ, amyloid κIV, amyloid λVI, amyloid γ, amyloid γ1), Aβ, IAPP, β₂M, AA, or AH amyloid protein) fibril formation, aggregation or deposition.

The pharmaceutical compositions of the invention may act to ameliorate the course of an amyloid associated disease using any of the following mechanisms (this list is meant to be illustrative and not limiting): slowing the rate of amyloid fibril formation or deposition; lessening the degree of amyloid deposition; inhibiting, reducing, or preventing amyloid fibril formation; inhibiting neurodegeneration or cellular toxicity induced by amyloid; inhibiting amyloid induced inflammation; enhancing the clearance of amyloid from the brain; enhancing degradation of Aβ in the brain; or favoring clearance of amyloid protein prior to its organization in fibrils.

“Modulation” of amyloid deposition includes both inhibition, as defined above, and enhancement of amyloid deposition or fibril formation. The term “modulating” is intended, therefore, to encompass prevention or stopping of amyloid formation or accumulation, inhibition or slowing down of further amyloid formation or accumulation in a subject with ongoing amyloidosis, e.g., already having amyloid deposition, and reducing or reversing of amyloid formation or accumulation in a subject with ongoing amyloidosis; and enhancing amyloid deposition, e.g., increasing the rate or amount of amyloid deposition in vivo or in vitro. Amyloid-enhancing compounds may be useful in animal models of amyloidosis, for example, to make possible the development of amyloid deposits in animals in a shorter period of time or to increase amyloid deposits over a selected period of time. Amyloid-enhancing compounds may be useful in screening assays for compounds which inhibit amyloidosis in vivo, for example, in animal models, cellular assays and in vitro assays for amyloidosis. Such compounds may be used, for example, to provide faster or more sensitive assays for compounds. Modulation of amyloid deposition is determined relative to an untreated subject or relative to the treated subject prior to treatment.

“Inhibition” of amyloid deposition includes preventing or stopping of amyloid formation, e.g., fibrillogenesis, clearance of amyloid, e.g., soluble Aβ from brain, inhibiting or slowing down of further amyloid deposition in a subject with amyloidosis, e.g., already having amyloid deposits, and reducing or reversing amyloid fibrillogenesis or deposits in a subject with ongoing amyloidosis. Inhibition of amyloid deposition is determined relative to an untreated subject, or relative to the treated subject prior to treatment, or, e.g., determined by clinically measurable improvement, e.g., or in the case of a subject with brain amyloidosis, e.g., an Alzheimer's or cerebral amyloid angiopathy subject, stabilization of cognitive function or prevention of a further decrease in cognitive function (i.e., preventing, slowing, or stopping disease progression), or improvement of parameters such as the concentration of Aβ or tau in the CSF.

As used herein, “treatment” of a subject includes the application or administration of a composition of the invention to a subject, or application or administration of a composition of the invention to a cell or tissue from a subject, who has an amyloid associated disease or condition, has a symptom of such a disease or condition, or is at risk of (or susceptible to) such a disease or condition, with the purpose of curing, healing, alleviating, relieving, altering, remedying, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; improving a subject's physical or mental well-being; or, in some situations, preventing the onset of dementia. The treatment or amelioration of symptoms can be based on objective or subjective parameters; including the results of a physical examination, a psychiatric evaluation, or a cognition test such as CDR, MMSE, ADAS-Cog, or another test known in the art. For example, the methods of the invention successfully treat a subject's dementia by slowing the rate of or lessening the extent of cognitive decline.

In one embodiment, the term “treating” includes maintaining a subject's CDR rating at its base line rating or at 0. In another embodiment, the term treating includes decreasing a subject's CDR rating by about 0.25 or more, about 0.5 or more, about 1.0 or more, about 1.5 or more, about 2.0 or more, about 2.5 or more, or about 3.0 or more. In another embodiment, the term “treating” also includes reducing the rate of the increase of a subject's CDR rating as compared to historical controls. In another embodiment, the term includes reducing the rate of increase of a subject's CDR rating by about 5% or more, about 10% or more, about 20% or more, about 25% or more, about 30% or more, about 40% or more, about 50% or more, about 60% or more, about 70% or more, about 80% or more, about 90% or more, or about 100%, of the increase of the historical or untreated controls.

In another embodiment, the term “treating” also includes maintaining a subject's score on the MMSE. The term “treating” includes increasing a subject's MMSE score by about 1, about 2, about 3, about 4, about 5, about 7.5, about 10, about 12.5, about 15, about 17.5, about 20, or about 25 points. The term also includes reducing the rate of the decrease of a subject's MMSE score as compared to historical controls. In another embodiment, the term includes reducing the rate of decrease of a subject's MMSE score by about 5% or less, about 10% or less, about 20% or less, about 25% or less, about 30% or less, about 40% or less, about 50% or less, about 60% or less, about 70% or less, about 80% or less, about 90% or less or about 100% or less, of the decrease of the historical or untreated controls.

In yet another embodiment, the term “treating” includes maintaining a subject's score on the ADAS-Cog. The term “treating” includes decreasing a subject's ADAS-Cog score by about 1 point or greater, by about 2 points or greater, by about 3 points or greater, by about 4 points or greater, by about 5 points or greater, by about 7.5 points or greater, by about 10 points or greater, by about 12.5 points or greater, by about 15 points or greater, by about 17.5 points or greater, by about 20 points or greater, or by about 25 points or greater. The term, also includes reducing the rate of the increase of a subject's ADAS-Cog score as compared to historical controls. In another embodiment, the term includes reducing the rate of increase of a subject's ADAS-Cog score by about 5% or more, about 10% or more, about 20% or more, about 25% or more, about 30% or more, about 40% or more, about 50% or more, about 60% or more, about 70% or more, about 80% or more, about 90% or more or about 100% of the increase of the historical or untreated controls.

In another embodiment, the term “treating” e.g., for AA or AL amyloidosis, includes an increase in serum creatinine, e.g., an increase of creatinine clearance of 10% or greater, 20% or greater, 50% or greater, 80% or greater, 90% or greater, 100% or greater, 150% or greater, 200% or greater. The term “treating” also may include remission of nephrotic syndrome (NS). It may also include remission of chronic diarrhea and/or a gain in body weight, e.g., by 10% or greater, 15% or greater, or 20% or greater.

Without wishing to be bound by theory, in some aspects the pharmaceutical compositions of the invention contain a compound that prevents or inhibits amyloid fibril formation, either in the brain or other organ of interest (acting locally) or throughout the entire body (acting systemically). Pharmaceutical compositions of the invention may be effective in controlling amyloid deposition either following their entry into the brain (following penetration of the blood brain barrier) or from the periphery. When acting from the periphery, a compound of a pharmaceutical composition may alter the equilibrium of amyloidogenic peptide between the brain and the plasma so as to favor the exit of amyloidogenic peptide from the brain. It may also favor clearance (or catabolism) of the amyloid protein (soluble), and then prevent amyloid fibril formation and deposition due to a reduction of the amyloid protein pool in a specific organ, e.g., liver, spleen, pancreas, kidney, joints, brain, etc. An increase in the exit of amyloidogenic peptide from the brain would result in a decrease in amyloidogenic peptide brain concentration and therefore favor a decrease in amyloidogenic peptide deposition. In particular, an agent may lower the levels of amyloid β peptides, e.g., both Aβ40 and Aβ42 in the CSF and the plasma, or the agent may lower the levels of amyloid β peptides, e.g., Aβ40 and Aβ42 in the CSF and increase it in the plasma. Alternatively, compounds that penetrate the brain could control deposition by acting directly on brain amyloidogenic peptide e.g., by maintaining it in a non-fibrillar form or favoring its clearance from the brain, by increasing its degradation in the brain, or protecting brain cells from the detrimental effect of amyloidogenic peptide. An agent can also cause a decrease of the concentration of the amyloid protein (i.e., in a specific organ so that the critical concentration needed to trigger amyloid fibril formation or deposition is not reached). Furthermore, the compounds described herein may inhibit or reduce an interaction between amyloid and a cell surface constituent, for example, a glycosaminoglycan or proteoglycan constituent of a basement membrane, whereby inhibiting or reducing this interaction produces the observed neuroprotective and cell-protective effects. For example, the compound may also prevent an amyloid peptide from binding or adhering to a cell surface, a process which is known to cause cell damage or toxicity. Similarly, the compound may block amyloid-induced cellular toxicity or microglial activation or amyloid-induced neurotoxicity, or inhibit amyloid induced inflammation. The compound may also reduce the rate or amount of amyloid aggregation, fibril formation, or deposition, or the compound lessens the degree of amyloid deposition. The foregoing mechanisms of action should not be construed as limiting the scope of the invention inasmuch as the invention may be practiced without such information.

The term “amyloid-β disease” (or “amyloid-O related disease,” which terms as used herein are synonymous) may be used for mild cognitive impairment; vascular dementia; early Alzheimer's disease; Alzheimer's disease, including sporadic (non-hereditary) Alzheimer's disease and familial (hereditary) Alzheimer's disease; age-related cognitive decline; cerebral amyloid angiopathy (“CAA”); hereditary cerebral hemorrhage; senile dementia; Down's syndrome; inclusion body myositis (“IBM”); or age-related macular degeneration (“ARMD”). According to certain aspects of the invention, amyloid-β is a peptide having 39-43 amino-acids, or amyloid-β is an amyloidogenic peptide produced from βAPP.

Mild cognitive impairment (“MCI”) is a condition characterized by a state of mild but measurable impairment in thinking skills, which is not necessarily associated with the presence of dementia. MCI frequently, but not necessarily, precedes Alzheimer's disease. It is a diagnosis that has most often been associated with mild memory problems, but it can also be characterized by mild impairments in other thinking skills, such as language or planning skills. However, in general, an individual with MCI will have more significant memory lapses than would be expected for someone of their age or educational background. As the condition progresses, a physician may change the diagnosis to “Mild-to-Moderate Cognitive Impairment,” as is well understood in this art.

Cerebral amyloid angiopathy (“CAA”) refers to the specific deposition of amyloid fibrils in the walls of leptomingeal and cortical arteries, arterioles and in capillaries and veins. It is commonly associated with Alzheimer's disease, Down's syndrome and normal aging, as well as with a variety of familial conditions related to stroke or dementia (see Frangione, et al., Amyloid: J. Protein Folding Disord. 8, Suppl. 1, 36-42 (2001)). CAA can occur sporadically or be hereditary. Multiple mutation sites in either Aβ or the APP gene have been identified and are clinically associated with either dementia or cerebral hemorrhage. Exemplary CAA disorders include, but are not limited to, hereditary cerebral hemorrhage with amyloidosis of Icelandic type (HCHWA-I); the Dutch variant of HCHWA (HCHWA-D; a mutation in Aβ); the Flemish mutation of Aβ; the Arctic mutation of Aβ; the Italian mutation of Aβ; the Iowa mutation of Aβ; familial British dementia; and familial Danish dementia. Cerebral amyloid angiopathy is known to be associated with cerebral hemorrhage (or hemorrhagic stroke).

Also, the invention relates to a method for preventing or inhibiting amyloid deposition in a subject. For example, such a method comprises administering to a subject a therapeutically effective amount of a compound capable of reducing the concentration of amyloid (e.g., AL amyloid protein (λ or κ-chain related, e.g, amyloid λ, amyloid κ, amyloid κIV, amyloid λVI, amyloid γ, amyloid γ1), Aβ, IAPP, β₂M, AA, AH amyloid protein, or other amyloids), such that amyloid accumulation or deposition is prevented or inhibited.

In another aspect, the invention relates to a method for preventing, reducing, or inhibiting amyloid deposition in a subject. For example, such a method comprises administering to a subject a therapeutically effective amount of a compound capable of inhibiting amyloid (e.g., AL amyloid protein (λ or κ-chain related, e.g., amyloid λ, amyloid κ, amyloid κIV, amyloid λVI, amyloid γ, amyloid γ1), Aβ, IAPP, β₂M, AA, AH amyloid protein, or other amyloids), such that amyloid deposition is prevented, reduced, or inhibited.

The invention also relates to a method for modulating, e.g., minimizing, amyloid-associated damage to cells, comprising the step of administering a compound capable of reducing the concentration of amyloid (e.g., AL amyloid protein (λ or κ-chain related, e.g., amyloid λ, amyloid κ, amyloid κIV, amyloid λVI, amyloid γ, amyloid γ1), Aβ, IAPP, β₂M, AA, AH amyloid protein, or another amyloid), such that said amyloid-associated damage to cells is modulated. In certain aspects of the invention, the methods for modulating amyloid-associated damage to cells comprise a step of administering a compound capable of reducing the concentration of amyloid or reducing the interaction of an amyloid with a cell surface.

The invention also includes a method for directly or indirectly preventing cell death in a subject, the method comprising administering to a subject a therapeutically effective amount of a compound capable of preventing amyloid (e.g., AL amyloid protein (λ or κ-chain related, e.g., amyloid λ, amyloid κ, amyloid κIV, amyloid λVI, amyloid γ, amyloid γ1), Aβ, IAPP, β₂M, AA, AH amyloid protein, or other amyloid) mediated events that lead, directly or indirectly, to cell death.

In an embodiment, the method is used to treat Alzheimer's disease (e.g. sporadic or familial AD). The method can also be used prophylactically or therapeutically to treat other clinical occurrences of amyloid-O deposition, such as in Down's syndrome individuals and in patients with cerebral amyloid angiopathy (“CAA”) or hereditary cerebral hemorrhage.

The invention also includes a method for treating convulsive disorders, including epilepsy.

In one embodiment, the invention provides a method for inhibiting epileptogenesis in a subject. The method includes the step of administering to a subject in need thereof an effective amount of an agent which modulates a process in a pathway associated with epileptogenesis, such that epileptogenesis is inhibited in the subject.

As noted above, upregulation of excitatory coupling between neurons, mediated by N-methyl-D-aspartate (NMDA) receptors, and downregulation of inhibitory coupling between neurons, mediated by gamma-amino-butyric acid (GABA) receptors, have both been implicated in epileptogenesis. Other processes in pathways associated with epileptogenesis include release of nitric oxide (NO), a neurotransmitter implicated in epileptogenesis; release of calcium (Ca2+), which may mediate damage to neurons when released in excess; neurotoxicity due to excess zinc (Zn2+); neurotoxicity due to excess iron (Fe2+); and neurotoxicity due to oxidative cell damage. Accordingly, in some embodiments, an agent to be administered to a subject to inhibit epileptogenesis is capable of inhibiting one or more processes in at least one pathway associated with epileptogenesis. For example, an agent useful for inhibition of epileptogenesis can reduce the release of, or attenuate the epileptogenic effect of, NO in brain tissue; antagonize an NMDA receptor; augment endogenous GABA inhibition; block voltage-gated ion channels; reduce the release of, reduce the free concentration of (e.g., by chelation), or otherwise reduce the epileptogenic effect of cations including Ca²⁺, Zn²⁺, or Fe²⁺; inhibit oxidative cell damage; or the like. In certain embodiments, an agent to be administered to a subject to inhibit epileptogenesis is capable of inhibiting at least two processes in at least one pathway associated with epileptogenesis.

In still another embodiment, the invention provides a method of inhibiting a convulsive disorder. The method includes the step of administering to a subject in need thereof an effective amount of a β-amino anionic compound such that the convulsive disorder is inhibited; with the proviso that the β-amino anionic compound is not β-alanine or taurine.

In another embodiment, the invention provides a method for inhibiting both a convulsive disorder and epileptogenesis in a subject. The method includes the step of administering to a subject in need thereof an effective amount of an agent which a) blocks sodium or calcium ion channels, or opens potassium or chloride ion channels; and b) has at least one activity selected from the group consisting of NMDA receptor antagonism; augmentation of endogenous GABA inhibition; calcium binding; iron binding; zinc binding; NO synthase inhibition; and antioxidant activity; such that epileptogenesis is inhibited in the subject.

The compounds of the invention may be used prophylactically or therapeutically in the treatment of disorders in which amyloid-beta peptide is abnormally deposited at non-neurological locations, such as treatment of IBM by delivery of the compounds to muscle fibers, or treatment of macular degeneration by delivery of the compound(s) of the invention to the basal surface of the retinal pigmented epithelium.

The present invention also provides a method for modulating amyloid-associated damage to cells, comprising the step of administering a compound capable of reducing the concentration of Aβ, or capable of minimizing the interaction of Aβ (soluble oligomeric or fibrillary) with the cell surface, such that said amyloid-associated damage to cells is modulated. In certain aspects of the invention, the methods for modulating amyloid-associated damage to cells comprise a step of administering a compound capable of reducing the concentration of Aβ or reducing the interaction of Aβ with a cell surface.

In accordance with the present invention, there is further provided a method for preventing cell death in a subject, said method comprising administering to a subject a therapeutically effective amount of a compound capable of preventing Aβ-mediated events that lead, directly or indirectly, to cell death.

The present invention also provides a method for modulating amyloid-associated damage to cells, comprising the step of administering a compound capable of reducing the concentration of IAPP, or capable of minimizing the interaction of IAPP (soluble oligomeric or fibrillary) with the cell surface, such that said amyloid-associated damage to cells is modulated. In certain aspects of the invention, the methods for modulating amyloid-associated damage to cells comprise a step of administering a compound capable of reducing the concentration of IAPP or reducing the interaction of IAPP with a cell surface.

In accordance with the present invention, there is further provided a method for preventing cell death in a subject, said method comprising administering to a subject a therapeutically effective amount of a compound capable of preventing IAPP-mediated events that lead, directly or indirectly, to cell death.

This invention also provides methods and compositions which are useful in the treatment of amyloidosis. The methods of the invention involve administering to a subject a therapeutic compound which inhibits amyloid deposition. Accordingly, the compositions and methods of the invention are useful for inhibiting amyloidosis in disorders in which amyloid deposition occurs. The methods of the invention can be used therapeutically to treat amyloidosis or can be used prophylactically in a subject susceptible to (hereditary) amyloidosis or identified as being at risk to develop amyloidosis, e.g., hereditary, or identified as being at risk to develop amyloidosis. In certain embodiments, the invention includes a method of inhibiting an interaction between an amyloidogenic protein and a constituent of basement membrane to inhibit amyloid deposition. The constituent of basement membrane is a glycoprotein or proteoglycan, e.g., heparan sulfate proteoglycan. A therapeutic compound used in this method may interfere with binding of a basement membrane constituent to a target binding site on an amyloidogenic protein, thereby inhibiting amyloid deposition.

In some aspects, the methods of the invention involve administering to a subject a therapeutic compound which inhibits amyloid deposition. “Inhibition of amyloid deposition,” includes the prevention of amyloid formation, inhibition of further amyloid deposition in a subject with ongoing amyloidosis and reduction of amyloid deposits in a subject with ongoing amyloidosis. Inhibition of amyloid deposition is determined relative to an untreated subject or relative to the treated subject prior to treatment. In an embodiment, amyloid deposition is inhibited by inhibiting an interaction between an amyloidogenic protein and a constituent of basement membrane. “Basement membrane” refers to an extracellular matrix comprising glycoproteins and proteoglycans, including laminin, collagen type IV, fibronectin, perlecan, agrin, dermatan sulfate, and heparan sulfate proteoglycan (HSPG). In one embodiment, amyloid deposition is inhibited by interfering with an interaction between an amyloidogenic protein and a sulfated glycosaminoglycan such as HSPG, dermatan sulfate, perlecan or agrin sulfate. Sulfated glycosaminoglycans are known to be present in all types of amyloids (see Snow, et al. Lab. Invest. 56, 120-23 (1987)) and amyloid deposition and HSPG deposition occur coincidentally in animal models of amyloidosis (see Snow, et al. Lab. Invest. 56, 665-75 (1987) and Gervais, F. et al. Curr. Med. Chem., 3, 361-370 (2003)). Consensus binding site motifs for HSPG in amyloidogenic proteins have been described (see, e.g., Cardin and Weintraub Arteriosclerosis 9, 21-32 (1989)).

In some cases, the ability of a compound to prevent or block the formation or deposition of amyloid may reside in its ability to bind to non-fibrillar, soluble amyloid protein and to maintain its solubility.

The ability of a therapeutic compound of the invention to inhibit an interaction between an amyloidogenic protein and a glycoprotein or proteoglycan constituent of a basement membrane can be assessed by an in vitro binding assay, such as that described in U.S. Pat. No. 5,164,295, the contents of which are hereby incorporated by reference. Alternatively, the ability of a compound to bind to an amyloidogenic protein or to inhibit the binding of a basement membrane constituent (e.g. HSPG) to an amyloidogenic protein (e.g. Aβ) can be measured using a mass spectrometry assay where soluble protein, e.g Aβ, IAPP, β₂M is incubated with the compound. A compound which binds to, e.g Aβ, will induce a change in the mass spectrum of the protein. Exemplary protocols for a mass spectrometry assay employing Aβ and IAPP can be found in the Examples, the results of which are provided in Table 5. The protocol can readily be modified to adjust the sensitivity of the data, e.g., by adjusting the amount of protein and/or compound employed. Thus, e.g., binding might be detected for test compounds noted as not having detectable binding employing less sensitive test protocols.

Alternative methods for screening compounds exist and can readily be employed by a skilled practitioner to provide an indication of the ability of test compounds to bind to, e.g., fibrillar Aβ. One such screening assay is an ultraviolet absorption assay. In an exemplary protocol, a test compound (20 μM) is incubated with 50 μM AP(1-40) fibers for 1 hour at 37° C. in Tris buffered saline (20 mM Tris, 150 mM NaCl, pH 7.4 containing 0.01 sodium azide). Following incubation, the solution is centrifuged for 20 minutes at 21,000 g to sediment the Aβ(1-40) fibers along with any bound test compound. The amount of test compound remaining in the supernatant can then be determined by reading the absorbance. The fraction of test compound bound can then be calculated by comparing the amount remaining in the supernatants of incubations with Aβ to the amount remaining in control incubations which do not contain Aβ fibers. Thioflavin T and Congo Red, both of which are known to bind to Aβ fibers, may be included in each assay run as positive controls. Before assaying, test compounds can be diluted to 40 μM, which would be twice the concentration in the final test, and then scanned using the Hewlett Packard 8453 UVNVIS spectrophotometer to determine if the absorbance is sufficient for detection.

In another embodiment, the invention pertains to a method for improving cognition in a subject suffering from an amyloid associated disease. The method includes administering an effective amount of a therapeutic compound of the invention, such that the subject's cognition is improved. The subject's cognition can be tested using methods known in the art such as the Clinical Dementia Rating (“CDR”), Mini-Mental State Examination (“MMSE”), and the Alzheimer's Disease Assessment Scale-Cognition (“ADAS-Cog”).

In another embodiment, the invention pertains to a method for treating a subject for an amyloid associated disease. The method includes administering a cognitive test to a subject prior to administration of a compound of the invention, administering an effective amount of a compound of the invention to the subject, and administering a cognitive test to the subject subsequent to administration of the compound, such that the subject is treated for the amyloid associated disease, wherein the subject's score on said cognitive test is improved.

“Improvement,” “improved” or “improving” in cognition is present within the context of the present invention if there is a statistically significant difference in the direction of normality between the performance of subjects treated using the methods of the invention as compared to members of a placebo group, historical control, or between subsequent tests given to the same subject.

In one embodiment, a subject's CDR is maintained at 0. In another embodiment, a subject's CDR is decreased (e.g., improved) by about 0.25 or more, about 0.5 or more, about 1.0 or more, about 1.5 or more, about 2.0 or more, about 2.5 or more, or about 3.0 or more. In another embodiment, the rate of increase of a subject's CDR rating is reduced by about 5% or more, about 10% or more, about 20% or more, about 25% or more, about 30% or more, about 40% or more, about 50% or more, about 60% or more, about 70% or more, about 80% or more, about 90% or more, or about 100% or more of the increase of the historical or untreated controls.

In one embodiment, a subject's score on the MMSE is maintained. Alternatively, the subject's score on the MMSE may be increased by about 1, about 2, about 3, about 4, about 5, about 7.5, about 10, about 12.5, about 15, about 17.5, about 20, or about 25 points. In another alternative, the rate of the decrease of a subject's MMSE score as compared to historical controls is reduced. For example, the rate of the decrease of a subject's MMSE score may be reduced by about 5% or more, about 10% or more, about 20% or more, about 25% or more, about 30% or more, about 40% or more, about 50% or more, about 60% or more, about 70% or more, about 80% or more, about 90% or more, or about 100% or more of the decrease of the historical or untreated controls.

In one embodiment, the invention pertains to a method for treating, slowing or stopping an amyloid associated disease associated with cognitive impairment, by administering to a subject an effective amount of a therapeutic compound of the invention, wherein the annual deterioration of the subject's cognition as measured by ADAS-Cog is less than 8 points per year, less the 6 points per year, less than 5 points per year, less than 4 points per year, or less than 3 points per year. In a further embodiment, the invention pertains to a method for treating, slowing or stopping an amyloid associated disease associated with cognition by administering an effective amount of a therapeutic compound of the invention such that the subject's cognition as measured by ADAS-Cog remains constant over a year. “Constant” includes fluctuations of no more than 2 points. Remaining constant includes fluctuations of two points or less in either direction. In a further embodiment, the subject's cognition improves by 2 points or greater per year, 3 points or greater per year, 4 point or greater per year, 5 points or greater per year, 6 points or greater per year, 7 points or greater per year, 8 points or greater per year, etc. as measured by the ADAS-Cog. In another alternative, the rate of the increase of a subject's ADAS-Cog score as compared to historical controls is reduced. For example, the rate of the increase of a subject's ADAS-Cog score may be reduced by about 5% or more, about 10% or more, about 20% or more, about 25% or more, about 30% or more, about 40% or more, about 50% or more, about 60% or more, about 70% or more, about 80% or more, about 90% or more or about 100% of the increase of the historical or untreated controls.

In another embodiment, the ratio of Aβ42:Aβ40 in the CSF or plasma of a subject decreases by about 15% or more, about 20% or more, about 25% or more, about 30% or more, about 35% or more, about 40% or more, about 45% or more, or about 50% or more. In another embodiment, the levels of Aβ in the subject's cerebrospinal fluid decrease by about 15% or more, about 25% or more, about 35% or more, about 45% or more, about 55% or more, about 75% or more, or about 90% or more.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values in these values and ranges may also be the upper or lower limits of a range.

Furthermore, the invention pertains to any novel chemical compound described herein. That is, the invention relates to novel compounds, and novel methods of their use as described herein, which are within the scope of the Formulae disclosed herein, and which are not disclosed in the cited Patents and Patent Applications.

Use of Compounds of the Invention in Imaging Methods

It has also been discovered that the binding properties of the compounds of the present invention can be combined with imaging properties of fluorine moieties to provide compounds that are not only useful for the treatment of diseases (e.g., amyloid-associated diseases and CNS diseases), but that can also be used as an NMR detectable agent for a number of diagnostic and therapeutic uses (e.g., detection of amyloid, diagnosis of disease and/or diagnosis of disease state).

Accordingly, the invention provides a detectable agent (e.g., a contrast agent, imaging probe or diagnostic reagent) that binds or otherwise associates with a moiety of interest (e.g., Aβ, IAPP and β₂M) in a subject or sample or tissue or cell, thus allowing detection of the compound and the moiety of interest. Use of such compounds can provide information such as the presence and location and density or amount of a moiety of interest (e.g., an amyloid). Such information can allow diagnosis of a disease or disease state or a predisposition of such a disease or disease state. Accordingly, the present invention provides methods of using the compounds of the invention to detect, diagnose, and monitor disease or a predisposition to a disease or disease state. These methods can be used with any of the subject populations described herein, to detect any of the amyloid proteins described and/or to treat any of the amyloid related diseases described herein. These methods may include employing any of the compounds described herein that include a fluorine moiety.

The compounds of the invention that include a fluorine moiety may be used as contrast agents, imaging probes and/or diagnostic reagents. For example, the compounds of the invention that include a fluorine moiety may be used in accordance with the method of the present invention to detect or locate amyloid and/or amyloid deposits. The compounds of the invention that include a fluorine moiety can be employed to enhance imaging, e.g., of amyloid fibril formation and/or the surrounding environment of amyloid.

The term “imaging probe” refers to a probe that can be employed in conjunction with an imaging technique. Exemplary probes may include the compounds of the invention comprising a ¹⁹F isotope (and/or another isotope which has properties which allow it to be detected by imaging techniques), which can be used in conjunction with imaging techniques such as Magnetic Resonance Imaging (MRI) or Magnetic Resonance Spectroscopy (MRS). Imaging probes can be used to image or probe biological or other structures.

The term “diagnostic reagent” refers to agents that can be employed to diagnose or aid in the diagnosis of a disease or disorder (e.g., an amyloid-related disease or disorder). By way of example, a diagnostic reagent can be employed to provide information regarding the stage or progression or regression of the disease or disorder and/or to identify particular locations of or localizations of disease or disorder related moieties (e.g., locations of or localizations of amyloid proteins).

The term “contrast agent” refers to agents that can enhance imaging of cells, organs, and other structures. In fluoroscopy, contrast agents are used to enhance the imaging of otherwise radiolucent tissues. Generally, fluoroscopic contrast agents work by x-ray absorption. For NMR or MRI image enhancement, contrast agents generally shorten either the T₁ or T₂ proton relaxation times, giving rise to intensity enhancement in appropriately weighted images.

The fluorinated compounds of the invention can include one, a plurality, or even a maximum number of chemically equivalent fluorines on one or more substituents resonating at one or only few frequencies, e.g., from trifluoromethyl functions. Spectral aspects of fluorinated compounds generally are known and described in the literature. See e.g., Sotak, C. H. et al., MAGN. RESON. MED. 29:188-195 (1993).

In one embodiment, the compounds of the invention that include a fluorine moiety are water soluble. This can enhance the functionality of the compounds of the invention in many biomedical settings, as it can, e.g., obviate the need for emulsifiers.

In one embodiment of the present invention, an effective amount of a formulation or composition comprising a fluorinated compound of the invention in a pharmaceutically acceptable carrier is administered to a patient, and the patient, or a portion of the patient, is imaged. The term “amount effective to provide a detectable NMR signal”, refers to a non-toxic amount of compound sufficient to allow detection or to enhance or alter a MRI image. The compound can be administered in an amount that permits detection of the compounds or structures of interest (e.g. amyloid protein or amyloid plaques) and/or enhance detection or visualization of these compounds or structures as well as the surrounding organs or tissues. In one embodiment, the patient is mammal, e.g., a human or non-human mammal. In another embodiment, an effective amount of compound is administered or introduced to a tissue, one or more cells, or a sample, e.g., that include a moiety of interest such as amyloid proteins.

The above methods can include the administration of additional agents or therapies, including agents that inhibit amyloid deposition that are not compounds of the invention. The administration may be staggered or contemporaneous with the administration of the fluorinated compounds of the invention. Accordingly, the method can be used, e.g., to assess the efficacy of such additional compounds by imaging a subject subsequent to the administration of the additional compound.

The compounds of the present invention may be administered by any suitable route described herein, including, for example, parenterally (including subcutaneous, intramuscular, intravenous, intradermal and pulmonary), for imaging of internal organs, tissues, tumors, and the like. It will be appreciated that the route be selected depending on the organs or tissues to be imaged.

In one embodiment, the compound is administered alone. In another embodiment, it is administered as a pharmaceutical formulation comprising at least one compound of the invention and one or more pharmaceutically acceptable carriers, diluents or excipients as described herein. The formulation can optionally include delivery systems such as emulsions, liposomes and microparticles. The pharmaceutical formulation may optionally include other diagnostic or therapeutic agents, including other contrast agents, probes and/or diagnostic agents. The compounds of the present invention may also be presented for use in the form of veterinary formulations, which may be prepared, for example, by methods that are conventional in the art.

Dosages of the fluorinated compounds of the invention can depend on the spin density, flow (diffusion and perfusion), susceptibility, and relaxivity (T1 and T2) of the compounds of the invention. Dosages of the compounds of the invention may be conveniently calculated in milligrams of ¹⁹F per kilogram of patient (abbreviated as mg ¹⁹F/kg). For example, for parenteral administration, typical dosages may be from about 50 to about 1000 mg ¹⁹F/kg, more preferably from about 100 to about 500 mg ¹⁹F/kg. The dosage may take into account other fluorinated compounds in the administered formula.

For methods of continuous administrations (e.g., intravenous), suitable rates of administration are known in the art. Typical rates of administration are about 0.5 to 5 mL of formulation per second, more preferably about 1-3 mL/s. Imaging may begin before or after commencing administration, continue during administration, and may continue after administration.

It will be appreciated that dosages, dosage volumes, formulation concentrations, rates of administration, and imaging protocols will be individualized to the particular patient and the examination sought, and may be determined by an experienced practitioner. Guidelines for selecting such parameters are known in the art. The Contrast Media Manual, (1992, R. W. Katzberg, Williams and Wilkins, Baltimore, Md.).

It is to be understood that the invention also is directed to use of the compounds and methods of the invention employing Magnetic Resonance Spectroscopy (MRS). MRS can be employed to identify structures and/or compounds in the immediate vicinity of the compounds of the invention. By analysis of the resonance frequency of the surrounding atoms, which are slightly different in different compounds because of the electron shielding unique to each compound, different compounds are identifiable with MRS.

Accordingly, in another aspect of the invention MRS is used, with or without other imaging techniques.

Synthesis of Compounds of the Invention

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here. Functional and structural equivalents of the compounds described herein and which have the same general properties, wherein one or more simple variations of substituents are made which do not adversely affect the essential nature or the utility of the compound are also included.

The compounds of the present invention may be readily prepared in accordance with the synthesis schemes and protocols described herein, as illustrated in the specific procedures provided. However, those skilled in the art will recognize that other synthetic pathways for forming the compounds of this invention may be used, and that the following is provided merely by way of example, and is not limiting to the present invention. See, e.g., “Comprehensive Organic Transformations” by R. Larock, VCH Publishers (1989). It will be further recognized that various protecting and deprotecting strategies will be employed that are standard in the art (See, e.g., “Protective Groups in Organic Synthesis” by Greene and Wuts). Those skilled in the relevant arts will recognize that the selection of any particular protecting group (e.g., amine and carboxyl protecting groups) will depend on the stability of the protected moiety with regards to the subsequent reaction conditions and will understand the appropriate selections.

Further illustrating the knowledge of those skilled in the art is the following sampling of the extensive chemical literature: “Chemistry of the Amino Acids” by J. P. Greenstein and M. Winitz, John Wiley & Sons, Inc., New York (1961); “Comprehensive Organic Transformations” by R. Larock, VCH Publishers (1989); T. D. Ocain, et al., J. Med. Chem. 31, 2193-99 (1988); E. M. Gordon, et al., J. Med. Chem. 31, 2199-10 (1988); “Practice of Peptide Synthesis” by M. Bodansky and A. Bodanszky, Springer-Verlag, New York (1984); “Protective Groups in Organic Synthesis” by T. Greene and P. Wuts (1991); “Asymmetric Synthesis: Construction of Chiral Molecules Using Amino Acids” by G. M. Coppola and H. F. Schuster, John Wiley & Sons, Inc., New York (1987); “The Chemical Synthesis of Peptides” by J. Jones, Oxford University Press, New York (1991); and “Introduction of Peptide Chemistry” by P. D. Bailey, John Wiley & Sons, Inc., New York (1992).

The synthesis of compounds of the invention is carried out in a solvent. Suitable solvents are liquids at ambient room temperature and pressure or remain in the liquid state under the temperature and pressure conditions used in the reaction. Useful solvents are not particularly restricted provided that they do not interfere with the reaction itself (that is, they preferably are inert solvents), and they dissolve a certain amount of the reactants. Depending on the circumstances, solvents may be distilled or degassed. Solvents may be, for example, aliphatic hydrocarbons (e.g., hexanes, heptanes, ligroin, petroleum ether, cyclohexane, or methylcyclohexane) and halogenated hydrocarbons (e.g., methylenechloride, chloroform, carbontetrachloride, dichloroethane, chlorobenzene, or dichlororbenzene); aromatic hydrocarbons (e.g., benzene, toluene, tetrahydronaphthalene, ethylbenzene, or xylene); ethers (e.g., diglyme, methyl-tert-butyl ether, methyl-tert-amyl ether, ethyl-tert-butyl ether, diethylether, diisopropylether, tetrahydrofuran or methyltetrahydrofurans, dioxane, dimethoxyethane, or diethyleneglycol dimethylether); nitrites (e.g., acetonitrile); ketones (e.g., acetone); esters (e.g., methyl acetate or ethyl acetate); and mixtures thereof.

In general, after completion of the reaction, the product is isolated from the reaction mixture according to standard techniques. For example, the solvent is removed by evaporation or filtration if the product is solid, optionally under reduced pressure. After the completion of the reaction, water may be added to the residue to make the aqueous layer acidic or basic and the precipitated compound filtered, although care should be exercised when handling water-sensitive compounds. Similarly, water may be added to the reaction mixture with a hydrophobic solvent to extract the target compound. The organic layer may be washed with water, dried over anhydrous magnesium sulphate or sodium sulphate, and the solvent is evaporated to obtain the target compound. The target compound thus obtained may be purified, if necessary, e.g., by recrystallization, reprecipitation, chromatography, or by converting it to a salt by addition of an acid or base.

The compounds of the invention may be supplied in a solution with an appropriate solvent or in a solvent-free form (e.g., lyophilized). In another aspect of the invention, the compounds and buffers necessary for carrying out the methods of the invention may be packaged as a kit, optionally including a container. The kit may be commercially used for treating or preventing amyloid associated diseases and/or CNS diseases according to the methods described herein and may include instructions for use in a method of the invention. Additional kit components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The term “container” includes any receptacle for holding the therapeutic compound. For example, in one embodiment, the container is the packaging that contains the compound. In other embodiments, the container is not the packaging that contains the compound, i.e., the container is a receptacle, such as a box or vial that contains the packaged compound or unpackaged compound and the instructions for use of the compound. Moreover, packaging techniques are well known in the art. It should be understood that the instructions for use of the therapeutic compound may be contained on the packaging containing the therapeutic compound, and as such the instructions form an increased functional relationship to the packaged product.

Pharmaceutical Preparations

In another embodiment, the present invention relates to pharmaceutical compositions comprising agents according to any of the Formulae herein for the treatment of an amyloid associated disease and/or a CNS disease, as well as methods of manufacturing such pharmaceutical compositions.

In general, the agents of the present invention may be prepared by the methods illustrated in the general reaction schemes as, for example, in the patents and patent applications refered to herein, or by modifications thereof, using readily available starting materials, reagents and conventional synthesis procedures. In these reactions, it is also possible to make use of variants which are in themselves known, but are not mentioned here. Functional and structural equivalents of the agents described herein and which have the same general properties, wherein one or more simple variations of substituents are made which do not adversely affect the essential nature or the utility of the agent are also included.

The agents of the invention may be supplied in a solution with an appropriate solvent or in a solvent-free form (e.g., lyophilized). In another aspect of the invention, the agents and buffers necessary for carrying out the methods of the invention may be packaged as a kit. The kit may be commercially used according to the methods described herein and may include instructions for use in a method of the invention. Additional kit components may include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The therapeutic agent may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

To administer the therapeutic agent by other than parenteral administration, it may be necessary to coat the agent with, or co-administer the agent with, a material to prevent its inactivation. For example, the therapeutic agent may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7, 27 (1984)).

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi.

Suitable pharmaceutically acceptable vehicles include, without limitation, any non-immunogenic pharmaceutical adjuvants suitable for oral, parenteral, nasal, mucosal, transdermal, intravascular (IV), intraarterial (IA), intramuscular (IM), and subcutaneous (SC) administration routes, such as phosphate buffer saline (PBS).

The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic agent in the required amount in an appropriate solvent with one or a combination of ingredients enumerated above, as required, followed by filtered sterilization. Generally, dispersions are prepared by incorporating the therapeutic agent into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient (i.e., the therapeutic agent) plus any additional desired ingredient from a previously sterile-filtered solution thereof.

The therapeutic agent can be orally administered, for example, with an inert diluent or an assimilable edible carrier. The therapeutic agent and other ingredients may also be enclosed in a hard or soft shell gelatin capsule, compressed into tablets, or incorporated directly into the subject's diet. For oral therapeutic administration, the therapeutic agent may be incorporated with excipients and used in the form of ingestible tablets, buccal tablets, troches, capsules, elixirs, suspensions, syrups, wafers, and the like. The percentage of the therapeutic agent in the compositions and preparations may, of course, be varied. The amount of the therapeutic agent in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic agent calculated to produce the desired therapeutic effect in association with the required pharmaceutical vehicle. The specification for the dosage unit forms of the invention are dictated by and directly dependent on (a) the unique characteristics of the therapeutic agent and the particular therapeutic effect to be achieved, and (b) the limitations inherent in the art of compounding such a therapeutic agent for the treatment of amyloid deposition in subjects.

The present invention therefore includes pharmaceutical formulations comprising the agents of the Formulae described herein, including pharmaceutically acceptable salts thereof, in pharmaceutically acceptable vehicles for aerosol, oral and parenteral administration. Also, the present invention includes such agents, or salts thereof, which have been lyophilized and which may be reconstituted to form pharmaceutically acceptable formulations for administration, as by intravenous, intramuscular, or subcutaneous injection. Administration may also be intradermal or transdermal.

In accordance with the present invention, an agent of the Formulae described herein, and pharmaceutically acceptable salts thereof, may be administered orally or through inhalation as a solid, or may be administered intramuscularly or intravenously as a solution, suspension or emulsion. Alternatively, the agents or salts may also be administered by inhalation, intravenously or intramuscularly as a liposomal suspension.

Pharmaceutical formulations are also provided which are suitable for administration as an aerosol, by inhalation. These formulations comprise a solution or suspension of the desired agent of any Formula herein, or a salt thereof, or a plurality of solid particles of the agent or salt. The desired formulation may be placed in a small chamber and nebulized. Nebulization may be accomplished by compressed air or by ultrasonic energy to form a plurality of liquid droplets or solid particles comprising the agents or salts. The liquid droplets or solid particles should have a particle size in the range of about 0.5 to about 5 microns. The solid particles can be obtained by processing the solid agent of any Formula described herein, or a salt thereof, in any appropriate manner known in the art, such as by micronization. The size of the solid particles or droplets will be, for example, from about 1 to about 2 microns. In this respect, commercial nebulizers are available to achieve this purpose.

A pharmaceutical formulation suitable for administration as an aerosol may be in the form of a liquid, the formulation will comprise a water-soluble agent of any Formula described herein, or a salt thereof, in a carrier which comprises water. A surfactant may be present which lowers the surface tension of the formulation sufficiently to result in the formation of droplets within the desired size range when subjected to nebulization.

Peroral compositions also include liquid solutions, emulsions, suspensions, and the like. The pharmaceutically acceptable vehicles suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Pharmaceutical compositions may also be coated by conventional methods, typically with pH or time-dependent coatings, such that the subject agent is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

Other compositions useful for attaining systemic delivery of the subject agents include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compositions of this invention can also be administered topically to a subject, e.g., by the direct laying on or spreading of the composition on the epidermal or epithelial tissue of the subject, or transdermally via a “patch”. Such compositions include, for example, lotions, creams, solutions, gels and solids. These topical compositions may comprise an effective amount, usually at least about 0.1%, or even from about 1% to about 5%, of an agent of the invention. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier may include pharmaceutically acceptable emollients, emulsifiers, thickening agents, solvents and the like.

In one embodiment, active agents are administered at a therapeutically effective dosage sufficient to inhibit amyloid deposition in a subject. A “therapeutically effective” dosage inhibits amyloid deposition by, for example, at least about 20%, or by at least about 40%, or even by at least about 60%, or by at least about 80% relative to untreated subjects. In the case of an Alzheimer's subject, a “therapeutically effective” dosage stabilizes cognitive function or prevents a further decrease in cognitive function (i.e., preventing, slowing, or stopping disease progression). The present invention accordingly provides therapeutic drugs. By “therapeutic” or “drug” is meant an agent having a beneficial ameliorative or prophylactic effect on a specific disease or condition in a living human or non-human animal.

In the case of AA or AL amyloidosis, the agent may improve or stabilize specific organ function. As an example, renal function may be stabilized or improved by 10% or greater, 20% or greater, 30% or greater, 40% or greater, 50% or greater, 60% or greater, 70% or greater, 80% or greater, or by greater than 90%.

In the case of IAPP, the agent may maintain or increase β-islet cell function, as determined by insulin concentration or the Pro-IAPP/IAPP ratio. In a further embodiment, the Pro-IAPP/IAPP ratio is increased by about 10% or greater, about 20% or greater, about 30% or greater, about 40% or greater, or by about 50%. In a further embodiment, the ratio is increased up to 50%. In addition, a therapeutically effective amount of the agent may be effective to improve glycemia or insulin levels.

In another embodiment, the active agents are administered at a therapeutically effective dosage sufficient to treat AA (secondary) amyloidosis and/or AL (primary) amyloidosis, by stabilizing renal finction, decreasing proteinuria, increasing creatinine clearance (e.g., by at least 50% or greater or by at least 100% or greater), remission of chronic diarrhea, or by weight gain (e.g., 10% or greater). In addition, the agents may be administered at a therapeutically effective dosage sufficient to improve nephrotic syndrome.

Furthermore, active agents may be administered at a therapeutically effective dosage sufficient to decrease deposition in a subject of amyloid protein, e.g., Aβ40 or Aβ42. A therapeutically effective dosage decreases amyloid deposition by, for example, at least about 15%, or by at least about 40%, or even by at least 60%, or at least by about 80% relative to untreated subjects.

In another embodiment, active agents are administered at a therapeutically effective dosage sufficient to increase or enhance amyloid protein, e.g., Aβ40 or Aβ42, in the blood, CSF, or plasma of a subject. A therapeutically effective dosage increases the concentration by, for example, at least about 15%, or by at least about 40%, or even by at least 60%, or at least by about 80% relative to untreated subjects.

In yet another embodiment, active agents are administered at a therapeutically effective dosage sufficient to maintain a subject's CDR rating at its base line rating or at 0. In another embodiment, the active agents are administered at a therapeutically effective dosage sufficient to decrease a subject's CDR rating by about 0.25 or more, about 0.5 or more, about 1.0 or more, about 1.5 or more, about 2.0 or more, about 2.5 or more, or about 3.0 or more. In another embodiment, the active agents are administered at a therapeutically effective dosage sufficient to reduce the rate of the increase of a subject's CDR rating as compared to historical or untreated controls. In another embodiment, the therapeutically effective dosage is sufficient to reduce the rate of increase of a subject's CDR rating (relative to untreated subjects) by about 5% or greater, about 10% or greater, about 20% or greater, about 25% or greater, about 30% or greater, about 40% or greater, about 50% or greater, about 60% or greater, about 70% or greater, about 80% or greater, about 90% or greater or about 100% or greater.

In yet another embodiment, active agents are administered at a therapeutically effective dosage sufficient to maintain a subject's score on the MMSE. In another embodiment, the active agents are administered at a therapeutically effective dosage sufficient to increase a subject's MMSE score by about 1, about 2, about 3, about 4, about 5, about 7.5, about 10, about 12.5, about 15, about 17.5, about 20, or about 25 points. In another embodiment, the active agents are administered at a therapeutically effective dosage sufficient to reduce the rate of the decrease of a subject's MMSE score as compared to historical controls. In another embodiment, the therapeutically effective dosage is sufficient to reduce the rate of decrease of a subject's MMSE score may be about 5% or less, about 10% or less, about 20% or less, about 25% or less, about 30% or less, about 40% or less, about 50% or less, about 60% or less, about 70% or less, about 80% or less, about 90% or less or about 100% or less, of the decrease of the historical or untreated controls.

In yet another embodiment, active agents are administered at a therapeutically effective dosage sufficient to maintain a subject's score on the ADAS-Cog. In another embodiment, the active agents are administered at a therapeutically effective dosage sufficient to decrease a subject's ADAS-Cog score by about 2 points or greater, by about 3 points or greater, by about 4 points or greater, by about 5 points or greater, by about 7.5 points or greater, by about 10 points or greater, by about 12.5 points or greater, by about 15 points or greater, by about 17.5 points or greater, by about 20 points or greater, or by about 25 points or greater. In another embodiment, the active agents are administered at a therapeutically effective dosage sufficient to reduce the rate of the increase of a subject's ADAS-Cog scores as compared to historical or untreated controls. In another embodiment, the therapeutically effective dosage is sufficient to reduce the rate of increase of a subject's ADAS-Cog scores (relative to untreated subjects) by about 5% or greater, about 10% or greater, about 20% or greater, about 25% or greater, about 30% or greater, about 40% or greater, about 50% or greater, about 60% or greater, about 70% or greater, about 80% or greater, about 90% or greater or about 100% or greater.

In another embodiment, active agents are administered at a therapeutically effective dosage sufficient to decrease the ratio of Aβ42:Aβ40 in the CSF or plasma of a subject by about 15% or more, about 20% or more, about 25% or more, about 30% or more, about 35% or more, about 40% or more, about 45% or more, or about 50% or more.

In another embodiment, active agents are administered at a therapeutically effective dosage sufficient to lower levels of Aβ in the CSF or plasma of a subject by about 15% or more, about 25% or more, about 35% or more, about 45% or more, about 55% or more, about 75% or more, or about 95% or more.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50, and usually a larger therapeutic index is more efficacious. While agents that exhibit toxic side effects may be used, care should be taken to design a delivery system that targets such agents to the site of affected tissue in order to minimize potential damage to unaffected cells and, thereby, reduce side effects.

It is understood that appropriate doses depend upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the subject. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses depend upon the potency. Such appropriate doses may be determined using the assays described herein. When one or more of these compounds is to be administered to an animal (e.g., a human), a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, and any drug combination.

The ability of an agent to inhibit amyloid deposition can be evaluated in an animal model system that may be predictive of efficacy in inhibiting amyloid deposition in human diseases, such as a transgenic mouse expressing human APP or other relevant animal models where Aβ deposition is seen or for example in an animal model of AA amyloidosis. Likewise, the ability of an agent to prevent or reduce cognitive impairment in a model system may be indicative of efficacy in humans. Alternatively, the ability of an agent can be evaluated by examining the ability of the agent to inhibit amyloid fibril formation in vitro, e.g., using a fibrillogenesis assay such as that described herein, including a ThT, CD, or EM assay. Also the binding of an agent to amyloid fibrils may be measured using a MS assay as described herein. The ability of the agent to protect cells from amyloid induced toxicity is determined in vitro using biochemical assays to determine percent cell death induced by amyloid protein. The ability of an agent to modulate renal function may also be evaluated in an appropriate animal model system.

The therapeutic agent of the invention may also be administered ex vivo to inhibit amyloid deposition or treat certain amyloid associated diseases, such as β₂M amyloidosis and other amyloidoses related to dialysis. Ex vivo administration of the therapeutic agents of the invention can be accomplished by contacting a body fluid (e.g., blood, plasma, etc.) with a therapeutic compound of the invention such that the therapeutic compound is capable of performing its intended function and administering the body fluid to the subject. The therapeutic compound of the invention may perform its function ex vivo (e.g., dialysis filter), in vivo (e.g., administered with the body fluid), or both. For example, a therapeutic compound of the invention may be used to reduce plasma β₂M levels and/or maintain β₂M in its soluble form ex vivo, in vivo, or both.

Prodrugs

The present invention is also related to prodrugs of the agents of the Formulae disclosed herein. Prodrugs are agents which are converted in vivo to active forms (see, e.g., R. B. Silverman, 1992, “The Organic Chemistry of Drug Design and Drug Action,” Academic Press, Chp. 8). Prodrugs can be used to alter the biodistribution (e.g., to allow agents which would not typically enter the reactive site of the protease) or the pharmacokinetics for a particular agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate agent which subsequently decomposes to yield the active agent. The prodrug moieties may be metabolized in vivo by esterases or by other mechanisms to carboxylic acids.

Examples of prodrugs and their uses are well known in the art (see, e.g., Berge, et al., “Pharmaceutical Salts”, J. Pharm. Sci. 66, 1-19 (1977)). The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

Pharmaceutically Acceptable Salts

Certain embodiments of the present agents can contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agents of the present invention. These salts can be prepared in situ during the final isolation and purification of the agents of the invention, or by separately reacting a purified agent of the invention in its free base form with a suitable organic or inorganic acid, and isolating the salt thus formed.

Representative salts include the hydrohalide (including hydrobromide and hydrochloride), sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, 2-hydroxyethanesulfonate, and laurylsulphonate salts and the like. See, e.g., Berge et al., “Pharmaceutical Salts”, J. Pharm. Sci. 66, 1-19 (1977).

In other cases, the agents of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention.

These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

“Pharmaceutically acceptable salts” also includes, for example, derivatives of agents modified by making acid or base salts thereof, as described further below and elsewhere in the present application. Examples of pharmaceutically acceptable salts include mineral or organic acid salts of basic residues such as amines; and alkali or organic salts of acidic residues such as carboxylic acids. Pharmaceutically acceptable salts include the conventional non-toxic salts or the quaternary ammonium salts of the parent agent formed, for example, from non-toxic inorganic or organic acids. Such conventional non-toxic salts include those derived from inorganic acids such as hydrochloric, hydrobromic, sulfuric, sulfamic, phosphoric, and nitric acid; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic acid. Pharmaceutically acceptable salts may be synthesized from the parent agent which contains a basic or acidic moiety by conventional chemical methods. Generally, such salts may be prepared by reacting the free acid or base forms of these agents with a stoichiometric amount of the appropriate base or acid in water or in an organic solvent, or in a mixture of the two.

All acid, salt, base, and other ionic and non-ionic forms of the compounds described are included as compounds of the invention. For example, if a compound is shown as an acid herein, the salt forms of the compound are also included. Likewise, if a compound is shown as a salt, the acid and/or basic forms are also included.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The contents of all references, issued patents, and published patent applications cited throughout this application are hereby incorporated by reference in their entireties. The invention is further illustrated by the following examples, which should not be construed as further limiting.

### EXAMPLES

### Example 1

### Synthesis of Library of Exemplary Compounds

Library compounds were synthesized in accordance with the following exemplary schemes:

Synthesis of library (Route 1):

Step 1 (deprotection): A solution of Fmoc-Gly-Wang resin (5 g, 5 mmol) in a fritted syringe was washed 4 times with DMF (30 mL). To cleave the Fmoc group, 35 mL of a 30 % piperidine/N-methylpyrrolidinone (NMP) solution was added to the resin and the suspension was shaken for 30 minutes. The reagents and solvent were filtered and the resin was washed 4 times with NMP (35 mL). A deep blue color on a Kaiser test was observed, indicating free amine.

Step 2 (Activation):

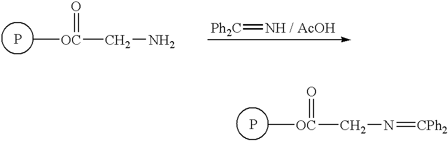

A solution of benzophenone imine (2.54 mL, 15 mmol) and glacial acetic acid (840 μL, 15 mmol) in NMP (35 mL) was prepared and the solution was introduced to the fritted syringe containing the free amino resin (Step 1, ˜5 g, ˜5 mmol). The suspension was shaken overnight at room temperature. The reagents and solvents were then removed by filtration. The resin was washed 4 times with DMF (30 mL), 4 times with methanol (35 mL), and once with DIEA (1N in methanol, 20 mL) for 30 min. The resin was then filtered and washed 4 times with DMF (30 mL), and 4 times with CH₂Cl₂ (30 mL), and subsequently dried overnight in vacuo.

Step 3 (Introduction of Block A):

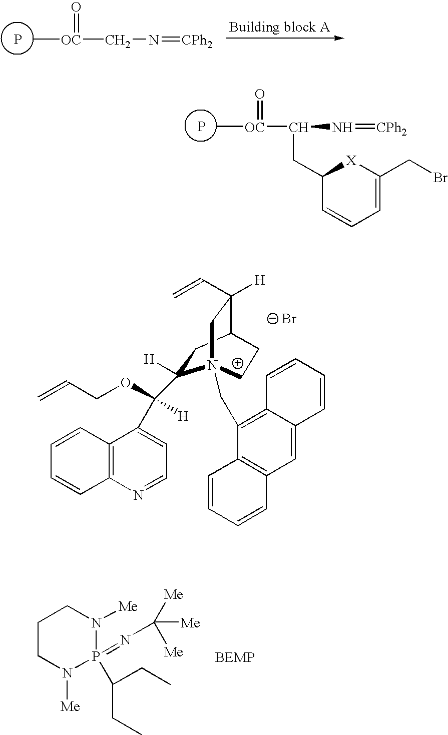

The benzophenone imine resin from Step 2 (2.3 g, ˜2.3 mmol), building block A as defined below (e.g., α,α-dibromo-m-xylene, 3.1 g, 11.7 mmol) and O-allyl-N-(9-anthracenylmethyl)cinchonidinium bromide (1.4 g, 2.3 mmol) were suspended in methylene chloride (30 mL). The suspension was shaken for 5 min and then cooled to −78° C. with a dry ice slurry in 2-propanol. The Dewar flask was fixed on Titer plate shaker with a foam lid to maintain the low temperature. The reaction mixture was shaken gently for 20 min at −78° C. 2-tert-Butylimino-2-diethylamino-1,3-dimethylperhydro-1,3,2-diazaphosphorine (BEMP, 3.3 mL, 11.4 mmol) was added via syringe. The reaction mixture was shaken at −78° C. for 5 h and then gradually warmed up to room temperature for 5 to 7 h. The reagents and solvents were then removed by filtration. The resin was washed 4 times with DMF (30 mL), 4 times with CH₂Cl₂ (30 mL), and 4 times with methanol (35 mL), and subsequently dried overnight in vacuo.

| Building block A | Products |
|---|---|
| 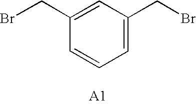 | 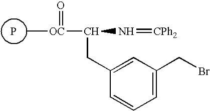 |
| 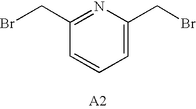 | 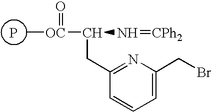 |

Step 4 (Coupling of Block C):

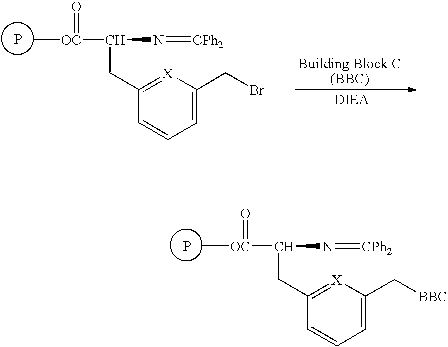

Each resin from Step 3 (50 mg each, ˜50 μmol) was distributed into 32 fritted syringes (Torvig, 50 mg each, ˜50), for a total of 64 syringes, and was swelled in NMP (1 mL) for 30 min. The solvent was removed from each syringe by filtration. Solutions of each of the sixteen building blocks listed below (10 mmol each) and DIEA (3.5 mL, 20 mmol) in NMP (10 mL) were prepared. 3 mL of solutions C1-C8 were added to the syringes containing the product incorporating building block A1, and 3 mL of solutions C9-C16 were added to the syringes containing the product incorporating building bock A2. The suspensions were then shaken for 20 h on a Titer Plate Shaker. The reaction mixtures were each filtered and washed 5 times with methylene chloride (5 mL), 3 times with THF (5 mL), three times with THF/H₂O (3/1 v/v, 5 mL), and three times with THF (5 mL) and the resins were dried overnight under vacuum.

| Building Block | Structure | Product # |
|---|---|---|
| 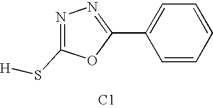 | 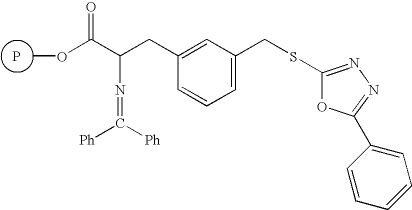 | Step-4-01 Step-4-02 Step-4-03 Step-4-04 |
| 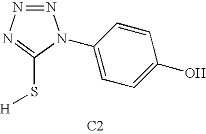 | 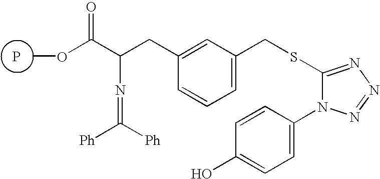 | Step-4-05 Step-4-06 Step-4-07 Step-4-08 |
| 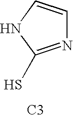 | 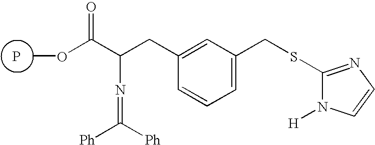 | Step-4-09 Step-4-10 Step-4-11 Step-4-12 |
| 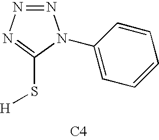 | 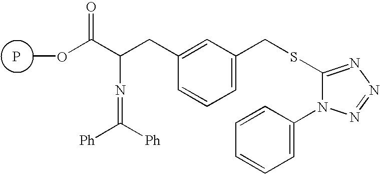 | Step-4-13 Step-4-14 Step-4-15 Step-4-16 |
| 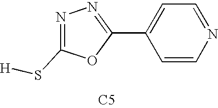 | 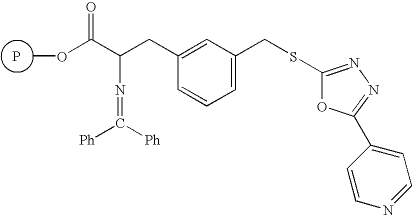 | Step-4-17 Step-4-18 Step-4-19 Step-4-20 |
| 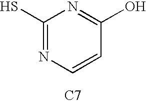 | 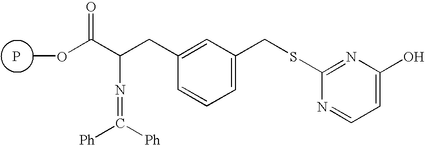 | Step-4-25 Step-4-26 Step-4-27 Step-4-28 |
| 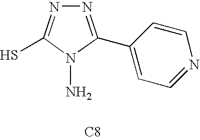 | 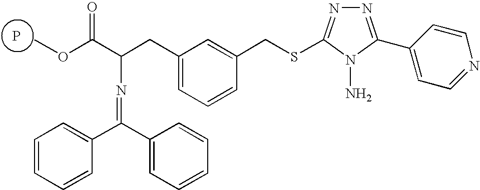 | Step-4-29 Step-4-30 Step-4-31 Step-4-32 |
| 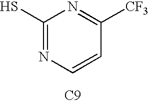 | 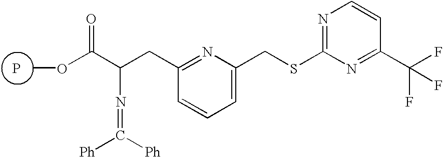 | Step-4-33 Step-4-34 Step-4-35 Step-4-36 |
| 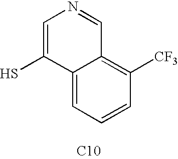 | 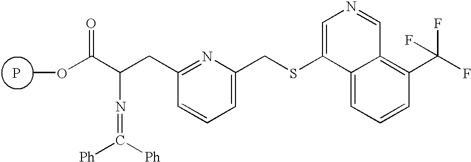 | Step-4-37 Step-4-38 Step-4-39 Step-4-40 |
| 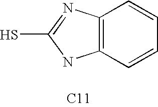 | 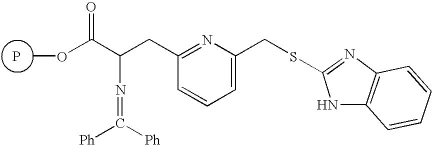 | Step-4-41 Step-4-42 Step-4-43 Step-4-44 |
| 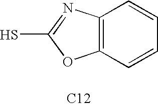 | 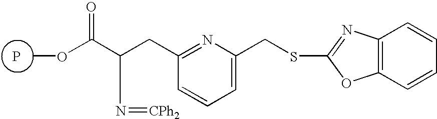 | Step-4-45 Step-4-46 Step-4-47 Step-4-48 |
| 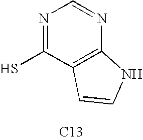 | 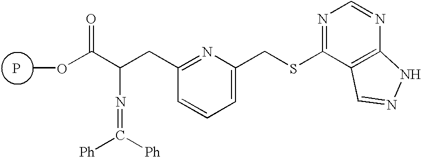 | Step-4-49 Step-4-50 Step-4-51 Step-4-52 |
| 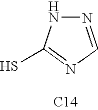 | 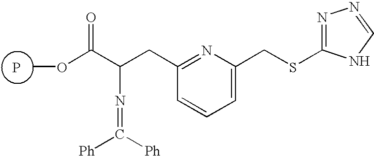 | Step-4-53 Step-4-54 Step-4-55 Step-4-56 |
| 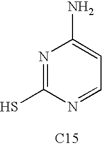 | 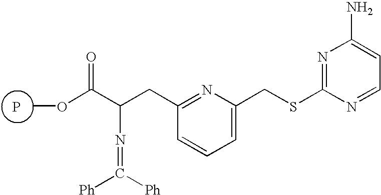 | Step-4-57 Step-4-58 Step-4-59 Step-4-60 |
| 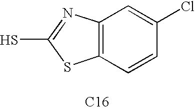 | 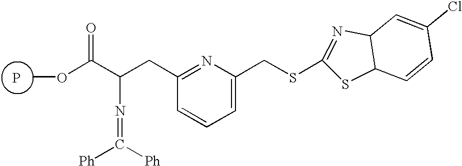 | Step-4-61 Step-4-62 Step-4-63 Step-4-64 |

Step 5 (Removal of Protecting Group):

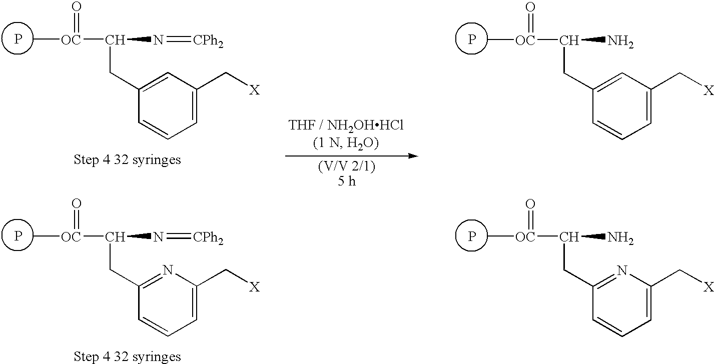

The resins from Step 4 (in their 64 original fritted syringes) were suspended in a 1N aqueous solution of NH₂OH.HCl/THF (1/2 v/v, 3 mL) and shaken for 5 h at ambient temperature. The reagents and solvents were then removed by filtration from each of the frits. The resin was washed 4 times with THF (2 mL), 4 times with DMF (2 mL), and once with DIEA (1N in DMF, 2 mL) for 30 min. The resins were then filtered and washed 4 times with DMF (2 mL), and 4 times with CH₂Cl₂ (2 mL), and subsequently dried in vacuo. A Kaiser test showed that resin was a deep blue color, indicating the free amine of the product.

Step 6, Coupling of Building Block D:
Part A:

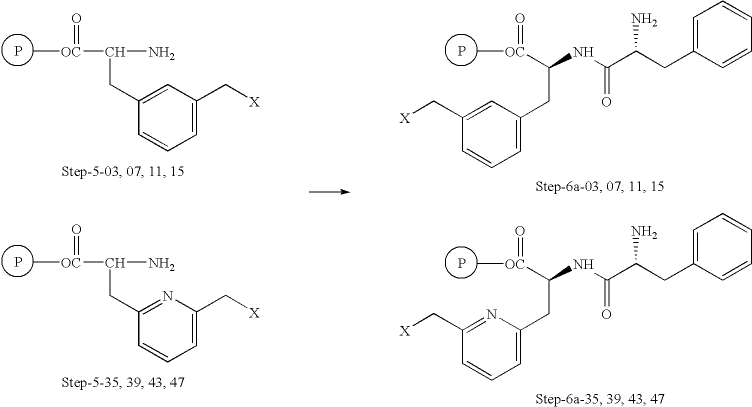

To a solution of Fmoc-D-Phe-OH (1.24 g, 3.2 mmol), PyBop (1.6 g, 3.08 mmol) and HOBt 5 (490 mg, 3.2 mmol) in DMA (anhydrous, 20 mL) was added DIEA (1.12 mL, 6.4 mmol). This solution was added to pre-swelled resins from Steps-5-03, 07, 11, 15, 35, 39, 43, and 47 in syringes. The suspensions were shaken at room temperature for 2 hours. The reagents and solvent were removed by filtration and each of the 8 resins were washed 4 times with DMF (3 mL) and 4 times with methylene chloride (3 mL) and the Fmoc was removed using the same procedure as in Step 1, above.
Part B:

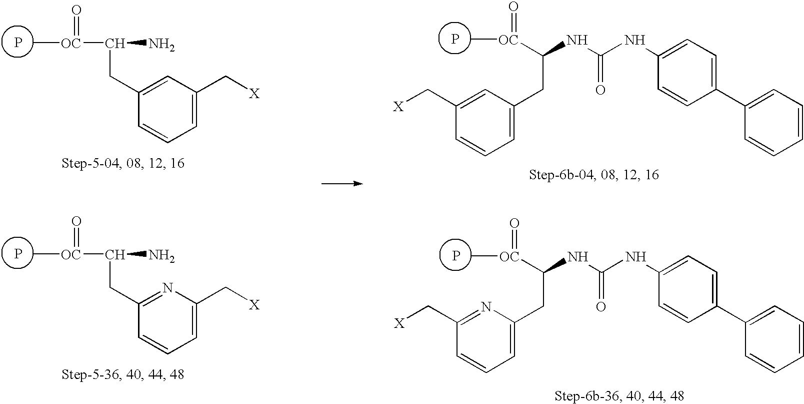

The resins from Steps-5-04, 08, 12, 16, 36, 40, 44, and 48 were suspended in methylene chloride (1 mL, anhydrous) in Torvig syringes for 5 min, and to each suspension was added a solution of 4-biphenylryl isocyanate (200 mg, ˜1 mmol) in DMF (anhydrous, 1 mL). Each suspension was shaken at room temperature overnight. The reagents and solvents were then removed by filtration and the resins were washed with MeOH and methylene chloride alternatively (3 mL each wash, 4 cycles).
Part C:

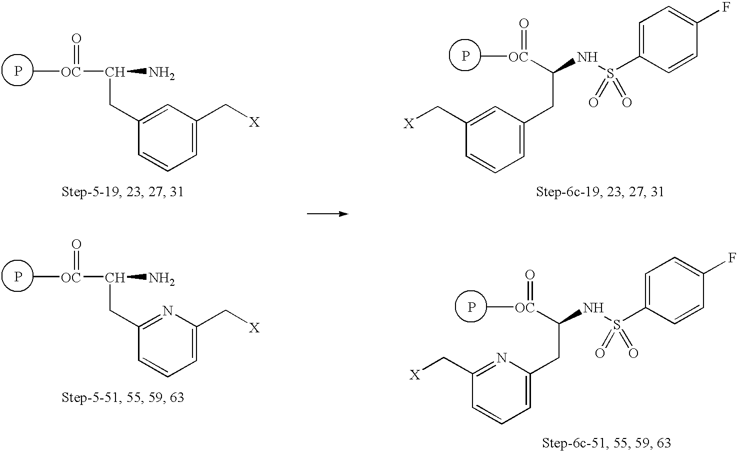

The resins from Steps-5-19, 23, 27, 31, 51, 55, 59, 63 were swelled in DMF (3 mL) for 30 min in their original fritted syringes. The majority of the solvent was removed by filtration. To each syringe was added 2.4 mL of a solution of 4-flurobenzenesulfonyl chloride (620 mg, 3.2 mmol) and N-methylmorpholine (700 μl, 6.4 mmol) in CH₂Cl₂ (20 mL). Each mixture was shaken overnight at ambient temperature. The reagents and solvents were removed by filtration. The resins were each washed 4 times with DMF (3 mL), and 4 times with CH₂Cl₂ (3 mL), and subsequently dried in vacuo.
Part D:

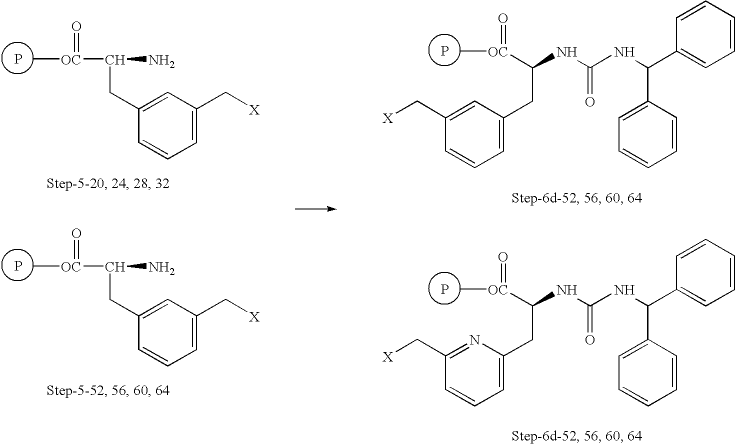

The resins from Steps-5-20, 24, 28, 32, 52, 56, 60, and 64 were suspended in methylene chloride (1 mL, anhydrous) in Torvig syringes for 5 min. To each suspension was added a solution of 4-diphenylmethyl isocyanate (210 mg, -1 mmol) in DMF (anhydrous, 1 mL). The suspensions were shaken at room temperature overnight. The reagents and solvents were removed by filtration and the resin was washed with MeOH and methylene chloride alternatively (3 mL each wash, 4 cycles).

Step 7a (Acid Cleavage):

| Starting Material | Product |
|---|---|
| Step-5-01 | Step-7-01 |
| Step-6a-03 | Step-7-03 |
| Step-6b-04 | Step-7-04 |
| Step-5-05 | Step-7-05 |
| Step-6a-07 | Step-7-07 |
| Step-6b-08 | Step-7-08 |
| Step-5-09 | Step-7-09 |
| Step-6a-11 | Step-7-11 |
| Step-6b-12 | Step-7-12 |
| Step-5-13 | Step-7-13 |
| Step-6a-15 | Step-7-15 |
| Step-6b-16 | Step-7-16 |
| Step-5-17 | Step-7-17 |
| Step-6c-19 | Step-7-19 |
| Step-6d-20 | Step-7-20 |
| Step-5-21 | Step-7-21 |
| Step-6c-23 | Step-7-23 |
| Step-6d-24 | Step-7-24 |
| Step-5-25 | Step-7-25 |
| Step-6c-27 | Step-7-27 |
| Step-6d-28 | Step-7-28 |
| Step-5-29 | Step-7-29 |
| Step-6c-31 | Step-7-31 |
| Step-6d-32 | Step-7-32 |
| Step-5-33 | Step-7-33 |
| Step-6a-035 | Step-7-35 |
| Step-6b-036 | Step-7-36 |
| Step-5-37 | Step-7-37 |
| Step-6a-39 | Step-7-39 |
| Step-6b-40 | Step-7-40 |
| Step-5-41 | Step-7-41 |
| Step-6a-43 | Step-7-43 |
| Step-6b-44 | Step-7-44 |
| Step-5-45 | Step-7-45 |
| Step-6a-47 | Step-7-47 |
| Step-6b-48 | Step-7-48 |
| Step-5-49 | Step-7-49 |
| Step-6c-51 | Step-7-51 |
| Step-6d-52 | Step-7-52 |
| Step-5-53 | Step-7-53 |
| Step-6c-55 | Step-7-55 |
| Step-6d-56 | Step-7-56 |
| Step-5-57 | Step-7-57 |
| Step-6c-59 | Step-7-59 |
| Step-6d-60 | Step-7-60 |
| Step-5-61 | Step-7-61 |
| Step-6c-63 | Step-7-63 |
| Step-6d-64 | Step-7-64 |

The resins shown above were each treated with TFA/Anisole/H₂O (95%/2.5%/2.5%, 1 mL each) for 5 min, and the filtrate was collected by filtration. The resins were again treated with TFA/Anisole/H₂O (95%/2.5%/2.5 %, 1 mL each) for 30 min. The filtrates from the same syringes were combined. To the filtrate was added cold ether (10 mL), the precipitate was centrifuged for 5 min at 4000 rpm, and the supernant was decanted. The precipitates were washed and centrifuged three additional times to remove possible impurities. ES-MASS indicated the correct molecular weight for the desired compounds.

Step 7b (Ammonia Cleavage):

| Starting Material | Product |
|---|---|
| Step-5-02 | Step-7-02 |
| Step-5-06 | Step-7-06 |
| Step-5-10 | Step-7-10 |
| Step-5-14 | Step-7-14 |
| Step-5-18 | Step-7-18 |
| Step-5-22 | Step-7-22 |
| Step-5-26 | Step-7-26 |
| Step-5-30 | Step-7-30 |
| Step-5-34 | Step-7-34 |
| Step-5-38 | Step-7-38 |
| Step-5-42 | Step-7-42 |
| Step-5-46 | Step-7-46 |
| Step-5-50 | Step-7-50 |
| Step-5-54 | Step-7-54 |
| Step-5-58 | Step-7-58 |
| Step-5-62 | Step-7-62 |

The resins shown above were each treated with ammonia in methanol (2 N solution, 2 mL) for 30 min, and the filtrate was collected by filtration. The resin was again treated with ammonia in methanol (2 N solution, 2 mL) for 2 h. The filtrates from the same syringes were combined. To the filtrate was added cold ether (10 mL), the precipitate was centrifuge for 5 min at 4000 rpm, and the supernant was decanted. For those syringes with no precipitation, hexanes were added. The precipitates were washed and centrifuged three additional times to remove the possible impurities.

The structures of the products from Route 1 are listed in the following table:

| Structure | Product | ID# | Structure | Product | ID# |
|---|---|---|---|---|---|
| 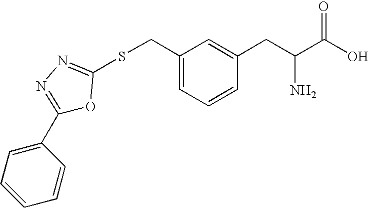 | Step-7-01 | 1 | 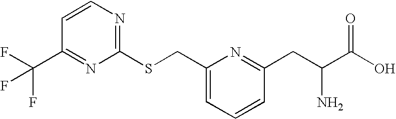 | Step-7-33 | 21 |
| 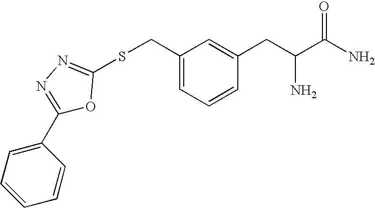 | Step-7-02 | 2 | 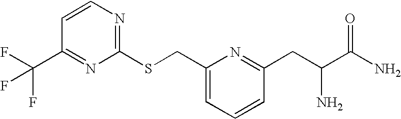 | Step-7-34 | 22 |
| 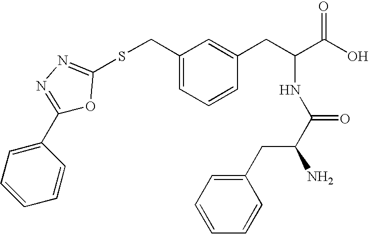 | Step-7-03 | 48 | 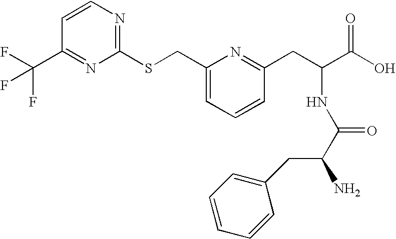 | Step-7-35 | 23 |
| 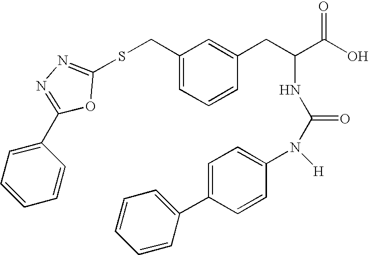 | Step-7-04 | 49 | 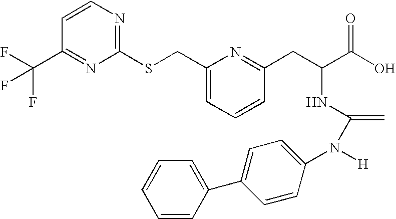 | Step-7-36 | 60 |
| 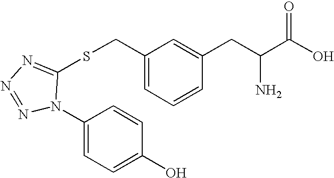 | Step-7-05 | 3 | 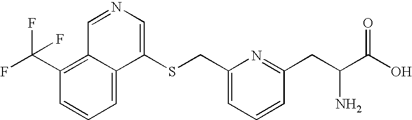 | Step-7-37 | 24 |
| 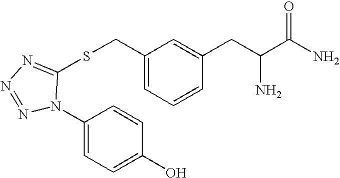 | Step-7-06 | 4 | 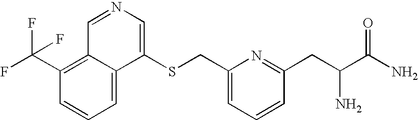 | Step-7-38 | 25 |
| 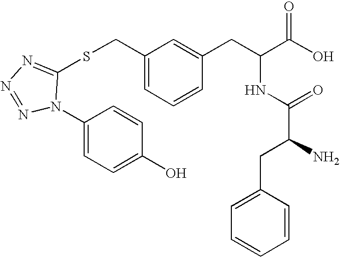 | Step-7-07 | 50 | 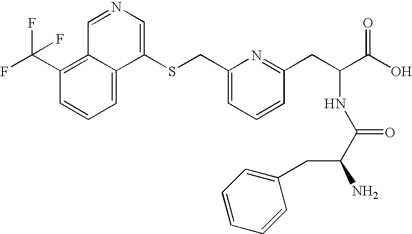 | Step-7-39 | 26 |
| 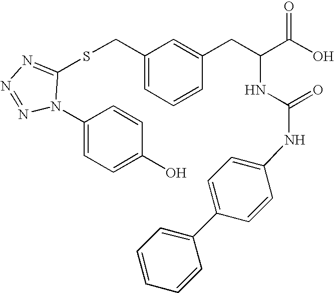 | Step-7-08 | 51 | 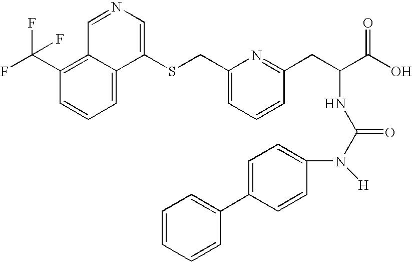 | Step-7-40 | 61 |
| 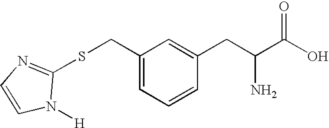 | Step-7-09 | 5 | 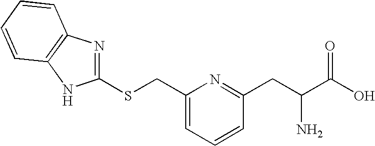 | Step-7-41 | 27 |
| 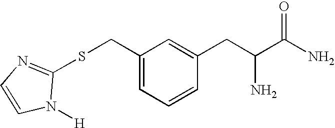 | Step-7-10 | 6 | 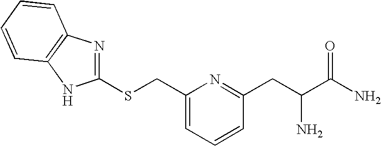 | Step-7-42 | 28 |
| 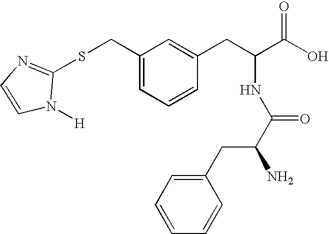 | Step-7-11 | 7 | 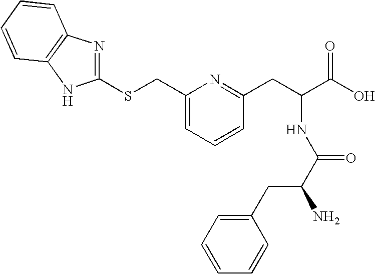 | Step-7-43 | 29 |
| 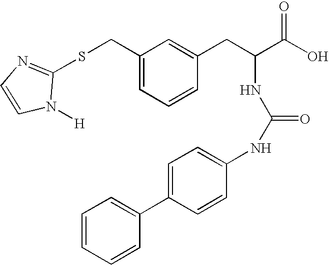 | Step-7-12 | 52 | 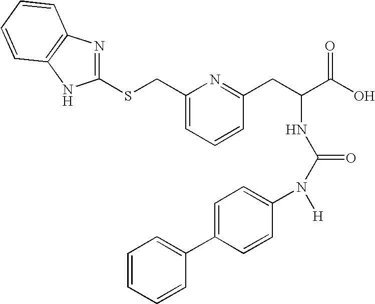 | Step-7-44 | 62 |
| 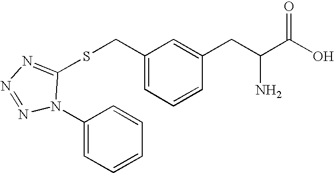 | Step-7-13 | 8 | 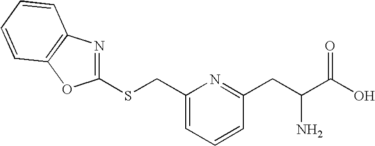 | Step-7-45 | 30 |
| 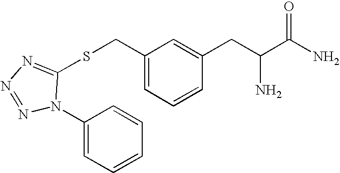 | Step-7-14 | 9 | 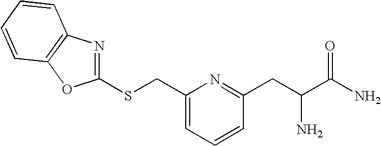 | Step-7-46 | 31 |
| 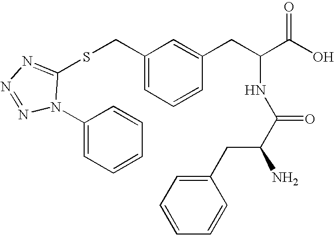 | Step-7-15 | 53 | 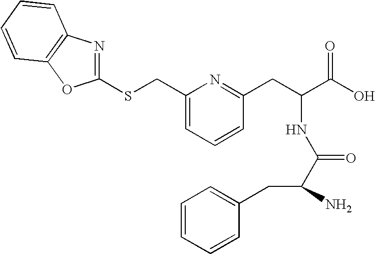 | Step-7-47 | 32 |
| 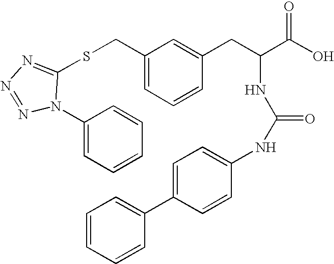 | Step-7-16 | 54 | 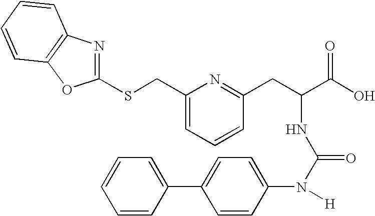 | Step-7-48 | 63 |
| 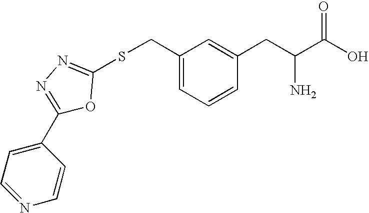 | Step-7-17 | 10 | 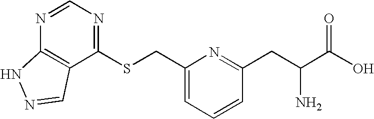 | Step-7-49 | 33 |
| 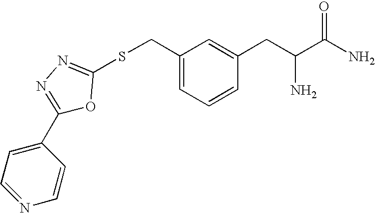 | Step-7-18 | 11 | 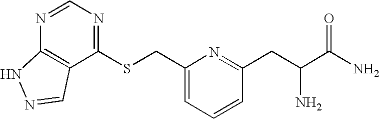 | Step-7-50 | 34 |
| 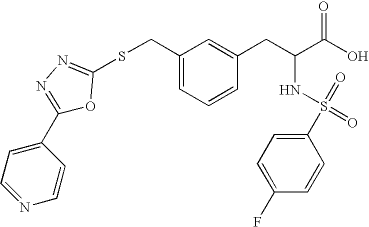 | Step-7-19 | 12 | 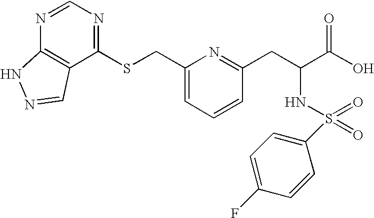 | Step-7-51 | 64 |
| 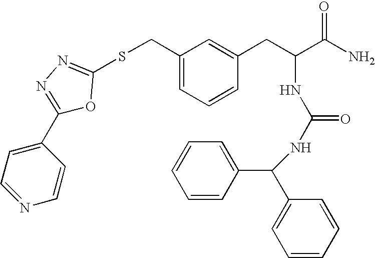 | Step-7-20 | 55 | 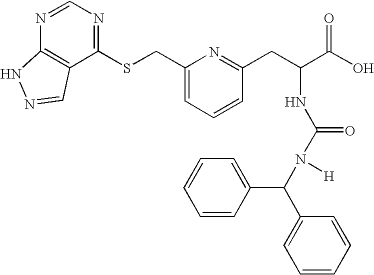 | Step-7-52 | 65 |
| 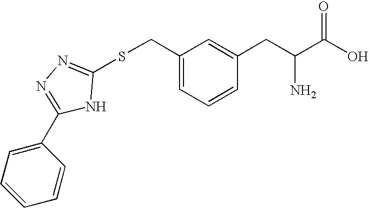 | Step-7-21 | 13 | 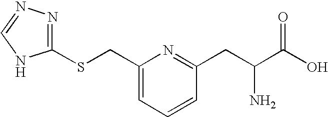 | Step-7-53 | 35 |
| 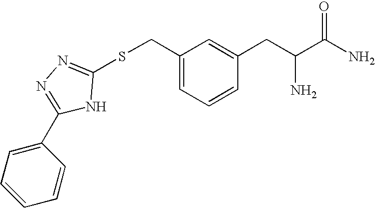 | Step-7-22 | 14 | 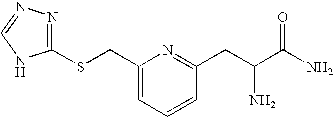 | Step-7-54 | 36 |
| 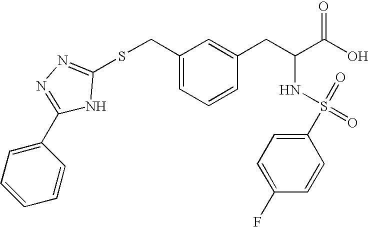 | Step-7-23 | 56 | 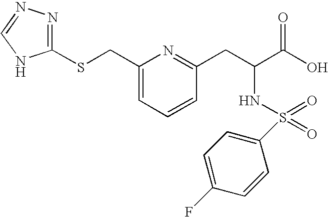 | Step-7-55 | 66 |
| 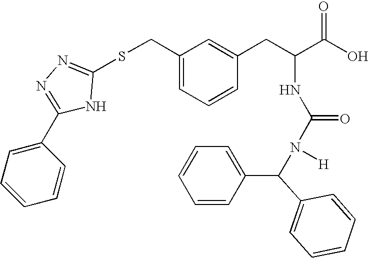 | Step-7-24 | 57 | 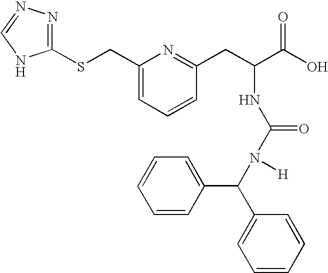 | Step-7-56 | 37 |
| 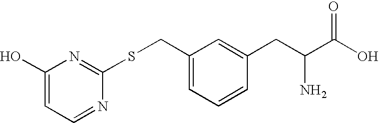 | Step-7-25 | 15 | 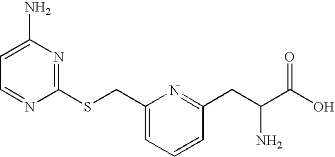 | Step-7-57 | 38 |
| 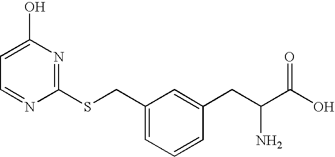 | Step-7-26 | 16 | 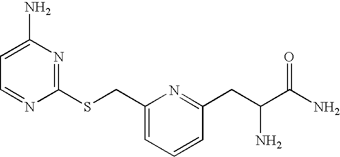 | Step-7-58 | 39 |
| 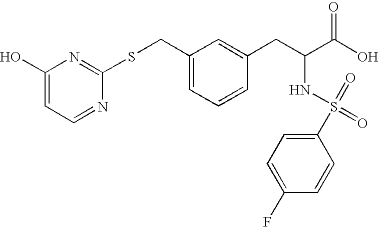 | Step-7-27 | 58 | 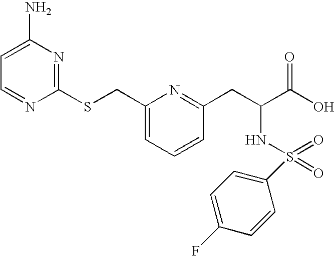 | Step-7-59 | 40 |
| 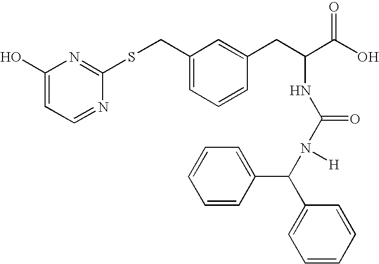 | Step-7-28 | 17 | 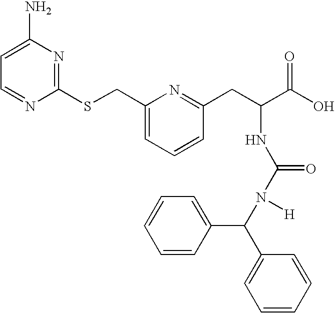 | Step-7-60 | 67 |
| 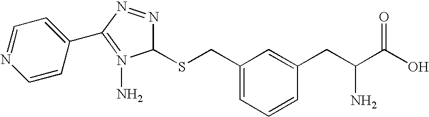 | Step-7-29 | 18 | 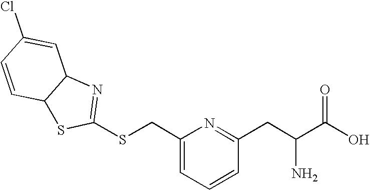 | Step-7-61 | 41 |
| 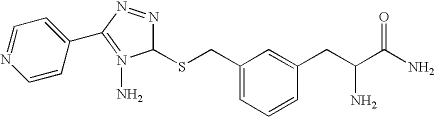 | Step-7-30 | 19 | 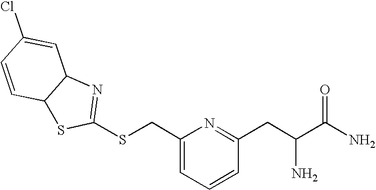 | Step-7-62 | 42 |
| 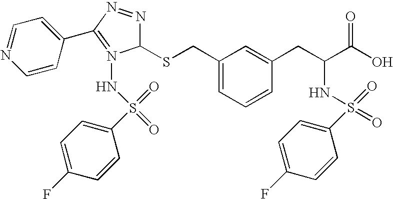 | Step-7-31 | 59 | 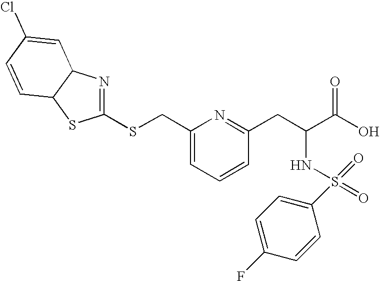 | Step-7-63 | 68 |
| 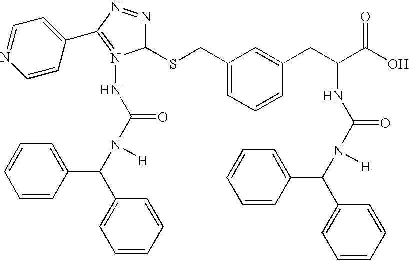 | Step-7-32 | 20 | 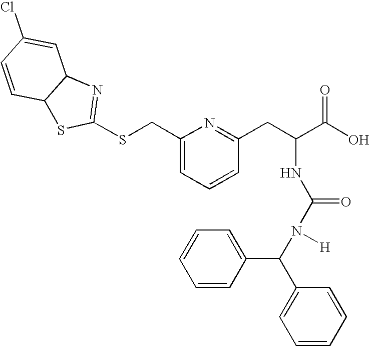 | Step-7-64 | 69 |

Synthesis of Library (Route 2):

Steps 1 and 2 were performed according to Route 1, above, except that 6 g (6 nmuol) of Fmoc-Gly-Wang resin was used instead of 5 g.

Step 3 (Introduction of Building Block A):

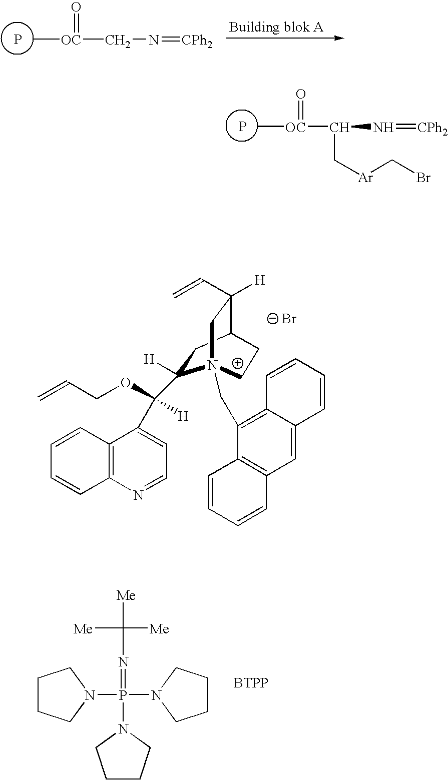

The benzophenone imine resin from Step 2 (1.5 g, ˜1.5 mmol), building block A as defined below (e.g., 2.0 g, 7.6 mmol of α,α-dibromo-m-xylene) and O-allyl-N-(9-anthracenylmethyl)cinchonidinium bromide (910 mg, 1.5 mmol) was suspended in methylene chloride (20 mL). The suspension was shaken for 5 min and then cooled to −78° C. with a dry ice slurry in 2-propanol. The Dewar flask was fixed on Titer plate shaker with a foam lid to maintain the low temperature. The reaction mixture was shaken gently for 20 min at −78° C. 2.3 mL (7.5 mmol) of tert-butylimino-tris(pyrrolidino)phosphorine (BTPP, a phosphazene base) was added via syringe. The reaction mixture was shaken at −78° C. for 5 h and then gradually warmed up to room temperature for 5 to 7 h. The reagents and solvents were then removed by filtration. The resin was washed 4 times with DMF (20 mL), 4 times with CH₂Cl₂ (20 mL), and 4 times with methanol (20 mL), and subsequently dried overnight in vacuo.

| Building block A | Products |
|---|---|
| 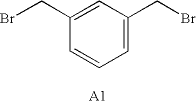 | 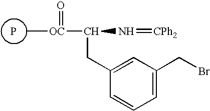 |
| 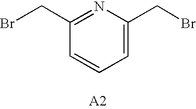 | 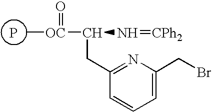 |
| 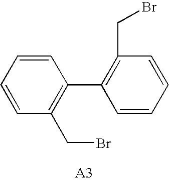 | 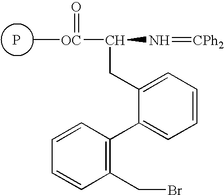 |
| 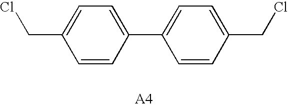 | 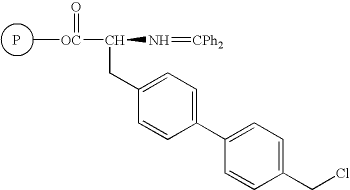 |

Step 4 (Coupling of Building Block B):

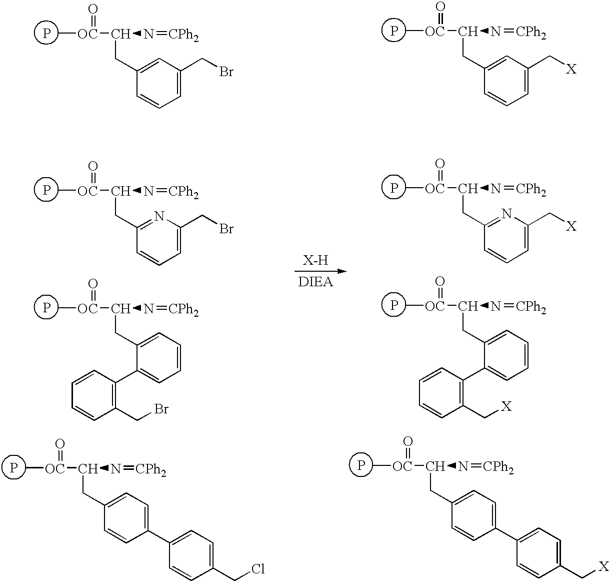

Each resin from Step 3 was distributed into 24 fritted syringes (Torvig, 50 mg each, 50 μmol), for a total of 96 syringes, and was swelled in NMP (1 mL) for 30 min. The solvent was removed by filtration. Twenty-four solutions of the building blocks listed below (10 mmol each) and DIBA (3.5 mL, 20 mmol) in NMP (10 mL) were prepared. 3 mL of the 24 solutions was added to the 24 syringes for each resin from Step 3, accordingly. The suspensions were then shaken for 20 h on a Titer Plate Shaker. The reaction mixture was filtered and washed 5 times with methylene chloride (5 mL), 3 times with THF (5 mL), 3 times THF/H₂O (3/1 v/v, 5 mL), and 3 times with THF (5 mL). The resins were then dried overnight under vacuum.

| Building | Structure of | Products |
|---|---|---|
| Block | Building Block | Step 4-# |
| B1 | 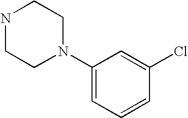 | 01, 02, 03, 04 |
| B2 | 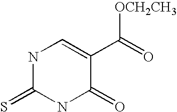 | 05, 06, 07, 08 |
| B3 | 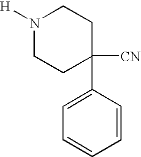 | 09, 10, 11, 12 |
| B4 | 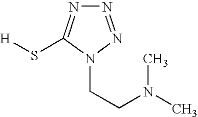 | 13, 14, 15, 16 |
| B5 | 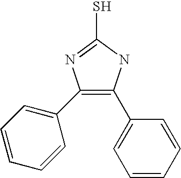 | 17, 18, 19, 20 |
| B6 | 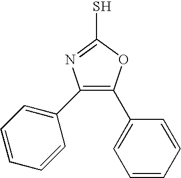 | 21, 22, 23, 24 |
| B7 | 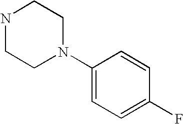 | 25, 26, 27, 28 |
| B8 | 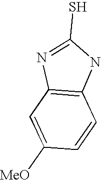 | 29, 30, 31, 32 |
| B9 | 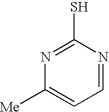 | 33, 34, 35, 36 |
| B10 | 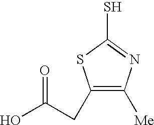 | 37, 38, 39, 40 |
| B11 | 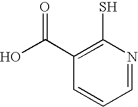 | 41, 42, 43, 44 |
| B12 | 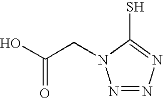 | 45, 46, 47, 48 |
| B13 | 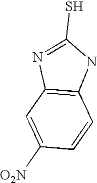 | 49, 50, 51, 52 |
| B14 | 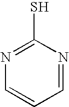 | 53, 54, 55, 56 |
| B15 | 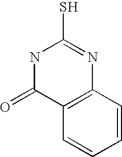 | 57, 58, 59, 60 |
| B16 | 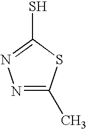 | 61, 62, 63, 64 |
| B17 | 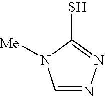 | 65, 66, 67, 68 |
| B18 | 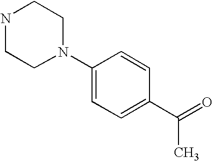 | 69, 70, 71, 72 |
| B19 | 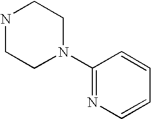 | 73, 74, 75, 76 |
| B20 | 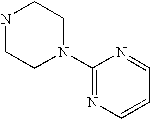 | 77, 78, 79, 80 |
| B21 | 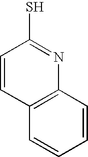 | 81, 82, 83, 84 |
| B22 | 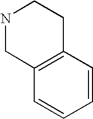 | 85, 86, 87, 88 |
| B23 | 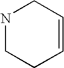 | 89, 90, 91, 92 |
| B24 | 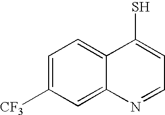 | 93, 94, 95, 96 |

Step 5 (Removal of Protecting Groups):

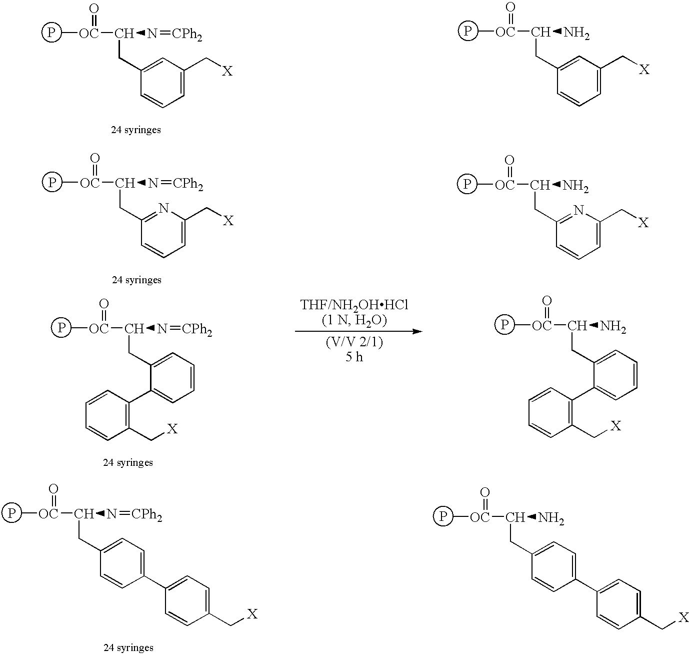

The resins from Step 4, in their 96 original fritted syringeswere each suspended in 1N aqueous solution of NH₂OH HCl/THF (v/v, 1/2, 3 mL) and shaken for 5 h at ambient temperature. The reagents and solvents were then removed by filtration from the frit. The resins were washed 4 times with THF (2 mL), 4 times with DMF (2 mL), and once with DIEA (1N in DMF, 2 mL) for 30 min. The resins were then filtered and washed 4 times with DMF (2 mL), and four times with CH₂Cl₂ (2 mL), and subsequently dried in vacuo. A Kaiser test showed that resin was in deep blue, which indicates the free amine of the product.

Step 6 (Cleavage to Obtain Products):

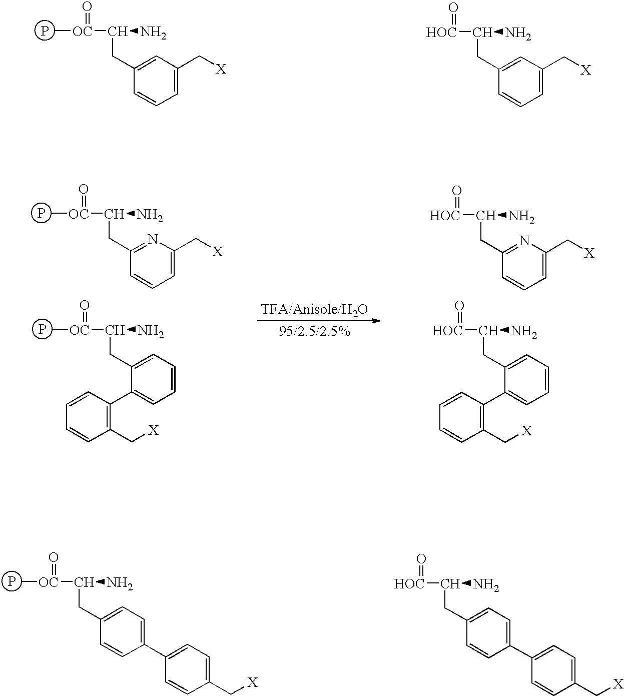

The resins from Step-5 (96 syringes, 50 mg each, 50 μmol) were each treated with TFA/Anisole/H₂O (95/2.5/2.5%, 1 mL each) for 5 min and the filtrate was collected by filtration. The resins were again treated with TFA/Anisole/H₂O (95/2.5/2.5%, 1 mL each) for 30 min. The filtrates from the same syringes were combined. To each of the filtrates was added cold ether (10 mL), the precipitate was centrifuged for 5 min at 4000 rpm, and the supernant was decanted. The precipitates were washed and centrifuged three additional times to remove the possible impurities. ES-MASS showed correct molecular weight of the desired compounds.

The structures of the products from Route 2 are listed in the following table:

| Structure | Product | ID# | Structure | Product | ID# |
|---|---|---|---|---|---|
| 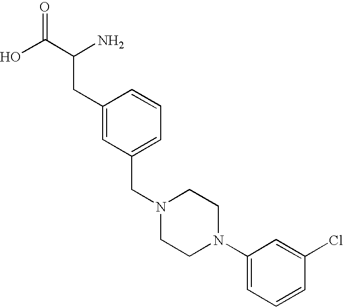 | Step-6-01 | 71 | 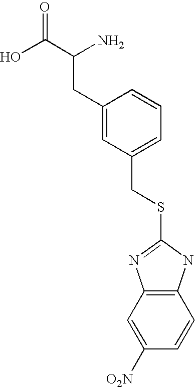 | Step-6-49 | 79 |
| 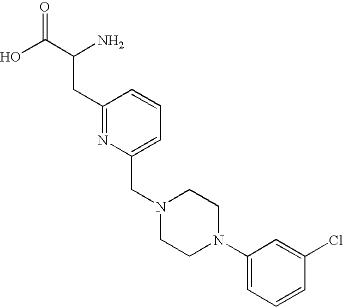 | Step-6-02 | 86 | 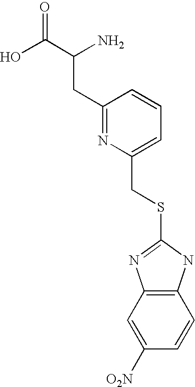 | Step-6-50 | 94 |
| 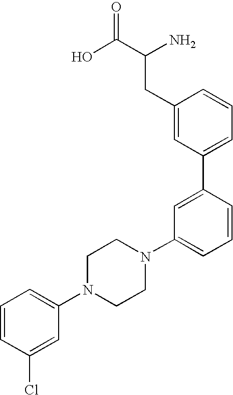 | Step-6-03 | 101 | 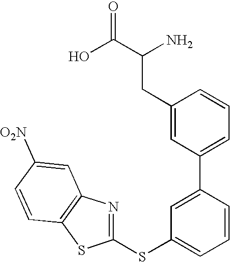 | Step-6-51 | 109 |
| 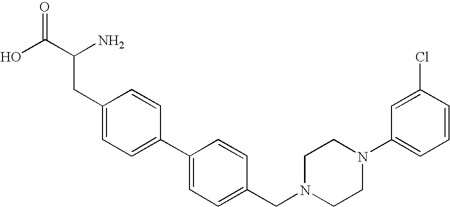 | Step-6-04 | 116 | 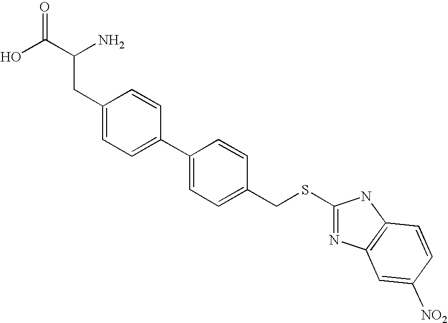 | Step-6-52 | 124 |
| 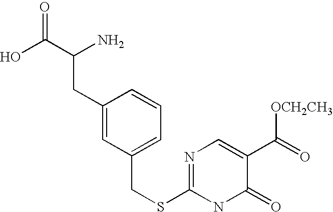 | Step-6-05 | 131 | 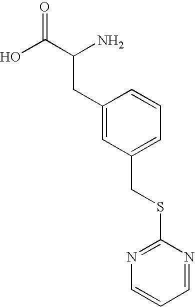 | Step-6-53 | 139 |
| 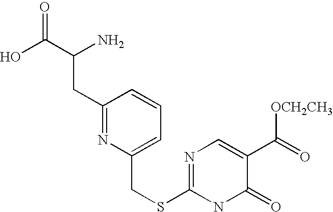 | Step-6-06 | 146 | 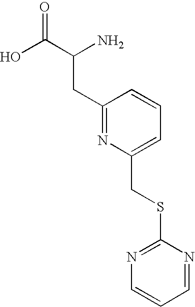 | Step-6-54 | 154 |
| 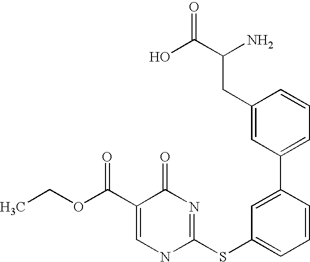 | Step-6-07 | 72 | 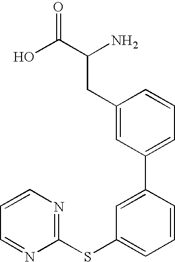 | Step-6-55 | 80 |
| 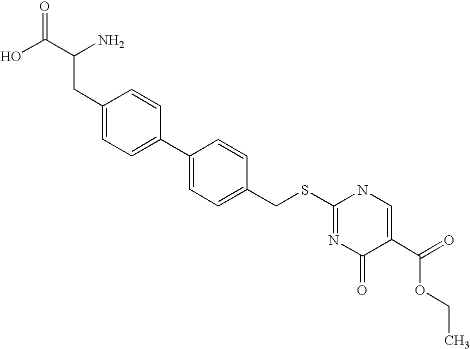 | Step-6-08 | 87 | 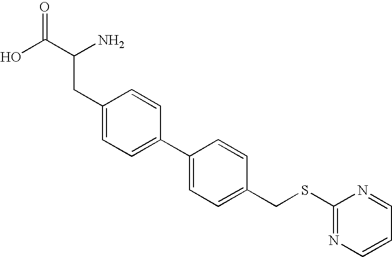 | Step-6-56 | 95 |
| 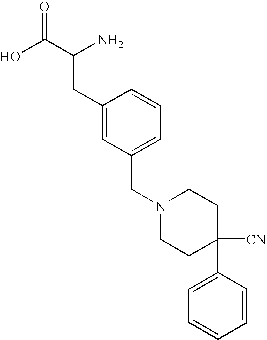 | Step-6-09 | 102 | 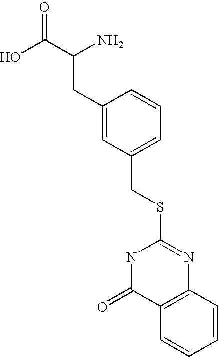 | Step-6-57 | 110 |
| 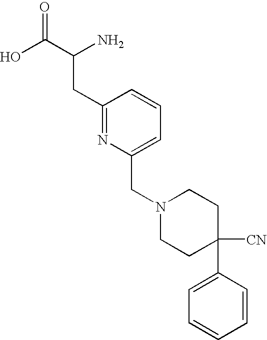 | Step-6-10 | 117 | 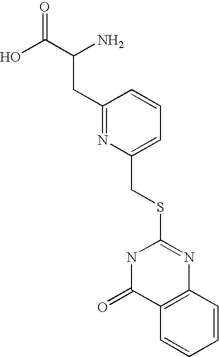 | Step-6-58 | 125 |
| 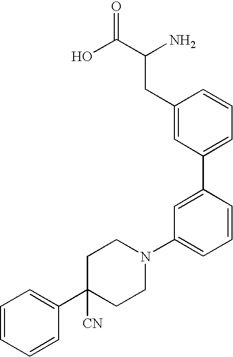 | Step-6-11 | 132 | 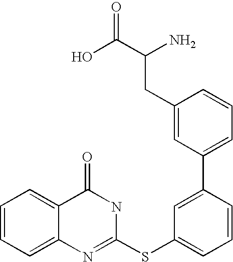 | Step-6-59 | 140 |
| 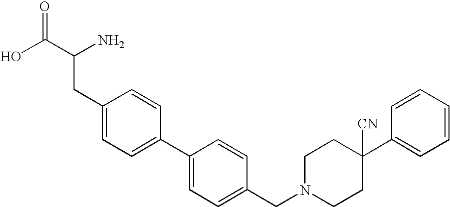 | Step-6-12 | 147 | 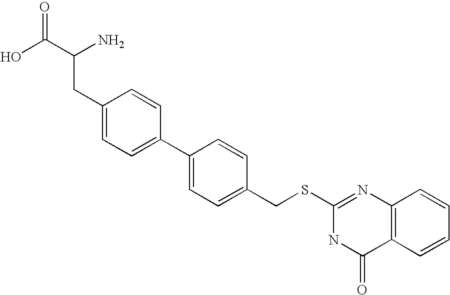 | Step-6-60 | 155 |
| 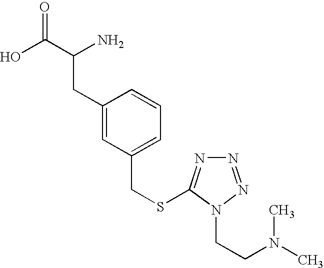 | Step-6-13 | 73 | 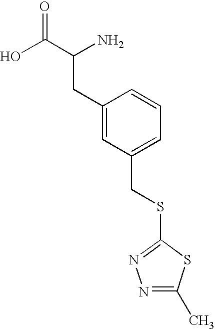 | Step-6-61 | 81 |
| 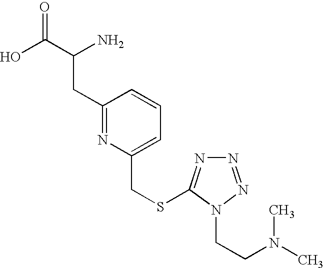 | Step-6-14 | 88 | 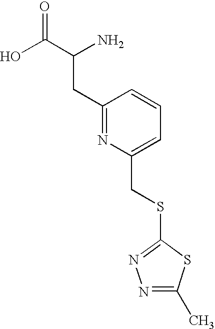 | Step-6-62 | 96 |
| 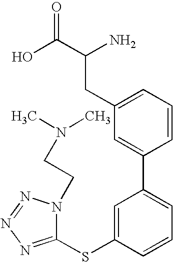 | Step-6-15 | 103 | 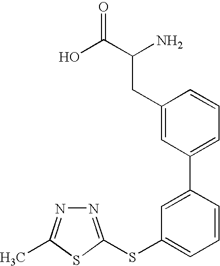 | Step-6-63 | 111 |
| 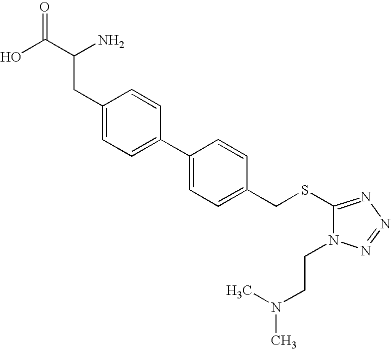 | Step-6-16 | 118 | 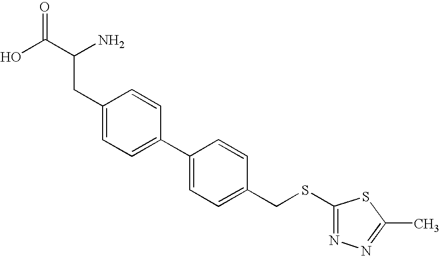 | Step-6-64 | 126 |
| 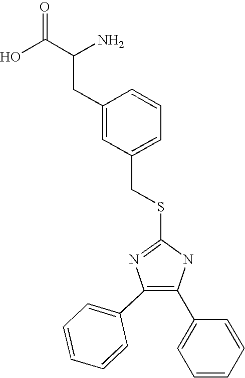 | Step-6-17 | 133 | 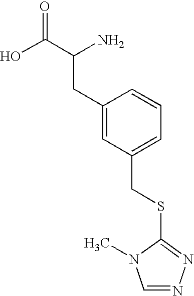 | Step-6-65 | 141 |
| 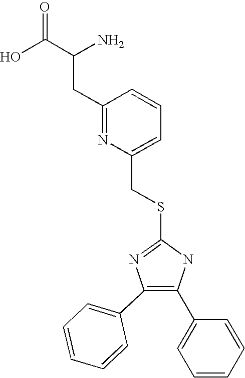 | Step-6-18 | 148 | 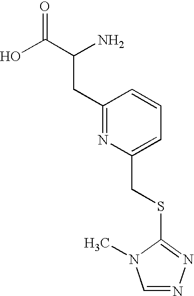 | Step-6-66 | 156 |
| 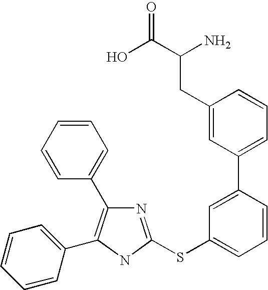 | Step-6-19 | 74 | 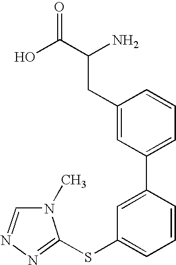 | Step-6-67 | 82 |
| 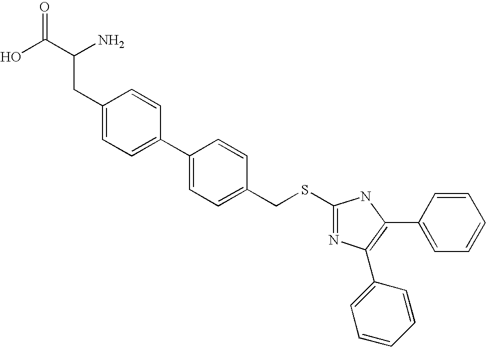 | Step-6-20 | 89 | 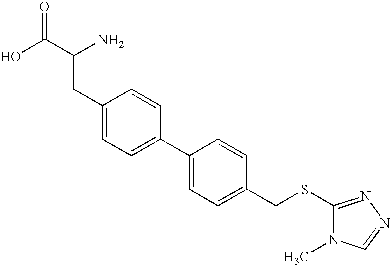 | Step-6-68 | 97 |
| 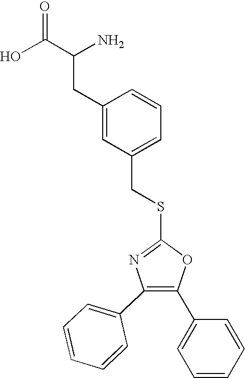 | Step-6-21 | 104 | 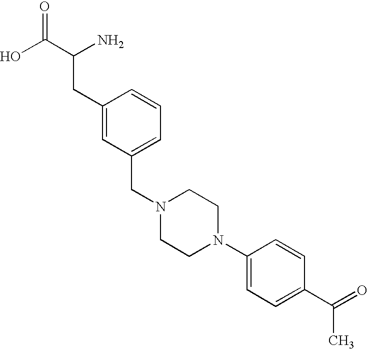 | Step-6-69 | 112 |
| 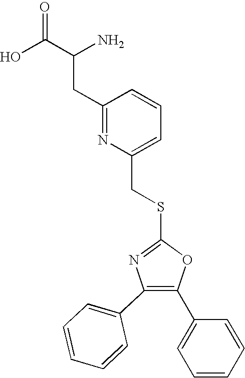 | Step-6-22 | 119 | 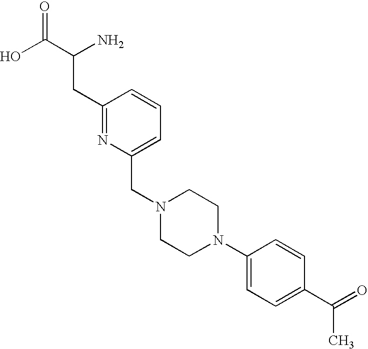 | Step-6-70 | 127 |
| 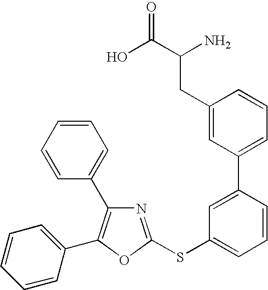 | Step-6-23 | 134 | 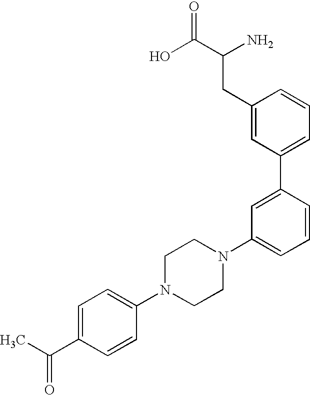 | Step-6-71 | 142 |
| 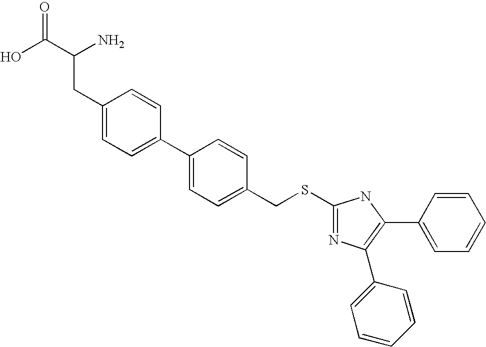 | Step-6-24 | 149 | 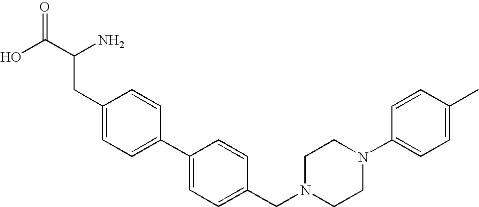 | Step-6-72 | 157 |
| 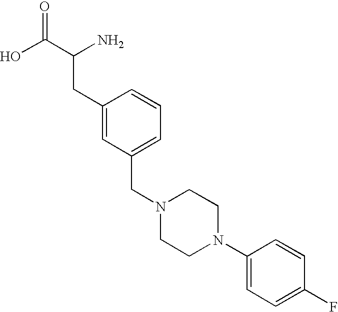 | Step-6-25 | 75 | 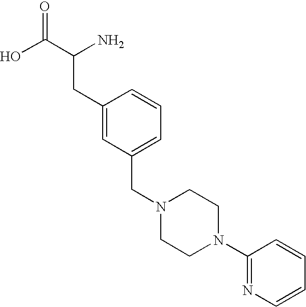 | Step-6-73 | 83 |
| 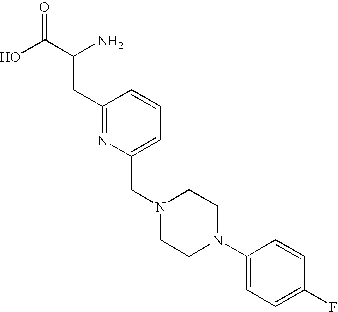 | Step-6-26 | 90 | 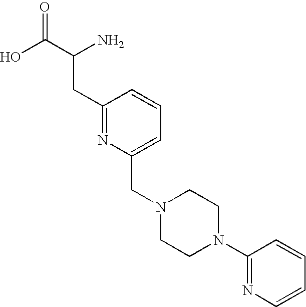 | Step-6-74 | 98 |
| 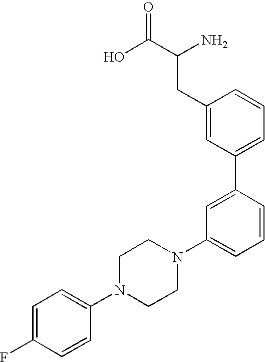 | Step-6-27 | 105 | 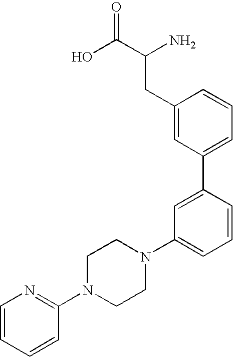 | Step-6-75 | 113 |
| 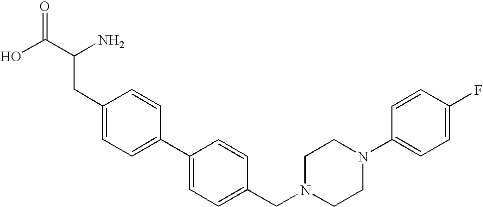 | Step-6-28 | 120 | 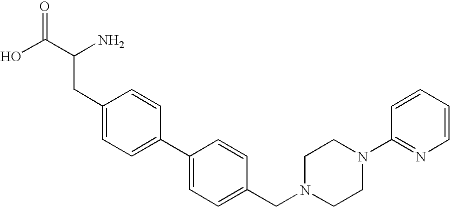 | Step-6-76 | 128 |
| 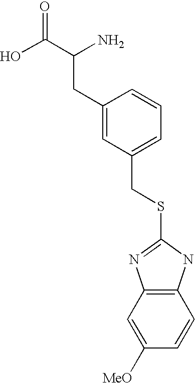 | Step-6-29 | 135 | 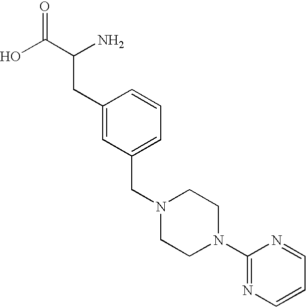 | Step-6-77 | 143 |
| 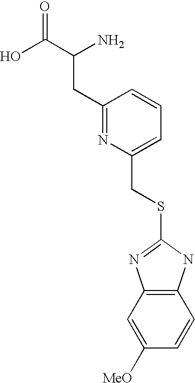 | Step-6-30 | 150 | 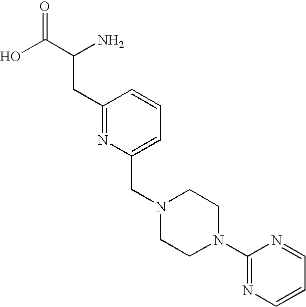 | Step-6-78 | 158 |
| 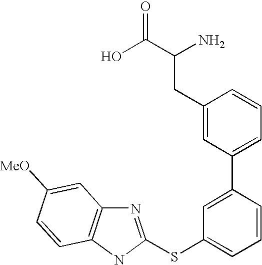 | Step-6-31 | 76 | 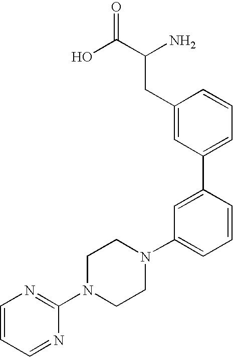 | Step-6-79 | 84 |
| 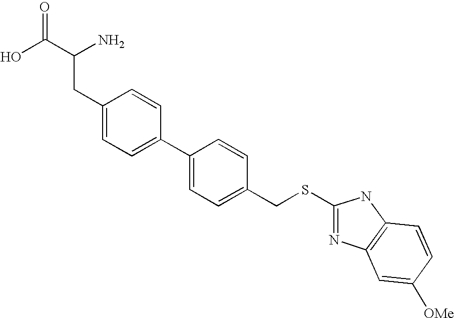 | Step-6-32 | 91 | 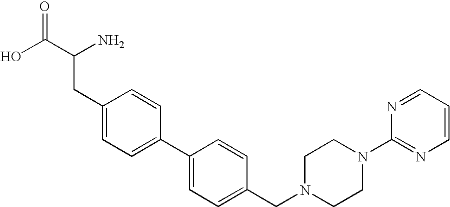 | Step-6-80 | 99 |
| 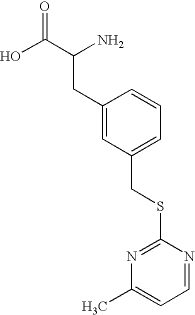 | Step-6-33 | 106 | 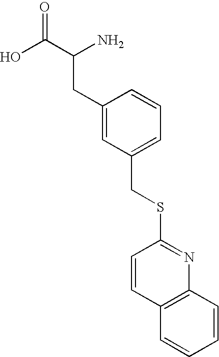 | Step-6-81 | 114 |
| 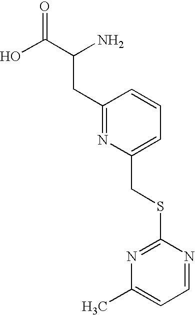 | Step-6-34 | 121 | 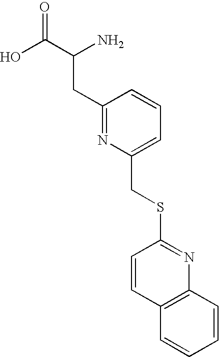 | Step-6-82 | 129 |
| 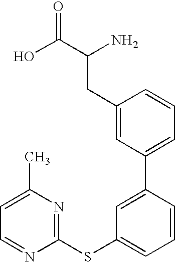 | Step-6-35 | 136 | 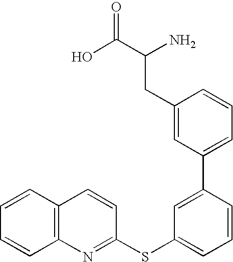 | Step-6-83 | 144 |
| 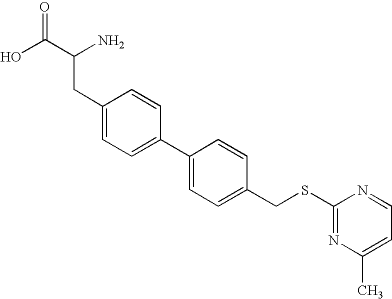 | Step-6-36 | 151 | 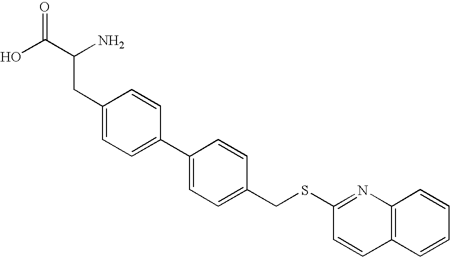 | Step-6-84 | 159 |
| 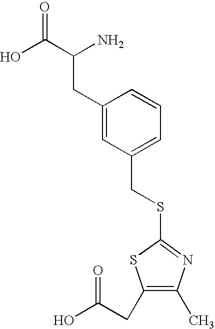 | Step-6-37 | 77 | 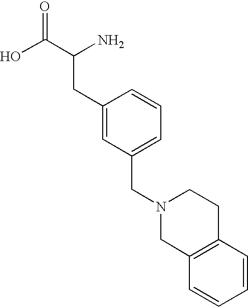 | Step-6-85 | 85 |
| 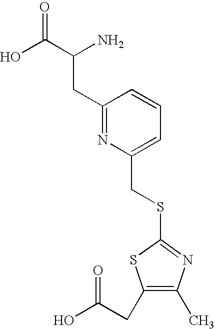 | Step-6-38 | 92 | 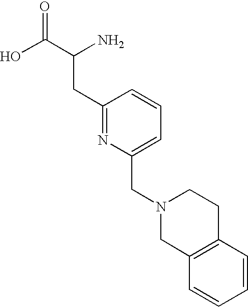 | Step-6-86 | 100 |
| 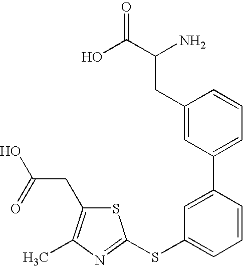 | Step-6-39 | 107 | 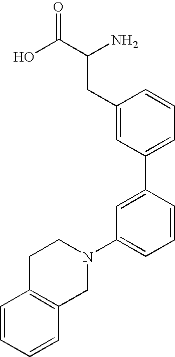 | Step-6-87 | 115 |
| 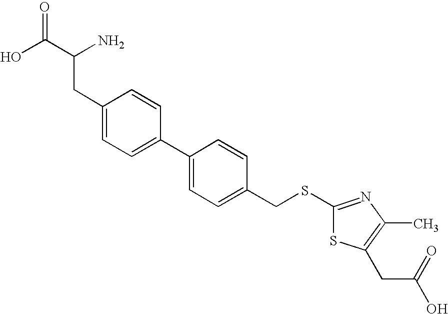 | Step-6-40 | 122 | 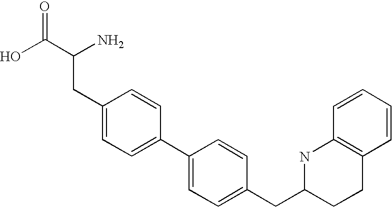 | Step-6-88 | 130 |
| 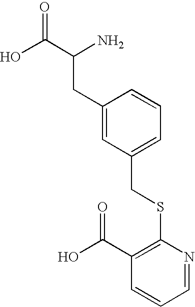 | Step-6-41 | 137 | 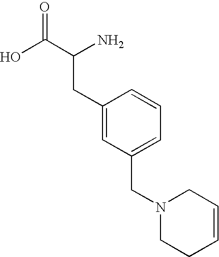 | Step-6-89 | 145 |
| 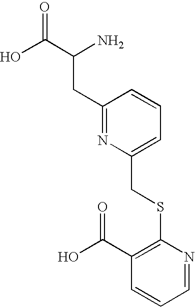 | Step-6-42 | 152 | 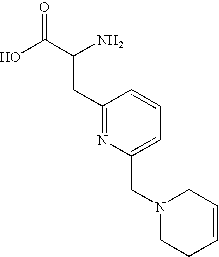 | Step-6-90 | 160 |
| 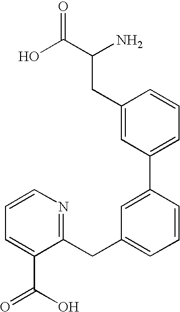 | Step-6-43 | 78 | 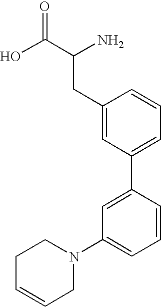 | Step-6-91 | 43 |
| 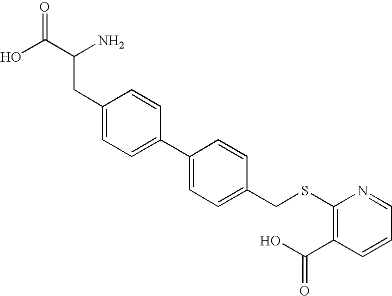 | Step-6-44 | 93 | 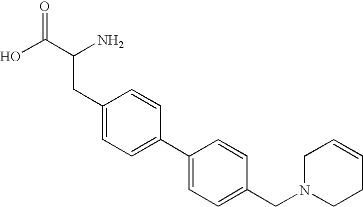 | Step-6-92 | 44 |
| 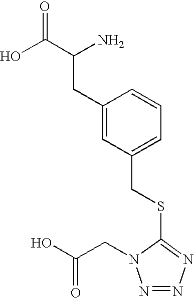 | Step-6-45 | 108 | 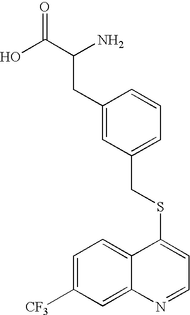 | Step-6-93 | 70 |
| 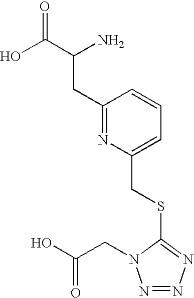 | Step-6-46 | 123 | 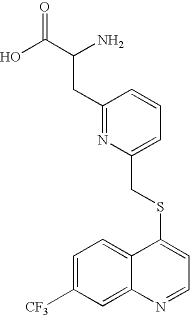 | Step-6-94 | 45 |
| 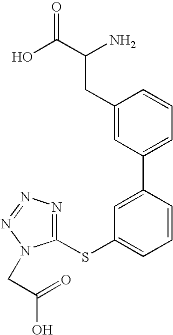 | Step-6-47 | 138 | 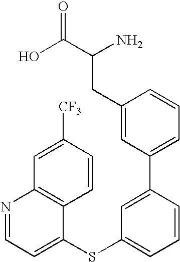 | Step-6-95 | 46 |
| 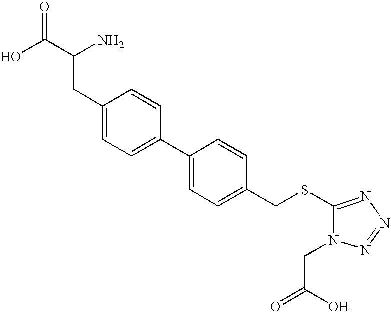 | Step-6-48 | 153 | 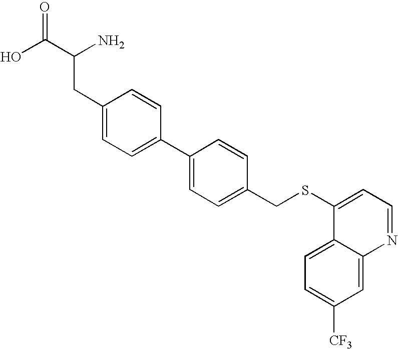 | Step-6-96 | 47 |

160 exemplary compounds are prepared at 1 mM in 1% DMSOIH₂O solution. Briefly, after dissolving the samples in 250 μL DMSO, 100 μL of each dissolved compound is added to 10 mL of water. The solutions are incubated at 37° C. for an overnight incubation period with shaking. After centrifugation, samples are soluble or partially soluble. MS analysis is conducted on all samples and samples are stored at −20° C. If only partially soluble, the supernatants of the compounds, not the whole solution, are stored at −20° C.

For cellular assays, the diluent of solutions initially prepared in 1% DMSO/H₂O is changed to a suitable physiologic buffer. A volume of 0.5 mL of a sterile concentrated 10× solution of PBS (without Ca⁺², Mg⁺²)-Glucose-HEPES-DMSO is added to 4.5 mL of aqueous solution. Solubility is visually verified, and pH is measured to ensure that the pH of the solution is neutral. Some compounds are estimated to be at neutral pH range in the same experimental conditions. The compound solutions are then filtered through a 0.22-μm filter unit, and 250 μL aliquots are placed in polypropylene tubes and stored at −20° C.

### Example 2

### Binding of Exemplary Compounds to the Brain L1 Transport System

Dilution of Library Compounds for Use in Competitive Binding Assay

Compound samples in PBS (without Ca⁺², Mg⁺²)-glucose 30 mM-HEPES 10 mM—DMSO 1% as prepared in Example 1 were thawed and left for at least 30 minutes at 20-23° C. before preparation of the following sub-dilutions for the competitive binding assay:
200 μL of the stock solution were added to 800 μL PBS (without Ca⁺², Mg⁺²)-Glucose-HEPES-1% DMSO (PBSD-1) [diluted 1:5 for a final 1:5 dilution]
100 μL (1/5 dilution above) were added to 900 μL PBSD-1 [diluted 1:10 for a final 1:50 dilution]
100 μL (1/50 dilution above) were added to 900 μL PBSD-1 [diluted 1:10 for a final 1:500 dilution]

These sub-dilutions were used immediately or stored overnight at 4° C. before the competitive binding assay. A volume of 45 μL of each of the compound dilutions (1:5, 1:50, 1:500) in PBSD-1 was added to the appropriate wells in the dilution plate.

Isolation of Rat Primary Cerebrovascular Endothelial Cells

Brains from sixty 24-day-old rats were dissected individually on a sterile lint moistened with ice cold Hanks' balanced salt solution (Gibco BRL, Grand Island, N.Y.) containing 10 mM HEPES (medium 1) supplemented with 0.1% BSA. Cerebellum, striatum, optic nerves, and brain stem (white matter) were removed. After a mid-sagittal section of the brain, the meninges and leptomeningeal debris were removed by rolling a sterile dry cotton swab on the cortices. (Ichikawa N, Naora K, Hirano H, Hashimoto M, Masumura S, and Iwamoto K (1996). Isolation and primary culture of rat cerebral microvascular endothelial cells for studying drug transport in vitro. J Pharmacol Toxicol Meth 36: 45-52.). Clean cortices were minced in pieces of ≈2 mm³ in 15 mL of ice-cold medium 1-0.1% BSA. The preparation was divided into 4 sterile pre-weighed tubes and centrifuged at 330× g at 20-25° C. for 5 min. Tubes were weighed and pre-warmed (37° C.) and medium 1 with 0.5% BSA containing 0.3% collagenase and 10 μg/mL DNAse 1 (Roche, Laval, Quebec, Canada) were added to each tube (1 mL/g of tissue).

The brain-collagenase mixture was vigorously agitated in a water bath at 37° C. for 90 min. Fifteen min before the end of the digestion, the tissue was homogenized using a 10-mL pipette until a creamy mixture was obtained (≈20 aspirations). Cells were washed by adding medium 1 with 0.1% BSA to the homogenate (26 mL/tube) and centrifuged at 100× g at 20-25° C. for 7 min. This washing step was repeated 3 more times, once for 5 min and twice for 3 min. Each pellet was re-suspended in 25 mL of a 15% dextran solution prepared in medium 1 with 0.1% BSA and centrifuged at 3200× g at 4° C. for 25 min to isolate vessels from neural tissue and dextran layers. Vascular pellets were re-suspended in 5 mL Ca⁺⁺—Mg⁺⁺-free-medium 1 with 0.1% BSA (medium 2) at 20-25° C. and transferred in a 50-mL tube. Remaining vessels were collected by rinsing the tubes and pooling the rinsing suspension. (Rupnick M A, Carey A, and Williams S K (1988). Phenotypic diversity in cultured cerebral microvascular endothelial cells in vitro. Cell Develop Biol 24: 435-444.)

The vascular preparation was filtered and rinsed (20 mL of medium 2) through a sterile 355-μm mesh. The 355-μm filtrate was sequentially filtered twice (20 mL of medium 2) through sterile 112-μm meshes and rinsed (Stanimirovic D B, Wong J, Ball R, and Durkin J P (1995). Free radical-induced endothelial membrane dysfunction at the site of the blood-brain barrier: relationship between lipid peroxidation, Na,K-ATPase activity, and ⁵¹Cr release. Neurochem Res 20:1417-1427.) and the latter filtrate was filtered and rinsed through a sterile 20-μm mesh. A final step of filtration and rinse was repeated through a double layer of 20-μm meshes. All 20-μm meshes, which retained the microcapillaries, were then transferred into a 50-mL tube containing 20 mL of a 0.1% collagenase/dispase (Roche) solution in medium 2 supplemented with 10 μg/mL DNAse 1 and 0.147 μg/mL tosyl-lysine-chloromethyl-ketone (Sigma Chemical Co., Oakville, Ontario, Canada) (medium 3). (Abbott N J, Hughes C C W, Revest P A, and Greenwood J (1992). Development and characterisation of a rat brain capillary endothelial culture: towards an in vitro blood-brain barrier. J Cell Sci 103: 23-37.) The tube was shaken vigorously to dislodge the capillaries from the meshes, which were then removed from the tube. During the digestion process, the microcapillary preparation was gently shaken in a water bath at 37° C. for 60 min. The preparation was again filtered and rinsed (20 mL of medium 2) through a double layer of 20-μm meshes. Meshes were soaked in 20 mL of medium 2, shaken, and removed. The microvessel preparation was then centrifuged at 330× g at 20-25° C. for 5 min. The pellet was re-suspended in 500 μL of culture medium consisting of high glucose Dulbecco's minimum essential medium (Wisent, Herndon, Va.) supplemented with amino acids (1×) (Sigma Chemical Co.), vitamins (1×) (Gibco BRL), antibiotics/antimycotics mixture (1×) (Gibco BRL), 20% FBS (Hyclone, Logan, Utah), 500 μg/mL of peptone (Sigma Chemical Co.), 100 μg/mL of endothelial cell growth supplement (Sigma Chemical Co.), and 50 μg/mL of heparin (Gibco BRL).

The microcapillary preparation was seeded onto a matrigel-coated (thin coating) 12-well plate (≈45 μL/well) (Becton Dickinson, Mississauga, Ontario, Canada) and incubated at 37° C. in a humidified 5% CO₂ atmosphere for 16 h. Non-adhering cells were then dislodged by pipetting 10-15 times the culture medium onto the well surface using a 1-mL pipette. When cellular debris clung to the well, the procedure was repeated using PBS (800 μL/well). After the addition of fresh culture medium, cell growth was monitored on a daily basis. On day 2 of culture, cells were washed with Ca⁺⁺—Mg⁺⁺-free-PBS, trypsinized, counted, and plated in culture medium at a density of 1×10⁵ cells/mL onto matrigel-coated flat bottom 96-well plates and onto a 48-well plate for the characterization of the general endothelial properties.

Characterization of Rat Primary Cerebrovascular Endothelial Cells

Endothelial cells in a 48 well plate as described above were tested for the uptake of Ac-LDL labeled with a fluorescent probe 1,1′-dioctoadecyl-3,3,3′,3′-tetramethyl-indocarbocyamine perchlorate (Dil-Ac-LDL) (Biomedical Technologies Inc., Stoughton, Me.), for von Willebrand factor expression (Dako Corporation, Carpinteria, Calif.), and for TRITC—labeled ConA uptake (Sigma Chemical Co.) according to manufacturer's specifications. Characterization of the cell preparation indicated that the isolation procedure resulted in enriched brain endothelial cell cultures that could be used to efficiently test the indirect ability of specific compounds to cross the BBB using active transporter systems such as the L1-system. On a routine basis, the characterization of RBEC was carried out in parallel to the binding of the compounds to the targeted L1-system carrier.

These results indicated that this method for isolating and culturing enriched primary endothelial cell retains the characteristics of the RBEC and the functionality of their endogenous transporters such as the L1-system carrier. The use of enriched RBEC cultures retaining their endothelial transporter system functionality allows the development of a rapid, reliable, and reproducible competitive binding assay to screen drugs. This medium throughput assay can be employed to identify compounds that bind, e.g., to the L1-system carrier and provide parameters to select CNS drug candidates designed to penetrate the brain using a specific active transporter.

Preparation of L-phenylalanine and α-,ethylamino isobutyric acid controls

L-phenylalanine (Sigma) and α-(Methylamino) isobutyric acid (MeAIB) (Sigma) were prepared at 2×10⁻² M by dissolving 0.033g and 0.023g per 10 mL of physiologic buffer, respectively. Both solutions were filtered using 0.22-μm membrane filter and a 5-ml syringe, aliquoted in 250 μL and frozen at −20° C.

Aliquots of L-phenylalanine and MeAIB were thawed at 20-23° C. and left to stand for a 30-minute incubation period. These controls were then diluted in a 96-well plate set for the dilution purpose and further addition of the radioactive isotope prior to the addition of the complete mixture onto cells.

L-phenylalanine and α-(Methylamino) isobutyric acid controls were diluted in wells by performing a 10-fold serial dilution of 45 μL of PBSD-1 with 50 μL stock solution (freshly thawed).

Preparation of the Radioactive Phenylalanine

L-[U-¹⁴C] Phenylalanine (Amersham Pharmacia Biotech UK Limited) was kept at 4° C. in its original package. The radiochemical batch analysis is as follows:

| | |
|---|---|
| Company: | Amersham Pharmacia |
| Code: | CFB.70 |
| Batch: | 133 |
| Pack Size: | 250 μCi |
| Pack Volume: | 5 mL |
| Specific Activity: | 17.4 GBq/mmol, 469 mCi/mmol |
| | 96.4 MBq/mg, 2.61 mCi/mg |
| Molecular Weight: | 165 (unlabelled) |
| | 180 (at this specific activity) |
| Radioactive Concentration: | 1.85 MBq/mL, 50 μCi/mL |

The concentration of L-[U-¹⁴C] Phenylalanine used in this assay was previously determined as the concentration at which there was a 50% maximum binding to the endothelial cell receptors. By estimating the sigmoidal curve fits of the raw data of several experiments using Sigma Plot program, it was estimated that the concentration required for half-maximal saturation of L1 transport system receptors by L-[U-¹⁴C] Phenylalanine was 7×10⁻⁹M/3.17 μCi/mL.

Since the radiolabeled phenylalanine was added on cells following a 2-fold dilution, a double concentration of the L-[U-¹⁴C] Phenylalanine solution was prepared at 14×10⁻⁹M/6.34 μCi/mL. The original L-[U-¹⁴C] Phenylalanine solution was diluted by a factor of 7.89 (50 μCi/mL stock concentration divided by 6.34 μCi/mL) in physiologic buffer (PBS with Ca⁺², Mg⁺²-HEPES (10 mM final)-glucose (30 mM final)).

A volume of 45 μL L-[U-¹⁴C] Phenylalanine (2×) was added to a volume of 45 μL of each compound dilution (1:5, 1:50, 1:500) in PBSD-1, which were previously distributed in a 96-well dilution plate.

Competitive Binding Assay Protocol

Subsequent to plating rat primary endothelial cells and culture media onto 96-well plates as described above, the cells were cultured for 6 days and the culture media was replaced every 3-4 days. The rat endothelial cells were then washed twice with warm physiologic buffer solution. A volume of 35 μL of the radio labeled L-[U-¹⁴C] Phenylaianine and the compound/control mixtures were added to cells and the plate was incubated for 5 minutes at 20-23 ° C. Cells were washed twice with cold physiologic buffer and 25 μL NaOH IN were added and incubated at 20-23° C. for 10 minutes. The plate was tapped gently on the side to ensure that all the cells detached. The cell lysate was then neutralised with the addition of 25 μL HCl 1N. The 50 μL mixture was transferred to a Wallac flexible plate (specific for radioactivity counting) and 200 μL of scintillation liquid (Opti-Phase Supermix, Wallac, UK), were added per well. The plate was sealed, vortexed and read on the Wallac β-counter.

Procedure for Radioactivity Counting

The plates containing the radioactive mixture were transferred to the Wallac β-counter plate holders. Wallac 1450 Microbeta (Wallac) Protocol #96 was the appropriate protocol for 96-well plates. Briefly, the Protocol #96 includes all the specifications required to detect a specific radioactive isotope in the Wallac β-counter. Liquid scintillation counting is a process in which the beta decay electron emitted by the radioactive isotope (in this case ¹⁴C) in the sample excites the solvent molecule, which in turn transfers the energy to the solute, or fluor. The energy emission of the solute (the light photon) is converted into an electrical signal (CPM or Counts per Minute) by a photomultiplier tube. Each well was counted for 2-minutes by three photomultiplier tubes simultaneously. The collected raw data, CCPM or Corrected Counts per Minute, were adjusted to the background and used in the compilation of the results.

Data Processing

The raw data obtained for the radioactivity counting, Corrected Counts per Minute (CCPM), indicates the amount of L-[U-¹⁴C] Phenylalanine radioactivity associated to cells and corrected for the background.

Data Analysis and Calculation

Mean and standard deviation for each replicate of each sample concentration was calculated. The percentage of the specific binding to L1 transport system on cells was also calculated as follows:

$$\frac{CCPM of ( sample + L - [ U - 14 C ] Phenvlalanine )}{CCPM of ( L - [ U - 14 C ] Phenylalanine )} \times 100 \%$$

The percentage of the specific binding was then compared to that of the L-phenylalanine reference control and the difference was evaluated by an arbitrarily scoring system.

Results

In order to objectively discriminate between the 160 phenylalanine-derivative compounds for their ability to bind to the brain L1 transport system, a scoring rank system was employed to compare binding of the compounds to the phenylalanine control. For each tested concentration, the difference in the percentage of the specific binding between each compound and the phenylalanine control was evaluated as the following:

For each tested concentration (10⁻⁶, 10⁻⁵, 10⁻⁴ M):
(%) specific binding of phenylalanine control−(%) specific binding of compound=X

if

| | |
|---|---|
| X < 0 | Rank 0 |
| 0 < X < 10 | Rank 1 |
| 10 < X < 20 | Rank 2 |
| X > 20 | Rank 3 |

For a given concentration, a compound having a difference of more than 20% compared to the phenylalanine means that this compound presents a higher ability to bind to the L1-system receptor than the phenylalanine itself. For the partially soluble compounds, the actual concentrations are unknown and underestimated and their ability to bind to the receptor may be therefore underestimated. Table 4, below, depicts the results with 127 of the compounds at the three concentrations tested. The remaining 33 (ID Nos. 2, 19, 23, 28, 40, 56, 58, 59, 65, 77, 78, 80, 83, 84, 85, 90, 100, 106, 107, 108, 128, 129, 130, 132, 133, 139, 140, 142, 143, 145, 150, 152, and 157) were ranked 0 for all 3 concentrations tested. Although this particular assay desmonstrated 12 compounds which were highly active (i.e., ranked 3 or above for 2 of the 3 concentrations tested), it is reasonable to believe that minor modifications in assay conditions or concentration would show that many more of the 160 tested compounds are also active.

TABLE 4
Comparison of binding affinities
of 127 test compounds with phenylalanine

| | | Comparison |
|---|---|---|
| | | with the |
| | Conc. | Binding of |
| I.D. # | (M) | Phenylalanine |
| 3. | 10⁻⁶ | 3 |
| | 10⁻⁵ | 3 |
| | 10⁻⁴ | 3 |
| 5. | 10⁻⁶ | 3 |
| | 10⁻⁵ | 3 |
| | 10⁻⁴ | 3 |
| 8. | 10⁻⁶ | 3 |
| | 10⁻⁵ | 3 |
| | 10⁻⁴ | 3 |
| 13. | 10⁻⁶ | 3 |
| | 10⁻⁵ | 3 |
| | 10⁻⁴ | 3 |
| 27. | 10⁻⁶ | 3 |
| | 10⁻⁵ | 3 |
| | 10⁻⁴ | 3 |
| 33. | 10⁻⁶ | 3 |
| | 10⁻⁵ | 3 |
| | 10⁻⁴ | 3 |
| 57. | 10⁻⁶ | 3 |
| | 10⁻⁵ | 3 |
| | 10⁻⁴ | 3 |
| 118. | 10⁻⁶ | 3 |
| | 10⁻⁵ | 3 |
| | 10⁻⁴ | 3 |
| 4. | 10⁻⁶ | 3 |
| | 10⁻⁵ | 3 |
| | 10⁻⁴ | 2 |
| 18. | 10⁻⁶ | 3 |
| | 10⁻⁵ | 3 |
| | 10⁻⁴ | 2 |
| 38. | 10⁻⁶ | 3 |
| | 10⁻⁵ | 3 |
| | 10⁻⁴ | 2 |
| 49. | 10⁻⁶ | 3 |
| | 10⁻⁵ | 3 |
| | 10⁻⁴ | 2 |
| 87. | 10⁻⁶ | 3 |
| | 10⁻⁵ | 3 |
| | 10⁻⁴ | 2 |
| 15. | 10⁻⁶ | 2 |
| | 10⁻⁵ | 3 |
| | 10⁻⁴ | 0 |
| 51. | 10⁻⁶ | 2 |
| | 10⁻⁵ | 3 |
| | 10⁻⁴ | 0 |
| 103. | 10⁻⁶ | 2 |
| | 10⁻⁵ | 2 |
| | 10⁻⁴ | 2 |
| 105. | 10⁻⁶ | 2 |
| | 10⁻⁵ | 2 |
| | 10⁻⁴ | 2 |
| 116. | 10⁻⁶ | 2 |
| | 10⁻⁵ | 2 |
| | 10⁻⁴ | 1 |
| 47. | 10⁻⁶ | 2 |
| | 10⁻⁵ | 2 |
| | 10⁻⁴ | 0 |
| 50. | 10⁻⁶ | 2 |
| | 10⁻⁵ | 2 |
| | 10⁻⁴ | 0 |
| 53. | 10⁻⁶ | 2 |
| | 10⁻⁵ | 2 |
| | 10⁻⁴ | 0 |
| 123. | 10⁻⁶ | 2 |
| | 10⁻⁵ | 2 |
| | 10⁻⁴ | 0 |
| 17. | 10⁻⁶ | 2 |
| | 10⁻⁵ | 1 |
| | 10⁻⁴ | 1 |
| 156. | 10⁻⁶ | 2 |
| | 10⁻⁵ | 1 |
| | 10⁻⁴ | 1 |
| 11. | 10⁻⁶ | 2 |
| | 10⁻⁵ | 1 |
| | 10⁻⁴ | 0 |
| 125. | 10⁻⁶ | 2 |
| | 10⁻⁵ | 1 |
| | 10⁻⁴ | 0 |
| 34. | 10⁻⁶ | 1 |
| | 10⁻⁵ | 0 |
| | 10⁻⁴ | 0 |
| 42. | 10⁻⁶ | 1 |
| | 10⁻⁵ | 0 |
| | 10⁻⁴ | 0 |
| 52. | 10⁻⁶ | 1 |
| | 10⁻⁵ | 0 |
| | 10⁻⁴ | 0 |
| 63. | 10⁻⁶ | 1 |
| | 10⁻⁵ | 0 |
| | 10⁻⁴ | 0 |
| 71. | 10⁻⁶ | 1 |
| | 10⁻⁵ | 0 |
| | 10⁻⁴ | 0 |
| 75. | 10⁻⁶ | 1 |
| | 10⁻⁵ | 0 |
| | 10⁻⁴ | 0 |
| 76. | 10⁻⁶ | 1 |
| | 10⁻⁵ | 0 |
| | 10⁻⁴ | 0 |
| 92. | 10⁻⁶ | 1 |
| | 10⁻⁵ | 0 |
| | 10⁻⁴ | 0 |
| 93. | 10⁻⁶ | 1 |
| | 10⁻⁵ | 0 |
| | 10⁻⁴ | 0 |
| 99. | 10⁻⁶ | 1 |
| | 10⁻⁵ | 0 |
| | 10⁻⁴ | 0 |
| 115. | 10⁻⁶ | 1 |
| | 10⁻⁵ | 0 |
| | 10⁻⁴ | 0 |
| 117. | 10⁻⁶ | 1 |
| | 10⁻⁵ | 0 |
| | 10⁻⁴ | 0 |
| 121. | 10⁻⁶ | 1 |
| | 10⁻⁵ | 0 |
| | 10⁻⁴ | 0 |
| 101. | 10⁻⁶ | 3 |
| | 10⁻⁵ | 3 |
| | 10⁻⁴ | 2 |
| 104. | 10⁻⁶ | 3 |
| | 10⁻⁵ | 3 |
| | 10⁻⁴ | 2 |
| 109. | 10⁻⁶ | 3 |
| | 10⁻⁵ | 3 |
| | 10⁻⁴ | 2 |
| 111. | 10⁻⁶ | 3 |
| | 10⁻⁵ | 3 |
| | 10⁻⁴ | 2 |
| 112. | 10⁻⁶ | 3 |
| | 10⁻⁵ | 3 |
| | 10⁻⁴ | 2 |
| 131. | 10⁻⁶ | 3 |
| | 10⁻⁵ | 3 |
| | 10⁻⁴ | 2 |
| 146. | 10⁻⁶ | 3 |
| | 10⁻⁵ | 3 |
| | 10⁻⁴ | 2 |
| 20. | 10⁻⁶ | 3 |
| | 10⁻⁵ | 3 |
| | 10⁻⁴ | 1 |
| 30. | 10⁻⁶ | 3 |
| | 10⁻⁵ | 2 |
| | 10⁻⁴ | 1 |
| 39. | 10⁻⁶ | 3 |
| | 10⁻⁵ | 2 |
| | 10⁻⁴ | 0 |
| 10. | 10⁻⁶ | 3 |
| | 10⁻⁵ | 1 |
| | 10⁻⁴ | 0 |
| 29. | 10⁻⁶ | 3 |
| | 10⁻⁵ | 1 |
| | 10⁻⁴ | 0 |
| 32. | 10⁻⁶ | 3 |
| | 10⁻⁵ | 1 |
| | 10⁻⁴ | 0 |
| 60. | 10⁻⁶ | 3 |
| | 10⁻⁵ | 0 |
| | 10⁻⁴ | 1 |
| 61. | 10⁻⁶ | 2 |
| | 10⁻⁵ | 0 |
| | 10⁻⁴ | 2 |
| 7. | 10⁻⁶ | 2 |
| | 10⁻⁵ | 0 |
| | 10⁻⁴ | 0 |
| 12. | 10⁻⁶ | 2 |
| | 10⁻⁵ | 0 |
| | 10⁻⁴ | 0 |
| 31. | 10⁻⁶ | 2 |
| | 10⁻⁵ | 0 |
| | 10⁻⁴ | 0 |
| 113. | 10⁻⁶ | 2 |
| | 10⁻⁵ | 0 |
| | 10⁻⁴ | 0 |
| 114. | 10⁻⁶ | 2 |
| | 10⁻⁵ | 0 |
| | 10⁻⁴ | 0 |
| 137. | 10⁻⁶ | 2 |
| | 10⁻⁵ | 0 |
| | 10⁻⁴ | 0 |
| 148. | 10⁻⁶ | 2 |
| | 10⁻⁵ | 0 |
| | 10⁻⁴ | 0 |
| 151. | 10⁻⁶ | 2 |
| | 10⁻⁵ | 0 |
| | 10⁻⁴ | 0 |
| 153. | 10⁻⁶ | 2 |
| | 10⁻⁵ | 0 |
| | 10⁻⁴ | 0 |
| 155. | 10⁻⁶ | 2 |
| | 10⁻⁵ | 0 |
| | 10⁻⁴ | 0 |
| 67. | 10⁻⁶ | 1 |
| | 10⁻⁵ | 3 |
| | 10⁻⁴ | 3 |
| 82. | 10⁻⁶ | 1 |
| | 10⁻⁵ | 3 |
| | 10⁻⁴ | 2 |
| 119. | 10⁻⁶ | 1 |
| | 10⁻⁵ | 3 |
| | 10⁻⁴ | 2 |
| 122. | 10⁻⁶ | 1 |
| | 10⁻⁵ | 0 |
| | 10⁻⁴ | 0 |
| 134. | 10⁻⁶ | 1 |
| | 10⁻⁵ | 0 |
| | 10⁻⁴ | 0 |
| 138. | 10⁻⁶ | 1 |
| | 10⁻⁵ | 0 |
| | 10⁻⁴ | 0 |
| 141. | 10⁻⁶ | 1 |
| | 10⁻⁵ | 0 |
| | 10⁻⁴ | 0 |
| 144. | 10⁻⁶ | 1 |
| | 10⁻⁵ | 0 |
| | 10⁻⁴ | 0 |
| 147. | 10⁻⁶ | 1 |
| | 10⁻⁵ | 0 |
| | 10⁻⁴ | 0 |
| 158. | 10⁻⁶ | 1 |
| | 10⁻⁵ | 0 |
| | 10⁻⁴ | 0 |
| 159. | 10⁻⁶ | 1 |
| | 10⁻⁵ | 0 |
| | 10⁻⁴ | 0 |
| 160. | 10⁻⁶ | 1 |
| | 10⁻⁵ | 0 |
| | 10⁻⁴ | 0 |
| 66. | 10⁻⁶ | 0 |
| | 10⁻⁵ | 3 |
| | 10⁻⁴ | 2 |
| 79. | 10⁻⁶ | 0 |
| | 10⁻⁵ | 3 |
| | 10⁻⁴ | 2 |
| 81. | 10⁻⁶ | 0 |
| | 10⁻⁵ | 3 |
| | 10⁻⁴ | 2 |
| 69. | 10⁻⁶ | 0 |
| | 10⁻⁵ | 3 |
| | 10⁻⁴ | 0 |
| 127. | 10⁻⁶ | 0 |
| | 10⁻⁵ | 2 |
| | 10⁻⁴ | 1 |
| 6. | 10⁻⁶ | 3 |
| | 10⁻⁵ | 0 |
| | 10⁻⁴ | 0 |
| 24. | 10⁻⁶ | 3 |
| | 10⁻⁵ | 0 |
| | 10⁻⁴ | 0 |
| 35. | 10⁻⁶ | 3 |
| | 10⁻⁵ | 0 |
| | 10⁻⁴ | 0 |
| 36. | 10⁻⁶ | 3 |
| | 10⁻⁵ | 0 |
| | 10⁻⁴ | 0 |
| 62. | 10⁻⁶ | 3 |
| | 10⁻⁵ | 0 |
| | 10⁻⁴ | 0 |
| 136. | 10⁻⁶ | 3 |
| | 10⁻⁵ | 0 |
| | 10⁻⁴ | 0 |
| 149. | 10⁻⁶ | 2 |
| | 10⁻⁵ | 3 |
| | 10⁻⁴ | 3 |
| 1. | 10⁻⁶ | 2 |
| | 10⁻⁵ | 3 |
| | 10⁻⁴ | 2 |
| 37. | 10⁻⁶ | 2 |
| | 10⁻⁵ | 3 |
| | 10⁻⁴ | 2 |
| 41. | 10⁻⁶ | 2 |
| | 10⁻⁵ | 3 |
| | 10⁻⁴ | 2 |
| 95. | 10⁻⁶ | 2 |
| | 10⁻⁵ | 3 |
| | 10⁻⁴ | 2 |
| 97. | 10⁻⁶ | 2 |
| | 10⁻⁵ | 3 |
| | 10⁻⁴ | 2 |
| 135. | 10⁻⁶ | 2 |
| | 10⁻⁵ | 3 |
| | 10⁻⁴ | 2 |
| 73. | 10⁻⁶ | 1 |
| | 10⁻⁵ | 3 |
| | 10⁻⁴ | 1 |
| 72. | 10⁻⁶ | 1 |
| | 10⁻⁵ | 2 |
| | 10⁻⁴ | 1 |
| 86. | 10⁻⁶ | 1 |
| | 10⁻⁵ | 2 |
| | 10⁻⁴ | 1 |
| 21. | 10⁻⁶ | 1 |
| | 10⁻⁵ | 2 |
| | 10⁻⁴ | 0 |
| 88. | 10⁻⁶ | 1 |
| | 10⁻⁵ | 1 |
| | 10⁻⁴ | 1 |
| 89. | 10⁻⁶ | 1 |
| | 10⁻⁵ | 1 |
| | 10⁻⁴ | 1 |
| 94. | 10⁻⁶ | 1 |
| | 10⁻⁵ | 1 |
| | 10⁻⁴ | 1 |
| 110. | 10⁻⁶ | 1 |
| | 10⁻⁵ | 1 |
| | 10⁻⁴ | 1 |
| 48. | 10⁻⁶ | 1 |
| | 10⁻⁵ | 1 |
| | 10⁻⁴ | 0 |
| 74. | 10⁻⁶ | 1 |
| | 10⁻⁵ | 1 |
| | 10⁻⁴ | 0 |
| 96. | 10⁻⁶ | 1 |
| | 10⁻⁵ | 1 |
| | 10⁻⁴ | 0 |
| 120. | 10⁻⁶ | 1 |
| | 10⁻⁵ | 1 |
| | 10⁻⁴ | 0 |
| 124. | 10⁻⁶ | 1 |
| | 10⁻⁵ | 1 |
| | 10⁻⁴ | 0 |
| 43. | 10⁻⁶ | 0 |
| | 10⁻⁵ | 2 |
| | 10⁻⁴ | 0 |
| 45. | 10⁻⁶ | 0 |
| | 10⁻⁵ | 2 |
| | 10⁻⁴ | 0 |
| 46. | 10⁻⁶ | 0 |
| | 10⁻⁵ | 2 |
| | 10⁻⁴ | 0 |
| 91. | 10⁻⁶ | 0 |
| | 10⁻⁵ | 1 |
| | 10⁻⁴ | 1 |
| 102. | 10⁻⁶ | 0 |
| | 10⁻⁵ | 1 |
| | 10⁻⁴ | 1 |
| 9. | 10⁻⁶ | 0 |
| | 10⁻⁵ | 1 |
| | 10⁻⁴ | 0 |
| 16. | 10⁻⁶ | 0 |
| | 10⁻⁵ | 1 |
| | 10⁻⁴ | 0 |
| 22. | 10⁻⁶ | 0 |
| | 10⁻⁵ | 1 |
| | 10⁻⁴ | 0 |
| 25. | 10⁻⁶ | 0 |
| | 10⁻⁵ | 1 |
| | 10⁻⁴ | 0 |
| 44. | 10⁻⁶ | 0 |
| | 10⁻⁵ | 1 |
| | 10⁻⁴ | 0 |
| 64. | 10⁻⁶ | 0 |
| | 10⁻⁵ | 1 |
| | 10⁻⁴ | 0 |
| 68. | 10⁻⁶ | 0 |
| | 10⁻⁵ | 1 |
| | 10⁻⁴ | 0 |
| 70. | 10⁻⁶ | 0 |
| | 10⁻⁵ | 1 |
| | 10⁻⁴ | 0 |
| 54. | 10⁻⁶ | 2 |
| | 10⁻⁵ | 3 |
| | 10⁻⁴ | 1 |
| 55. | 10⁻⁶ | 2 |
| | 10⁻⁵ | 3 |
| | 10⁻⁴ | 1 |
| 14. | 10⁻⁶ | 2 |
| | 10⁻⁵ | 3 |
| | 10⁻⁴ | 0 |
| 126. | 10⁻⁶ | 1 |
| | 10⁻⁵ | 1 |
| | 10⁻⁴ | 0 |
| 26. | 10⁻⁶ | 1 |
| | 10⁻⁵ | 0 |
| | 10⁻⁴ | 0 |
| 98. | 10⁻⁶ | 0 |
| | 10⁻⁵ | 1 |
| | 10⁻⁴ | 0 |
| 154. | 10⁻⁶ | 0 |
| | 10⁻⁵ | 0 |
| | 10⁻⁴ | 1 |

### Example 3

### Measurement of Intrinsic Compound Toxicity

Dilution of Library Compounds for Use in Toxicity Study

Compound samples in PBS (without Ca⁺², Mg⁺²)-glucose 3 mM-HEPES 10 mM—DMSO 1%, as prepared in Example 1, were thawed and left for at least 30 minutes at 20-23° C. before preparation of the following sub-dilutions for the compound toxicity assay:
100 μL of stock compound solution were added to 900 μL PBSD-1 [diluted 1:10 for a final 1:10 dilution]
100 μL (1/10 dilution above) were added to 900 μL PBSD-1 [diluted 1:10 for a final 1:100 dilution]
   Culture of HUVEC

Endothelial cells from human umbilical cord (HUV-EC-C or HUVEC) were purchased form American Type Culture Collection (ATCC, CRL-1730) and cultured according to the manufacturer's protocol. A 1-mL frozen aliquot of sub-cultured cells was thawed in a 37° C. water bath and centrifuged following addition of 5 mL of medium. After re-suspension in 5 mL medium, cells were seeded in a TC80 cm² flask pre-coated with 0.1% gelatin. Culture medium was replaced every 3-4 days and cells monitored until confluency was reached.

Preparation of Camptothecin Control

A sterile stock solution of 0.5 mM of camptothecin (Sigma) was prepared by dissolving a weighed amount of 0.0085 g in 50 mL of double distilled water at 37° C. in a water bath. The solution was vortexed, then filtered through a 0.22-μm filter unit and kept at 4° C. for the duration of the toxicity assays.

From the stock solution, concentrations of 60, 75, 80, 100, 150, 200 and 250 nM of camptothecin were prepared in culture diluent containing 1% DMSO.

Cellular Toxicity Assay Protocol

HUVEC were cultured for 4 days in gelatin-coated 96-well plates, following seeding with 40 μL/well of a cellular suspension of 1×105 cells/mL. Culture media was replaced every 3-4 days of culture until confluency was reached.

On the day of the toxicity assay, the conditioned culture media was removed from the wells and 90 μL of culture medium containing 1% DMSO was distributed in the plate. A volume of 10 μL of the stock solution, 1:10 and 1:100 dilutions of the compounds in PBS-glucose-HEPES-DMSO 1% were added to the appropriate wells (100-μL total volume/well). A volume of 100 μL of camptothecin dilutions and culture medium containing 1% DMSO was also distributed into the plate. Cells were incubated at 37° C. of 24 hours and then 10 μL of the tetrazolium salt WST-1 solution were added to cells and incubated for an extra 90 minutes at 37° C. The absorbance that was associated to the cellular viability was measured at 450nm on the SpectraFluor Tecan reader, and the raw data processed.

Results

The intrinsic cellular toxicity of the compounds was determined for each concentration of every compound on HUVEC. The viability percentage was assessed (OD sample/OD control) ×100% and any value <75% viability was considered toxic. The following table, Table 5, lists the few compounds that presented at least one toxic concentration. None of the 21 compounds ranked as highly effective to bind to L1 transport system induced cellular toxicity.

TABLE 5
Intrinsic cellular toxicity of exemplary compounds of the present invention

| ID# | Conc. (M) | Viability (%) |
|---|---|---|
| 30 | 10⁻⁴ | 110% |
| | 10⁻⁵ | 112% |
| | 10⁻⁶ | 8% |
| 36 | 10⁻⁴ | 97% |
| | 10⁻⁵ | 66% |
| | 10⁻⁶ | 99% |
| 37 | 10⁻⁴ | 94% |
| | 10⁻⁵ | 50% |
| | 10⁻⁶ | 87% |
| 65 | 10⁻⁴ | 107% |
| | 10⁻⁵ | 68% |
| | 10⁻⁶ | 109% |
| 66 | 10⁻⁴ | 117% |
| | 10⁻⁵ | 114% |
| | 10⁻⁶ | 67% |

### Example 4

### Synthesis of Exemplary Compounds

Compounds were synthesized in accordance with the following exemplary schemes:
Synthesis of Exemplary Compounds (Route 1):

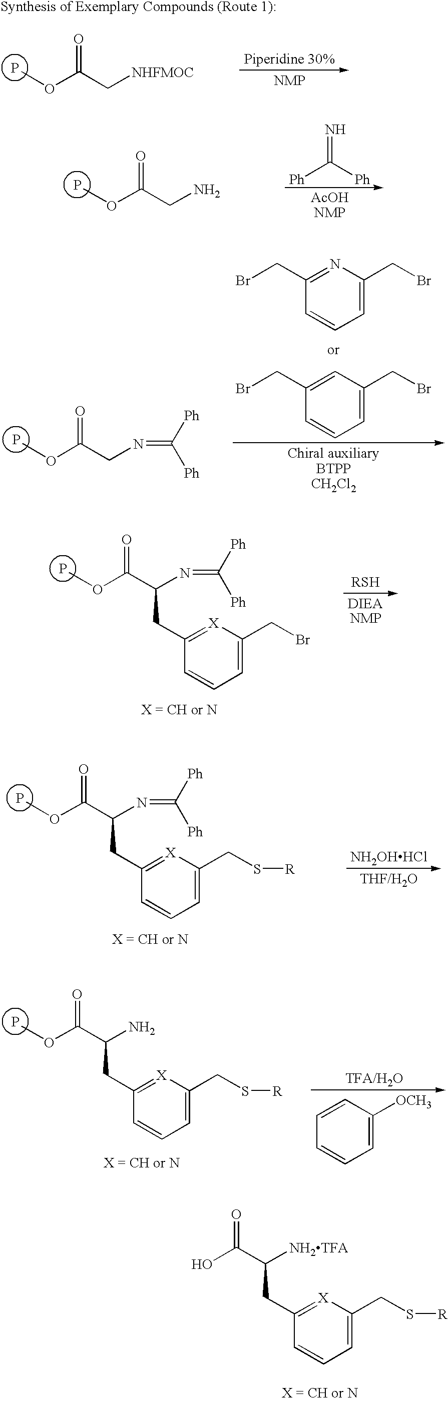

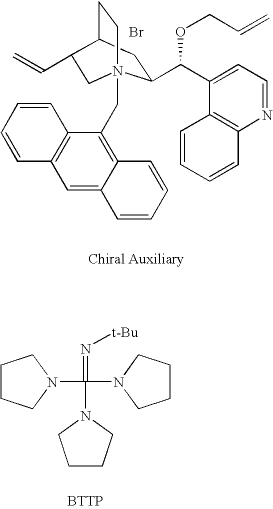

Step 1: Fmoc-Gly-Wang resin (5 mmol) was washed with DMF (4×25 mL), 25% piperidine/DMF (1×25 mL, 3 min and 1×25 mL 17 min) and DMF (4×25 mL). To cleave the Fmoc group, 25 mL of a 30% piperidine/N-methylpyrrolidinone (NMP) solution was added to the resin and the suspension was shaken for 30 minutes. The reagents and solvent were filtered and the resin was washed with NMP (4×25 mL).

Step 2: Directly to the resin was added benzopheneone imine (25 mmol) and acetic acid (AcOH, 25 mmol) in 25 mL of NMP. The reaction was shaken overnight. The reagents and solvent were filtered and the resin was washed with DMF (4×25 mL), H₂O (4×25 mL), MeOH (4×25 mL), MeOH/N,N-diisopropylethylamine (DIEA) (10/1, 3×22 mL) and CH₂Cl₂ (4×25 mL). The resin was then dried in vacuo.

Step 3: The resin (5 mmol), α,α-dibromoxylene or 2,6-bis(bromomethyl)pyridine (25 mmol) and o-allyl-N-(9-anthracenylmethyl) cinchonidinium bromide (5 mmol) were mixed in 25 mL of anhydrous CH₂Cl₂. The suspension was slowly stirred at room temperature for 5 minutes. It was then cooled to −78 ° C. and stirred for 20 additional minutes. Phospozene base t-Bu-tris(tetramethylene) (BTPP, 25 mmol) was added and the suspension was shaken for 4 hours at −78° C. and one hour at −15 ° C. The reagents and solvent were filtered and the resin was washed with DMF (4×25 mL), H₂O (4×25 mL), DMF/H₂O (4×25 mL), CH₂Cl₂ (4×25 mL) and Et₂O (4×25 mL). The resin was then dried in vacuo.

Step 4: The resin (1 mmol) was swelled in 5 mL of NMP. The suspension was shaken for 30 minutes and a solution of thiol (5 mmol) and DIEA (12 mmol) in 5 mL of NMP was added. The suspension was shaken for 22 hours at room temperature. The reagents and solvent were filtered and the resin was washed with CH₂Cl₂ (4×10 mL), THF (4×10 mL), THF/H₂O (4×10 mL) and THF (4×10 mL).

Step 5: The resin was then suspended in 10 mL of 1N NH₂OH.HCl in THF/H₂O (2/1). The mixture was shaken for 5 hours at room temperature. The reagents and solvent were filtered and the resin was washed with THF (4×10 mL) and NMP (4×10 mL). To the resin was added 1N DIEA (1.8 mL DIEA and 8.2 mL of NMP). The suspension was shaken 30 minutes at room temperature. The reagents and solvent were filtered and the resin was washed with NMP (4×10 mL) and CH₂Cl₂ (4×10 mL).

Step 6: The resin was suspended in a mixture of TFA/H₂O/Anisole (95%/2.5%/2.5%). The suspension was shaken for 30 minutes and the solvent was recovered in a flask. The resin was washed with TFA (10 mL). Filtrates were combined and the volume of solvent was reduced to 1/8 of the initial volume. A few drops of Et₂O were added to the solution, and the product was precipitated with hexanes. The suspension was centrifuged and the supernatant was removed. The residual solvent was removed with a stream of N₂. The above precipitation procedure was repeated with the supernatant. The products were combined and purified by preparative HPLC.

NMR Results for Exemplary Compounds Synthesized by Route 1

3-{-1-[(4-methoxyphenyl)-tetrazole]-5-yl-sulfanylmethyl}-L-phenylalanine, trifluoroacetic acid salt, white solid, 22% overall yield, [α]_{D}=−8.5 ° (in H₂0). ¹H NMR (D₂O, 500 MHz) δ ppm 7.24 (d, 2H, J=8.3 Hz), 7.10 (t, 1H, J=7.6 Hz), 7.08 (m, 3H), 7.00 (d, 2H, J=8.3 Hz), 4.29 (s, 2H), 3.83 (t, 1H, J=6.1 Hz), 3.81 (dd, 1H, J=4.9 Hz, 14.2 Hz), 2.95 (dd, 1H, J=7.8 Hz, 14.2 Hz). ¹³C (D₂O, 125 MHz) δ ppm 170.00, 169.97, 161.52, 154.50, 137.18, 135.02, 130.19, 129.36, 128.90, 128.46, 126.17, 125.99, 114.82, 55.06, 36.76, 36.01. ES-MS 386 (M+1).

3-[3-(1-phenyl-1H-tetrazol-5-ylsulfanylmethyl)]-L-phenylalanine, trifluoroacetic acid salt, white solid, 18% overall yield, [α]_{D}=−1.5 ° (in H₂0). ¹H NMR (D₂O, 500 MHz) δ ppm 7.48 (m, 3H), 7.32 (d, 2H, J=8.3 Hz), 7.18 (t, 1H, J=7.6 Hz), 7.13 (d, 1H, J=7.8 Hz), 7.08 (m, 2H), 4.32 (s, 2H), 4.02 (t, 1H, J=6.6 Hz), 3.10 (dd, 1H, J=5.9 Hz, 14.6 Hz), 2.98 (dd, 1H, J=7.6 Hz, 14.4 Hz). ES-MS 356 (M+1).

3-[3-(1H-benzimidazol-2-ylsulfanylmethyl)]-L-phenylalanine, trifluoroacetic acid salt, white solid, 16% overall yield, [α]_{D}=−3.9° (in H₂0). ¹H NMR (D₂O, 500 MHz) δ ppm 7.48 (dd, 2H, J=3.4 Hz, 6.1 Hz), 7.36 (dd, 2H, J=3.2 Hz, 6.1 Hz), 4.37 (s, 2H), 3.71 (t, 1H, J=6.4 Hz), 2.89 (dd, 1H, J=6.4 Hz, 14.6 Hz), 2.83 (dd, 1H, J=7.3 Hz, 14.6 Hz). ES-MS 328 (M+1).

3-[3-(5-phenyl-2H-[1,2,4]triazol-3-ylsulfanylmethyl)]-L-phenylalanine, tri-fluoroacetic acid salt, white solid, 39% overall yield, [α]_{D}=−3.2° (in H₂0). ¹H NMR (D₂O, 500 MHz) δ ppm 7.70 (m, 2H), 7.42 (m, 3H), 7.16 (t, 1H, J=8.1 Hz), 7.10 (d, 2H, J=6.4 Hz), 7.02 (m, 2H), 4.15 (s, 2H), 3.97 (t, 1H, J=5.6 Hz), 3.07 (dd, 1H, J=5.9 Hz, 14.6 Hz), 2.94 (dd, 1H, J=7.8 Hz, 14.6 Hz). ES-MS 355 (M+1).

2-amino-3-[6-(1H-benzimidazol-2-ylsuaanylmethyl)-pyridin-2-yl]-L-propio-nic acid, trifluoroacetic acid salt, light yellow solid, 6% overall yield, [α]_{D}=+5.7° (in H₂0). ¹H NMR (D₂O, 500 MHz) δ ppm 7.74 (t, 1H, J=7.8 Hz), 7.66 (m, 2H), 7.42 (m, 2H), 7.31 (d, 1H, J=7.8 Hz), 7.27 (d, 1H, J=7.8 Hz), 4.57 (s, 2H), 4.00 (t, 1H, J=6.3 Hz), 3.22 (d, 2H, J=5.9 Hz). ¹³C (D₂O, 125 MHz) δ ppm 172.81, 155.13, 153.83, 141.06, 132.04, 126,54, 124,68, 123,27, 113,48, 70.01, 53.05, 38,28, 35.92. ES-MS 329 (M+1).

2-amino-3-[6-(1H-pyrazolo-[3,4-d]pyrimidin-4-ylsulfanylmethyl)-pyridin-2-yl]-L-propionic acid, trifluoroacetic acid salt, light yellow solid, 2% overall yield, [α]_{D}=−5.0° (in H₂0). ¹H NMR (D₂O, 500 MHz) δ ppm 8.56 (s, 1H), 8.20 (t, 1H, J=8.1 Hz), 8.13 (s, 1H), 7.89 (d, 1H, J=7.8 Hz), 7.65 (d, 1H, J=7.8 Hz), 4.78 (s, 2H), 4.19 (t, 1H, J=7.1 Hz), 3.48 (m, 2H). ¹³C (D₂O, 125 MHz) δ ppm 171.35, 164.58, 154.09, 153.65, 151.38, 146.07, 132.79, 126.25, 126.06, 111.49, 52.57, 34.173, 30.73. ES-MS 331 (M+1).

2-amino-3-[3-(1H-imidazol-2-ylsulfanylmethyl)-phenyl]-L-propionic acid, trifluoroacetic acid salt, light yellow solid, 7% overall yield, [α]_{D}=−3.6° (in H₂0). ¹H NMR (D₂O, 500 MHz) δ ppm 7.72 (m, 2H), 7.54 (t, 1H, J=7.6 Hz), 7.36 (m, 3H), 6.95 (d, 1H, J=7.3 Hz), 5.02 (m, 1H), 4.92 (m, 1H), 4.78 (t, 1H, J=5.1 Hz), 3.05 (dd, 1H, J=5.9 Hz, 14.6 Hz), 3.01 (dd, 1H, J=6.8 Hz, 14.6 Hz). ¹³C (D₂O, 125 MHz) δ ppm 172.84, 163.34, 163.05, 137.95, 137.08, 135.59, 129.75, 129.69, 129.23, 128.16, 128.57, 121.52, 117.74, 115.43, 55.28, 39.82, 35.96. ES-MS 278 (M+1).

2-amino-3-[3-(4-hydroxypyrrimidin-2-ylsulanylmethyl)-phenyl]-L-propionic acid, trifluoroacetic acid salt, white solid, 7% overall yield, [α]_{D}=−4.2° (in H₂0). ¹H NMR (D₂O, 500 MHz) δ ppm 7.70 (d, 1H, J=6.8 Hz), 7.30 (m, 1H), 7.24 (m, 2H), 7.14 (d, 1H, J=7.3 Hz), 6.10 (d, 1H, J=6.8 Hz), 4.32 (s, 2H), 4.06 (t, 1H, J=5.6 Hz), 3.16 (dd, 1H, J=5.9 Hz, 14.6 Hz), 3.05 (dd, 1H, J=7.8 Hz, 14.6 Hz). ¹³C (D₂O, 125 MHz) δ ppm 172.32, 149 137.54, 135.02, 130.02, 129.67, 128.90, 128.55, 109.52, 54.85, 35.85, 34.30. ES-MS 306 (M+1).

2-amino-3-[3-(4-trifluoromethylpyrrimidin-2-ylsulfanylmethyl)-phenyl]-L-propionic acid, trifluoroacetic acid salt, light yellow solid, 3% overall yield, [α]_{D}=+2.2° (in H₂0). ¹H NMR (D₂O, 500 MHz) δ ppm 8.65 (d, 1H, J=4.4 Hz), 8.18 (t, 1H, J=7.8 Hz), 7.92 (d, 1H, J=7.8 Hz), 7.62 (d, 1H, J=7.8 Hz), 7.40 (d, 1H, J=4.4 Hz), 4.59 (s, 2H), 4.15 (t, 1H, J=7.1 Hz), 3.44 (m, 2H). ¹³C (D₂O, 125 MHz) δ ppm 170.96, 170.36, 160.98, 155.64, 155.35, 154.11, 150.69, 146.51, 126.15, 126.40, 13.92, 52.37, 33.70, 32.16. ES-MS 359 (M+1).

2-amino-3-[6-(6-chlorobenzothiazol-2-ylsuaianylmethyl)-pyridin-2-yl-L-pro-pionic acid, trifluoroacetic acid salt, light yellow solid, 7% overall yield, [α]_{D}=−3.1° (in H₂0). ¹H NMR (CDCl₃, 500 MHz) δ ppm 7.95 (t, 1H, J=7.8 Hz), 7.78 (s, 1H), 7.74 (d, 1H, J=7.8 Hz), 7.58 (d, 1H, J=7.8 Hz), 7.22 (d, 1H, J=8.3 Hz), 4.78 (s, 2H), 4.49 (m, 1H), 3.66 (m, 2H). ¹³C (CDCl₃, 125 MHz) δ ppm 167.15, 154.65, 154.00, 153.53, 148.80, 142.97, 133.57, 132.57, 125.21, 124.87, 121.96, 121.64, 53.30, 35.29, 33.99. ES-MS 380 (M+1).

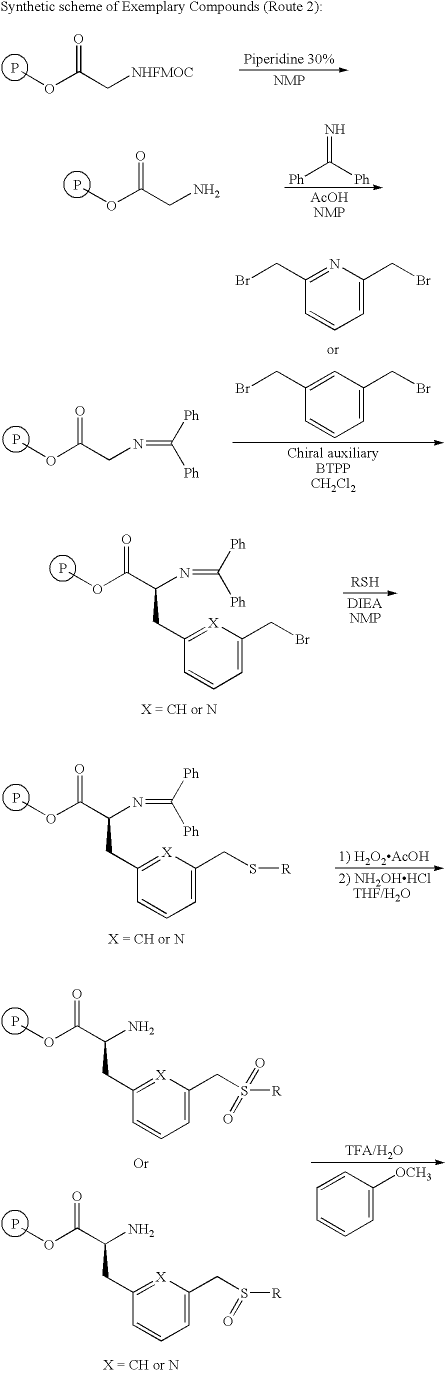

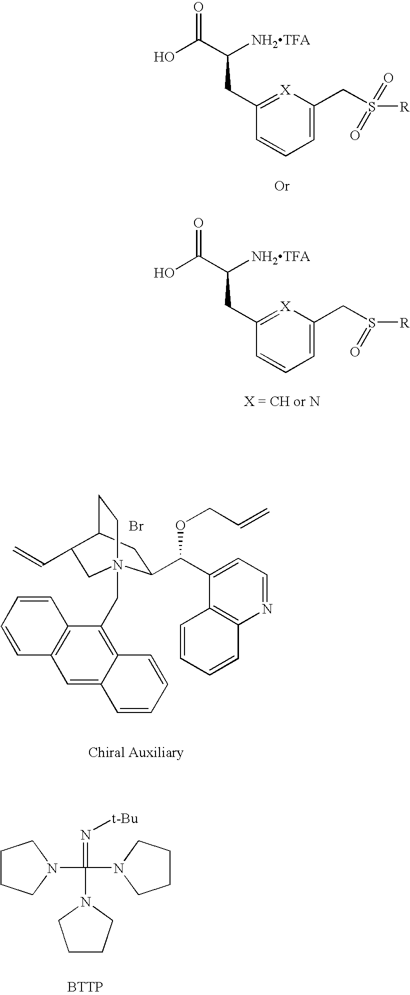

Steps 1 through 4 as described in Route 1 were followed.

Step 5: The resin (1 mmol) was suspended in a solution of 22.5 mL of acetic acid (AcOH) and 2.5 mL of H₂O₂ (35% wt in water). The suspension was shaken for 18 hours and the reagents and solvent were filtered. The resin was washed with EtOH (4×10 mL) and THF (4×10 mL).

Step 6 as described in Route 1 was followed.

NMR Results for Exemplary Compounds Synthesized by Route 2

2-amino-3-[6-(1H-benzimidazol-2-sulfonylmethyl)-pyridin-2-yl]-L-propionic acid, trifluoroacetic acid salt, light yellow solid, 6% overall yield, [α]_{D}=+1.0° (in H₂0). ¹H NMR (Acetone, 500 MHz) δ ppm 7.72 (m, 2H), 7.54 (t, 1H, J=7.6 Hz), 7.36 (m, 3H), 6.95 (d, 1H, J=7.3 Hz), 5.02 (m, 1H), 4.92 (m, 1H), 4.78 (t, 1H, J=5.1 Hz), 3.67 (m, 2H). ¹³C (D₂O, 125 MHz) δ ppm 170.61, 154.88, 145.28, 144.20, 138.72, 136.64, 125.10, 124.29, 123.89, 115.82, 55.35, 51.16, 34.23. ES-MS 361 (M+1).

3-{-1-[(4-methoxyphenyl)-tetrazole]-5-yl-sulfinylmethyl)-L-phenylalanine, trifluoroacetic acid salt, light yellow solid, 7% overall yield, [α]_{D}=−2.7° (in H₂0). ¹H NMR (D₂O, 500 MHz) δ ppm 7.14 (m, 2H), 7.07 (dd, 2H, J=2.2 Hz, 9.0 Hz), 6.94 (dd, 2H, J=2.2 Hz, 9.0 Hz), 6.82 (m, 2H), 5.04 (m, 2H), 3.95 (m, 1H), 3.76 (s, 3H), 3.01 (m, 1H), 2.88 (m, 1H). ¹³C (D₂O, 125 MHz) δ ppm 172.07, 161.112, 135.56, 131.28, 130.80, 130.10, 129.81, 129.76, 127,42, 126.31, 124.95, 115.14, 59.85, 55.95, 54.62, 35.69. ES-MS 402 (M+1).

3-[3-(5-phenyl-2H-[1,2,4]triazol-3-ylsulfinylmethyl)]-L-phenylalanine, tri-fluoroacetic acid salt, light yellow solid, 2% overall yield, [α]_{D}=−8.5° (in H20). ¹H NMR (D₂O, 500 MHz) δ ppm 7.75 (m, 2H), 7.47 (m, 3H), 7.16 (m, 2H), 6.94 (m, 2H), 4.48 (s, 2H), 4.30 (m, 1H), 3.95 (m, 1H), 3.36 (dd, 2H, J=5.3 Hz, 14.6 Hz), 2.96 (m, 1H). ¹³C (D₂O, 125 MHz) δ ppm 172.06, 161.94, 157.99, 135.11, 131.94, 131.49, 131.418, 129.98, 129.68, 129.61, 128.85, 126.94, 125.42, 58.96, 54.732, 35.72. ES-MS 371 (M+1).

2-amino-3-[3-(1H-imidazol-2-ylsulfinylmethyl)-phenyl]-L-propionic acid, tri-fluoroacetic acid salt, white solid, 4% overall yield, [α]_{D}=−2.0° (in H₂0). ¹H NMR (D₂O, 500 MHz) δ ppm 7.22 (m, 4H), 6.92 (d, 1H, J=7.3 Hz), 6.89 (s, 1H), 4.62 (s, 2H), 4.05 (t, 1H, J=6.5 Hz), 3.13 (dd, 1H, J=5.9 Hz, 14.6 Hz), 3.00 (dd, 1H, J=7.6 Hz, 14.0 Hz). ¹³C (D₂O, 125 MHz) δ ppm 171.87, 139.54, 135.23, 131.69, 130.46, 130.36, 129,76, 127.68, 126.46, 61.54, 54.53, 35.61. ES-MS 310 (M+1).

2-amino-3-[3-(1H-imidazol-2-ylsulfinylmethyl)-phenyl]-L-propionic acid, tri-fluoroacetic acid salt, white solid, 4% overall yield, [α]_{D}=−2.0° (in H₂0). ¹H NMR (D₂O, 500 MHz) δ ppm 7.22 (m, 4H), 6.92 (d, 1H, J=7.3 Hz), 6.89 (s, 1H), 4.62 (s, 2H), 4.05 (t, 1H, J=6.5 Hz), 3.13 (dd, 1H, J=5.9 Hz, 14.6 Hz), 3.00 (dd, 1H, J=7.6 Hz, 14.0 Hz). ¹³C (D₂O, 125 MHz) δ ppm 171.87, 139.54, 135.23, 131.69, 130.46, 130.36, 129,76, 127.68, 126.46, 61.54, 54.53, 35.61. ES-MS 310 (M+1).

3-[3-(1-phenyl-1H-tetrazol-5-ylsulfinylmethyl)]-L-phenylalanine, trifluoro-acetic acid salt, white solid, 27% overall yield, [α]_{D}=−2.1° (in H₂0). ¹H NMR (D₂O, 500 MHz) δ ppm 7.44 (m, 1H), 7.36 (m, 2H), 7.10 (m, 4H), 4.58 (m, 1H), 4.49 (m, 1H), 3.94 (m, 1H), 2.96 (m, 1H), 2.85 (m, 1H). ¹³C (D₂O, 125 MHz) δ ppm 171.72, 156.50, 135.28, 131.75, 131.13, 131.05, 130.56, 129.86, 129.59, 127.31, 124.43, 59.52, 54.28, 35.40. ES-MS 372 (M+1).

3-[3-(1H-benzimidazol-2-ylsulfonylmethyl)]-L-phenylalanine, trifluoroacetic acid salt, white solid, 2% overall yield, [α]_{D}=−2.0° (in H₂0). ¹H NMR (D₂O, 500 MHz) δ ppm 7.57 (dd, 2H, J=3.4 Hz, 6.4 Hz), 7.37 (dd, 2H, J=3.4 Hz, 6.1 Hz), 7.11 (m,2H), 6.96 (d, 1H, J=7.7 Hz), 6.72 (s, 1H), 4.72 (s, 1H), 3.54 (t, 1H, J=6.2 Hz), 2.85 (dd, 1H, J=5.9 Hz, 14.6 Hz), 2.71 (dd, 1H, J=7.6 Hz, 14.4 Hz). ¹³C (D₂O, 125 MHz) δ ppm 172.22, 145.31, 137.66, 135.53, 131.53, 130.47, 130.24, 129.73, 127.37, 125.97, 116.87, 61.64, 54.878, 35.58. ES-MS 360 (M+1).

3-[3-(1-phenyl-1H-tetrazol-5-ylsulfinylmethyl)]-L-phenylalanine, trifluoro-acetic acid salt, light yellow solid, 6% overall yield, [α]_{D}=−10.1° (in H₂0). ¹H NMR (D₂O, 500 MHz) δ ppm 7.64 (t, 1H, J=7.8 Hz), 7.55 (dd, 1H, J=3.9 Hz, 7.6 Hz), 7.49 (t, 2H, J=7.6 Hz), 7.36 (m,2H), 7.06 (dd, 1H, J=7.8 Hz, 14.2 Hz), 4.73 (m, 2H), 4.09 (t, 1H, J=5.4 Hz), 3.10 (m, 2H). ¹³C (D₂O, 125 MHz) δ ppm 171.94, 156.74, 155.89, 147.30, 139.85, 131.87, 130.35, 124.98, 124.61, 60.79, 59.87, 52.59, 35.68. ES-MS 373 (M+1).

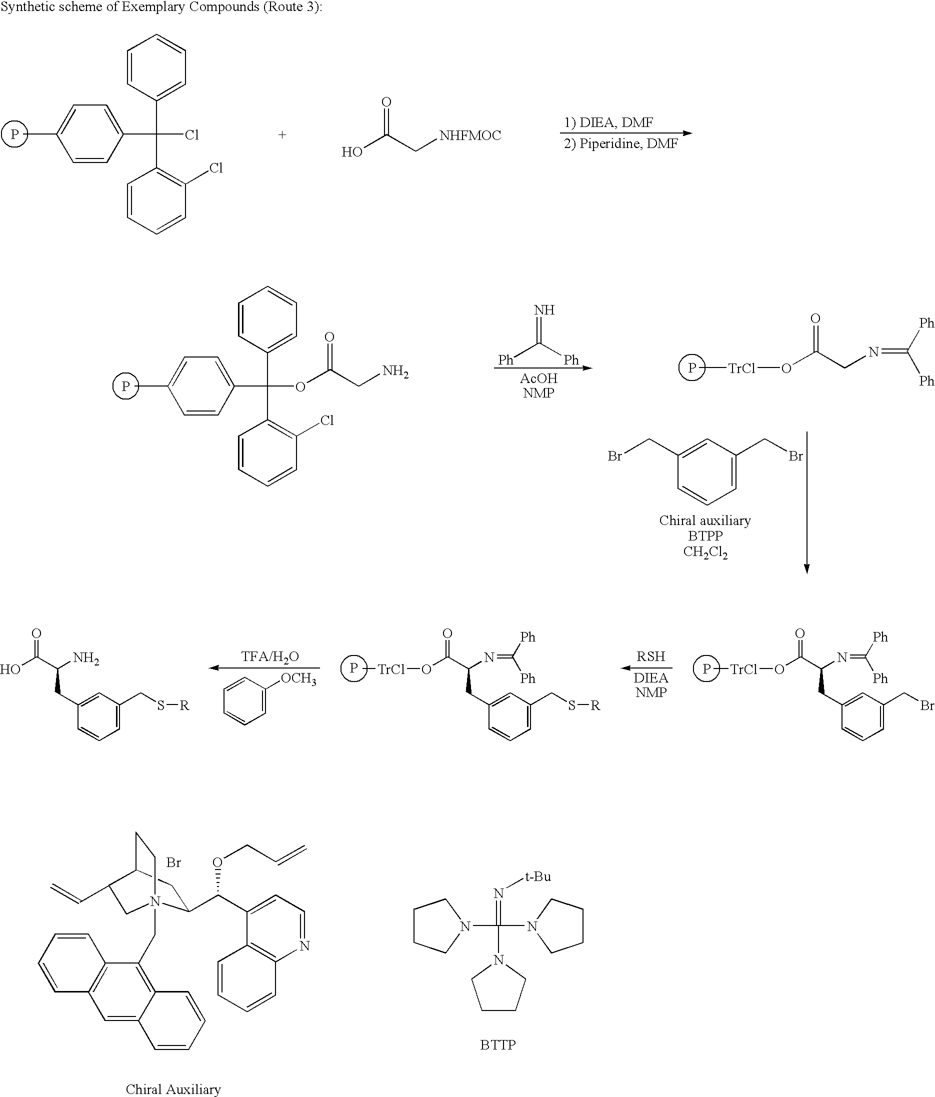

Step 1: Fmoc-Gly-OH (5.3 mmol) was dissolved in 44 mL of anhydrous CH₂Cl₂ and 6 mL of DMF. The solution was added to 6.6 mmol of 2-chlorotritylchloride resin with DIEA (21.2 mmol, 4 eq relative to the amino acid). The suspension was shaken for 30 min. The reagents and solvent were filtered. The resin was washed with CH₂Cl₂/MeOH/DIEA (17/2/1, 3×20 mL), CH₂Cl₂ (3×20 mL), DMF (2×20 mL), CH₂Cl₂ (2×20 mL) and MeOH (2×20 mL). The resin was dried in vacuo over KOH. To cleave the Fmoc group the resin was swelled in 5% piperidine in DMF/CH₂Cl₂ (20 mL, 1/1). The suspension was shaken for 10 min. The reagents and solvent were filtered. 20% piperidine in DMF (20 mL) was added to the resin. The suspension was shaken for 15 min. The reagents and solvent were filtered. The resin was washed with DMF (3×20 mL) and CH₂Cl₂ (3×20 mL).

Step 2: The resin (5.3 mmol) was swelled in 50 mL of NMP. The suspension was shaken for 5 min and the solvent was filtered. To the resin was added a solution of benzophenone imine (53.0 mmol) and AcOH (50.0 mmol) in 40 mL of NMP. The reaction was shaken overnight. The reagents and solvent were filtered and the resin was washed with DMF (4×10 mL), H₂O (4×10 mL), MeOH (4×10 mL), MeOH/N,N-diisopropylethylamine (DIEA) (10/1, 4×11 mL) and CH₂Cl₂ (4×10 mL). The resin was dried in vacuo.

Step 3: The resin (4.5 mmol), α,α-dibromoxylene (22.5 mmol) and the o-allyl-N-(9-anthracenylmethyl)cinchonidinium bromide (4.5 mmol) were mixed in 40 mL of anhydrous CH₂Cl₂. The suspension was shaken at r.t. for 5 min. It was then cooled to −50° C. (acetonitrile/dry ice bath) and stirred for 20 min. Phospozene base t-Bu-tris(tetramethylene) (BTPP, 22.5 mmol) was added. The suspension was stirred overnight at −78° C. The reagents and solvent were filtered and the resin was washed with DMF (4×10 mL), DMF/H₂O (4×20 mL) and CH₂Cl₂ (4×10 mL). The resin was dried in vacuo.

Step 4: The resin (1.0 mmol) was swelled in 10 mL of NMP. The suspension was shaken for 5 min and the solvent was filtered. A solution of thiol (5.6 mmol) and DIEA (13.5 mmol) in 10 mL of NMP was added. The suspension was shaken overnight at r.t. The reagents and solvent were filtered. The resin was washed with CH₂Cl₂ (4×10 mL), THF (4×10 mL), THF/H₂O (4×10 mL) and THF (4×10 mL),

Step 5: The resin was suspended in a mixture of TFA/H₂O/Anisole (95%/2.5%/2.5%, (10 mL). The suspension was shaken for 1 h. The solvent was recovered in a flask. The resin was washed with TFA (10 mL). The filtrates were combined and the solvent was evaporated. The product was precipitated with cold Et₂O. The suspension was centrifuged and the supernatant was removed. The solvent was removed of the solid with a stream of N₂. The same procedure was repeated twice with the supernatant. The products were combined and purified by preparative HPLC.

NMR Results for Exemplary Compounds Synthesized by Route 3

3-{-1-[(4-hydroxyphenyl)-tetrazole]-5-yl-sulfanylmethyl)-L-phenylalanine, trifluoroacetic acid salt, white solid, 43% overall yield, [α]_{D}=−1.1° (in H₂0). ¹H NMR (D₂O, 500 MHz) δ ppm 7.12 (m, 8H), 6.86 (d, 1H, J=8.8 Hz), 4.28 (s, 2H), 3.99 (t, 1H, J=6.6 Hz), 3.09 (dd, 1H, J=5.6 Hz, 14.4 Hz), 2.96 (dd, 1H, J=5.9 Hz, 14.6 Hz). ¹³C (D₂O, 125 MHz) δ ppm 174.08, 157.97, 136.95, 135.13, 129.88, 129.68, 129.18, 128.31, 126.62, 116.43, 54.84, 37.37, 35.86. ES-MS 372 (M+1).

3-[3-(5-pyridin-4-yl-[1,3,4]oxadiazol-2-ylsulfanylmethyl)]-L-phenylalanine, trifluoroacetic acid salt, light yellow solid, 10% overall yield, [α]_{D}=−1.7° (in H₂0). ¹H NMR (D₂O, 500 MHz) δ ppm 8.86 (d, 2H, J=6.8 Hz), 8.38 (d, 2H, J=6.8 Hz), 7.37 (d, 1H, J=7.3 Hz), 7.33 (s, 1H), 7.27 (t, 1H, J=7.8 Hz), 7.13 (d, 1H, J=7.8 Hz), 4.85 (s, 2H), 4.07 (t, 1H, J=5.9 Hz), 3.16 (dd, 1H, J=5.9 Hz, 14.2 Hz), 3.07 (dd, 1H, J=7.1 Hz, 14.2 Hz). ¹³C (D₂O, 125 MHz) δ ppm 172.23, 168.87, 162.56, 143.30, 138.60, 136.88, 135.22, 129.96, 129.77, 129.31, 128.516, 123.87, 54.79, 36.05, 35.84. ES-MS 357 (M+1).

3-{1-[2-(dimethylamino)ethyl]-1H-tetrazole-5-yl-sulanylmethyl)-L-phenyl-alanine, trifluoroacetic acid salt, white solid, 38% overall yield, [α]_{D}=−0.8° (in H₂0). ¹H NMR (D₂O, 500 MHz) δ ppm 7.48 (m, 2H), 7.11 (m, 2H), 4.51 (t, 1H, J=5.9 Hz), 7.33 (s, 1H), 7.27 (t, 1H, J=7.8 Hz), 7.13 (d, 1H, J=7.8 Hz), 4.34 (s, 2H), 4.14 (t, 1H, J=6.8 Hz), 3.46 (t, 2H, J=6.1 Hz), 3.14 (dd, 1H, J=6.1 Hz, 14.4 Hz), 3.05 (dd, 1H, J=7.3 Hz, 14.6 Hz). ¹³C (D₂O, 125 MHz) δ ppm 171.41, 154.74, 137.27, 134.96, 129.97, 129.91, 129.39, 128.55, 54.91, 54.19, 43.29, 42.36, 37.72, 35.57. ES-MS 351 (M+1).

3-[3-(5-pyridin-4-yl-4H-[1,2,4]triazol-3-ylsulfanylmethyl)]-L-phenylalanine, trifluoroacetic acid salt, white solid, 28% overall yield, [α]_{D}=−1.1° (in H₂0). ¹H NMR (D₂O, 500 MHz) δ ppm 8.74 (d, 2H, J=6.8 Hz), 8.37 (d, 2H, J=6.8 Hz), 7.11 (m, 4H), 4.23 (s, 2H), 3.98 (t, 1H, J=5.9 Hz), 3.08 (dd, 1H, J=5.4 Hz, 14.4 Hz), 2.99 (dd, 1H, J=7.3 Hz, 14.6 Hz). ¹³C(D₂O, 125 MHz) δ ppm 178.39, 172.36, 157.70, 154.15, 146.37, 142.16, 137.85, 135.11, 129.78, 129.56, 128.93, 128.38, 123.60, 54.88, 37.85, 35.83. ES-MS 356 (M+1).

### Example 5

### Binding of Exemplary Compounds to the Brain L1 Transport System

Compounds as synthesized above in Example 4 were diluted and tested for binding to the brain L1 transport system as described in Example 2, also above.

For each concentration (10⁻⁶, 10⁻⁵, and 10⁻⁴), the binding of ¹⁴C-labeled phenylalanine in the presence of the test compound (expressed as the % of the binding in absence of competition) was subtracted from the corresponding value measured in the presence of same concentration of phenylalanine (reference competition). The difference (in %), Δ, was expressed as a primary score (which represents the binding affinity proximity of a test compound to the phenylalanine binding curve). The primary score was converted to a numerical rating scale as the following:

3: Δ>10%, significantly higher binding affinity than phenylalanine

2: 10% ≧Δ≧−10%, similar binding affinity to phenylalanine

1: −10>Δ>−50%, lower binding affinity than phenylalanine

0: Δ≦50%, no binding or very low binding affinity

Results, shown in Table 6 below, indicate that 5 compounds exhibit a significantly higher binding affinity than phenylalanine, 4 compounds exhibit a similar binding affinity to phenylalanine, 9 compounds exhibit a binding affinity lower than than of phenylalanine, but still bind significantly to the transporter, and 2 compounds exhibit no binding or very low binding affinity.

TABLE 6
Results of L1 transport system binding study

| Structure | Status of Binding* |
|---|---|
| 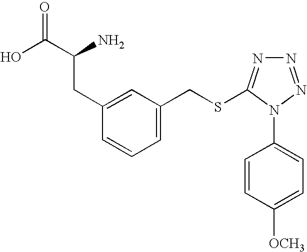 | 3 |
| 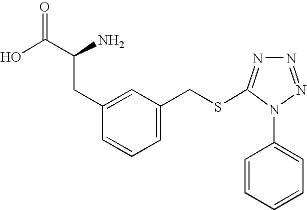 | 3 |
| 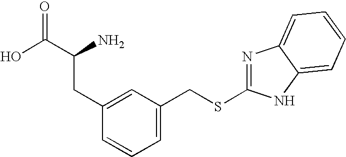 | 1 |
| 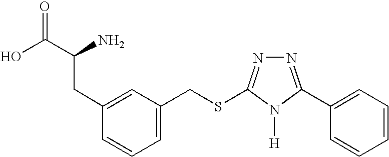 | 2 |
| 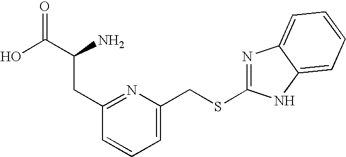 | 1 |
| 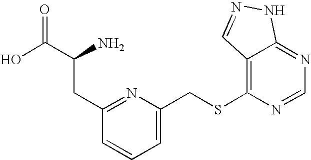 | 0 |
| 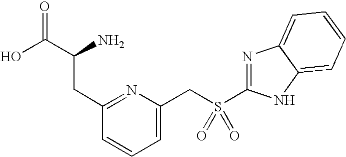 | 0 |
| 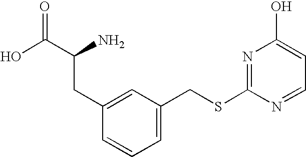 | 1 |
| 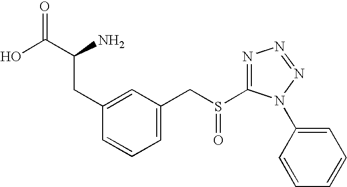 | 3 |
| 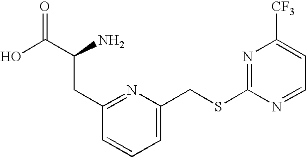 | 1 |
| 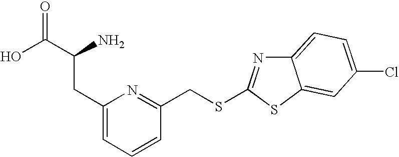 | 1 |
| 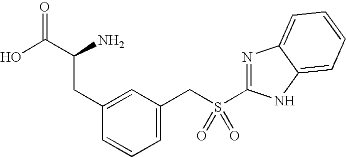 | 2 |
| 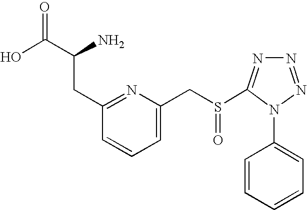 | 1 |
| 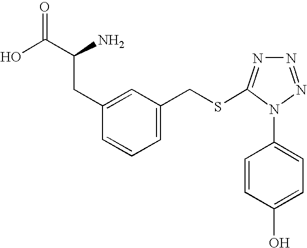 | 3 |
| 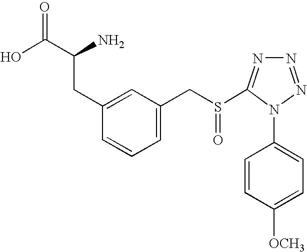 | 3 |
| 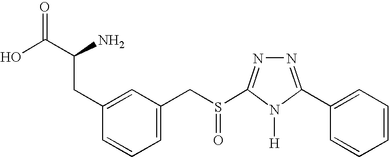 | 1 |
| 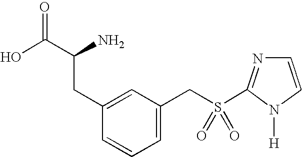 | 1 |
| 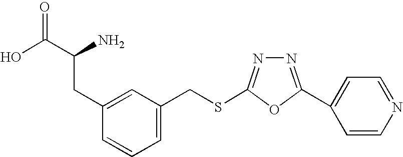 | 2 |
| 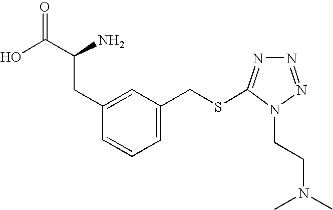 | 1 |
| 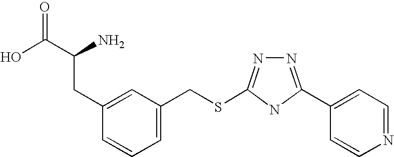 | 2 |

3: Δ > 10%, significantly higher binding affinity than phenylalanine
2: 10% ≧ Δ ≧ −10% similar binding affinity to phenylalanine
1: −10 > Δ > −50%, lower binding affinity than phenylalanine
0: Δ ≦ −50%, no binding or very low binding affinity

### Example 6

### Binding for Exemplary Compounds to Aβ40

The binding ability between the compounds synthesized in Example 4 and Aβ40 in an aqueous solution is tested. The binding ability is attributed semi-quantitatively from the intensities of peptide-compound complex peaks observed in the Electrospray Mass Spectrum.

In the MS assay for Aβ40, samples are prepared as aqueous solutions adding 20% ethanol if necessary to solubilize in water. The stock solution of the peptide contains 50 μm Aβ40. In a typical experiment, 100 μM of an exemplary compound as prepared in Example 4 and 20 μM of solubilized Aβ40 are used. The ratio of the compound: peptide is 5:1. The pH value of each sample is adjusted to 7.4 (±0.2) by addition of 0.1% aqueous sodium hydroxide. The solutions are then analyzed by electrospray ionization mass spectrometry using a Waters ZQ 4000 mass spectrometer. Samples are introduced by direct infusion at a flow-rate of 25 μL/min within 2 hr. after sample preparation. The source temperature is kept at 70° C. and the cone voltage is 20 V for all the analysis. Data are processed using Masslynx 3.5 software. Aβ 1-40 (M.W.=4329) alone at 20 μM is analyzed at pH 7.32 as a control. Sodium clusters, which are typical of this system at +3 and +4 at m/z 1111.0 and 889.1 regions, may be observed. The MS assay gives data on the ability of compounds to bind to soluble Aβ, whereas the ThT, EM and CD assays give data on inhibition of fibrillogenesis.

The results from the assay for binding to Aβ are summarized in Table 7. In Table 7, a blank box means that a value was not determined for that compound in that assay.

TABLE 7
Results of Aβ 1-40 binding study

| Structure | MS Results |
|---|---|
| 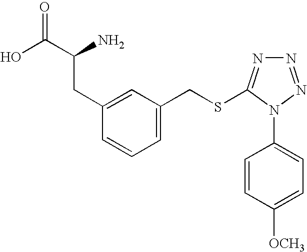 | + |
| 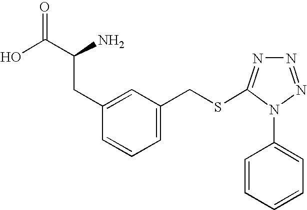 | + |
| 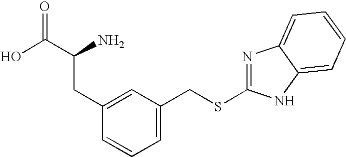 | + |
| 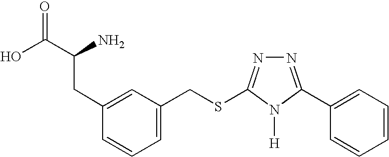 | + |
| 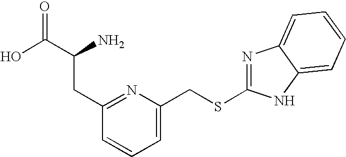 | + |
| 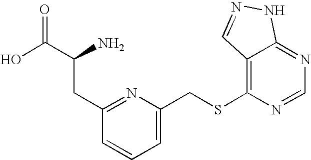 | + |
| 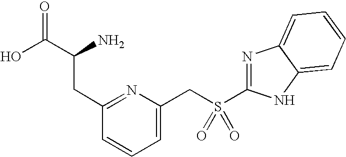 | + |
| 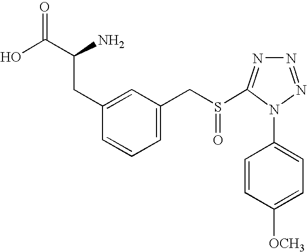 | 0 |
| 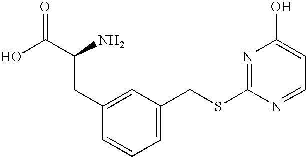 | 0 |
| 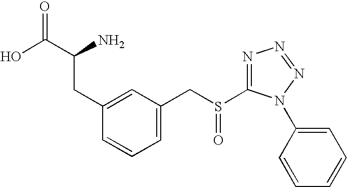 | + |
| 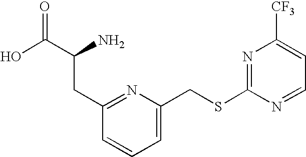 | Compound insoluble |
| 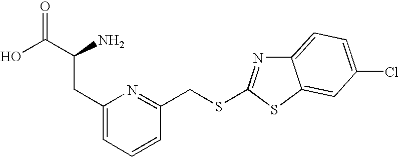 | Compound Insoluble |
| 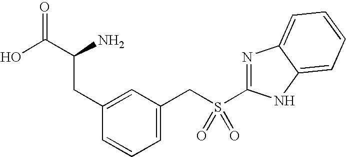 | ++ |
| 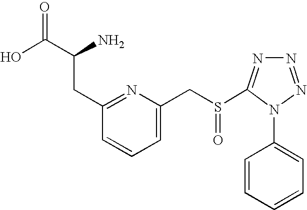 | 0 |
| 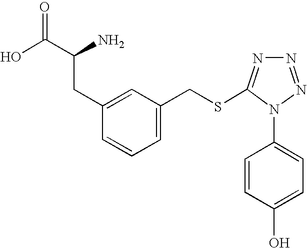 | + |
| 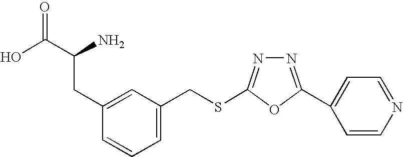 | + |
| 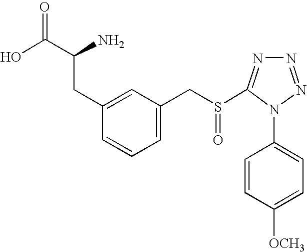 | + |
| 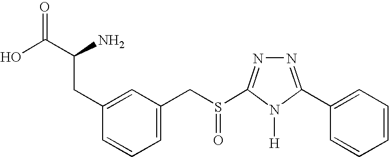 | 0 |
| 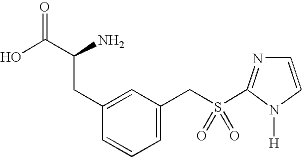 | + |
| 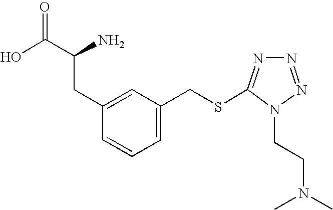 | |
| 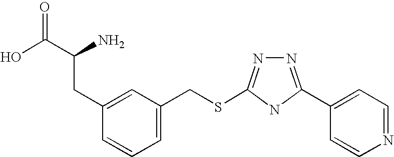 | 0 |
| | + |

+++ = Strong (70 and higher % of free peptide); ++ = Moderate (50-70% of free peptide); + = Weak (25-50% of free peptide); 0 = None

### Example 7

### Binding of Exemplary Compounds to IAPP

The binding ability between the compounds synthesized in Example 4 and IAPP in an aqueous solution is tested. The binding ability is attributed semi-quantitatively from the intensities of peptide-compound complex peaks observed in the Electrospray Mass Spectrum.

In the MS assay for IAPP, samples are prepared as both aqueous solutions and as 20% ethanol in water solutions, including 100 μM of an exemplary compound as prepared in. Example 4 and 20 μM of solubilized IAPP. The stock solution contains 30 μM IAPP and the initial pH is 3.8. Generally, IAPP precipitates out of solution at concentrations higher than 50 μM and pH higher than ˜6 as soon as a test compound is mixed with the peptide. The pH value of each sample, therefore, is adjusted to 7.4 (35 0.2) by addition of 0.1% aqueous sodium hydroxide. The solutions aer then analyzed by electrospray ionization mass spectrometry using a Waters ZQ 4000 mass spectrometer. Samples are introduced by direct infusion at a flow-rate of 25 μL/min within 2 hr. after sample preparation. The source temperature is kept at 70° C. and the cone voltage is 20 V for all the analysis. Data are processed using Masslynx 3.5 software. IAPP (MW 3903.4) alone at 20 μM is analyzed at pH 7.32 as a control. Sodium clusters, which are typical of this system at +3 and +4 at m/z 1301.9 and 976.7 regions, may be observed. The results from the assay for binding to IAPP are summarized in Table 8. In Table 8, a blank box means that a value was not determined for that compound in that assay.

TABLE 8
Results of IAPP binding study

| | MS Results | |
|---|---|---|
| Structure | Water | 20% Ethanol |
| 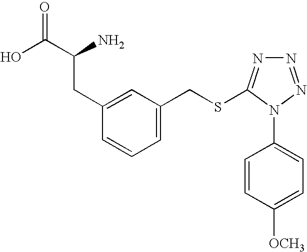 | | +++ |
| 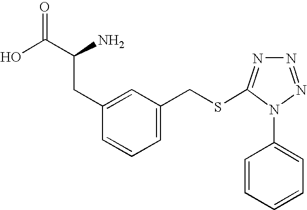 | + | ++ |
| 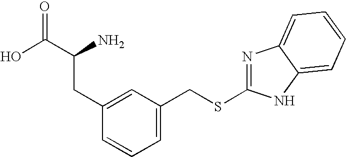 | 0 | + |
| 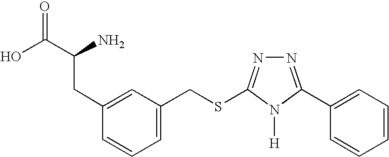 | + | |
| 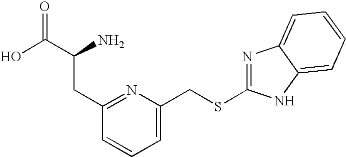 | 0 | |
| 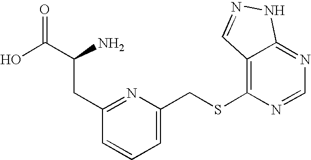 | 0 | |
| 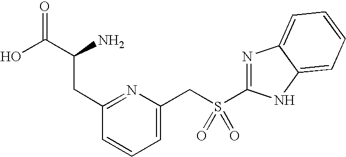 | 0 | |
| 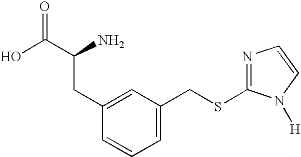 | 0 | |
| 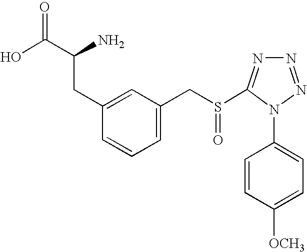 | + | |
| 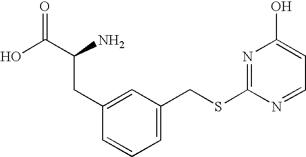 | 0 | |
| 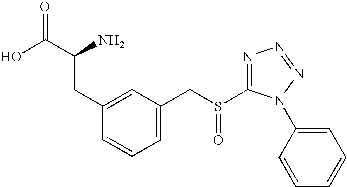 | ++ | ++ |
| 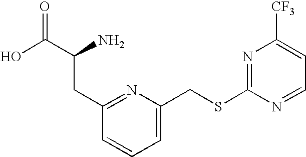 | insoluble | |
| 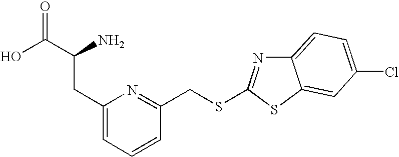 | +++ | |
| 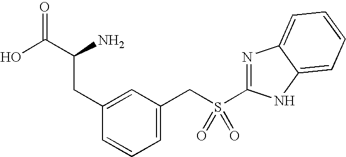 | + | |
| 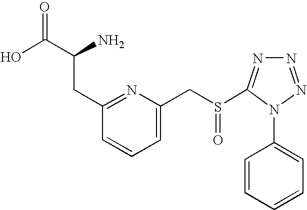 | 0 | ++ |
| 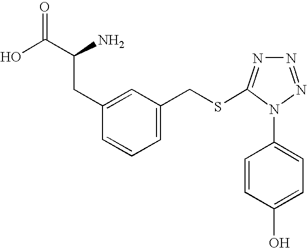 | ++ | +++ |
| 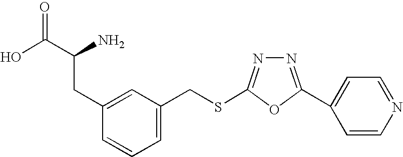 | ++ | + |
| 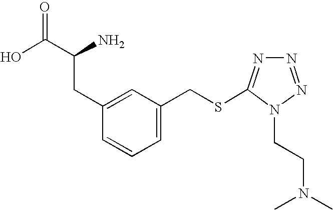 | + | + |
| 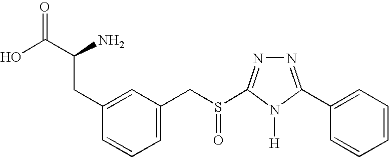 | 0 | |
| 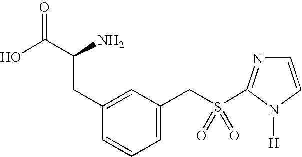 | 0 | |
| 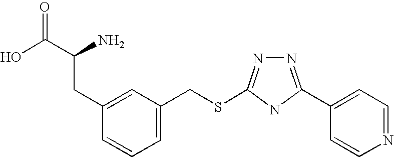 | + | |

+++ = Strong (50 and higher % of free peptide); ++ = Moderate (30-50% of free peptide); + = Weak (15-30% of free peptide); 0 = None

### Example 8

### Apolipoprotein E-Aβ Interaction Assay

The level of interaction between Apolipoprotein E and Aβ was measured for five inventive compounds to determine whether, under the specific conditions of the present example, the compounds would inhibit the interaction. Nunc-Immuno Maxisorp 96-well microtiter plates were coated with 1 μM HFIP-disaggregated Aβ in 0.1 M NaHCO₃ pH 9.6 for 2 hours and 15 minutes at 37°, washed two times in TBS (100 mM Tris-HCl, pH 7.5, 150 mM NaCl), and wells were blocked with 1% fatty-acid free BSA in TBS overnight at 4°.

Test compounds were prepared in either TBS or DMSO at a final concentration of either 2 mM or 10 mM respectively. Recombinant ApoE (Fitzgerald Industries Int.) was prepared in 700 mM NH₄HCO₃ at a final concentration of 0.44 mg/mL to prevent monomer assembly and stored as aliquots at −20°. 3.41 μg/mL of purified ApoE was pre-incubated in the presence of 200 μM test compounds, all in triplicate, in 1% BSA/TBS in a 96-well transfer plate for one hour and then added to the Aβ-coated wells for an additional two hours with gentle shaking at 37° to allow ApoE/Aβ association. Plates were washed three times in TBS to remove excess ApoE and incubated first with 0.125 μg/mL mouse monoclonal anti-ApoE antibody (BD Bioscience) for 1 hour, washed and then incubated with 0.26 μg/mL horse-radish peroxidase conjugated goat anti-IgG antibody (Pierce) for 1 hour in 1% BSA/TBS-T (0.05% Tween-20). After washing, wells were incubated with Sure Blue™ TMB-1 peroxidase substrate (KPL) for 30 minutes. The reaction was stopped using 1N HCl. Absorbance values at 450 nm were measured using TECAN plate reader and reflect the amount of ApoE bound to Aβ in the wells. Data were expressed as a percentage of ApoE/Aβ complexes by arbitrarily setting ApoE/Aβ alone at 100%. All compounds were tested at least twice.

TABLE 9
Results of Apolipoprotein E-Aβ interaction study

| Structure | Aβ-ApoE % Complex |
|---|---|
| 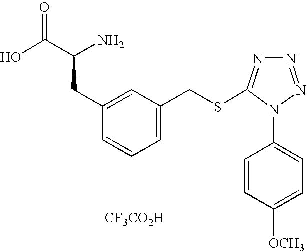 | 98 97 |
| 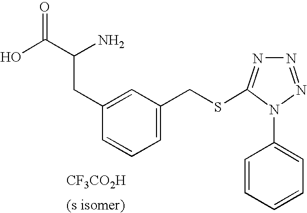 | 107 97 |
| 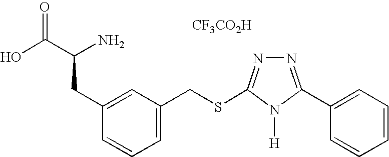 | 88 94 |
| 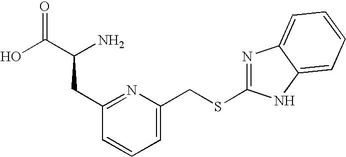 | 94 112 |
| 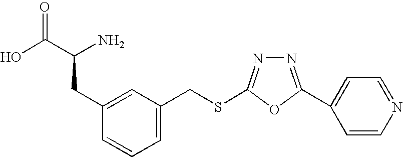 | 101 94 |

The results indicate that the five compounds tested had minimal effect on the interaction between Apolipoprotein E and Aβ under these conditions. It is to be understood, however, that these compounds may exhibit effectiveness under other conditions, for example different concentrations of compound, amyloid and/or Apolipoprotein E.

### Example 9

### Hoechst Staining and Caspase Assays

Materials

The following items were purchased from their respective companies and used without further purification unless otherwise stated:

| Item | Company | Catalogue # |
|---|---|---|
| SH-SY5Y, human neuroblastoma | American Type | CRL-2266 |
| cell line, established from a subline | Culture Collection | |
| of SK-N-SH | (ATCC) | |
| Fetal Bovine Serum (FBS) | Gibco | 10099-141 |
| Eagle's Minimum Essential | Sigma | 4655 |
| Medium (EMEM) | | |
| Ham's F12 Nutrient mixture with | Gibco | 11765-054 |
| L-Glutamine | | |
| MEM non-essential amino acids | Gibco | 1140-050 |
| Trypsin/EDTA (2.5 g Trypsin and | Gibco | 25200-056 |
| 0.38 g EDTA-4Na/L in HBSS | | |
| without Ca⁺⁺ and Mg⁺⁺) | | |
| Paraformaldehyde (PFA) | Electron Microscopy | 15714 |
| | Science (EMB) | |
| Methanol | Fisher | A452-4 |
| Phosphate Buffered Saline (PBS) | Gibco | 14040-133 |
| Hoechst Dye 33342 | Molecular Probes | H-3570 |
| | | (10 mg/mL |
| | | in water) |
| Water | Sigma | W-3500 |
| Prolong Gold Anti-fade Reagent | Molecular Probes | P36930 |
| Caspase-Glo 3/7 Assay | Promega | G8092 |
| FlexStation II 384 | Molecular Devises | |

Maintenance of Human Undifferentiated Neuroblastoma SH-SY5Y

SH-SY5Y cells were cultured and sub-cultured according to ATCC's recommendations. Cells were grown in a culture medium containing 10% fetal bovine serum (FBS), 1× non-essential amino acids in a 1:1 mixture of Eagle's minimum essential medium and Ham's F12 medium.

For passage, cells were trypsinized with 0.25% (w/vol) Trypsin/Ethylene-diaminetetraacetic (EDTA) for 5 min at 37° C., and then centrifuged for 5 min at 300× g (GS-6R Beckman Centrifuge). The pellet was resuspended in the culture medium and the cell density was adjusted.

Preparation of Aβ₁₋₄₂

Synthetic Aβ₁₋₄₂ is purchased from American Peptide Company, Sunnyvale, Calif. To eliminate the aggregated material that may be found in synthetic Aβ ₁₋₄₂ peptide preparations, a disaggregation/filtration procedure is used. Briefly, the Aβ₁₋₄₂ powder is dissolved in HFIP in a glass-flask at a maximal concentration of 200 μM. The solution is sonicated for 30 minutes and then filtered through an ANOTOP 25 (20 nM filter). The exact concentration of the solution is calculated by measuring the optical density at 280 nM. The soluble Aβ ₁₋₄₂ solution is then evaporated to remove the HFIP and resuspended in a buffer containing 0.04 M Tris-HCl , 0.3 M NaCl, pH 7.4, at a final concentration of 120 μM. This solution is stored frozen for later use.

Preparation of NRM compounds

| | | | Stock |
|---|---|---|---|
| Compound # | MW | Diluents | Concentration |
| 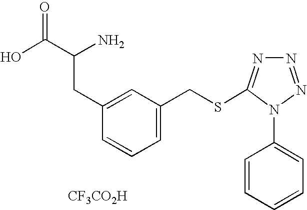 | 355.45 | PBS 1% DMSO | 10 mM |
| 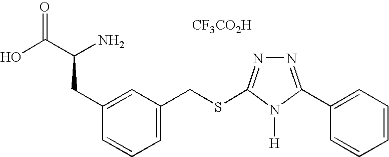 | 354.45 | PBS 1% DMSO | 10 mM |
| 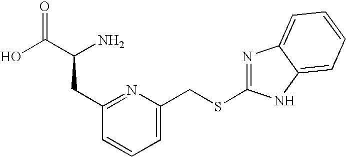 | 328.41 | PBS 1% DMSO | 10 mM |

The compounds listed above were dissolved in phosphate buffered saline (PBS) (without calcium and magnesium), 1% dimethyl sulfoxide (DMSO), pH 7.4, filtered through a 0.22 μm syringe filter, aliquoted and stored at −80° C. until use.

SH-SY5Y Treatment

For Hoechst staining, SH-SY5Y cells were seeded on glass coverslips in a 24-well plate at a density of 3×10⁵ cells/well. Treatments were performed the next day. Cells were incubated for 24 hours with 10 μM Aβ₁₋₄₂, diluted (in the culture medium) from the 120 μM stock in the presence or absence of 200 μM of the desired compound (1:20 Aβ:drug ratio).

For caspase assay, SH-SY5Y cells were plated on 96 well plates coated with collagen I at a density of 1×10⁵ cells by well. Sixteen to seventeen hours before assay, the medium was changed to EMEM/F12 containing 1% FBS. Cells were incubated for 24 hours with 10 μM Aβ₁₋₄₂, diluted (in the culture medium) from the 120 μM stock in the presence or absence of varying concentrations of desired compound (1:20, 1:5 and 1:1 Aβ:drug ratio).

Hoechst Staining

The stock solution of Hoechst 33342 was diluted to 100 μg/ml in water and stored at 2-8° C. SH-SY5Y neuroblastoma were incubated for 10 to 60 minutes with 500 μl of Hoechst solution at a final concentration of 2 μg/ml in the culture medium. Cells were washed 3 times with PBS and fixed in 4% PFA for 30 minutes at room temperature. After 3 washes in PBS, the coverslips were mounted onto glass slides using prolong anti-fade reagent.

Counting Method and Data Analysis

Nuclear morphology was observed using an Olympus fluorescent microscope IX50 equipped with an Olympus Camera (20× objectif and a bandpass filter (Ex/Em: 355 nm/465 nm). Live cells and cells considered morphologically apoptotic were counted. Apoptotic nuclei of undifferentiated SH-SY5Y appear condensed and occasionally fragmented (representative pictures of Hoechst staining are in FIGS. 1A-1B for vehicle and 2A-2B for Aβ).

Five random fields were captured for each condition in a blinded fashion. Apoptotic and normal nuclei in each field were quantified by manual examination. The data are expressed as a percentage of toxicity, corresponding to the number of apoptotic cells divided by total cell number (apoptotic+non apoptotic cells). The total number of cells counted in each condition ranged from 120 to 550.

The Figures were generated with SigmaPlot software. Student t-test (Excel software) was used to compare the % toxicity in Aβ treatment in presence of compound to the Aβ treatment alone, using the average obtained from all experiments. A significance level of p<0.05 was considered for the t-test.

The second compound prepared, 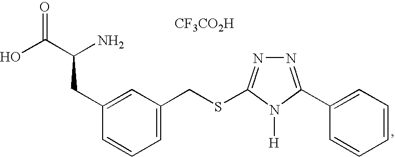
was neuroprotective against Aβ-induced cellular apoptosis at DNA level (showed 22.5% inhibition). The other two compounds had no effect on Aβ-induced cellular apoptosis at DNA level in this particular Hoechst staining assay. It is to be understood, however, that these compounds may exhibit effectiveness under other conditions, for example different concentrations of compound, different cell types, e.g., neuroblastoma cells and/or different assay conditions.
Caspase 3/7 Assay

Following SH-SY5Y treatment, 80 μl of Caspase-Glo™ 3/7 reagent were added in each well and incubated for 30 minutes at room temperature. The luminescence was measured in each well on the FlexStation. The results indicate that each of the three compounds tested had no effect on Aβ-induced caspases 3/7. It is to be understood, however, that these compounds may exhibit effectiveness against Aβ-induced caspases under other conditions, for example different concentrations of compound, different cells, e.g., neuroblastoma cells, different concentrations of Caspase-Glo™, and/or different reagents

### Prospective Example

### Effects of Short and Long Term Treatment in Adult Transgenic CRND8 Mice Overexpressing βAPP

Short Term

APP transgenic mice, TgCRND8, expressing the human amyloid precursor protein (hAPP) develop a pathology resembling Alzheimer's disease. In particular, high levels of Aβ40 and Aβ42 have been documented in the plasma and the brain of these animals at 8-9 weeks of age, followed by early accumulation of amyloid plaques similar to the senile plaques observed in AD patients. These animals also display progressive cognitive deficits that parallel the appearance of degenerative changes. See, e.g., (Chishti, et al., J. Biol. Chem. 276, 21562-70 (2001).

The short term therapeutic effect of compounds of the invention will be studied. These compounds will be administered over a 14 or 28 day period at the end of which the levels of Aβ peptides in the plasma and brain of TgCRND8 animals will be determined.

Methods

Male and female APP transgenic mice will be given daily subcutaneous or oral administrations of a test compound for 14 or 28 days. Baseline animals at 9±1 weeks of age will be used to determine the Aβ levels in the plasma and brain of transgenic animals at the initiation of treatment.

Starting at 9 weeks of age (±1 week) animals will receive daily administration of their respective treatment for a period of 14 or 28 days. Control groups will receive only water or methylcellulose. At the end of the treatment periods, plasma and perfused brains will be collected for quantification of soluble and insoluble Aβ levels.

Sample Collection

At 9±1 weeks of age for the Baseline group, and at the end of the treatment period (14 or 28 days) for the treated groups, at 24 hours after the last compound administration, animals will be sacrificed and samples collected. An approximate blood volume of 500 μl will be collected under general anaesthesia from the orbital sinus and kept on ice until centrifugation at 4° C. at a minimum speed of 3,000 rpm for 10 minutes. Plasma samples will immediately be frozen and stored at −80 ° C. pending analysis. After intracardiac saline perfusion the brains will be removed, frozen, and stored at −80° C. awaiting analysis.

Measurements of Aβ Levels

Brains will be weighed frozen and homogenized with 4 volumes of ice cold 50 mM Tris-Cl pH 8.0 buffer with protease inhibitor cocktail (4 mL of buffer for 1 g of wet brain). Samples will be spun at 15000 g for 20 minutes and the supernatants will be transferred to fresh tubes. One hundred fifty (150) μl from each supernatant will be mixed with 250 μl of 8M guanidine-HCL/50 mM Tris-HCL pH 8.0 (ratio of 0.6 vol supernatant: 1 vol 8M guanidium/Tris-HCL 50 mM pH8.0) and 400 μL 5 M guanidium/Tris-HCL 50 mM pH8.0 will be added. The tubes will be vortexed for 30 seconds and frozen at −80° C. In parallel, pellets will be treated with 7 volumes of 5 M guanidine-HCL/50 mM Tris-HCL pH 8.0 (7 mL of guanidine for 1 g of wet brain), vortexed for 30 seconds and frozen at −80° C. Samples will be thawed at room temperature, sonicated at 80° C. for 15 minutes and frozen again. This cycle will be repeated 3 times to ensure homogeneity and samples will be returned to −80° C. pending analysis.

Aβ levels will be evaluated in plasma and brain samples by ELISA using Human Aβ40 and Aβ42 Fluorometric ELISA kits from Biosource (Cat. No. 89-344 and 89-348) according to manufacturer's recommended procedures. In short, samples will be thawed at room temperature, sonicated for 5 minutes at 80° C. (sonication for brain homogenates; no sonication for plasma samples) and kept on ice. Aβ peptides will be captured using 100 μl of the diluted samples to the plate and incubated without shaking at 4° C. overnight. The samples will be aspirated and the wells will be rinsed 4 times with wash buffer obtained from the Biosource ELISA kit. The anti-Aβ40 or anti-Aβ42 rabbit polyclonal antiserum (specific for the Aβ40 or Aβ42 peptide) will be added (100 βl) and the plate will be incubated at room temperature for 2 hours with shaking. The wells will be aspirated and washed 4 times before adding 100 βl of the alkaline phosphatase labeled anti-rabbit antibody and incubating at room temperature for 2 hours with shaking. The plates will then be rinsed 5 times and the fluorescent substrate (100 βl) will be added to the plate. The plate will be incubated for 35 minutes at room temperature and read using a titer plate reader at an excitation wavelength of 460 nm and emission at 560 nm.

Compounds will be scored based on their ability to modulate levels of Aβ peptides in the plasma and the cerebral soluble/insoluble levels in the brain. Levels of Aβ observed in the plasma and brain of treated animals will be normalized using values from control groups and ranked according to the strength of the pharmacological effect.

Long Term

Transgenic mice, TgCRND8, as those used in the short term treatment, overexpress a human APP gene with the Swedish and Indiana mutations leading to the production of high levels of the amyloid peptides and to the development of an early-onset, aggressive development of brain amyloidosis. The high levels of Aβ peptides and the relative overabundance of Aβ₄₂ compared to Aβ₄₀ are believed to be associated with the severe and early degenerative pathology observed. The pattern of amyloid deposition, presence of dystrophic neuritis, and cognitive deficit has been well documented in this transgenic mouse line. The levels of Aβ peptides in the brain of these mice increase dramatically as the animals' age. While the total amyloid peptide levels increase from ˜1.6×10⁵ pg/g of brain to ˜3.8×10⁶ between 9 and 17 weeks of age.

While the early deposition of amyloid in this model allows the rapid testing of compounds in a relatively short time frame, the aggressivity of this model and the high levels of Aβ peptides renders therapeutic assessment in the longer term a more difficult task.

The long-term therapeutic effects of compounds of the present invention on cerebral amyloid deposition and β-amyloid (Aβ) levels in the plasma and in the brains of transgenic mice, TgCRND8, expressing the human amyloid precursor protein (hAPP) will be studied. These compounds will be administered over a 4, 8 or 16 week period at the end of which the levels of Aβ peptides in the plasma and brain of TgCRND8 animals will be determined. Steady-state pharmacokinetic profile will also be evaluated using plasma samples. The goal of this study will be to evaluate the efficacy of the compounds at modulating the progression of the amyloidogenic process in the brain and in the plasma of a transgenic mouse model of Alzheimer's disease (AD).

Methods

Male and female transgenic mice will be given daily subcutaneous or oral administrations of the appropriate compounds for 4, 8 or 16 weeks. Baseline animals at 9±1 weeks of age will be used to determine the extent of cerebral amyloid deposits and Aβ levels in the plasma and brain of naive transgenic animals at the initiation of treatment.

Starting at 9 weeks of age (±1 week) animals will receive daily administration of their respective treatment for a period of 4, 8 or 16 weeks. Control groups will receive only water or methylcellulose. At the end of the treatment periods, plasma and perfused brains will be collected for quantification of Aβ levels.

Samples will be collected and Aβ levels will be measured as described above in the short term treatment study. Compounds will be scored based on their ability to modulate levels of Aβ peptides in the plasma and the cerebral soluble/insoluble levels in the brain. Levels of Aβ observed in the plasma and brain of treated animals will be compared to that of control groups and ranked according to the strength of the pharmacological effect.

1. A compound of Formula I:
A-Y-Q
wherein:
Q is a blood brain barrier transport vector;
Y is a direct bond or a linker group;
A is selected from the group consisting of hydrogen, alkyl, alkyloxy, alkenyl, alkenyloxy, alkynyl, alkynyloxy, carbocyclic, heterocyclic, bicyclic, aryl, heteroaryl, fused-ring aryl or heteroaryl, aryloxy, arylalkyl, arylalkyloxy, alkylcarbonyl, arylcarbonyl, alkoxycarbonyl, thiazolyl, triazolyl, imidazolyl, benzothiazolyl, benzoimidazolyl, 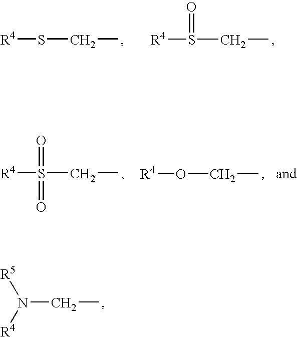
each of which may be optionally substituted; and
R⁴ and R⁵ together with the nitrogen form a 5 or 6 membered heterocyclic ring, or are each independently selected from the group consisting of hydrogen, alkyl, alkyloxy, alkenyl, alkenyloxy, alkynyl, alkynyloxy, cycloalkyl, aryl, aryloxy, arylalkyl, arylalkyloxy, alkylcarbonyl, arylcarbonyl, alkoxycarbonyl, thiazolyl, triazolyl, imidazolyl, benzothiazolyl, and benzoimidazolyl, each of which may be optionally substituted;
or a pharmaceutically acceptable salt, ester or prodrug thereof.

2. The compound of claim 1, wherein Q is a large neutral amino acid moiety or analog thereof.

3. The compound of claim 1, wherein the compound is of Formula (II): 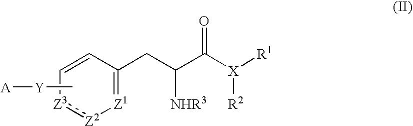
wherein:
X is selected from the group consisting of oxygen, nitrogen, and sulfur;
Y is a direct bond or a linker group;
Z¹, Z², Z³ are each independently selected from the group consisting of C, CH, CH₂, P, N, NH, S, and absent;
R¹ and R² are independently absent or selected from the group consisting of hydrogen, alkyl, cycloalkyl, alkenyl, alkylnyl, aryl, arylalkyl, and acyl;
R³ is selected from the group consisting of hydrogen, alkyl, aryl, amido, arylamido, alkylcarbonyl, arylcarbonyl, arylaminocarbonyl, alkoxycarbonyl, alkanesulfonyl, arenesulfonyl, cycloalkanesulfonyl, and heteroarenesulfonyl, each of which may be optionally substituted;
A is selected from the group consisting of a hydrogen, alkyl, alkyloxy, alkenyl, alkenyloxy, alkynyl, alkynyloxy, carbocyclic, heterocyclic, bicyclic, aryl, heteroaryl, fused-ring aryl or heteroaryl, aryloxy, arylalkyl, arylalkyloxy, alkylcarbonyl, arylcarbonyl, alkoxycarbonyl, thiazolyl, triazolyl, imidazolyl, benzothiazolyl, benzoimidazolyl, 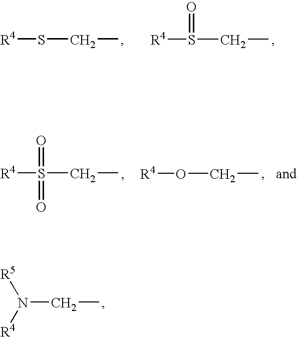
each of which may be optionally substituted; and
R⁴ and R⁵ together with the nitrogen form a 5 or 6 membered heterocyclic ring or are each independently selected from the group consisting of hydrogen, alkyl, alkyloxy, alkenyl, alkenyloxy, alkynyl, alkynyloxy, cycloalkyl, aryl, aryloxy, arylalkyl, arylalkyloxy, alkylcarbonyl, arylcarbonyl, alkoxycarbonyl, thiazolyl, triazolyl, imidazolyl, benzothiazolyl, and benzoimidazolyl, each of which may be optionally substituted;
or a pharmaceutically acceptable salt, ester or prodrug thereof.

4. The compound of claim 3, wherein X is selected from the group consisting of oxygen, and nitrogen.

5. The compound of claim 3, wherein Z¹, Z², and Z³ are each independently N, C or CH.

6. The compound of claim 3, wherein Y is a direct bond.

7. The compound of claim 3, wherein Y is a linker group selected from the group consisting of a disulfide bond, an ether linkage, a thioether linkage, an alkylene or alkenylene linkage, an amino or a hydrozino linkage, an ester-based linkage, a thioester linkage, an amide bond, an acid-labile linkage, and a Schiff base linkage.

8. The compound of claim 3, wherein R¹ and R² are independently absent or hydrogen.

9. The compound of claim 3, wherein R³ is selected from the group consisting of hydrogen, arylamido, arylaminocarbonyl and arenesulfonyl, each of which may be optionally substituted.

10. The compound of claim 3, wherein each A is independently selected from the group consisting of 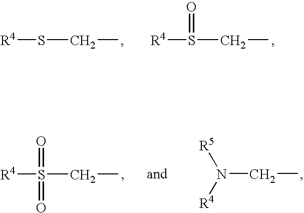
each of which may be optionally substituted.

11. The compound of claim 3, wherein R⁴ and R⁵ are each independently selected from the group consisting of cycloalkyl, aryl, aryloxy, arylalkyl, arylalkyloxy, alkylcarbonyl, arylcarbonyl, alkoxycarbonyl, thiazolyl, triazolyl, imidazolyl, benzothiazolyl, and benzoimidazolyl, each of which may be optionally substituted.

12. The compound of claim 3, wherein R⁴ and R⁵ together with the nitrogen form a 6 membered ring optionally interrupted with one or more additional heteroatoms.

13. The compound of Formula (II), wherein said compound is at least one compound selected from the group consisting of compounds in Table 1, and pharmaceutically acceptable salts, esters, and prodrugs thereof.

14. A compound of Formula (II), wherein the compound is at least one compound selected from the group consisting of compounds in Table 2 and pharmaceutically acceptable salts, esters, and prodrugs thereof.

15. The compound of claim 1, wherein the compound is not a compound of Table 3.

16. The use of a compound according to claim 1, or a pharmaceutically acceptable salt, ester, or prodrug thereof, in the preparation of a medicament for the treatment or prevention of a CNS disease or an amyloid associated disease.

17. The use of a compound according to claim 1, or a pharmaceutically acceptable salt, ester, or prodrug thereof, in the preparation of a medicament for the treatment or prevention of Alzheimer's disease or an amyloid associated disease.

18. A pharmaceutical composition for the treatment or prevention of a CNS disease or an amyloid associated disease comprising a compound according to claim 1, or a pharmaceutically acceptable salt, ester, or prodrug thereof.

19. A pharmaceutical composition comprising a compound according to claim 1, or a pharmaceutically acceptable salt, ester, or prodrug thereof.

20-22. (canceled)

23. A kit for use in treating a CNS disease or an amyloid associated disease comprising a compound of claim 1 or depicted in the Tables, or a pharmaceutically acceptable salt, ester, or prodrug thereof, and instructions for use in the method of the instant invention.

24. A method of treating or preventing a CNS disease or an amyloid associated disease in a subject comprising administering to a subject in need thereof, a compound of claim 1 or depicted in the Tables, or a pharmaceutically acceptable salt, ester, or prodrug thereof, in an amount effective to treat or prevent a CNS disorder or an amyloid associated disease.

25. The method according to claim 24, wherein amyloid fibril formation or deposition, neurodegeneration, or cellular toxicity is reduced or inhibited upon administration of said compound.

26. The method according to claim 24, wherein said subject is a human.

27. A method for treating Alzheimer's Disease in a subject comprising administering to a subject an effective amount of a therapeutic compound of claim 1 or depicted in the Tables, or a pharmaceutically acceptable salt, ester, or prodrug thereof, such that Alzheimer's Disease is treated.

28. A method for treating an Aβ-related disease in a subject having amyloid deposits, the method comprising administering to said subject an effective amount of a therapeutic compound of claim 1 or depicted in the Tables, or a pharmaceutically acceptable salt, ester, or prodrug thereof, such that the Aβ-related disease is treated.

29-33. (canceled)

34. A method for preventing, slowing, or stopping disease progression comprising administering to a subject an effective amount of a compound of claim 1 or depicted in the Tables, or a pharmaceutically acceptable salt, ester, or prodrug thereof, such that said disease progression is prevented slowed, or stopped.

35. (canceled)

36. A bifunctional compound comprising a BBB transport vector and a moiety for the treatment of a CNS disorder or an amyloid associated disease, or a pharmacologically acceptable salt thereof.

37. The compound of claim 36, wherein the BBB transporter vector is a large neutral amino acid or a large neutral amino acid analog.

38. A method for treating a subject for a CNS disorder or an amyloid associated disease, comprising:
coadministration of any of the compounds of claim 1 or depicted in the Tables with an agent that enhances penetration of the blood brain barrier, such that the CNS disorder or amyloid associated disease is treated.

39. The method of claim 38, wherein the agent that enhances penetration of the blood brain barrier is L-arginine.
